SEQUENCE LISTING (6 sequences)

SEQ ID NO: 1 - protein, 67 aa, Artificial Sequence
  Description of Artificial Sequence Synthetic polypeptide
HTIHVGFDAVTGEFTGMPEQWARLLQTSNITKSEQKKNPQAVLDVLEFYNSKKTSNSQKYMSFTDKS

SEQ ID NO: 2 - protein, 8 aa, Artificial Sequence
  Description of Artificial Sequence Synthetic peptide
RKKRRQRR

SEQ ID NO: 3 - protein, 11 aa, Artificial Sequence
  Description of Artificial Sequence Synthetic peptide
YARAAARQARA

SEQ ID NO: 4 - protein, 11 aa, Artificial Sequence
  Description of Artificial Sequence Synthetic peptide
THRLPRRRRRR

SEQ ID NO: 5 - protein, 11 aa, Artificial Sequence
  Description of Artificial Sequence Synthetic peptide
GGRRARRRRRR

SEQ ID NO: 6 - protein, 17 aa, Artificial Sequence
  Description of Artificial Sequence Synthetic peptide
PPVIAPREHTKSVYTRS

(19) United States
(12) Patent Application Publication
(10) Pub. No.: US 2013/0096115 A1
(43) Pub. Date: Apr. 18, 2013

(54) METHODS FOR TREATING AUTISM

(71) Applicants: Jay Lichter, Rancho Santa Fe, CA (US); David Campbell, San Diego, CA (US); Benedikt Vollrath, San Diego, CA (US); Sergio G. Durón, San Diego, CA (US)

(72) Inventors: Jay Lichter, Rancho Santa Fe, CA (US); David Campbell, San Diego, CA (US); Benedikt Vollrath, San Diego, CA (US); Sergio G. Durón, San Diego, CA (US)

(73) Assignee: AFRAXIS, INC., La Jolla, CA (US)

(21) Appl. No.: 13/519,299

(22) PCT Filed: Dec. 21, 2010
(86) PCT No.: PCT/US2010/061640

§ 371 Date: Nov. 21, 2012

Related U.S. Application Data
(60) Provisional application No. 61/290,480, filed on Dec. 28, 2009.

(51) Int. Cl. C07D 471/04; C07D 401/04; C07D 413/14; C07D 401/14

(52) U.S. Cl. CPC C07D 471/04; C07D 401/14; C07D 401/04; C07D 413/14
USPC 514/234.2; 514/252.16; 514/252.18; 514/275; 514/235.8

(57) ABSTRACT

Provided herein are PAK inhibitors. Also provided herein are compositions and methods for treating an individual suffering from autism.

### CROSS REFERENCE

This application claims priority to U.S. Provisional Application No. 61/290,480, entitled, “Methods for Treating Austism,” filed on Dec. 28, 2009, the contents of which are incorporated by reference in its entirety.

### BACKGROUND OF THE INVENTION

Autism spectrum disorders (ASD) are neuropsychological conditions characterized by widespread abnormalities of social interactions and communication, as well as restricted interests and repetitive behaviors.

### SUMMARY OF THE INVENTION

Described herein are p21-activated kinase (PAK) inhibitors that alleviate, ameliorate, delay onset of, inhibit progression of, or reduce the severity of at least one of the symptoms associated with autism. In certain cases, autism is diagnosed is based upon certain behavior characteristics. In some embodiments, the PAK inhibitors described herein alleviate, ameliorate, delay onset of, inhibit progression of, or reduce the severity of at least one symptom associated with autism. In some embodiments, the PAK inhibitors described herein alleviate, ameliorate, delay onset of, inhibit progression of, or reduce the severity of compulsive behavior associated with autism. In some embodiments, the PAK inhibitors described herein alleviate, ameliorate, delay onset of, inhibit progression of, or reduce the severity of the ritualistic behavior associated with autism. In some embodiments, the PAK inhibitors described herein alleviate, ameliorate, delay onset of, inhibit progression of, or reduce the severity of the restricted behavior associated with autism. In some embodiments, the PAK inhibitors described herein alleviate, ameliorate, delay onset of inhibit progression of, or reduce the severity of the stereotypy associated with autism. In some embodiments, the PAK inhibitors described herein alleviate, ameliorate, delay onset of, inhibit progression of, or reduce the severity of the “sameness” associated with autism. In some embodiments, the PAK inhibitors described herein alleviate, ameliorate, delay onset of, inhibit progression of, or reduce the severity of the self-injury behavior associated with autism. In some embodiments, the PAK inhibitors described herein alleviate, ameliorate, delay onset of, inhibit progression of, or reduce the severity of one or more behavioral symptoms associated with autism.

In some embodiments, PAK inhibitors described herein provide therapeutic benefit to an individual suffering from autism that is non-responsive to other putative autism therapies, e.g., treatment with serotonin re-uptake inhibitors (e.g., clomipramine, fluvoxamine and fluoxetine), anti-psychotic medications (e.g., clozapine, respiridone, olanzapine, quietiapine or the like), and stimulants.

In some instances, PAK inhibition modulates dendritic spine morphogenesis. In some instances, PAK inhibitors modulate spine morphogenesis thereby modulating loss of synapses associated with autism. In some instances, aberrant spine morphogenesis (e.g., abnormal spine density, length, thickness, shape or the like) is associated with pathogenesis of autism. In some instances, administration of a PAK inhibitor to individuals diagnosed with or suspected of having autism reduces, stabilizes or reverses abnormalities in dendritic spine morphology, density, and/or synaptic function, including but not limited to abnormal spine density, spine size, spine shape, spine plasticity, spine motility or the like. In some instances, administration of PAK inhibitors to individuals diagnosed with or suspected of having autism reduces, stabilizes or reverses depression of synaptic function caused by tau protein-related neuropathological events (e.g., the formation of dendritic neurofibrillary tangles (NFT)). In some instances, administration of PAK inhibitors to individuals diagnosed with or suspected of having autism reduces, stabilizes or reverses depressions of synaptic function caused by beta-amyloid protein.

In some embodiments, the methods of treatment provided herein comprise administering a PAK inhibitor to an individual with two or more or the following symptoms: (i) insistence on sameness or resistance to change; (ii) difficulty in expressing needs; (iii) repeating words or phrases in place of normal, responsive language; (iv) laughing, crying, showing distress for reasons not apparent to others; (v) prefers to be alone or aloof manner; (vi) tantrums; (vii) difficulty in mixing with others; (viii) may not want to cuddle or be cuddled; (ix) little or no eye contact; (x) unresponsive to normal teaching methods; (xi) sustained odd play; (xii) apparent over-sensitivity or under-sensitivity to pain; (xiii) little or no real fears of danger; (xiv) noticeable physical over-activity or extreme under-activity; (xv) uneven gross/fine motor skills; and/or (xvi) non-responsiveness to verbal cues.

In other embodiments, the methods of treatment provided herein comprise administering a PAK inhibitor to an individual with three or more or the following symptoms: (i) insistence on sameness or resistance to change; (ii) difficulty in expressing needs; (iii) repeating words or phrases in place of normal, responsive language; (iv) laughing, crying, showing distress for reasons not apparent to others; (v) prefers to be alone or aloof manner; (vi) tantrums; (vii) difficulty in mixing with others; (viii) may not want to cuddle or be cuddled; (ix) little or no eye contact; (x) unresponsive to normal teaching methods; (xi) sustained odd play; (xii) apparent over-sensitivity or under-sensitivity to pain; (xiii) little or no real fears of danger; (xiv) noticeable physical over-activity or extreme under-activity; (xv) uneven gross/fine motor skills; and/or (xvi) non-responsiveness to verbal cues.

In some embodiments, the methods of treatment provided herein alleviate, ameliorate, delay onset of, inhibit progression of, or reduce the severity of one or more symptoms associated with Autism Disorder.

In other embodiments, the methods of treatment provided herein alleviate, ameliorate, delay onset of, inhibit progression of, or reduce the severity of one or more symptoms associated with Asperger's Disorder.

In other embodiments, the methods of treatment provided herein alleviate, ameliorate, delay onset of, inhibit progression of, or reduce the severity of one or more symptoms associated with Childhood Disintegrative Disorder.

In other embodiments, the methods of treatment provided herein alleviate, ameliorate, delay onset of, inhibit progression of, or reduce the severity of one or more symptoms associated with Rett's Disorder.

In some embodiments, the methods of treatment provided herein alleviate, ameliorate, delay onset of, inhibit progression of, or reduce the severity of one or more behavioral symptoms. In some embodiments, the behavioral symptom is selected from the group consisting of: (i) insistence on sameness or resistance to change; (ii) difficulty in expressing needs; (iii) repeating words or phrases in place of normal, responsive language; (iv) laughing, crying, showing distress for reasons not apparent to others; (v) prefers to be alone or aloof manner; (vi) tantrums; (vii) difficulty in mixing with others; (viii) may not want to cuddle or be cuddled; (ix) little or no eye contact; (x) unresponsive to normal teaching methods; (xi) sustained odd play; (xii) apparent over-sensitivity or under-sensitivity to pain; (xiii) little or no real fears of danger; (xiv) noticeable physical over-activity or extreme under-activity; (xv) uneven gross/fine motor skills; and/or (xvi) non-responsiveness to verbal cues. In some embodiments, the behavioral symptom is selected from the group consisting of compulsive behavior, ritualistic behavior, restricted behavior, stereotypy, sameness, or self-injury.

In some embodiments, the p21-activated kinase (PAK) inhibitor modulates dendritic spine morphology or synaptic function. In some embodiments, the p21-activated kinase (PAK) inhibitor modulates dendritic spine density. In some embodiments, the p21-activated kinase (PAK) inhibitor modulates dendritic spine length. In some embodiments, the p21-activated kinase (PAK) inhibitor modulates dendritic spine neck diameter. In some embodiments, the p21-activated kinase (PAK) inhibitor modulates dendritic spine shape. In some embodiments, the p21-activated kinase (PAK) inhibitor increases the number of mushroom-shaped dendritic spines. In some embodiments, the p21-activated kinase (PAK) inhibitor modulates dendritic spine head volume. In some embodiments, the p21-activated kinase (PAK) inhibitor modulates the ratio of the number of mature spines to the number of immature spines. In some embodiments, the p21-activated kinase (PAK) inhibitor modulates the ratio of the spine head volume to spine length.

In some embodiments, the p21-activated kinase (PAK) inhibitor modulates synaptic function. In some embodiments, the p21-activated kinase (PAK) inhibitor normalizes or partially normalizes aberrant baseline synaptic transmission associated with autism. In some embodiments, the p21-activated kinase (PAK) inhibitor normalizes or partially normalizes or partially normalizes or partially normalizes aberrant synaptic plasticity associated with autism. In some embodiments, the p21-activated kinase (PAK) inhibitor normalizes or partially normalizes aberrant long term depression (LTD) associated with autism. In some embodiments, the p21-activated kinase (PAK) inhibitor normalizes or partially normalizes aberrant long term potentiation (LTP) associated with autism.

In some embodiments, a therapeutically effective amount of a p21-activated 15 kinase (PAK) inhibitor causes substantially complete inhibition of one or more p21-activated kinases.

In some embodiments, a therapeutically effective amount of a p21-activated kinase (PAK) inhibitor causes partial inhibition of one or more p21-activated kinases.

In some embodiments, the compound of Formula I inhibits one or more of PAK1, PAK2, PAK3, PAK-4, PAK5, or PAK6. In some embodiments, the p21-activated kinase (PAK) inhibitor is a Group I PAK inhibitor. In some embodiments, the p21-activated kinase (PAK) inhibitor inhibits one or more of PAK1, PAK2 or PAK3. In some embodiments, the p21-activated kinase (PAK) inhibitor inhibits PAK1 and PAK3. In some embodiments, the p21-activated kinase (PAK) inhibitor inhibits PAK1 and PAK2. In some embodiments, the p21-activated kinase (PAK) inhibitor inhibits PAK2 and PAK3. In some embodiments, the p21-activated kinase (PAK) inhibitor inhibits PAK1. In some embodiments, the p21-activated kinase (PAK) inhibitor inhibits PAK2. In some embodiments, the p21-activated kinase (PAK) inhibitor inhibits PAK3.

In some embodiments, the methods described herein further comprise administration of a second therapeutic agent. In some embodiments, the second therapeutic agent is an acetylcholinesterase inhibitor, an antioxidant, memantine or minocycline.

In some embodiments, the administration of a therapeutically effective amount of a p21-activated kinase (PAK) inhibitor to an individual in need thereof, wherein administration of the p21-activated kinase (PAK) inhibitor alleviates, inhibits the progression of, or reduces the severity of one or more symptoms associated with autism as measured by the Aberrant Behavior Checklist (ABC), the Ritvo-Freeman Real Life Rating Scale, or the compulsions scale from the Children's Yale-Brown Obsessive Compulsive Scale (CY-BOCS).

In some embodiments, methods are provided for reducing, stabilizing, or reversing neuronal withering and/or loss of synaptic function associated with autism comprising administering to an individual in need thereof a therapeutically effective amount of an agent that modulates dendritic spine morphology or synaptic function. In some embodiments, the neuronal withering and/or loss of synaptic function is induced by beta-amyloid protein, or hydrolysis products thereof, neurofibrillary tangles, or hyperphosphorylated tau protein. In some embodiments, the neuronal withering or loss of synaptic function is associated with dimers or oligomers of beta-amyloid protein. In some embodiments, the neuronal withering or loss of synaptic function is associated with neurofibrillary tangles. In some embodiments, the neuronal withering or loss of synaptic function is associated with hyperphosphorylated tau protein.

In some embodiments, the agent that modulates dendritic spine morphology or synaptic function modulates dendritic spine density. In some embodiments, the agent that modulates dendritic spine morphology or synaptic function modulates dendritic spine length. In some embodiments, the agent that modulates dendritic spine morphology or synaptic function modulates dendritic spine neck diameter. In some embodiments, the agent that modulates dendritic spine morphology or synaptic function modulates dendritic spine shape. In some embodiments, the agent that modulates dendritic spine morphology or synaptic function increases the number of mushroom-shaped dendritic spines. In some embodiments, the agent that modulates dendritic spine morphology or synaptic function modulates dendritic spine head diameter. In some embodiments, the agent that modulates dendritic spine morphology or synaptic function modulates the ratio of the number of mature spines to the number of immature spines. In some embodiments, the agent that modulates dendritic spine morphology or synaptic function modulates the ratio of the spine head volume to spine length.

In some embodiments, the agent that modulates dendritic spine morphology or synaptic function normalizes or partially normalizes aberrant baseline synaptic transmission associated with autism. In some embodiments, the agent that modulates dendritic spine morphology or synaptic function normalizes or partially normalizes aberrant synaptic plasticity associated with autism. In some embodiments, the agent that modulates dendritic spine morphology or synaptic function normalizes or partially normalizes aberrant long term depression (LTD) associated with autism. In some embodiments, the agent that modulates dendritic spine morphology or synaptic function normalizes or partially normalizes aberrant long term potentiation (LTP) associated with autism.

In some embodiments, the methods for reducing, stabilizing, or reversing neuronal withering and/or loss of synaptic function associated with autism comprise administration of a therapeutically effective amount of a p21-activated kinase (PAK) inhibitor to an individual in need thereof alleviates, inhibits the progression of, or reduces the severity of one or more symptoms associated with autism as measured by the Aberrant Behavior Checklist (ABC), the Ritvo-Freeman Real Life Rating Scale, or the compulsions scale from the Children's Yale-Brown Obsessive Compulsive Scale (CY-BOCS).

In some embodiments, the agent that modulates dendritic spine morphology or synaptic function is a p21-activated kinase (PAK) inhibitor.

In other embodiments, methods are provided for reducing, stabilizing or reversing atrophy or degeneration of nervous tissue in the brain associated with autism comprising administering to an individual in need thereof a therapeutically effective amount of an agent that modulates dendritic spine morphology or synaptic function. In some embodiments the atrophy or degeneration of nervous tissue in the brain associated with autism modulates dendritic spine morphology or synaptic function. In some embodiments, the agent that modulates dendritic spine density modulates dendritic spine length. In some embodiments, the agent that modulates dendritic spine morphology or synaptic function modulates dendritic spine length. In some embodiments, the agent that modulates dendritic spine morphology or synaptic function modulates dendritic spine neck diameter. In some embodiments, the agent that modulates dendritic spine morphology or synaptic function modulates dendritic spine shape. In some embodiments, the agent that modulates dendritic spine morphology or synaptic function increases the number of mushroom-shaped dendritic spines. In some embodiments, the agent modulates dendritic spine head diameter. In some embodiments, the agent that modulates dendritic spine morphology or synaptic function modulates the ratio of the number of mature spines to the number of immature spines. In some embodiments, the agent that modulates dendritic spine morphology or synaptic function modulates the ratio of the spine head diameter to spine length.

In some embodiments the agent that modulates dendritic spine morphology or synaptic function normalizes or partially normalizes aberrant baseline synaptic transmission associated with autism. In some embodiments, the agent that modulates dendritic spine morphology or synaptic function normalizes or partially normalizes aberrant synaptic plasticity. In some embodiments, the agent that modulates dendritic spine morphology or synaptic function normalizes or partially normalizes aberrant long term depression (LTD) associated with autism. In some embodiments, the agent that modulates dendritic spine morphology or synaptic function normalizes or partially normalizes aberrant long term potentiation (LTP) associated with autism. In some embodiments, the agent that modulates dendritic spine morphology or synaptic function normalizes or partially normalizes deficits in memory, executive function, or language. In some embodiments, the agent that modulates dendritic spine morphology or synaptic function is a p21-activated kinase (PAK) inhibitor.

In other embodiments, methods are provided for reducing, stabilizing or reversing atrophy or degeneration of nervous tissue in the brain associated with autism comprising administration of the p21-activated kinase (PAK) inhibitor to an individual in need thereof, wherein administration of the p21-activated kinase (PAK) inhibitor to an individual in need thereof alleviates, inhibits the progression of, or reduces the severity of one or more symptoms associated with autism as measured by the Aberrant Behavior Checklist (ABC), the Ritvo-Freeman Real Life Rating Scale, or the compulsions scale from the Children's Yale-Brown Obsessive Compulsive Scale (CY-BOCS).

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 describes illustrative LTP recorded in C57/black 6 mice temporal cortex slices in the presence of 1 μM Compound 7.

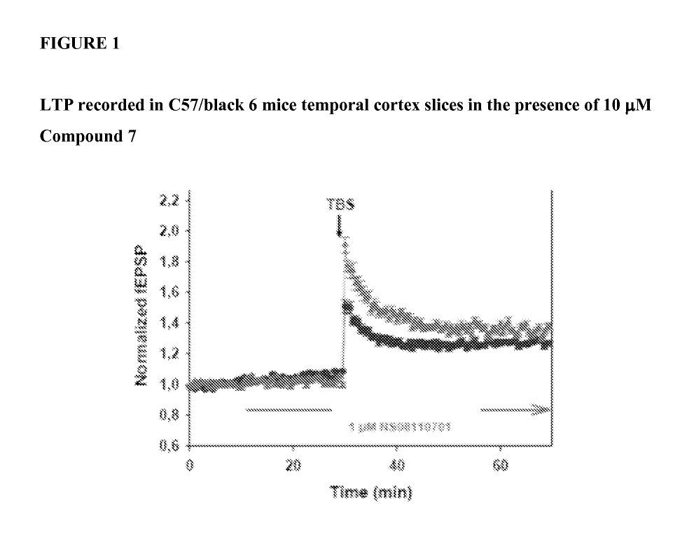

FIG. 2 describes illustrative LTP recorded in C57/black 6 mice temporal cortex slices in the presence of 1 μM Compound 1.

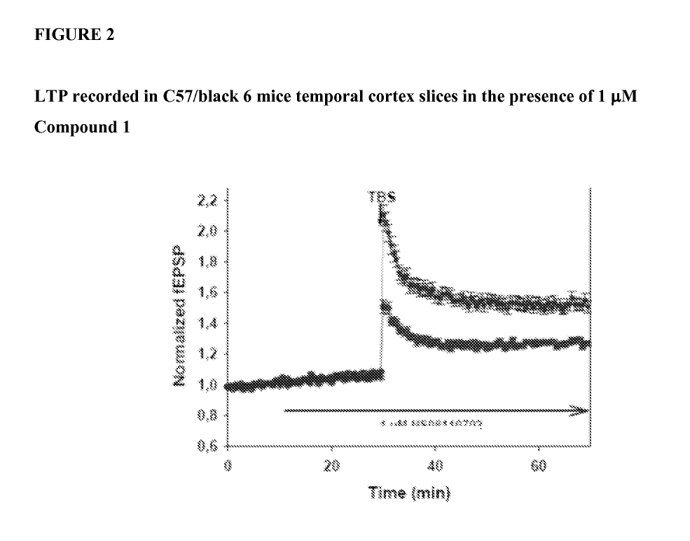

FIG. 3 describes illustrative shapes of dendritic spines.

### DETAILED DESCRIPTION OF THE INVENTION

Provided herein are methods for treatment of autism comprising administration of PAK inhibitors described herein. Autism is a complex neurodevelopmental disability characterized by widespread abnormalities of social interactions and communication, as well as restricted interests and repetitive behaviors. In some instances, the PAK inhibitors described herein (e.g., compounds of Formula I-XXIII) alleviate, ameliorate, delay onset of, inhibit progression of, or reduce the severity of at least one symptom associated with autism. In some embodiments, the PAK inhibitors described herein modulate dendritic spine morphology, dendritic spine density and/or synaptic function thereby reducing, stabilizing or reversing aberrant dendritic spine morphogenesis (e.g., abnormal spine density, length, thickness, shape or the like) associated with pathogenesis of autism.

Described herein are PAK inhibitors and compositions thereof that alleviate, ameliorate, delay onset of, inhibit progression of, or reduce the severity of, or reverse some or all symptoms associated with autism. Also described herein are methods of treating autism comprising the administration of PAK inhibitors and/or compositions thereof to individuals in need thereof that alleviate, stabilize or reverse some or all of the loss of synaptic function associated with autism. Described herein is the use of PAK inhibitors (e.g., compounds of Formula I-XXIII) in the manufacture of a medicament for the treatment of autism. Described herein is the use of PAK inhibitors (e.g., compounds of Formula I-XXIII) in the manufacture of a medicament for modulating (e.g., stabilizing, alleviating or reversing) aberrant spine morphology and/or aberrant synaptic function that is associated with autism.

In some embodiments, the PAK inhibitors described herein (e.g., compounds of Formula I-XXIII) alleviate, stabilize or reverse symptoms of autism in an individual that is non-responsive to other putative autism therapies. In some embodiments, PAK inhibitors described herein (e.g., compounds of Formula I-XXIII) are administered in combination with a second therapeutic agent (e.g., an anti-psychotic agent) and provide an improved therapeutic outcome compared to therapy with the second therapeutic agent alone.

In some instances, autism is associated with abnormal dendritic spine morphology, spine size, spine plasticity, spine motility, spine density and/or abnormal synaptic function. In some instances, PAK kinase activity has been implicated in defective spine morphogenesis, maturation, and maintenance. Described herein are methods for suppressing or reducing PAK activity by administering a PAK inhibitor (e.g., compounds of Formula I-XXIII) for rescue of defects in spine morphology, size, plasticity spine motility and/or density associated with autism as described herein. Accordingly, in some embodiments, the methods described herein are used to treat an individual suffering from autism wherein the condition is associated with abnormal dendritic spine density, spine size, spine plasticity, spine morphology, spine plasticity, and/or spine motility or a combination thereof.

In some embodiments, a p21-activated kinase inhibitor described herein (e.g., compounds of Formula I-XXIII) modulates abnormalities in dendritic spine morphology and/or synaptic function that are associated with autism. In some embodiments, modulation of dendritic spine morphology and/or synaptic function alleviates, halts or delays the progression of the behavioral symptoms (e.g., compulsive behavior, ritualistic behavior, restricted behavior, stereotypy, “sameness”, and/or self-injury) associated with autism.

### Autism

Autism is a complex neurodevelopmental disability that interferes with, among other things, the normal development of the brain in the areas of social interaction and communication skills. It typically appears during the first three years of life and is the result of neurodevelopmental disorders which affect the functioning of the brain.

Autism is generally characterized as one of five disorders within the umbrella term Autism Spectrum Disorders (ASD), a category of neurological disorders characterized by severe and pervasive impairment in several areas of development, including social interaction and communications skills (DSM-IV-TR), which affects about 6 of every 1000 children. The five disorders are: (i) Autistic Disorder (classic autism), (ii) Asperger's Disorder, (iii) Childhood Disintegrative Disorder (CDD), (iv) Rett's Disorder (Rett Syndrome), and (v) PDD—Not Otherwise Specified (PDD-NOS). Specific diagnostic criteria for each of these disorders can be found in the Diagnostic & Statistical Manual of Mental Disorders (DSM-IV-TR) as distributed by the American Psychiatric Association (APA). Of the Autism Spectrum Disorders, Autistic disorder is the most common, affecting an estimated 1 in approximately 200 births, and is approximately four times more prevalent in boys than girls.

In some instances, the following behavioral traits or symptoms, as identified by the Autism Society of America (ASA), may be present in persons with autism: (i) insistence on sameness or resistance to change; (ii) difficulty in expressing needs (i.e. uses gestures or pointing instead of words); (iii) repeating words or phrases in place of normal, responsive language; (iv) laughing, crying, showing distress for reasons not apparent to others; (v) prefers to be alone or aloof manner; (vi) tantrums; (vii) difficulty in mixing with others; (viii) may not want to cuddle or be cuddled; (ix) little or no eye contact; (x) unresponsive to normal teaching methods; (xi) sustained odd play (e.g., spins objects and/or inappropriate attachments to objects); (xii) apparent over-sensitivity or under-sensitivity to pain; (xiii) little or no real fears of danger; (xiv) noticeable physical over-activity or extreme under-activity; (xv) uneven gross/fine motor skills; and/or (xvi) non-responsiveness to verbal cues (i.e., acts as if deaf although hearing tests in normal range).

While there is no single known cause for autism, in some instances, autism may be caused by abnormalities in brain structure or function. In some instances, development of autism is associated with a genetic component. The theory of a genetic basis of the disorder is supported by the fact that familial and twin studies indicate that Autism Spectrum Disorders is one of the most genetic of the neuropsychiatric disorders. Studies have shown the importance of certain genes that are involved in the formation and maintenance of the connections between neurons in the development and progression of autism. Included in these genes are CDH9 and CDH10 (genes encoding cadherins), CNTNAP2 (a gene encoding a type of neurexin), NLGN3 and NLGN4 (genes encoding neuroligins), and the SHANK family of genes (which encode scaffold proteins).

In some instances, cellular changes in brain cells contribute to pathogenesis of autism. In some instances, an abnormality in dendritic spine density in the brain can contribute to the pathogenesis of autism. In some instances, a decrease in density of large spines can contribute to the pathogenesis of autism. In some instances, an abnormality in dendritic spine morphology can contribute to the pathogenesis of autism. In some'instances, a decrease in size of spine heads reduces the probability of a spine bearing a synapse. In some instances, an abnormality in synaptic function contributes to the pathogenesis of autism. In some instances, an abnormality in dendritic spine density and/or dendritic morphology and/or synaptic function is associated with activation of p21-activated kinase (PAK). In some instances, modulation of PAK activity (e.g., inhibition or partial inhibition of PAK) alleviates, reverses or reduces abnormalities in dendritic spine morphology and/or dendritic spine density and/or synaptic function associated with autism.

### Dendritic Spines

A dendritic spine is a small membranous protrusion from a neuron's dendrite that serves as a specialized structure for the formation, maintenance, and/or function of synapses. Dendritic spines vary in size and shape. In some instances, spines have a bulbous head (the spine head) of varying shape, and a thin neck that connects the head of the spine to the shaft of the dendrite. In some instances, spine numbers and shape are regulated by physiological and pathological events. In some instances, a dendritic spine head is a site of synaptic contact. In some instances, a dendritic spine shaft is a site of synaptic contact.

In some instances, mature spines have variably-shaped bulbous tips or heads, ˜0.5-2 μm in diameter, connected to a parent dendrite by thin stalks 0.04-1 μm long. In some instances, average spine density ranges from 0.5 to 10 spines per micrometer length of dendrite, and varies with maturational stage of the spine and/or the neuronal cell. In some instances, small-headed spines have head volume <0.05 μm³) medium-size headed spines have head volumes of 0.05 μm³-0.1 μm³ and large-headed spines have head volumes of >0.1 μm³.

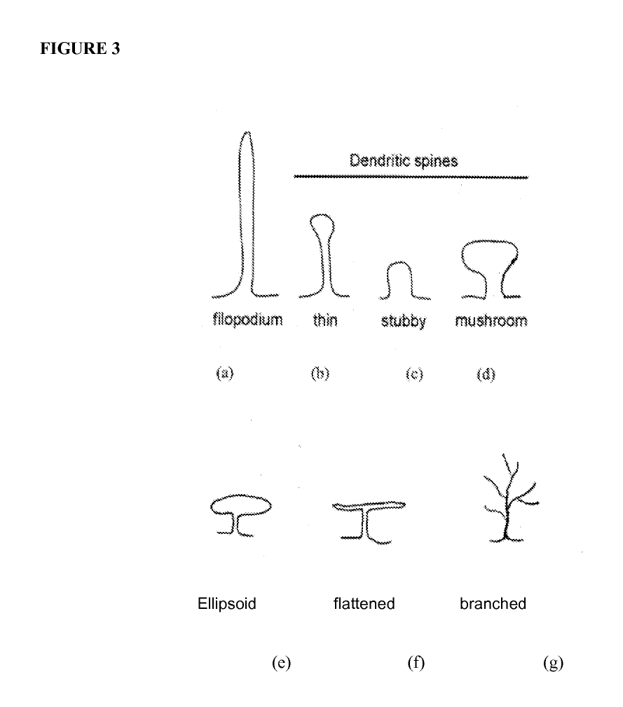

FIG. 3 shows examples of different shapes of dendritic spines. Dendritic spines are “plastic.” In other words, spines are dynamic and continually change in shape, volume, and number. In some instances, spines change in shape, volume, length, thickness or number in a few hours. In some instances, spines change in shape, volume, length, thickness or number occurs within a few minutes. In some instances, spines change in shape, volume, length, thickness or number occurs in response to synaptic transmission and/or induction of synaptic plasticity. By way of example, dendritic spines are headless (filopodia as shown, for example, in FIG. 3a), thin (for example, as shown in FIG. 3b), stubby (for example as shown in FIG. 3c), mushroom-shaped (have door-knob heads with thick necks, for example as shown in FIG. 3d), ellipsoid (have prolate spheroid heads with thin necks, for example as shown in FIG. 3e), flattened (flattened heads with thin neck, for example as shown in FIG. 30 or branched (for example as shown in FIG. 3g). In some instances, the shape of the dendritic spine head determines synaptic function. In some instances, dendritic spines with larger spine head diameter form more stable synapses compared with dendritic spines with smaller head diameter. In some instances, a mushroom-shaped spine head is associated with normal or partially normal synaptic function. In some instances, a mushroom-shaped spine head is a healthier (e.g., having normal or partially normal synapses) as compared to a spine head that is stubby or flat or thin. In some instances, inhibition or partial inhibition of PAK activity results in an increase in spine head diameter and/or spine head volume and/or reduction of spine length, thereby normalizing or partially normalizing synaptic function in individuals suffering or suspected of suffering from autism.

p21-Activated Kinases (PAKs)

The PAKs constitute a family of serine-threonine kinases that are composed of “conventional”, or Group I PAKs, that includes PAK1, PAK2, and PAK3, and “non-conventional”, or Group II PAKs, that includes PAK-4, PAK5, and PAK6. See, e.g., Zhao et al. (2005), Biochem J, 386:201-214. These kinases function downstream of the small GTPases Rac and/or Cdc42 to regulate multiple cellular functions, including dendritic morphogenesis and maintenance (see, e.g., Ethell et al (2005), Prog in Neurobiol, 75:161-205; Penzes et al (2003), Neuron, 37:263-274), motility, morphogenesis, angiogenesis, and apoptosis, (see, e.g., Bokoch et al., 2003, Annu. Rev. Biochem., 72:743; and Hofmann et al., 2004, J. Cell Sci., 117:4343). GTP-bound Rac and/or Cdc42 bind to inactive PAK, releasing steric constraints imposed by a PAK autoinhibitory domain and/or permitting PAK phosphorylation and/or activation. Numerous phosphorylation sites have been identified that serve as markers for activated PAK.

In some instances, upstream effectors of PAK include, but are not limited to, TrkB receptors; NMDA receptors; adenosine receptors; estrogen receptors; integrins, EphB receptors; CDK5, FMRP; Rho-family GTPases, including Cdc42, Rac (including but not limited to Rac1 and Rac2), Chp, TC10, and Wrnch-1; guanine nucleotide exchange factors (“GEFs”), such as but not limited to GEFT, α-p-21-activated kinase interacting exchange factor (aPIX), Kalirin-7, and Tiam1; G protein-coupled receptor kinase-interacting protein 1 (GIT1), and sphingosine.

In some instances, downstream effectors of PAK include, but are not limited to, substrates of PAK kinase, such as Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), Ras, Raf, Mek, p47phox, BAD, caspase 3, estrogen and/or progesterone receptors, RhoGEF, GEF-H1, NET1, Gαz, phosphoglycerate mutase-B, RhoGDI, prolactin, p41Arc, cortactin, and/or Aurora-A (See, e.g., Bokoch et al., 2003, Annu. Rev. Biochem., 72:743; and Hofmann et al., 2004, J. Cell Sci., 117:4343). Other substances that bind to PAK in cells include CIB; sphingolipids; lysophosphatidic acid, G-protein β and/or γ subunits; PIX/COOL; GIT/PKL; Nef; Paxillin; NESH; SH3-containing proteins (e.g. Nck and/or Grb2); kinases (e.g. Akt, PDK1, PI 3-kinase/p85, CdkS, Cdc2, Src kinases, Abl, and/or protein kinase A (PKA)); and/or phosphatases (e.g. phosphatase PP2A, POPX1, and/or POPX2).

### PAK Inhibitors

Described herein are PAK inhibitors that treat one or more symptoms associated with autism. Also described herein are pharmaceutical compositions comprising a PAK inhibitor (e.g., a PAK inhibitor compound described herein) for treatment of one or more symptoms of autism. Also described herein is the use of a PAK inhibitor for manufacture of a medicament for treatment of one or more symptoms of autism. In some embodiments, PAK inhibitors and compositions thereof treat, alleviate, halt or delay the progression one or more of the behavioral symptoms associated with autism (e.g., compulsive behavior, ritualistic behavior, restricted behavior, stereotypy, “sameness”, and/or self-injury).

In some embodiments, the PAK inhibitors described herein alleviate, ameliorate, delay onset of, inhibit progression of, or reduce the severity of, one or more of the following behavioral traits or symptoms: (i) insistence on sameness or resistance to change; (ii) difficulty in expressing needs (i.e. uses gestures or pointing instead of words); (iii) repeating words or phrases in place of normal, responsive language; (iv) laughing, crying, showing distress for reasons not apparent to others; (v) prefers to be alone or aloof manner; (vi) tantrums; (vii) difficulty in mixing with others; (viii) may not want to cuddle or be cuddled; (ix) little or no eye contact; (x) unresponsive to normal teaching methods; (xi) sustained odd play (e.g., spins objects and/or inappropriate attachments to objects); (xii) apparent over-sensitivity or under-sensitivity to pain; (xiii) little or no real fears of danger; (xiv) noticeable physical over-activity or extreme under-activity; (xv) uneven gross/fine motor skills; and/or (xvi) non-responsiveness to verbal cues (i.e., acts as if deaf although hearing tests in normal range).

In some embodiments, the PAK inhibitors described herein alleviate, ameliorate, delay onset of, inhibit progression of, or reduce the severity of compulsive behavior associated with autism. In some embodiments, the PAK inhibitors described herein alleviate, ameliorate, delay onset of, inhibit progression of, or reduce the severity of ritualistic behavior associated with autism. In some embodiments, the PAK inhibitors described herein alleviate, ameliorate, delay onset of, inhibit progression of, or reduce the severity of restricted behavior associated with autism. In some embodiments, the PAK inhibitors described herein alleviate, ameliorate, delay onset of, inhibit progression of, or reduce the severity of stereotypy associated with autism. In some embodiments, the PAK inhibitors described herein alleviate, ameliorate, delay onset of, inhibit progression of, or reduce the severity of “sameness” associated with autism. In some embodiments, the PAK inhibitors described herein alleviate, ameliorate, delay onset of, inhibit progression of, or reduce the severity of self-injury behavior associated with autism.

In some embodiments, the PAK inhibitor is a Group I PAK inhibitor that inhibits, for example, one or more Group I PAK polypeptides, for example, PAK1, PAK2, and/or PAK3. In some embodiments, the PAK inhibitor is a PAK1 inhibitor. In some embodiments, the PAK inhibitor is a PAK2 inhibitor. In some embodiments, the PAK inhibitor is a PAK3 inhibitor. In some embodiments, the PAK inhibitor is a mixed PAK1/PAK3 inhibitor. In some embodiments, the PAK inhibitor inhibits all three Group I PAK isoforms (PAK1, 2 and PAK3) with equal or similar potency. In some embodiments, the PAK inhibitor is a Group II PAK inhibitor that inhibits one or more Group II PAK polypeptides, for example PAK-4, PAK5, and/or PAK6. In some embodiments, the PAK inhibitor is a PAK-4 inhibitor. In some embodiments, the PAK inhibitor is a PAK5 inhibitor. In some embodiments, the PAK inhibitor is a PAK6 inhibitor.

In some embodiments, a PAK inhibitor described herein reduces or inhibits the activity of one or more of PAK1, PAK2 and/or PAK3 while not affecting the activity of PAK-4, PAK5 and/or PaK6. In some embodiments, a PAK inhibitor described herein reduces or inhibits the activity of one or more of PAK1, PAK2, PAK3, and/or PAK-4. In some embodiments, a PAK inhibitor described herein reduces or inhibits the activity of one or more of PAK1, PAK2, PAK3, and/or one or more of PAK-4, PAK5 and/or PAK6. In some embodiments, a PAK inhibitor described herein is a substantially complete inhibitor of one or more PAKs. As used herein, “substantially complete inhibition” means, for example, >95% inhibition of one or more targeted PAKs. In other embodiments, “substantially complete inhibition” means, for example, >90% inhibition of one or more targeted PAKs. In some other embodiments, “substantially complete inhibition” means, for example, >80% inhibition of one or more targeted PAKs. In some embodiments, a PAK inhibitor described herein is a partial inhibitor of one or more PAKs. As used herein, “partial inhibition” means, for example, between about 40% to about 60% inhibition of one or more targeted PAKs. In other embodiments, “partial inhibition” means, for example, between about 50% to about 70% inhibition of one or more targeted PAKs.

In some embodiments, a PAK inhibitor suitable for the methods described herein is a compound having the structure of Formula I or pharmaceutically acceptable salt or N-oxide thereof:

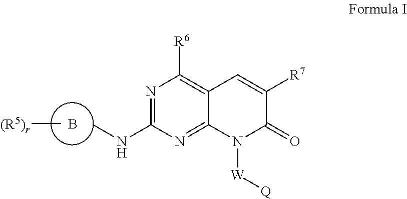

wherein:
W is a bond;
R⁶ is —CN, —OH, substituted or unsubstituted alkoxy, —N(R¹⁰)₂, substituted or unsubstituted heteroalkyl, substituted or unsubstituted cycloalkyl, substituted or unsubstituted aryl or substituted or unsubstituted heteroaryl;
R⁷ is halogen, —CN, —OH, substituted or unsubstituted alkoxy, —C(═O)N(R¹⁰)₂, CO₂R¹⁰, —N(R¹)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl, or substituted or unsubstituted cycloalkyl or heterocycloalkyl fused to ring A;
ring A is substituted or unsubstituted aryl or heteroaryl substituted with 0-4 R⁴;
   each R⁴ is independently halogen, —CN, —NO₂, —OH, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
   each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
ring B is aryl or heteroaryl substituted with R⁵;
   each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8.

In some embodiments, a PAK inhibitor suitable for the methods described herein is a compound having the structure of Formula II or pharmaceutically acceptable salt or N-oxide thereof:

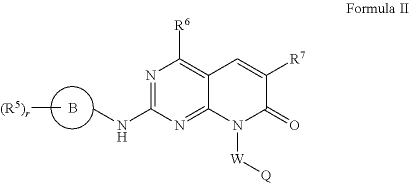

wherein:
W is a bond;
R⁶ is substituted or unsubstituted heteroalkyl, substituted or unsubstituted cycloalkyl, substituted or unsubstituted aryl or substituted or unsubstituted heteroaryl;
R⁷ is H, halogen, —CN, —OH, substituted or unsubstituted alkyl, substituted or unsubstituted alkoxy, —C(═O)N(R¹⁰)₂, —CO₂R¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl, or substituted or unsubstituted cycloalkyl or heterocycloalkyl fused to ring A;
ring A is substituted or unsubstituted aryl or heteroaryl substituted with 0-4 R⁴;
   each R⁴ is independently halogen, —CN, —NO₂, —OH, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O) R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is 1-1 or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
ring B is aryl or heteroaryl substituted with R⁵;
   each R⁵ is independently halogen, —CN, —NO₂, —OH, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8.

In some embodiments, a PAK inhibitor suitable for the methods described herein is a compound having the structure of Formula III or pharmaceutically acceptable salt or N-oxide thereof:

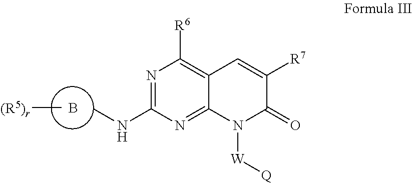

wherein:
W is a bond;
R⁶ is H, or halogen;
R⁷ is acyl, substituted or unsubstituted heteroalkyl, substituted or unsubstituted cycloalkyl, substituted or unsubstituted heterocycloalkyl or substituted or unsubstituted heteroaryl;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl, or substituted or unsubstituted cycloalkyl or heterocycloalkyl fused to ring A;
ring A is substituted or unsubstituted aryl or heteroaryl substituted with 0-4 R⁴;
   each R⁴ is independently halogen, —CN, —NO₂, —OH, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, N(R¹⁰), —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
ring B is aryl or heteroaryl substituted with R⁵;
   each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰), —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8.

In some embodiments, a PAK inhibitor suitable for the methods described herein is a compound having the structure of Formula IV or pharmaceutically acceptable salt or N-oxide thereof:

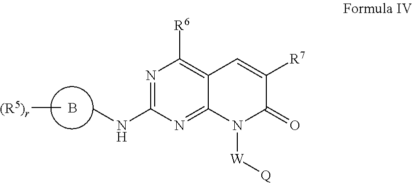

wherein:
W is a bond;
R⁶ is substituted or unsubstituted alkyl;
R⁷ is substituted or unsubstituted heteroalkyl, substituted or unsubstituted cycloalkyl, or substituted or unsubstituted heterocycloalkyl;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl, or substituted or unsubstituted cycloalkyl or heterocycloalkyl fused to ring A;
ring A is substituted or unsubstituted aryl or heteroaryl substituted with 0-4 R⁴;
   each R⁴ is independently halogen, —CN, —NO₂, —OH, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
ring B is aryl or heteroaryl substituted with R⁵;
   each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)N(R¹⁰), —NR¹⁰C(═O)OR¹⁰, —NR¹⁰)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8.

In some embodiments, a PAK inhibitor suitable for the methods described herein is a compound having the structure of Formula V or pharmaceutically acceptable salt or N-oxide thereof:

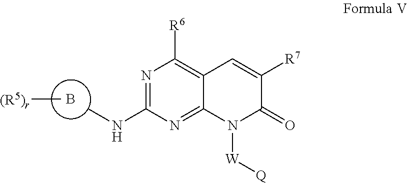

wherein:
W is a bond;
R⁶ is H, or halogen;
R⁷ is H, halogen, CN, OH, substituted or unsubstituted alkyl, substituted or unsubstituted alkoxy, —C(═O)N(R¹⁰)₂, CO₂R¹⁰, N(R¹⁰)₂, acyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Q is substituted or unsubstituted cycloalkyl or heterocycloalkyl fused to ring A;
ring A is substituted or unsubstituted aryl or heteroaryl substituted with 0-4 R⁴;
   each R⁴ is independently halogen, —CN, —NO₂, —OH, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —N(R¹⁰)₂, —C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
ring B is aryl or heteroaryl substituted with R⁵;
   each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁹, —N(R¹⁰)₂, —C(═O)N(R¹⁰)₂, —NR¹⁰C(═O)R¹⁹, —NR¹⁰C(═O)OR¹⁹, —NR¹⁰C(═O)N(R¹⁹)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8.

In some embodiments, the compound of Formula V has the structure of Formula VI:

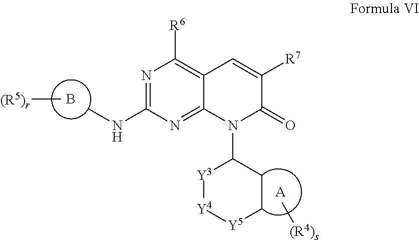

wherein:

each of Y³, Y⁴ and Y⁵ are independently N—R^{1a}, CR¹R², SO₂, or C═O;

R^{1a} is H or substituted or unsubstituted alkyl;

R¹ and R² are each independently H or substituted or unsubstituted alkyl.

In some embodiments, the compound of Formula V has the structure of

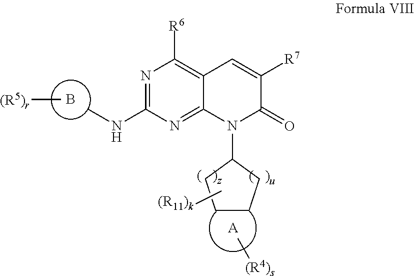

wherein:

ring A is an aryl or heteroaryl substituted with R⁴;
each R⁴ is independently halogen, —CN, —NO₂, —OH, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R⁹, —NR¹⁰C(═O)OR⁹, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
   R⁸ is H or substituted or unsubstituted alkyl;
   R⁹ is substituted or unsubstituted alkyl, substituted or unsubstituted cycloalkyl, substituted or unsubstituted aryl or substituted or unsubstituted heteroaryl;
   each R¹⁰ is independently H, substituted or unsubstituted alkyl, substituted or unsubstituted cycloalkyl, substituted or unsubstituted aryl or substituted or unsubstituted heteroary, or two R¹⁰ together with the nitrogen to which they are attached form a heterocycle;
each R¹ is independently H, halogen, substituted or unsubstituted alkyl, substituted or unsubstituted alkoxy, or two R¹ together with the carbon atom to which they are attached form C═O;

s is 0-4;

k is 1-4;

z is 0 or 1;

u is 1, 2 or 3;

provided that z+u≠1;

ring B is an aryl or heteroaryl substituted with R⁵;
each R⁵ is independently halogen, —CN, —NO₂, —OH, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R⁹, —NR¹⁰C(═O)OR⁹, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;

r is 0-8;

R⁶ is H, or halogen;

R⁷ is H, halogen, CN, OH, substituted or unsubstituted alkyl, substituted or unsubstituted alkoxy, —C(═O)N(R¹⁰)₂, CO₂R¹⁰, N(R¹⁰)₂, acyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, ring A is a heteroaryl ring. In some embodiments, ring A is a phenyl ring.

In some embodiments, the compound of Formula VIII has a structure of Formula VIIIA, Formula VIIIB, Formula VIIIC, Formula VIIID, Formula VIIIE, Formula VIIIF, Formula VIIICG or Formula VIIIH:

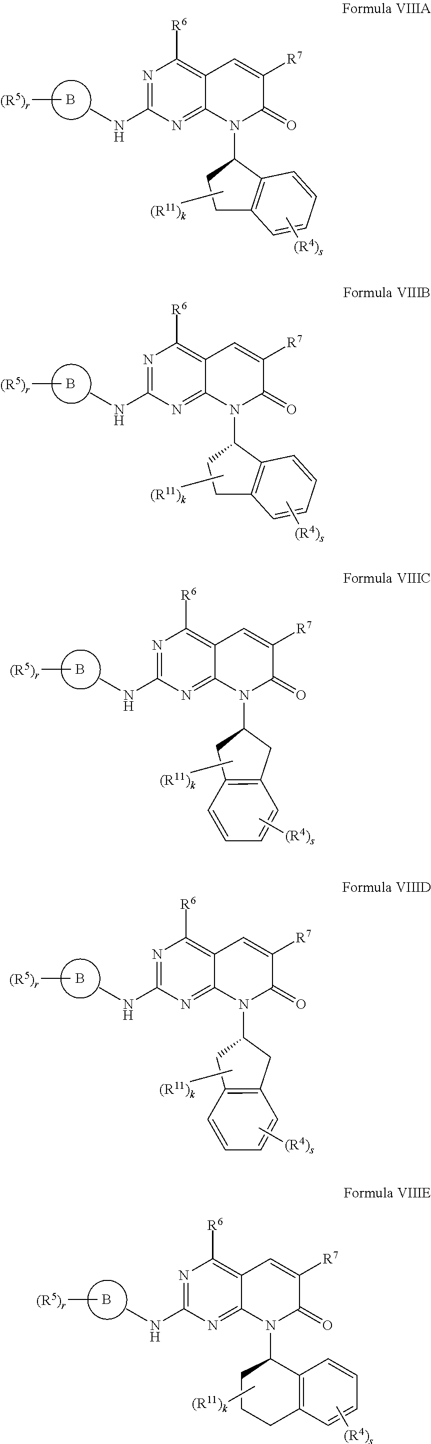

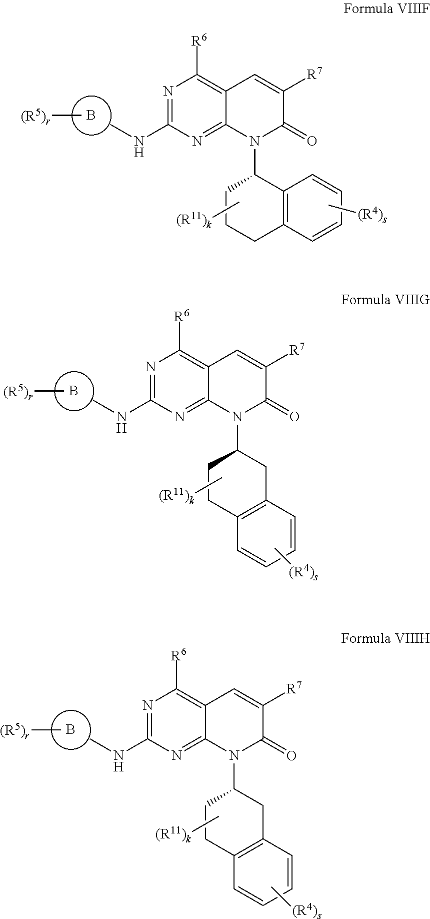

In some embodiments, R¹¹ is H, halogen or substituted or unsubstituted alkyl. In some embodiments, R¹¹ is H.

In some embodiments, a PAK inhibitor suitable for the methods described herein is a compound having the structure of Formula IX or pharmaceutically acceptable salt or N-oxide thereof:

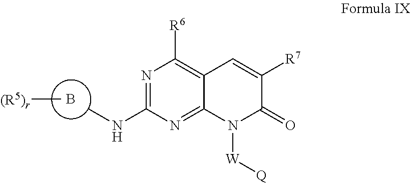

wherein:
W is a bond;
R⁶ is H;

R⁷ is

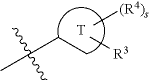

ring T is aryl, heteroaryl, cycloalkyl or heterocycloalkyl substituted with R³ and R⁴;
R³ is a substituted or unsubstituted aryl, substituted or unsubstituted heteroaryl, substituted or unsubstituted cycloalkyl or substituted or unsubstituted heterocycloalkyl attached to ring T via a carbon atom;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl, or substituted or unsubstituted cycloalkyl or heterocycloalkyl fused to ring A;
ring A is substituted or unsubstituted aryl or heteroaryl substituted with 0-4 R⁴;
   each R⁴ is independently halogen, —CN, —NO₂, —OH, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
   R⁸ is H or substituted or unsubstituted alkyl;
   R⁹ is substituted or unsubstituted alkyl, substituted or unsubstituted cycloalkyl, substituted or unsubstituted aryl or substituted or unsubstituted heteroaryl
   each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
s is 0-4;
ring B is aryl or heteroaryl substituted with R⁵;
   each R⁵ is independently halogen, —CN, —NO₂, —OH, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;

r is 0-8.

In some embodiments, a PAK inhibitor suitable for the methods described herein is a compound having the structure of Formula X or pharmaceutically acceptable salt or N-oxide thereof:

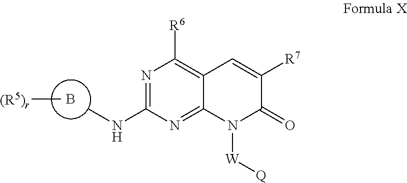

W is a bond;
R⁶ is H, halogen, —CN, —OH, substituted or unsubstituted alkoxy, —N(R¹⁰)₂, substituted or unsubstituted heteroalkyl, substituted or unsubstituted cycloalkyl, substituted or unsubstituted aryl or substituted or unsubstituted heteroaryl;
R⁷ is H, halogen, —CN, —OH, substituted or unsubstituted alkyl, substituted or unsubstituted alkoxy, —C(═O)N(R¹⁰)₂, —CO₂R¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
Q is

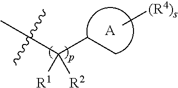

R¹ is H or substituted or unsubstituted alkyl;
R² is substituted or unsubstituted alkyl, or R¹ and R² together with the carbon to which they are attached form a C₃-C₆ cycloalkyl ring;
p is 1, 2 or 3;
ring A is aryl substituted with R⁴;
R³ is halogen, —CN, —NO₂, —OH, —OCF₃, —OCF₂H, —CF₃, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R⁹R¹⁰, NR¹⁰C(═O)OR⁹OR⁹, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
   each R⁴ is independently halogen, —CN, —NO₂, —OH, —OCF₃, —OCF₂H, —CF₃, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
s is 0-4;
ring B is aryl or heteroaryl substituted with R⁵;
   each R⁵ is independently halogen, —CN, —NO₂, —OH, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8.

In some embodiments, a compound of Formula X is a compound wherein
W is a bond;

R⁶ is H, halogen, —CN, —OH, substituted or unsubstituted alkoxy, —N(R¹⁰)₂, substituted or unsubstituted heteroalkyl, substituted or unsubstituted cycloalkyl, substituted or unsubstituted aryl or substituted or unsubstituted heteroaryl;
R⁷ is H, halogen, —CN, —OH, substituted or unsubstituted alkyl, substituted or unsubstituted alkoxy, —C(═O)N(R¹⁰)₂, —CO₂R¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
Q is.

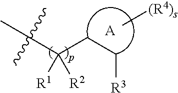

R¹ is H or substituted or unsubstituted alkyl;

R² is substituted or unsubstituted alkyl, or R¹ and R² together with the carbon to which they are attached form a C₃-C₆ cycloalkyl ring;
p is 1, 2 or 3;
ring A is aryl substituted with R³ and R⁴;
R³ is halogen, —CN, —NO₂, —OH, —OCF₃, —OCF₂H, —CF₃, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)₂R⁹R¹⁰, —NR¹⁰C(═O)OR⁹OR⁹, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
   each R⁴ is independently halogen, —CN, —NO₂, —OH, —OCF₂H, —CF₃, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
s is 0-4;
ring B is aryl or heteroaryl substituted with R⁵;
   each R⁵ is independently halogen, —CN, —NO₂, —OH, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8.

In some embodiments, a compound of Formula X has the structure of Formula XA or Formula XB:

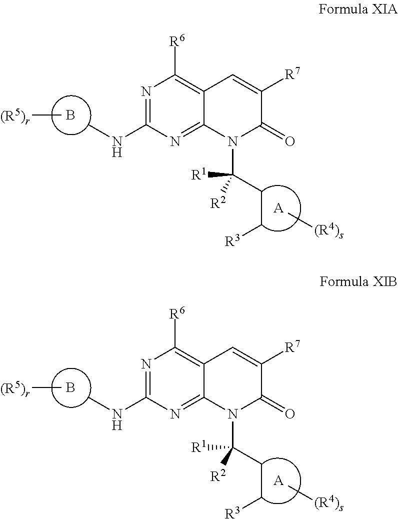

In some embodiments, the compound of Formula X has the structure of

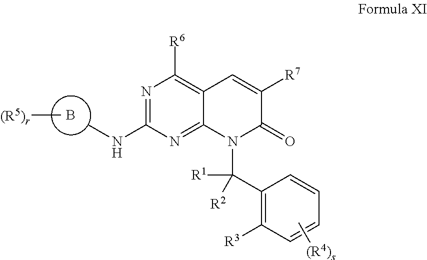

wherein:

R¹⁰ is H or substituted or unsubstituted alkyl;

R² is substituted or unsubstituted alkyl; and

R³ is halogen, alkyl, fluoroalkyl, alkoxy, fluoroalkoxy, or SR⁸.

In some embodiments, the compound of Formula (XI) has the structure of Formula (XIIA) or Formula (XIIB):

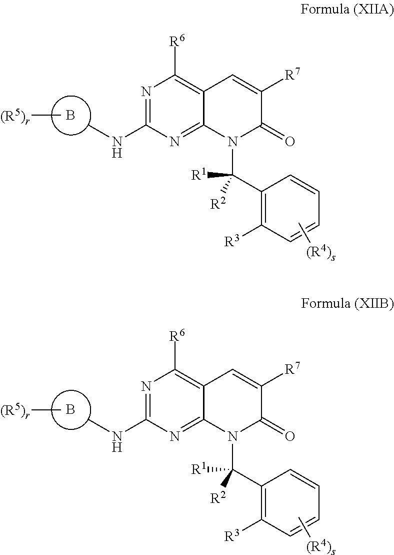

In some embodiments, a PAK inhibitor suitable for the methods described herein is a compound having the structure of Formula XIII or pharmaceutically acceptable salt or N-oxide thereof:

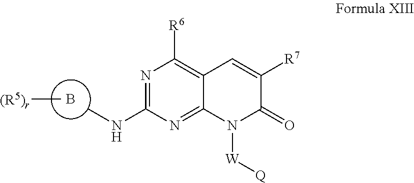

wherein:
W is a bond;
R⁶ is H, halogen, —CN, —OH, substituted or unsubstituted alkyl, substituted or unsubstituted alkoxy, —N(R¹⁰)₂, substituted or unsubstituted heteroalkyl, substituted or unsubstituted cycloalkyl, substituted or unsubstituted aryl or substituted or unsubstituted heteroaryl;
R⁷ is H, halogen, —CN, —OH, substituted or unsubstituted alkyl, substituted or unsubstituted alkoxy, —C(═O)N(R¹⁰)₂, —CO₂R¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
Q is

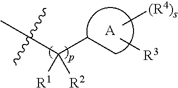

R¹ and R² are each independently H or substituted or unsubstituted alkyl; or R¹ and R² together with the carbon to which they are attached form a C₃-C₆ cycloalkyl ring;
p is 1, 2 or 3;
ring A is aryl substituted with R³ and R⁴;
R³ is a substituted or unsubstituted heteroaryl, substituted or unsubstituted cycloalkyl or substituted or unsubstituted heterocycloalkyl attached to ring A via a carbon atom;
   each R⁴ is independently halogen, —CN, —NO₂, —OH, —OCF₃, —OCF₂H, —CF₃, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
s is 0-4;
ring B is aryl or heteroaryl substituted with R⁵;
   each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8.

In some embodiments, a PAK inhibitor suitable for the methods described herein is a compound having the structure of Formula XIV or pharmaceutically acceptable salt or N-oxide thereof:

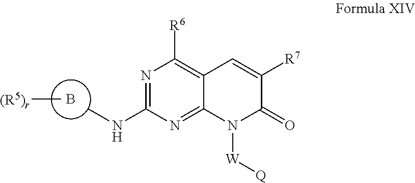

wherein:
W is O;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, or substituted or unsubstituted heteroarylalkyl;
ring B is aryl or heteroaryl substituted with R⁵;
   each R⁵ is independently halogen, —CN, —NO₂, —OH, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8;
R⁶ is halogen, —CN, —OH, substituted or unsubstituted alkyl, substituted or unsubstituted alkoxy, substituted or unsubstituted heteroalkyl, —N(R¹⁰)₂, substituted or unsubstituted cycloalkyl, substituted or unsubstituted aryl or substituted or unsubstituted heteroaryl;
R⁷ is H, halogen, —CN, —OH, acyl, substituted or unsubstituted alkyl, substituted or unsubstituted alkoxy, —C(═O)N(R¹⁰)₂, —CO₂R¹⁰, —N(R¹⁰)₂, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In some embodiments, the compound of Formula XIV has the structure of Formula XV:

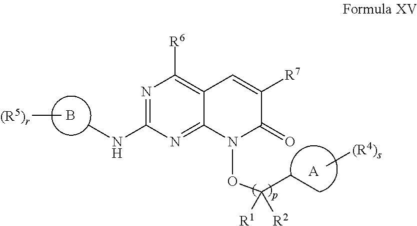

wherein:
p is 0, 1, 2 or 3;
R¹ and R² are each independently H or substituted or unsubstituted alkyl; or R¹ and R² together with the carbon to which they are attached form a C₃-C₆ cycloalkyl ring.

In some embodiments, ring A is an aryl ring. In some embodiments, ring A is a phenyl or naphthyl ring. In some embodiments, ring A is a heteroaryl ring. In some embodiments, ring A is a heterocycloalkyl ring. In some embodiments, ring A is a cycloalkyl ring.

In some embodiments, a PAK inhibitor suitable for the methods described herein is a compound having the structure of Formula XVI or pharmaceutically acceptable salt or N-oxide thereof:

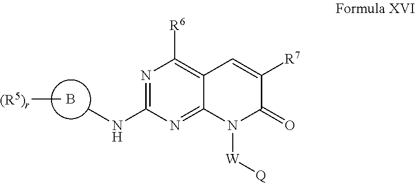

wherein:
W is N—R^{1a};
R^{1a} is H or substituted or unsubstituted alkyl;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, or substituted or unsubstituted heteroarylalkyl;
ring B is aryl or heteroaryl substituted with R⁵;
   each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8;
R⁶ is H, halogen, —CN, —OH, substituted or unsubstituted alkyl, substituted or unsubstituted alkoxy, substituted or unsubstituted heteroalkyl, —N(R¹⁰)₂, substituted or unsubstituted cycloalkyl, substituted or unsubstituted aryl or substituted or unsubstituted heteroaryl;
R⁷ is H, halogen, —CN, —OH, acyl, substituted or unsubstituted alkyl, substituted or unsubstituted alkoxy, —C(═O)N(R¹⁰)₂, —CO₂R¹⁰, —N(R¹⁰)₂, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In some embodiments, the compound of Formula XVI has the structure of Formula XVII:

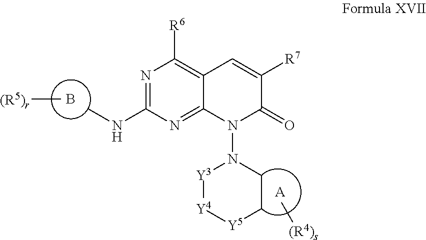

wherein:

each of Y³, Y⁴ and Y⁵ are independently N—R^{1a}, CR¹R², SO₂, or C═O;

R^{1a} is H or substituted or unsubstituted alkyl;

R¹ and R² are each independently H or substituted or unsubstituted alkyl.

In some embodiments, a compound of Formula XVI has the structure of formula XVIII:

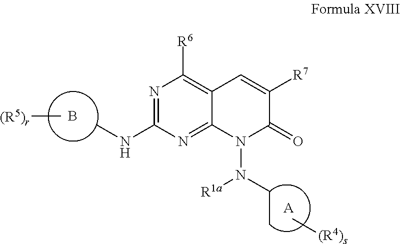

In some embodiments, a compound of Formula XVI has the structure of formula XIX:

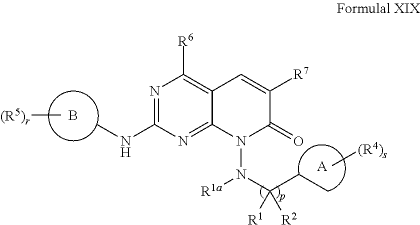

wherein:
p is 1, 2 or 3;
R¹ and R² are each independently H or substituted or unsubstituted alkyl; or R¹ and R² together with the carbon to which they are attached form a C₃-C₆ cycloalkyl ring.

In some embodiments, ring A is a heteroaryl ring. In some embodiments, ring A is an aryl ring. In some embodiments, ring A is a heterocycloalkyl ring. In some embodiments, ring A is a cycloalkyl ring.

In some embodiments, the compound of Formula XVI has the structure of Formula XX:

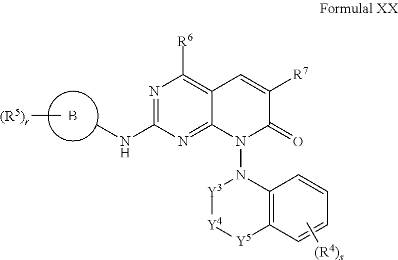

wherein:

each of Y³, Y⁴ and Y⁵ are independently N—R^{1a}, CR¹R², SO₂, or C═O;

R^{1a} is H or substituted or unsubstituted alkyl;

R¹ and R² are each independently H or substituted or unsubstituted alkyl.

In some embodiments, the compound of Formula XVI has the structure of Formula XXIA, Formula XXIB, Formula XXIC or Formula XXID:

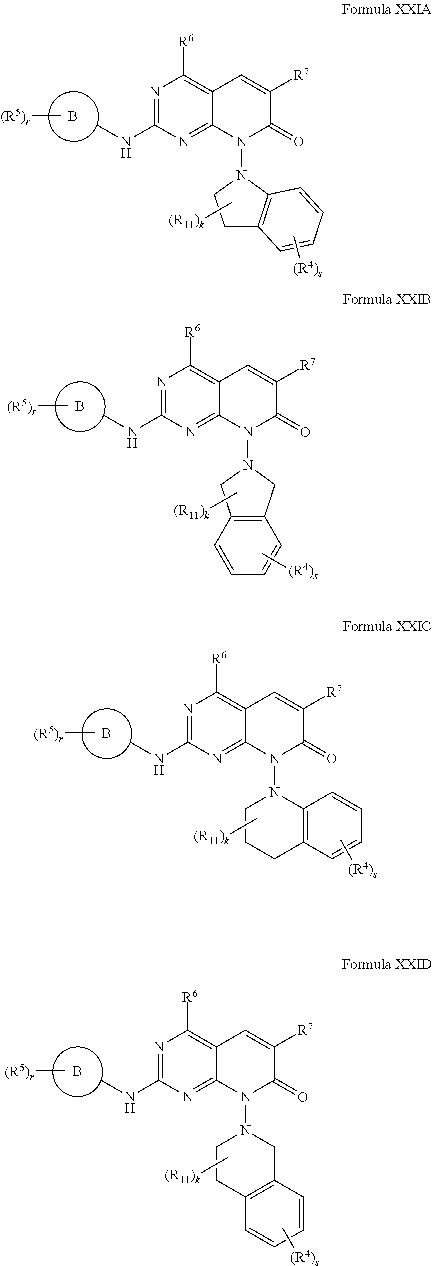

wherein:
each R¹¹ is independently H, halogen, substituted or unsubstituted alkyl, substituted or unsubstituted alkoxy, or two R¹¹ together with the carbon atom to which they are attached form C═O; and k is 1-4.

In some embodiments, a PAK inhibitor is a compound having the structure of Formula XXII, or pharmaceutically acceptable salt or N-oxide thereof:

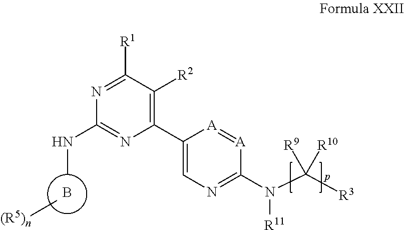

wherein:
R¹ and R² are each independently H, halogen, CN, substituted or unsubstituted alkyl, substituted or unsubstituted alkoxy;
R³ is H, —OH, —OR⁶, —SR⁶, —S(═O)₂R⁷, —CO₂R⁸, N(R⁸)₂, substituted or unsubstituted alkyl, substituted or unsubstituted cycloalkyl, substituted or unsubstituted heterocycloalkyl;
   R⁶ is H or substituted or unsubstituted alkyl;
   R⁷ is substituted or unsubstituted alkyl, substituted or unsubstituted cycloalkyl, substituted or unsubstituted aryl or substituted or unsubstituted heteroaryl;
   each R⁸ is independently H, substituted or unsubstituted alkyl, substituted or unsubstituted cycloalkyl, substituted or unsubstituted aryl or substituted or unsubstituted heteroaryl, or two R⁸ together with the nitrogen to which they are attached form a substituted or unsubstituted heterocycle;
each A is independently N or C—R⁴;
   each R⁴ is independently H, halogen, CN, substituted or unsubstituted alkyl, substituted or unsubstituted alkoxy;
ring B is aryl or heteroaryl substituted with R⁵;
   each R⁵ is independently halogen, —CN, —NO₂, —OH, —SR⁶, —S(═O)R⁷, S(═O)₂R⁷, —NHS(═O)₂R⁷, —C(═O)R⁷, —OC(═O)R⁷, —CO₂R⁸, N(R⁸)₂, C(═O)N(R⁸)₂, —NHC(═O)R⁷, NHC(═O)OR⁷, —NHC(═O)N(R⁸)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
n is 1-8;
R⁹ and R¹⁰ are each independently H, halogen, or substituted or unsubstituted alkyl;
p is 1-5; and
R¹¹ is H or substituted or unsubstituted alkyl.

In some embodiments, a PAK inhibitor is a compound of Formula XXIII:

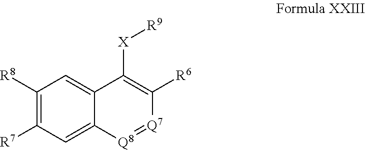

wherein:
R⁶ is H, halo, hydroxy, cyano, substituted or unsubstituted alkyl, or substituted or unsubstituted alkoxy,
R⁷ is substituted or unsubstituted alkyl, substituted or unsubstituted alkoxy, substituted or unsubstituted alkylamino, C(═O)—N(R¹⁰)₂, C(═O)—O(R¹⁰), S(O)ₘ—N(R¹⁰)₂C(═O)R¹⁰, OC(═O)(R¹⁰), N(R¹⁰)₂S(O)ₘR¹⁰, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each R¹⁰ is independently H, substituted or unsubstituted alkyl; substituted or unsubstituted cycloalkyl, or substituted or unsubstituted alkylcycloalkyl; and m is 1-2;
R⁸ is H, halo, hydroxy, cyano, substituted or unsubstituted alkyl, substituted or unsubstituted alkoxy, substituted or unsubstituted alkylamino, C(═O)—N(R¹⁰)₂ᵢ C(═O)—O(R¹⁰), S(O)ₘ—N(R¹⁰)₂, N(R¹⁰)₂C(═O)R¹⁰, OC(═O)(R¹⁰), N(R¹⁰)₂S(O)ₘR¹⁰;
R⁹ is substituted or unsubstituted aryl, substituted or unsubstituted heteroaryl, substituted or unsubstituted cycloalkyl, or substituted or unsubstituted heterocycloalkyl;
Q⁷, Q⁸ are independently N or C—R⁶;
X is O, N—R¹¹ or C(R¹¹)₂, wherein each R¹¹ is independently H, hydroxy, substituted or unsubstituted alkyl; or two R¹¹ taken together are (═O) or (═NR¹²); wherein R¹² is H, hydroxy, substituted or unsubstituted alkyl, or substituted or unsubstituted alkoxy;
provided that when Q⁸ is N, Q⁷ is CH, R⁷, R⁸ are alkoxy, and R⁶ is cyano, R⁹ is not 2,4-dichloroanilino; or a pharmaceutically acceptable salt thereof.

In some embodiments, PAK inhibitors described herein include, by way of example, N′-(5-(2-(3,4,5-trimethoxyphenylamino)pyrimidin-4-yl)pyridin-2-yl)ethane-1,2-diamine (Compound 1), N′-(5-(2-(4-(4-methylpiperazin-1-yl)phenylamino)pyrimidin-4-yl)pyridin-2-yl)ethane-1,2-diamine (Compound 2), N-(4-(4-methylpiperazin-1-yl)phenyl)-4-(6-(2-(piperidin-1-yl)ethylamino)pyridin-3-yl)pyrimidin-2-amine (Compound 3), 2-(4-(4-methylpiperazin-1-yl)phenylamino)-8-(2-(trifluoromethylthio)benzyl)pyrido[2,3-d]pyrimidin-7(8H)-one (Compound 4), 2-(4-(4-methylpiperazin-1-yl)phenylamino)-8-(1,2,3,4-tetrahydronaphthalen-1-yl)pyrido[2,3-d]pyrimidin-7(8H)-one (Compound 5), 6-(2,6-dichlorophenyl)-8-methoxy-2-(4-(4-methylpiperazin-1-yl)phenylamino)pyrido[2,3-d]pyrimidin-7(8H)-one (Compound 6), 8-cyclopentyl-2-(4-(4-methylpiperazin-1-yl)phenylamino)pyrido[2,3-d]pyrimidin-7(8H)-one (Compound 7), 4-(2-chloro-4-methylphenylamino)-6-methoxy-7-(3-(4-methylpiperazin-1-yl)propoxy)quinoline-3-carbonitrile (Compound 8), (S)—N-(2-(dimethylamino)-1-phenylethyl)-6,6-dimethyl-3-(thieno[2,3-d]pyrimidin-4-ylamino)-4,6-dihydropyrrolo[3,4-c]pyrazole-5(2H)-carboxamide (Compound 9) 4-(2,4-dichlorophenylamino)-6-methoxy-7-(3-(4-methylpiperazin-1-yl)propoxy)quinoline-3-carbonitrile (Compound 10) or the like.

In some embodiments, PAK inhibitors include (S)-1-(4-benzyl-6-((5-cyclopropyl-1H-pyrazol-3-yl)methyl)pyrimidin-2-yl)azetidine-2-carboxamide (Compound 11), (S)-2-(3,5-difluorophenyl)-4-(piperidin-3-ylamino)thieno[3,2-c]pyridine-7-carboxamide (Compound 12), or the like.

In certain instances, PAK inhibitors also include, e.g., compounds described in U.S. Pat. Nos. 5,863,532, 6,191,169, 6,248,549, and 6,498,163; U.S. Patent Applications 200200045564, 20020086390, 20020106690, 20020142325, 20030124107, 20030166623, 20040091992, 20040102623, 20040208880, 200500203114, 20050037965, 20050080002, and 20050233965, 20060088897; EP Patent Publication 1492871; PCT patent publication WO 9902701; PCT patent publication WO 2008/047307; Kumar et al., (2006), Nat. Rev. Cancer, 6:459; and Eswaran et al., (2007), Structure, 15:201-213, all of which are incorporated herein by reference for disclosure of kinase inhibitors and PAK inhibitors therein.

In certain instances, small molecule PAK inhibitors include BMS-387032; SNS-032; CHI4-258; TKI-258; EKB-569; JNJ-7706621; PKC-412; staurosporine; SU-14813; sunitinib; N-(3-chloro-4-fluoro-phenyl)-7-methoxy-6-(3-morpholin-4-ylpropoxy)quinazolin-4-amine (gefitinib), VXX-680, MK-0457, combinations thereof; or salts, prodrugs thereof.

In some embodiments, the PAK inhibitor is a polypeptide comprising an amino acid sequence about 80% to about 100% identical, e.g., about 85%, about 90%, about 92%, about 93%, about 95%, about 96%, v97%, about 98%, about 99%, or any other percent from about 80% to about 100% identical the following amino acid sequence: HTIHVGFDAVTGEFTGMPEQWARLLQTSNITKSEQKKNPQAVLDVLEFYNSKKTSNSQ KYMSFTDKS

The above sequence corresponds to the PAK autoinhibitory domain (PAD) polypeptide amino acids 83-149 of PAK1 polypeptide as described in, e.g., Zhao et al (1998). In some embodiments, the PAK inhibitor is a fusion protein comprising the above-described PAD amino acid sequence. In some embodiments, in order to facilitate cell penetration the fusion polypeptide (e.g., N-terminal or C-terminal) further comprises a polybasic protein transduction domain (PTD) amino acid sequence, e.g.: RKKRRQRR; YARAAARQARA; THRLPRRRRRR; or GGRRARRRRRR.

In some embodiments, in order to enhance uptake into the brain, the fusion polypeptide further comprises a human insulin receptor antibody as described in U.S. patent application Ser. No. 11/245,546.

In some embodiments, the PAK inhibitor is peptide inhibitor comprising a sequence at least about 60% to about 100%, e.g., about 65%, about 70%, about 75%, about 80%, about 85%, about 90%, about 92%, about 93%, about 95%, about 96%, about 97%, about 98%, about 99%, or any other percent from about 60% to about 100% identical the following amino acid sequence: PPVIAPREHTKSVYTRS as described in, e.g., Zhao et al (2006), Nat Neurosci, 9(2):234-242. In some embodiments, the peptide sequence further comprises a PTD amino acid sequence as described above.

In some embodiments, the PAK inhibitor is a polypeptide comprising an amino acid sequence at least about 80% to about 100%, e.g., about 85%, about 90%, about 92%, about 93%, about 95%, about 96%, about 97%, about 98%, about 99%, or any other percent from about 80% to about 100% identical to the FMRP1 protein (GenBank Accession No. Q06787), where the polypeptide is able to bind with a PAK (for example, PAK1, PAK2, PAK3, PAK-4, PAK5 and/or PAK6). In some embodiments, the PAK inhibitor is a polypeptide comprising an amino acid sequence at least about 80% to about 100%, e.g., about 85%, about 90%, about 92%, about 93%, about 95%, about 96%, about 97%, about 98%, about 99%, or any other percent from about 80% to about 100% identical to the FMRP1 protein (GenBank Accession No. Q06787), where the polypeptide is able to bind with a Group I PAK, such as, for example PAK1 (see, e.g., Hayashi et al (2007), Proc Natl Acad Sci USA, 104(27):11489-11494. In some embodiments, the PAK inhibitor is a polypeptide comprising a fragment of human FMRP1 protein with an amino acid sequence at least about 80% to about 100%, e.g., about 85%, about 90%, about 92%, about 93%, about 95%, about 96%, about 97%, about 98%, about 99%, or any other percent from about 80% to about 100% identical to the sequence of amino acids 207-425 of the human FMRP1 protein (i.e., comprising the KH1 and KH2 domains), where the polypeptide is able to bind to PAK1.

In some embodiments, the PAK inhibitor comprises a polypeptide comprising an amino acid sequence at least about 80% to about 100%, e.g., about 85%, about 90%, about 92%, about 93%, about 95%, about 96%, about 97%, about 98%, about 99%, or any other percent from about 80% to about 100% identical to at least five, at least ten at least twenty, at least thirty, at least forty, at least fifty, at least sixty, at least seventy, at least eighty, at least ninety contiguous amino acids of the huntingtin (htt) protein (GenBank Accession No. NP 002102, gi 90903231), where the polypeptide is able to bind to a Group I PAK (for example, PAK1, PAK2, and/or PAK3). In some embodiments, the PAK inhibitor comprises a polypeptide comprising an amino acid sequence at least about 80% to about 100%, e.g., about 85%, about 90%, about 92%, about 93%, about 95%, about 96%, about 97%, about 98%, about 99%, or any other percent from about 80% to about 100% identical to at least a portion of the huntingtin (htt) protein (GenBank Accession No. NP 002102, gi 90903231), where the polypeptide is able to bind to PAK1. In some embodiments, the PAK inhibitor is a polypeptide comprising a fragment of human huntingtin protein with an amino acid sequence at least about 80% to about 100%, e.g., about 85%, about 90%, about 92%, about 93%, about 95%, about 96%, about 97%, about 98%, about 99%, or any other percent from about 80% to about 100% identical to a sequence of at least five, at least ten, at least twenty, at least thirty, at least forty, at least fifty, at least sixty, at least seventy, at least eighty, at least ninety, or at least 100 contiguous amino acids of the human huntingtin protein that is outside of the sequence encoded by exon 1 of the htt gene (i.e., a fragment that does not contain poly glutamate domains), where the polypeptide binds a PAK. In some embodiments, the PAK inhibitor is a polypeptide comprising a fragment of human huntingtin protein with an amino acid sequence at least 80% identical to a sequence of the human huntingtin protein that is outside of the sequence encoded by exon 1 of the htt gene (i.e., a fragment that does not contain poly glutamate domains), where the polypeptide binds PAK1.

### Upstream Regulators of p21 Activated Kinases

In certain embodiments, an indirect PAK modulator (e.g., an indirect PAK inhibitor) affects the activity of a molecule that acts in a signaling pathway upstream of PAK (upstream regulators of PAK). Upstream effectors of PAK include, but are not limited to: TrkB receptors; NMDA receptors; EphB receptors; adenosine receptors; estrogen receptors; integrins; FMRP; Rho-family GTPases, including Cdc42, Rac (including but not limited to Rac1 and Rac2), CDK5, PI3 kinases, NCK, PDK1, EKT, GRB2, Chp, TC10, Tcl, and Wrch-1; guanine nucleotide exchange factors (“GEFs”), such as but not limited to GEFT, members of the Dbl family of GEFs, p21-activated kinase interacting exchange factor (PIX), DEF6, Zizimin 1, Vav1, Vav2, Dbs, members of the DOCK180 family, Kalirin-7, and Tiam1; G protein-coupled receptor kinase-interacting protein 1 (GIT1), CIB1, filamin A, Etk/Bmx, and sphingosine.

Modulators of NMDA receptor include, but are not limited to, 1-aminoadamantane, dextromethorphan, dextrorphan, ibogaine, ketamine, nitrous oxide, phencyclidine, riluzole, tiletamine, memantine, neramexane, dizocilpine, aptiganel, remacimide, 7-chlorokynurenate, DCKA (5,7-dichlorokynurenic acid), kynurenic acid, 1-aminocyclopropanecarboxylic acid (ACPC), AP7 (2-amino-7-phosphonoheptanoic acid), APV (R-2-amino-5-phosphonopentanoate), CPPene (3-[(R)-2-carboxypiperazin-4-yl]-prop-2-enyl-1-phosphonic acid); (+)-(1S,2S)-1-(4-hydroxy-phenyl)-2-(4-hydroxy-4-phenylpiperidino)-1-pro-panol; (1S,2S)-1-(4-hydroxy-3-methoxyphenyl)-2-(4-hydroxy-4-phenylpiperi-dino)-1-propanol; (3R,4S)-3-(4-(4-fluorophenyl)-4-hydroxypiperidin-1-yl-)-chroman-4,7-diol; (1R*, 2R*)-1-(4-hydroxy-3-methylphenyl)-2-(4-(4-fluoro-phenyl)-4-hydroxypiperidin-1-yl)-propan-1-ol-mesylate; and/or combinations thereof.

Modulators of estrogen receptors include, and are not limited to, PPT (4,4′,4″-(4-Propyl-[1H]-pyrazole-1,3,5-triyl)trisphenol); SKF-82958 (6-chloro-7,8-dihydroxy-3-allyl-1-phenyl-2,3,4,5-tetrahydro-1H-3-benzazepine); estrogen; estradiol; estradiol derivatives, including but not limited to 17-β estradiol, estrone, estriol, ERβ-131, phytoestrogen, MK 101 (bioNovo); VG-1010 (bioNovo); DPN (diarylpropiolitrile); ERB-041; WAY-202196; WAY-214156; genistein; estrogen; estradiol; estradiol derivatives, including but not limited to 17-β estradiol, estrone, estriol, benzopyrans and triazolo-tetrahydrofluorenones, disclosed in U.S. Pat. No. 7,279,499, and Parker et al., Bioorg. & Med. Chem. Ltrs. 16: 4652-4656 (2006), each of which is incorporated herein by reference for such disclosure.

Modulators of TrkB include by way of example, neutorophic factors including BDNF and GDNF. Modulators of EphB include XL647 (Exelixis), EphB modulator compounds described in WO/2006081418 and US Appl. Pub. No. 20080300245, incorporated herein by reference for such disclosure, or the like.

Modulators of integrins include by way of example, ATN-161, PF-04605412, MEDI-522, Volociximab, natalizumab, Volociximab, Ro 27-2771, Ro 27-2441, etaracizumab, CNTO-95, JSM6427, cilengitide, R411 (Roche), EMD 121974, integrin antagonist, compounds described in J. Med. Chem., 2002, 45 (16), pp 3451-3457, incorporated herein by reference for such disclosure, or the like.

Adenosine receptor modulators include, by way of example, theophylline, 8-Cyclopentyl-1,3-dimethylxanthine (CPX), 8-Cyclopentyl-1,3-dipropylxanthine (DPCPX), 8-Phenyl-1,3-dipropylxanthine, PSB 36, istradefylline, SCH-58261, SCH-442,416, ZM-241,385, CVT-6883, MRS-1706, MRS-1754, PSB-603, PSB-0788, PSB-1115, MRS-1191, MRS-1220, MRS-1334, MRS-1523, MRS-3777, MRE3008F20, PSB-10, PSB-11, VUF-5574, N6-Cyclopentyladenosine, CCPA, 2′-MeCCPA, GR 79236, SDZ WAG 99, ATL-146e, CGS-21680, Regadenoson, 5′-N-ethylcarboxamidoadenosine, BAY 60-6583, LUF-5835, LUF-5845, 2-(1-Hexynyl)-N-methyladenosine, CF-101 (IB-MECA), 2-Cl-IB-MECA, CP-532,903, MRS-3558, Rosuvastatin, KW-3902, SLV320, mefloquine, regadenoson, or the like.

In some embodiments, compounds reducing PAK levels decrease PAK transcription or translation or reduce RNA or protein levels. In some embodiments, a compound that decreases PAK levels is an upstream effector of PAK. In some embodiments, a compound that decreases PAK levels is an upstream effector of PAK. In some embodiments, exogenous expression of the activated forms of the Rho family GTPases Chp and cdc42 in cells leads to increased activation of PAK while at the same time increasing turnover of the PAK protein, significantly lowering its level in the cell (Hubsman et al. (2007) Biochem. J. 404: 487-497). PAK clearance agents include agents that increase expression of one or more Rho family GTPases and/or one or more guanine nucleotide exchange factors (GEFs) that regulate the activity of Rho family GTPases, in which overexpression of a Rho family GTPase and/or a GEF results in lower levels of PAK protein in cells. PAK clearance agents also include agonists of Rho family GTPases, as well as agonists of GTP exchange factors that activate Rho family GTPases, such as but not limited to agonists of GEFs of the Dbl family that activate Rho family GTPases.

Overexpression of a Rho family GTPase is optionally by means of introducing a nucleic acid expression construct into the cells or by administering a compound that induces transcription of the endogenous gene encoding the GTPase. In some embodiments, the Rho family GTPase is Rac (e.g., Rac1, Rac2, or Rac3), cdc42, Chp, TC10, Tcl, or Wrnch-1. For example, a Rho family GTPase includes Rac1, Rac2, Rac3, or cdc42. A gene introduced into cells that encodes a Rho family GTPase optionally encodes a mutant form of the gene, for example, a more active form (for example, a constitutively active form, Hubsman et al. (2007) Biochem. J. 404: 487-497). In some embodiments, a PAK clearance agent is, for example, a nucleic acid encoding a Rho family GTPase, in which the Rho family GTPase is expressed from a constitutive or inducible promoter. PAK levels in some embodiments are reduced by a compound that directly or indirectly enhances expression of an endogenous gene encoding a Rho family GTPase.

A PAK clearance agent in some embodiments is a Rho family GTPase agonist, or is a compound that directly or indirectly increases the activation level of one or more Rho family GTPases. In some embodiments a PAK clearance agent is a compound that increases the level of an activated Rho family GTPase, such as, but not limited to, Rac or cdc42. The compound is, as nonlimiting examples, a compound that modifies a Rho family GTPase such that it is constitutively activated, or a compound that binds or modifies a Rho family GTPase to increase the longevity or stability of its activated (GTP bound) state. Activating mutations of Rho family GTPases are known (Hubsman et al. (2007) Biochem. J. 404: 487-497), as are bacterial toxins such as E. coli necrotizing factors 1 and 2 (CNF1 and CNF2) and Bordetella bronchiseptica dermonecrotizing toxin (DNT) that modify Rho family GTPases to cause their constitutive activation (Fiorentini et al. (2003) Cell Death and Differentiation 10:147-152). Toxins such as CNF1, CNF2, and DNT, fragments thereof that increase the activity of a Rho family GTAPase, or peptides or polypeptides that increase the activity of a Rho family GTAPase having an amino acid sequence at least 80% to 100%, e.g., 85%, 90%, 92%, 93%, 95%, 96%, 97%, 98%, 99%, or any other percent from about 80% to about 100% identical to a sequence of at least ten, at least twenty, at least thirty, at least forty, at least fifty, at least sixty, at least seventy, at least eighty, at least ninety, or at least 100 contiguous amino acids of the toxin are also used as PAK clearance agents. Small molecule inhibitors designed to mimic the effect of activating mutations of GTPases that are upstream regulators of PAK or designed to mimic the effect of bacterial toxins that activate GTPases that bind and activate PAK are also included as compounds that down-regulate PAK levels.

In some embodiments, the inhibitor is a compound that inhibits post-translational modification of a Rho family GTPase. For example, in some embodiments a compound that inhibits prenylation of small Rho-family GTPases such as Rho, Rac, and cdc42 is used to increase GTPase activity and thereby reduce the amount of PAK in the cell. In some embodiments, a compound that decreases PAK levels is a bisphosphonate compound that inhibits prenylation of Rho-family GTPases such as cdc42 and Rac, in which nonprenylated GTPases have higher activity than their prenylated counterparts (Dunford et al. (2006) J. Bone Miner. Res. 21: 684-694; Reszka et al. (2004) Mini Rev. Med. Chem. 4: 711-719).

In some embodiments, the PAK inhibitor is a compound that directly or indirectly decreases the activation or activity of the upstream effectors of PAK. For example, in some embodiments a compound that inhibits the GTPase activity of the small Rho-family GTPases such as Rac and cdc42 thereby reduce the activation of PAK kinase. In some embodiments, the compound that decreases PAK activation is by secramine that inhibits cdc42 activation, binding to membranes and GTP in the cell (Pelish et al. (2005) Nat. Chem. Biol. 2: 39-46). In some embodiments, PAK activation is decreased by EHT 1864, a small molecule that inhibits Rac1, Rac1b, Rac2 and Rac3 function by preventing binding to guanine nucleotide association and engagement with downstream effectors (Shutes et al. (2007) J. Biol. Chem. 49: 35666-35678). In some embodiments, PAK activation is also decreased by the NSC23766 small molecule that binds directly to Rac1 and prevents its activation by Rac-specific RhoGEFs (Gao et al. (2004) Proc. Natl. Acad. Sci. U.S.A. 101: 7618-7623). In some embodiments, PAK activation is also decreased by the 16 kDa fragment of prolactin (16k PRL), generated from the cleavage of the 23 kDa prolactin hormone by matrix metalloproteases and cathepsin D in various tissues and cell types. 16k PRL down-regulates the Ras-Tiam1-Rac1-Pak1 signaling pathway by reducing Rac1 activation in response to cell stimuli such as wounding (Lee et al. (2007) Cancer Res 67:11045-11053). In some embodiments, PAK activation is decreased by inhibition of NMDA and/or AMPA receptors. Examples of modulators of AMPA receptors include and are not limited to CNQX (6-cyano-7-nitroquinoxaline-2,3-dione); NBQX (2,3-dihydroxy-6-nitro-7-sulfamoyl-benzo[f]quinoxaline-2,3-dione); DNQX (6,7-dinitroquinoxaline-2,3-dione); kynurenic acid; 2,3-dihydroxy-6-nitro-7-sulfamoylbenzo-[f]quinoxaline quinoxaline or AMPAkines. Examples of modulators of NMDA receptors include and are not limited to ketamine, MK801, memantine, PCP or the like. In some embodiments, PAK activation is decreased by inhibition of TrkB activation. In some embodiments, PAK activation is decreased by inhibition of BDNF activation of TrkB. In some embodiments, the PAK inhibitor is an antibody to BDNF. In some embodiments, PAK activation is decreased by inhibition of TrkB receptors; NMDA receptors; EphB receptors; adenosine receptors; estrogen receptors; integrins; Rho-family GTPases, including Cdc42, Rac (including but not limited to Rac1 and Rac2), CDK5, PI3 kinases, NCK, PDK1, EKT, GRB2, Chp, TC10, Tcl, and Wrch-1; guanine nucleotide exchange factors (“GEFs”), such as but not limited to GEFT, members of the Dbl family of GEFs, p21-activated kinase interacting exchange factor (PIX), DEF6, Zizimin 1, Vav1, Vav2, Dbs, members of the DOCK180 family, Kalirin-7, and Tiam1; G protein-coupled receptor kinase-interacting protein 1 (GIT1), CIB1, filamin A, Etk/Bmx, and/or binding to FMRP and/or sphingosine.

In some embodiments a compound that decreases PAK levels in the cell is a compound that directly or indirectly increases the activity of a guanine exchange factor (GEF) that promotes the active state of a Rho family GTPase, such as an agonist of a GEF that activates a Rho family GTPase, such as but not limited to, Rac or cdc42. Activation of GEFs is also effected by compounds that activate TrkB, NMDA, or EphB receptors.

In some embodiments, a PAK clearance agent is a nucleic acid encoding a GEF that activates a Rho family GTPase, in which the GEF is expressed from a constitutive or inducible promoter. In some embodiments, a guanine nucleotide exchange factor (GEF), such as but not limited to a GEF that activates a Rho family GTPase is overexpressed in cells to increase the activation level of one or more Rho family GTPases and thereby lower the level of PAK in cells. GEFs include, for example, members of the Dbl family of GTPases, such as but not limited to, GEFT, PIX (e.g., alphaPIX, betaPIX), DEF6, Zizimin 1, Vav1, Vav2, Dbs, members of the DOCK180 family, hPEM-2, FLJ00018, kalirin, Tiam1, STEF, DOCK2, DOCK6, DOCK7, DOCK9, Asf, EhGEF3, or GEF-1. In some embodiments, PAK levels are also reduced by a compound that directly or indirectly enhances expression of an endogenous gene encoding a GEF. A GEF expressed from a nucleic acid construct introduced into cells is in some embodiments a mutant GEF, for example a mutant having enhanced activity with respect to wild type.

The clearance agent is optionally a bacterial toxin such as Salmonella typhinmurium toxin SpoE that acts as a GEF to promote Cdc42 nucleotide exchange (Buchwald et al. (2002) EMBO J. 21: 3286-3295; Schlumberger et al. (2003) J. Biological Chem. 278: 27149-27159). Toxins such as SopE, fragments thereof, or peptides or polypeptides having an amino acid sequence at least about 80% to about 100%, e.g., about 85%, about 90%, about 92%, about 93%, about 95%, about 96%, about 97%, about 98%, about 99%, or any other percent from about 80% to about 100% identical to a sequence of at least five, at least ten, at least twenty, at least thirty, at least forty, at least fifty, at least sixty, at least seventy, at least eighty, at least ninety, or at least 100 contiguous amino acids of the toxin are also optionally used as downregulators of PAK activity. The toxin is optionally produced in cells from nucleic acid constructs introduced into cells.

### Modulators of Upstream Regulators of PAKs

In some embodiments, a modulator of an upstream regulator of PAKs is an indirect inhibitor of PAK. In certain instances, a modulator of an upstream regulator of PAKs is a modulator of PDK1. In some instances, a modulator of PDK1 reduces of inhibits the activity of PDK1. In some instances a PDK1 inhibitor is an antisense compound (e.g., any PDK1 inhibitor described in U.S. Pat. No. 6,124,272, which PDK1 inhibitor is incorporated herein by reference). In some instances, a PDK1 inhibitor is a compound described in e.g., U.S. Pat. Nos. 7,344,870, and 7,041,687, which PDK1 inhibitors are incorporated herein by reference. In some embodiments, an indirect inhibitor of PAK is a modulator of a PI3 kinase. In some instances a modulator of a PI3 kinase is a PI3 kinase inhibitor. In some instances, a PI3 kinase inhibitor is an antisense compound (e.g., any PI3 kinase inhibitor described in WO 2001/018023, which PI3 kinase inhibitors are incorporated herein by reference). In some instances, an inhibitor of a PI3 kinase is 3-morpholino-5-phenylnaphthalen-1(4H)-one (LY294002), or a peptide based covalent conjugate of LY294002, (e.g., SF1126, Semaphore pharmaceuticals). In certain embodiments, an indirect inhibitor of PAK is a modulator of Cdc42. In certain embodiments, a modulator of Cdc42 is an inhibitor of Cdc42. In certain embodiments, a Cdc42 inhibitor is an antisense compound (e.g., any Cdc42 inhibitor described in U.S. Pat. No. 6,410,323, which Cdc42 inhibitors are incorporated herein by reference). In some instances, an indirect inhibitor of PAK is a modulator of GRB2. In some instances, a modulator of GRB2 is an inhibitor of GRB2. In some instances a GRB2 inhibitor is a GRb2 inhibitor described in e.g., U.S. Pat. No. 7,229,960, which GRB2 inhibitor is incorporated by reference herein. In certain embodiments, an indirect inhibitor of PAK is a modulator of NCK. In certain embodiments, an indirect inhibitor of PAK is a modulator of ETK. In some instances, a modulator of ETK is an inhibitor of ETK. In some instances an ETK inhibitor is a compound e.g., (-Cyano-(3,5-di-t-butyl-4-hydroxy)thiocinnamide (AG 879).

In some embodiments the PAK inhibitors, binding molecules, and clearance agents provided herein are administered to an individual suffering from autism to alleviate, halt or delay the loss of dendritic spine density in an individual. A pharmacological composition comprising a therapeutically effective amount of at least one of the compounds disclosed herein, including: a PAK transcription inhibitor, a PAK clearance agent, an agent that binds PAK to prevent its interaction with one or more cellular or extracellular proteins, and a PAK antagonist. In some specific embodiments, the pharmacological composition comprises a therapeutically effective amount of at least one of the compounds chosen from the group consisting of: a PAK transcription inhibitor, PAK clearance agent, an agent that binds a PAK to prevent its interaction with one or more cellular proteins, and a PAK antagonist. An individual is an animal, and is preferably a mammal, preferably human.

In other methods PAK inhibitors binding molecules, and clearance agents provided herein are administered to an individual suffering from autism to reverse some or all defects in dendritic spine morphology, spine size, spine motility and/or spine plasticity in a subject having, or suspected of having, autism. The method includes: administering to an individual a pharmacological composition comprising a therapeutically effective amount of at least one of the compounds chosen from the group consisting of: a PAK transcription inhibitor, a PAK clearance agent, an agent that binds PAK to prevent its interaction with one or more cellular or extracellular proteins, and a PAK antagonist. In some specific embodiments, the pharmacological composition comprises a therapeutically effective amount of at least one of the compounds chosen from the group consisting of: a Group 1 PAK transcription inhibitor, a Group 1 PAK clearance agent, an agent that binds a Group 1 PAK to prevent its interaction with one or more cellular proteins, and a Group 1 PAK antagonist. An individual is an animal, and is preferably a mammal, preferably human.

In some embodiments, indirect PAK inhibitors act by decreasing transcription and/or translation of PAK. A PAK inhibitor, in some embodiments, decreases transcription and/or translation of a PAK. For example, in some embodiments, modulation of PAK transcription or translation occurs through the administration of specific or non-specific inhibitors of PAK transcription or translation. In some embodiments, proteins or non-protein factors that bind the upstream region of the PAK gene or the 5′ UTR of a PAK mRNA are assayed for their affect on transcription or translation using transcription and translation assays (see, for example, Baker, et al. (2003) J. Biol. Chem. 278: 17876-17884; Jiang et al. (2006) J. Chromatography A 1133: 83-94; Novoa et al. (1997) Biochemistry 36: 7802-7809; Brandi et al. (2007) Methods Enzymol. 431: 229-267). PAK inhibitors include DNA or RNA binding proteins or factors that reduce the level of transcription or translation or modified versions thereof. In other embodiments, a PAK inhibitor is a modified form (e.g., mutant form or chemically modified form) of a protein or other compound that positively regulates transcription or translation of PAK, in which the modified form reduces transcription or translation of PAK. In yet other embodiments, a transcription or translation inhibitor is an antagonist of a protein or compound that positively regulates transcription or translation of PAK, or is an agonist of a protein that represses transcription or translation.

Regions of a gene other than those upstream of the transcriptional start site and regions of an mRNA other than the 5′ UTR (such as but not limited to regions 3′ of the gene or in the 3′ UTR of an mRNA, or regions within intron sequences of either a gene or mRNA) also include sequences to which effectors of transcription, translation, mRNA processing, mRNA transport, and mRNA stability bind. In some embodiments, a PAK inhibitor is a clearance agent comprising a polypeptide having homology to an endogenous protein that affects mRNA processing, transport, or stability, or is an antagonist or agonist of one or more proteins that affect mRNA processing, transport, or turnover, such that the inhibitor reduces the expression of PAK protein by interfering with PAK mRNA transport or processing, or by reducing the half-life of PAK mRNA. In some embodiments, PAK clearance agents interfere with transport or processing of a PAK mRNA, or by reducing the half-life of a PAK mRNA.

For example, PAK clearance agents decrease RNA and/or protein half-life of a PAK isoform, for example, by directly affecting mRNA and/or protein stability. In certain embodiments, PAK clearance agents cause PAK mRNA and/or protein to be more accessible and/or susceptible to nucleases, proteases, and/or the proteasome. In some embodiments, PAK inhibitors decrease the processing of PAK mRNA thereby reducing PAK activity. For example, PAK inhibitors function at the level of pre-mRNA splicing, 5′ end formation (e.g. capping), 3′ end processing (e.g. cleavage and/or polyadenylation), nuclear export, and/or association with the translational machinery and/or ribosomes in the cytoplasm. In some embodiments, PAK inhibitors cause a decrease in the level of PAK mRNA and/or protein, the half-life of PAK mRNA and/or protein by at least about 5%, at least about 10%, at least about 20%, at least about 30%, at least about 40%, at least about 50%, at least about 60%, at least about 80%, at least about 90%, at least about 95%, or substantially 100%.

In some embodiments, the PAK inhibitor is a clearance agent that comprises one or more RNAi or antisense oligonucleotides directed against one or more PAK isoform RNAs. In some embodiments, the PAK inhibitor comprises one or more ribozymes directed against one or more PAK isoform RNAs. The design, synthesis, and use of RNAi constructs, antisense oligonucleotides, and ribozymes are found, for example, in Dykxhoorn et al. (2003) Nat. Rev. Mol. Cell. Biol. 4: 457-467; Hannon et al. (2004) Nature 431: 371-378; Sarver et al. (1990) Science 247:1222-1225; Been et al. (1986) Cell 47:207-216). In some embodiments, nucleic acid constructs that induce triple helical structures are also introduced into cells to inhibit transcription of the PAK gene (Helene (1991) Anticancer Drug Des. 6:569-584).

For example, a PAK inhibitor that is a clearance agent is in some embodiments an RNAi molecule or a nucleic acid construct that produces an RNAi molecule. An RNAi molecule comprises a double-stranded RNA of at least about seventeen bases having a 2-3 nucleotide single-stranded overhangs on each end of the double-stranded structure, in which one strand of the double-stranded RNA is substantially complementary to the target PAK RNA molecule whose downregulation is desired. “Substantially complementary” means that one or more nucleotides within the double-stranded region are not complementary to the opposite strand nucleotide(s). Tolerance of mismatches is optionally assessed for individual RNAi structures based on their ability to downregulate the target RNA or protein. In some embodiments, RNAi is introduced into the cells as one or more short hairpin RNAs (“shRNAs”) or as one or more DNA constructs that are transcribed to produce one or more shRNAs, in which the shRNAs are processed within the cell to produce one or more RNAi molecules.

Nucleic acid constructs for the expression of siRNA, shRNA, antisense RNA, ribozymes, or nucleic acids for generating triple helical structures are optionally introduced as RNA molecules or as recombinant DNA constructs. DNA constructs for reducing gene expression are optionally designed so that the desired RNA molecules are expressed in the cell from a promoter that is transcriptionally active in mammalian cells, such as, for example, the SV40 promoter, the human cytomegalovirus immediate-early promoter (CMV promoter), or the pol III and/or pol II promoter using known methods. For some purposes, it is desirable to use viral or plasmid-based nucleic acid constructs. Viral constructs include but are not limited to retroviral constructs, lethiviral constructs, or based on a pox virus, a herpes simplex virus, an adenovirus, or an adeno-associated virus (AAV).

In other embodiments, a PAK inhibitor is a polypeptide that decreases the activity of PAK. In some embodiments, a PAK inhibitor is a polypeptide that decreases the activity of a PAK. Protein and peptide inhibitors of PAK are optionally based on natural substrates of PAK, e.g., Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), cortactin, cofilin, Ras, Raf, Mek, p47(phox), BAD, caspase 3, estrogen and/or progesterone receptors, NET1, Gαz, phosphoglycerate mutase-B, RhoGDI, prolactin, p41Arc, cortactin, and/or Aurora-A. In some embodiments, a PAK inhibitor is based on a sequence of PAK itself, for example, the autoinhibitory domain in the N-terminal portion of the PAK protein that binds the catalytic domain of a partner PAK molecule when the PAK molecule is in its homodimeric state (Zhao et al. (1998) Mol. Cell. Biol. 18:2153-2163; Knaus et al. (1998) J. Biol. Chem. 273: 21512-21518; Hofman et al. (2004) J. Cell Sci. 117: 4343-4354). In some embodiments, polypeptide inhibitors of PAK comprise peptide mimetics, in which the peptide has binding characteristics similar to a natural binding partner or substrate of PAK.

In some embodiments, provided herein are compounds that downregulate PAK protein level. In some embodiments, the compounds described herein activate or increase the activity of an upstream regulator or downstream target of PAK. In some embodiments, compounds described herein downregulate protein level of a PAK. In some instances compounds described herein reduce at least one of the symptoms related autism by reducing the amount of PAK in a cell. In some embodiments a compound that decreases PAK protein levels in cells also decreases the activity of PAK in the cells. In some embodiments a compound that decreases PAK protein levels does not have a substantial impact on PAK activity in cells. In some embodiments a compound that increases PAK activity in cells decreases PAK protein levels in the cells.

In some embodiments, a compound that decreases the amount of PAK protein in cells decreases transcription and/or translation of PAK or increases the turnover rate of PAK mRNA or protein by modulating the activity of an upstream effector or downstream regulator of PAK. In some embodiments, PAK expression or PAK levels are influenced by feedback regulation based on the conformation, chemical modification, binding status, or activity of PAK itself. In some embodiments, PAK expression or PAK levels are influenced by feedback regulation based on the conformation, chemical modification, binding status, or activity of molecules directly or indirectly acted on by PAK signaling pathways. As used herein “binding status” refers to any or a combination of whether PAK, an upstream regulator of PAK, or a downstream effector of PAK is in a monomeric state or in an oligomeric complex with itself, or whether it is bound to other polypeptides or molecules. For example, a downstream target of PAK, when phosphorylated by PAK, in some embodiments directly or indirectly downregulates PAK expression or decrease the half-life of PAK mRNA or protein. Downstream targets of PAK include but are not limited to: Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), Ras, Raf, Mek, p47^{phox}, BAD, caspase 3, estrogen and/or progesterone receptors, NET1, Gαz, phosphoglycerate mutase-B, RhoGDI, prolactin, p41^{Arc}, cortactin, and/or Aurora-A. Downregulators of PAK levels include downstream targets of PAK or fragments thereof in a phosphorylated state and downstream targets of PAK or fragments thereof in a hyperphosphorylated state.

A fragment of a downstream target of PAK includes any fragment with an amino acid sequence at least about 80% to about 100%, e.g., about 85%, about 90%, about 92%, about 93%, about 95%, about 96%, about 97%, about 98%, about 99%, or any other percent from about 80% to about 100% identical to a sequence of at least five, at least ten, at least twenty, at least thirty, at least forty, at least fitly, at least sixty, at least seventy, at least eighty, at least ninety, or at least 100 contiguous amino acids of the downstream regulator, in which the fragment of the downstream target of PAK is able to downregulate PAK mRNA or protein expression or increase turnover of PAK mRNA or protein. In some embodiments, the fragment of a downstream regulator of PAK comprises a sequence that includes a phosphorylation site recognized by PAK, in which the site is phosphorylated.

In some embodiments, a compound that decreases the level of PAK includes a peptide, polypeptide, or small molecule that inhibits dephosphorylation of a downstream target of PAK, such that phosphorylation of the downstream target remains at a level that leads to downregulation of PAK levels.

In some embodiments, PAK activity is reduced or inhibited via activation and/or inhibition of an upstream regulator and/or downstream target of PAK. In some embodiments, the protein expression of a PAK is downregulated. In some embodiments, the amount of PAK in a cell is decreased. In some embodiments a compound that decreases PAK protein levels in cells also decreases the activity of PAK in the cells. In some embodiments a compound that decreases PAK protein levels does not decrease PAK activity in cells. In some embodiments a compound that increases PAK activity in cells decreases PAK protein levels in the cells.

In some embodiments, a PAK inhibitor is a small molecule. As referred to herein, a “small molecule” is an organic molecule that is less than about 5 kilodaltons (kDa) in size. In some embodiments, the small molecule is less than about 4 kDa, 3 kDa, about 2 kDa, or about 1 kDa. In some embodiments, the small molecule is less than about 800 daltons (Da), about 600 Da, about 500 Da, about 400 Da, about 300 Da, about 200 Da, or about 100 Da. In some embodiments, a small molecule is less than about 4000 g/mol, less than about 3000 g/mol, 2000 g/mol, less than about 1500 g/mol, less than about 1000 g/mol, less than about 800 g/mol, or less than about 500 g/mol. In some embodiments, small molecules are non-polymeric. Typically, small molecules are not proteins, polypeptides, polynucleotides, oligonucleotides, polysaccharides, glycoproteins, or proteoglycans, but include peptides of up to about 40 amino acids. A derivative of a small molecule refers to a molecule that shares the same structural core as the original small molecule, but which is prepared by a series of chemical reactions from the original small molecule. As one example, a pro-drug of a small molecule is a derivative of that small molecule. An analog of a small molecule refers to a molecule that shares the same or similar structural core as the original small molecule, and which is synthesized by a similar or related route, or art-recognized variation, as the original small molecule.

In certain embodiments, compounds described herein have one or more chiral centers. As such, all stereoisomers are envisioned herein. In various embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieve in any suitable manner, including by way of non-limiting example, by resolution of the racemic form by recrystallization techniques, by synthesis from optically-active starting materials, by chiral synthesis, or by chromatographic separation using a chiral stationary phase. In some embodiments, mixtures of one or more isomer are utilized as the therapeutic compound described herein. In certain embodiments, compounds described herein contain one or more chiral centers. These compounds are prepared by any means, including enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, chromatography, and the like.

In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenenesulfonate, mesylate and the like. Furthermore, pharmaceutically acceptable salts include, by way of non-limiting example, alkaline earth metal salts (e.g., calcium or magnesium), alkali metal salts (e.g., sodium-dependent or potassium), ammonium salts and the like.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference for such disclosure). General methods for the preparation of compound as described herein are modified by the use of appropriate reagents and conditions, for the introduction of the various moieties found in the formulae as provided herein. As a guide the following synthetic methods are utilized.

Compounds described herein are synthesized starting from compounds that are available from commercial sources or that are prepared using procedures outlined herein.

Formation of Covalent Linkages by Reaction of an Electrophile with a Nucleophile

The compounds described herein are modified using various electrophiles and/or nucleophiles to form new functional groups or substituents. Table A entitled “Examples of Covalent Linkages and Precursors Thereof” lists selected non-limiting examples of covalent linkages and precursor functional groups which yield the covalent linkages. Table A is used as guidance toward the variety of electrophiles and nucleophiles combinations available that provide covalent linkages. Precursor functional groups are shown as electrophilic groups and nucleophilic groups.

TABLE A
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

### Use of Protecting Groups

In the reactions described, it is necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. In some embodiments it is contemplated that each protective group be removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl in the presence of amines blocked with acid labile groups such as t-butyl carbamate or with carbamates that are both acid and base stable but hydrolytically removable.

In some embodiments carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a Pd⁰-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups are selected from:

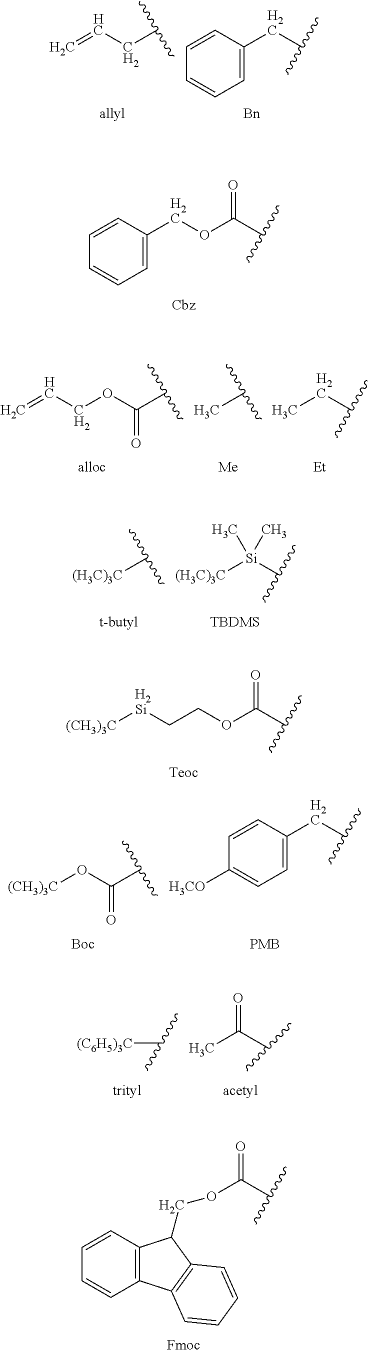

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### CERTAIN DEFINITIONS

As used herein the term “Treatment”, “treat”, or “treating” includes achieving a therapeutic benefit and/or a prophylactic benefit. Therapeutic benefit is meant to include eradication or amelioration of the underlying disorder or condition being treated. For example, in an individual with autism, therapeutic benefit includes alleviation, or partial and/or complete halting of the progression of the disorder, or partial or complete reversal of the disorder. Also, a therapeutic benefit is achieved with the eradication or amelioration of one or more of the physiological or psychological symptoms associated with the underlying condition such that an improvement is observed in the patient, notwithstanding the fact that the patient is still affected by the condition. A prophylactic benefit of treatment includes prevention of a condition, retarding the progress of a condition, or decreasing the likelihood of occurrence of a condition. As used herein, “treat”, “treating” or “treatment” includes prophylaxis.

As used herein, the phrase “abnormal spine size” refers to dendritic spine volumes or dendritic spine surface areas (e.g., volumes or surface areas of the spine heads and/or spine necks) associated with autism that deviate significantly relative to spine volumes or surface areas in the same brain region (e.g., the CA1 region, the prefrontal cortex) in a normal individual (e.g., a mouse, rat, or human) of the same age; such abnormalities are determined as appropriate, by methods including, e.g., tissue samples, relevant animal models, post-mortem analyses, or other model systems.

The phrase “defective spine morphology” or “abnormal spine morphology” or “aberrant spine morphology” refers to abnormal dendritic spine shapes, volumes, surface areas, length, width (e.g., diameter of the neck), spine head diameter, spine head volume, spine head surface area, spine density, ratio of mature to immature spines, ratio of spine volume to spine length, or the like that is associated with autism relative to the dendritic spine shapes, volumes, surface areas, length, width (e.g., diameter of the neck), spine density, ratio of mature to immature spines, ratio of spine volume to spine length, or the like observed in the same brain region in a normal individual (e.g., a mouse, rat, or human) of the same age; such abnormalities or defects are determined as appropriate, by methods including, e.g., tissue samples, relevant animal models, post-mortem analyses, or other model systems.

The phrase “abnormal spine function” or “defective spine function” or “aberrant spine function” refers to a defect of dendritic spines to undergo stimulus-dependent morphological or functional changes (e.g., following activation of AMPA and/or NMDA receptors, LTP, LTD, etc) associated with autism as compared to dendritic spines in the same brain region in a normal individual of the same age. The “defect” in spine function includes, e.g., a reduction in dendritic spine plasticity, (e.g., an abnormally small change in dendritic spine morphology or actin re-arrangement in the dendritic spine), or an excess level of dendritic plasticity, (e.g., an abnormally large change in dendritic spine morphology or actin re-arrangement in the dendritic spine). Such abnormalities or defects are determined as appropriate, by methods including, e.g., tissue samples, relevant animal models, post-mortem analyses, or other model systems.

The phrase “abnormal spine motility” refers to a significant low or high movement of dendritic spines associated with autism as compared to dendritic spines in the same brain region in a normal individual of the same age. Any defect in spine morphology (e.g., spine length, density or the like) or synaptic plasticity or synaptic function (e.g., LTP, LTD or the like) or spine motility occurs in any region of the brain, including, for example, the frontal cortex, the hippocampus, the amygdala, the CA1 region, the prefrontal cortex or the like. Such abnormalities or defects are determined as appropriate, by methods including, e.g., tissue samples, relevant animal models, post-mortem analyses, or other model systems.

As used herein, the phrase “biologically active” refers to a characteristic of any substance that has activity in a biological system and/or organism. For instance, a substance that, when administered to an organism, has a biological effect on that organism is considered to be biologically active. In particular embodiments, where a protein or polypeptide is biologically active, a portion of that protein or polypeptide that shares at least one biological activity of the protein or polypeptide is typically referred to as a “biologically active” portion.

As used herein, the term “effective amount” is an amount, which when administered systemically, is sufficient to effect beneficial or desired results, such as beneficial or desired clinical results, stabilized behavior, or other desired effects. An effective amount is also an amount that produces a prophylactic effect, e.g., an amount that delays, reduces, or eliminates the appearance of a pathological or undesired condition associated with autism. An effective amount is optionally administered in one or more administrations. In terms of treatment, an “effective amount” of a composition described herein is an amount that is sufficient to palliate, alleviate, ameliorate, stabilize, reverse or slow the progression of autism. An “effective amount” includes any PAK inhibitor described herein used alone or in conjunction with one or more agents used to treat a disease or disorder. An “effective amount” of a therapeutic agent as described herein will be determined by a patient's attending physician or other medical care provider. Factors which influence what a therapeutically effective amount will be include, the absorption profile (e.g., its rate of uptake into the brain) of the PAK inhibitor, time elapsed since the initiation of disease, and the age, physical condition, existence of other disease states, and nutritional status of an individual being treated. Additionally, other medication the patient is receiving, e.g., antipsychotic drugs used in combination with a PAK inhibitor, will typically affect the determination of the therapeutically effective amount of the therapeutic agent to be administered.

As used herein, the term “inhibitor” refers to a molecule which is capable of inhibiting (including partially inhibiting or allosteric inhibition) one or more of the biological activities of a target molecule, e.g., a p21-activated kinase. Inhibitors, for example, act by reducing or suppressing the activity of a target molecule and/or reducing or suppressing signal transduction. In some embodiments, a PAK inhibitor described herein causes substantially complete inhibition of one or more PAKs. In some embodiments, the phrase “partial inhibitor” refers to a molecule which can induce a partial response for example, by partially reducing or suppressing the activity of a target molecule and/or partially reducing or suppressing signal transduction. In some instances, a partial inhibitor mimics the spatial arrangement, electronic properties, or some other physicochemical and/or biological property of the inhibitor. In some instances, in the presence of elevated levels of an inhibitor, a partial inhibitor competes with the inhibitor for occupancy of the target molecule and provides a reduction in efficacy, relative to the inhibitor alone. In some embodiments, a PAK inhibitor described herein is a partial inhibitor of one or more PAKs. In some embodiments, a PAK inhibitor described herein is an allosteric modulator of PAK.

In some embodiments, a PAK inhibitor described herein blocks the p21 binding domain of PAK. In some embodiments, a PAK inhibitor described herein blocks the ATP binding site of PAK. In some embodiments, a PAK inhibitor is a “Type II” kinase inhibitor. In some embodiment a PAK inhibitor stabilizes PAK in its inactive conformation. In some embodiments, a PAK inhibitor stabilizes the “DFG-out” conformation of PAK.

In some embodiments, PAK inhibitors reduce, abolish, and/or remove the binding between PAK and at least one of its natural binding partners (e.g., Cdc42 or Rac). In some instances, binding between PAK and at least one of its natural binding partners is stronger in the absence of a PAK inhibitor (by e.g., 90%, 80%, 70%, 60%, 50%, 40%, 30% or 20%) than in the presence of a PAK inhibitor. Alternatively or additionally, PAK inhibitors inhibit the phosphotransferase activity of PAK, e.g., by binding directly to the catalytic site or by altering the conformation of PAK such that the catalytic site becomes inaccessible to substrates. In some embodiments, PAK inhibitors inhibit the ability of PAK to phosphorylate at least one of its target substrates, e.g., LIM kinase 1 (LIMK1), myosin light chain kinase (MLCK), myosin light chain, cortactin; or itself. PAK inhibitors include inorganic and/or organic compounds.

In some embodiments, PAK inhibitors described herein increase dendritic spine length. In some embodiments, PAK inhibitors described herein decrease dendritic spine length. In some embodiments, PAK inhibitors described herein increase dendritic neck diameter. In some embodiments, PAK inhibitors described herein decrease dendritic neck diameter. In some embodiments, PAK inhibitors described herein increase dendritic spine head diameter. In some embodiments, PAK inhibitors described herein decrease dendritic spine head diameter. In some embodiments, PAK inhibitors described herein increase dendritic spine head volume. In some embodiments, PAK inhibitors described herein decrease dendritic spine head volume. In some embodiments, PAK inhibitors described herein increase dendritic spine surface area. In some embodiments, PAK inhibitors described herein decrease dendritic spine surface area. In some embodiments, PAK inhibitors described herein increase dendritic spine density. In some embodiments, PAK inhibitors described herein decrease dendritic spine density. In some embodiments, PAK inhibitors described herein increase the number of mushroom shaped spines. In some embodiments, PAK inhibitors described herein decrease the number of mushroom shaped spines.

In some embodiments, a PAK inhibitor suitable for the methods described herein is a direct PAK inhibitor. In some embodiments, a PAK inhibitor suitable for the methods described herein is an indirect PAK inhibitor. In some embodiments, a PAK inhibitor suitable for the methods described herein decreases PAK activity relative to a basal level of PAK activity by about 1.1 fold to about 100 fold, e.g., to about 1.2 fold, about 1.5 fold, about 1.6 fold, about 1.7 fold, about 2.0 fold, about 3.0 fold, about 5.0 fold, about 6.0 fold, about 7.0 fold, about 8.5 fold, about 9.7 fold, about 10 fold, about 12 fold, about 14 fold, about 15 fold, about 20 fold, about 30 fold, about 40 fold, about 50 fold, about 60 fold, about 70 fold, about 90 fold, about 95 fold, or by any other amount from about 1.1 fold to about 100 fold relative to basal PAK activity. In some embodiments, the PAK inhibitor is a reversible PAK inhibitor. In other embodiments, the PAK inhibitor is an irreversible PAK inhibitor. Direct PAK inhibitors are optionally used for the manufacture of a medicament for treating autism.

In some embodiments, a PAK inhibitor used for the methods described herein has in vitro ED₅₀ for PAK activation of less than about 100 μM (e.g., less than about 10 μM, less than about 5 μM, less than about 4 μM, less than about 3 μM, less than about 1 μM, less than about 0.8 μM, less than about 0.6 less than about 0.5 μM, less than about 0.4 μM, less than about 0.3 μM, less than about 0.2 μM, less than about 0.1 less than about 0.08 μM, less than about 0.06 μM, less than about 0.05 μM, less than about 0.04 μM, less than about 0.03 μM, less than about 0.02 μM, less than about 0.01 μM, less than about 0.0099 μM, less than about 0.0098 μM, less than about 0.0097 μM, less than about 0.0096 μM, less than about 0.0095 μM, less than about 0.0094 μM, less than about 0.0093 μM, less than about 0.00092, or less than about 0.0090 μM).

As used herein, synaptic function refers to synaptic transmission and/or synaptic plasticity, including stabilization of synaptic plasticity. As used herein, “defect in synaptic plasticity” or “aberrant synaptic plasticity” refers to abnormal synaptic plasticity following stimulation of that synapse. In some embodiments, a defect in synaptic plasticity is a decrease in LTP. In some embodiments, a defect in synaptic plasticity is an increase in LTD. In some embodiments, a defect in synaptic plasticity is erratic (e.g., fluctuating, randomly increasing or decreasing) synaptic plasticity. In some instances, measures of synaptic plasticity are LTP and/or LTD (induced, for example, by theta-burst stimulation, high-frequency stimulation for LTP, low-frequency (1 Hz) stimulation for LTD) and LTP and/or LTD after stabilization. In some embodiments, stabilization of LTP and/or LTD occurs in any region of the brain including the frontal cortex, the hippocampus, the prefrontal cortex, the amygdala or any combination thereof.

As used herein “stabilization of synaptic plasticity” refers to stable LTP or LTD following induction (e.g., by theta-burst stimulation, high-frequency stimulation for LTP, low-frequency (1 Hz) stimulation for LTD).

“Aberrant stabilization of synaptic transmission” (for example, aberrant stabilization of LTP or LTD), refers to failure to establish a stable baseline of synaptic transmission following an induction paradigm (e.g., by theta-burst stimulation high-frequency stimulation for LTP, low-frequency (1 Hz) stimulation for LTD) or an extended period of vulnerability to disruption by pharmacological or electrophysiological means

As used herein “synaptic transmission” or “baseline synaptic transmission” refers to the EPSP and/or IPSP amplitude and frequency, neuronal excitability or population spike thresholds of a normal individual (e.g., an individual not suffering from autism) or that predicted for an animal model for a normal individual. As used herein “aberrant synaptic transmission” or “defective synaptic transmission” refers to any deviation in synaptic transmission compared to synaptic transmission of a normal individual or that predicted for an animal model for a normal individual. In some embodiments, an individual suffering from autism has a defect in baseline synaptic transmission that is a decrease in baseline synaptic transmission compared to the baseline synaptic transmission in a normal individual or that predicted for an animal model for a normal individual. In some embodiments, an individual suffering from autism has a defect in baseline synaptic transmission that is an increase in baseline synaptic transmission compared to the baseline synaptic transmission in a normal individual or that predicted for an animal model for a normal individual.

As used herein “sensorimotor gating” is assessed, for example, by measuring prepulse inhibition (PPI) and/or habituation of the human startle response. In some embodiments, a defect in sensorimotor gating is a deficit in sensorimotor gating. In some embodiments, a defect in sensorimotor gating is an enhancement of sensorimotor gating.

As used herein, “normalization of aberrant synaptic plasticity” refers to a change in aberrant synaptic plasticity in an individual suffering from, suspected of having, or pre-disposed to autism to a level of synaptic plasticity that is substantially the same as the synaptic plasticity of a normal individual or to that predicted from an animal model for a normal individual. As used herein, substantially the same means, for example, about 90% to about 110% of the measured synaptic plasticity in a normal individual or to that predicted from an animal model for a normal individual. In other embodiments, substantially the same means, for example, about 80% to about 120% of the measured synaptic plasticity in a normal individual or to that predicted from an animal model for a normal individual. In yet other embodiments, substantially the same means, for example, about 70% to about 130% of the synaptic plasticity in a normal individual or to that predicted from an animal model for a normal individual. As used herein, “partial normalization of aberrant synaptic plasticity” refers to any change in aberrant synaptic plasticity in an individual suffering from, suspected of having, or pre-disposed to autism that trends towards synaptic plasticity of a normal individual or to that predicted from an animal model for a normal individual. As used herein “partially normalized synaptic plasticity” or “partially normal synaptic plasticity” is, for example, ±about 25%, ±about 35%, ±about 45%, ±about 55%, ±about 65%, or ±about 75% of the synaptic plasticity of a normal individual or to that predicted from an animal model for a normal individual. In some embodiments, normalization or partial normalization of aberrant synaptic plasticity in an individual suffering from, suspected of having, or pre-disposed to autism is lowering of aberrant synaptic plasticity where the aberrant synaptic plasticity is higher than the synaptic plasticity of a normal individual or to that predicted from an animal model for a normal individual. In some embodiments, normalization or partial normalization of aberrant synaptic plasticity in an individual suffering from, suspected of having, or pre-disposed to autism is an increase in aberrant synaptic plasticity where the aberrant synaptic plasticity is lower than the synaptic plasticity of a normal individual or to that predicted from an animal model for a normal individual. In some embodiments, normalization or partial normalization of synaptic plasticity in an individual suffering from, suspected of having, or pre-disposed to autism is a change from an erratic (e.g., fluctuating, randomly increasing or decreasing) synaptic plasticity to a normal (e.g. stable) or partially normal (e.g., less fluctuating) synaptic plasticity compared to the synaptic plasticity of a normal individual or to that predicted from an animal model for a normal individual. In some embodiments, normalization or partial normalization of synaptic plasticity in an individual suffering from, suspected of having, or pre-disposed to autism is a change from a non-stabilizing synaptic plasticity to a normal (e.g., stable) or partially normal (e.g., partially stable) synaptic plasticity compared to the synaptic plasticity of a normal individual or to that predicted from an animal model for a normal individual.

As used herein, “normalization of aberrant baseline synaptic transmission” refers to a change in aberrant baseline synaptic transmission in an individual suffering from, suspected of having, or pre-disposed to autism to a level of baseline synaptic transmission that is substantially the same as the baseline synaptic transmission of a normal individual or to that predicted from an animal model for a normal individual. As used herein, substantially the same means, for example, about 90% to about 110% of the measured baseline synaptic transmission in a normal individual or to that predicted from an animal model for a normal individual. In other embodiments, substantially the same means, for example, about 80% to about 120% of the measured baseline synaptic transmission in a normal individual or to that predicted from an animal model for a normal individual. In yet other embodiments, substantially the same means, for example, about 70% to about 130% of the measured baseline synaptic transmission in a normal individual or to that predicted from an animal model for a normal individual. As used herein, “partial normalization of aberrant baseline synaptic transmission” refers to any change in aberrant baseline synaptic transmission in an individual suffering from, suspected of having, or pre-disposed to autism that trends towards baseline synaptic transmission of a normal individual or to that predicted from an animal model for a normal individual. As used herein “partially normalized baseline synaptic transmission” or “partially normal baseline synaptic transmission” is, for example, ±about 25%, ±about 35%, ±about 45%, ±about 55%, ±about 65%, or ±about 75% of the measured baseline synaptic transmission of a normal individual or to that predicted from an animal model for a normal individual. In some embodiments, normalization or partial normalization of aberrant baseline synaptic transmission in an individual suffering from, suspected of having, or pre-disposed to autism is lowering of aberrant baseline synaptic transmission where the aberrant baseline synaptic transmission is higher than the baseline synaptic transmission of a normal individual or to that predicted from an animal model for a normal individual. In some embodiments, normalization or partial normalization of aberrant baseline synaptic transmission in an individual suffering from, suspected of having, or pre-disposed to autism is an increase in aberrant baseline synaptic transmission where the aberrant baseline synaptic transmission is lower than the baseline synaptic transmission of a normal individual or to that predicted from an animal model for a normal individual. In some embodiments, normalization or partial normalization of baseline synaptic transmission in an individual suffering from, suspected of having, or pre-disposed to autism is a change from an erratic (e.g., fluctuating, randomly increasing or decreasing) baseline synaptic transmission to a normal (e.g. stable) or partially normal (e.g., less fluctuating) baseline synaptic transmission compared to the baseline synaptic transmission of a normal individual or to that predicted from an animal model for a normal individual. In some embodiments, normalization or partial normalization of aberrant baseline synaptic transmission in an individual suffering from, suspected of having, or pre-disposed to autism is a change from a non-stabilizing baseline synaptic transmission to a normal (e.g., stable) or partially normal (e.g., partially stable) baseline synaptic transmission compared to the baseline synaptic transmission of a normal individual or to that predicted from an animal model for a normal individual.

As used herein, “normalization of aberrant synaptic function” refers to a change in aberrant synaptic function in an individual suffering from, suspected of having, or pre-disposed to autism to a level of synaptic function that is substantially the same as the synaptic function of a normal individual or to that predicted from an animal model for a normal individual. As used herein, substantially the same means, for example, about 90% to about 110% of the synaptic function in a normal individual or to that predicted from an animal model for a normal individual. In other embodiments, substantially the same means, for example, about 80% to about 120% of the synaptic function in a normal individual or to that predicted from an animal model for a normal individual. In yet other embodiments, substantially the same means, for example, about 70% to about 130% of the synaptic function in a normal individual or to that predicted from an animal model for a normal individual. As used herein, “partial normalization of aberrant synaptic function” refers to any change in aberrant synaptic function in an individual suffering from, suspected of having, or pre-disposed to autism that trends towards synaptic function of a normal individual or to that predicted from an animal model for a normal individual. As used herein “partially normalized synaptic function” or “partially normal synaptic function” is, for example, ±about 25%, t about 35%, ±about 45%, ±about 55%, ±about 65%, or ±about 75% of the measured synaptic function of a normal individual or to that predicted from an animal model for a normal individual. In some embodiments, normalization or partial normalization of aberrant synaptic function in an individual suffering from, suspected of having, or pre-disposed to autism is lowering of aberrant synaptic function where the aberrant synaptic function is higher than the synaptic function of a normal individual or to that predicted from an animal model for a normal individual. In some embodiments, normalization or partial normalization of aberrant synaptic function in an individual suffering from, suspected of having, or pre-disposed to autism is an increase in aberrant synaptic function where the aberrant synaptic function is lower than the synaptic function of a normal individual or to that predicted from an animal model for a normal individual. In some embodiments, normalization or partial normalization of synaptic function in an individual suffering from, suspected of having, or pre-disposed to autism is a change from an erratic (e.g., fluctuating, randomly increasing or decreasing) synaptic function to a normal (e.g. stable) or partially normal (e.g., less fluctuating) synaptic function compared to the synaptic function of a normal individual or to that predicted from an animal model for a normal individual. In some embodiments, normalization or partial normalization of aberrant synaptic function in an individual suffering from, suspected of having, or pre-disposed to autism is a change from a non-stabilizing synaptic function to a normal (e.g., stable) or partially normal (e.g., partially stable) synaptic function compared to the synaptic function of a normal individual or to that predicted from an animal model for a normal individual.

As used herein, “normalization of aberrant long term potentiation (LTP)” refers to a change in aberrant LTP in an individual suffering from, suspected of having, or pre-disposed to autism to a level of LTP that is substantially the same as the LTP of a normal individual or to that predicted from an animal model for a normal individual. As used herein, substantially the same means, for example, about 90% to about 110% of the LTP in a normal individual or to that predicted from an animal model for a normal individual. In other embodiments, substantially the same means, for example, about 80% to about 120% of the LTP in a normal individual or to that predicted from an animal model for a normal individual. In yet other embodiments, substantially the same means, for example, about 70% to about 130% of the LTP in a normal individual or to that predicted from an animal model for a normal individual. As used herein, “partial normalization of aberrant LTP” refers to any change in aberrant LTP in an individual suffering from, suspected of having, or pre-disposed to autism that trends towards LTP of a normal individual or to that predicted from an animal model for a normal individual. As used herein “partially normalized LIP” or “partially normal LTP” is, for example, ±about 25%, ±about 35%, ±about 45%, ±about 55%, ±about 65%, or ±about 75% of the measured LTP of a normal individual or to that predicted from an animal model for a normal individual. In some embodiments, normalization or partial normalization of aberrant LTP in an individual suffering from, suspected of having, or pre-disposed to autism is lowering of aberrant LTP where the aberrant LTP is higher than the LTP of a normal individual or to that predicted from an animal model for a normal individual. In some embodiments, normalization or partial normalization of aberrant LTP in an individual suffering from, suspected of having, or pre-disposed to autism is an increase in aberrant LTP where the aberrant LTP is lower than the LTP of a normal individual or to that predicted from an animal model for a normal individual. In some embodiments, normalization or partial normalization of LTP in an individual suffering from, suspected of having, or pre-disposed to autism is a change from an erratic (e.g., fluctuating, randomly increasing or decreasing) LTP to a normal (e.g. stable) or partially normal (e.g., less fluctuating) LTP compared to the LTP of a normal individual or to that predicted from an animal model for a normal individual. In some embodiments, normalization or partial normalization of aberrant LTP in an individual suffering from, suspected of having, or pre-disposed to autism is a change from a non-stabilizing LTP to a normal (e.g., stable) or partially normal (e.g., partially stable) LTP compared to the LTP of a normal individual or to that predicted from an animal model for a normal individual.

As used herein, “normalization of aberrant long term depression (LTD)” refers to a change in aberrant LTD in an individual suffering from, suspected of having, or pre-disposed to autism to a level of LTD that is substantially the same as the LTD of a normal individual or to that predicted from an animal model for a normal individual. As used herein, substantially the same means, for example, about 90% to about 110% of the LTD in a normal individual or to that predicted from an animal model for a normal individual. In other embodiments, substantially the same means, for example, about 80% to about 120% of the LTD in a normal individual or to that predicted from an animal model for a normal individual. In yet other embodiments, substantially the same means, for example, about 70% to about 130% of the LTD in a normal individual or to that predicted from an animal model for a normal individual. As used herein, “partial normalization of aberrant LTD” refers to any change in aberrant LTD in an individual suffering from, suspected of having, or pre-disposed to autism that trends towards LTD of a normal individual or to that predicted from an animal model for a normal individual. As used herein “partially normalized LTD” or “partially normal LTD” is, for example, ±about 25%, ±about 35%, ±about 45%, ±about 55%, ±about 65%, or ±about 75% of the measured LTD of a normal individual or to that predicted from an animal model for a normal individual. In some embodiments, normalization or partial normalization of aberrant LTD in an individual suffering from, suspected of having, or pre-disposed to autism is lowering of aberrant LTD where the aberrant LTD is higher than the LTD of a normal individual or to that predicted from an animal model for a normal individual. In some embodiments, normalization or partial normalization of aberrant LTD in an individual suffering from, suspected of having, or pre-disposed to autism is an increase in aberrant LTD where the aberrant LTD is lower than the LTD of a normal individual or to that predicted from an animal model for a normal individual. In some embodiments, normalization or partial normalization of LTD in an individual suffering from, suspected of having, or pre-disposed to autism is a change from an erratic (e.g., fluctuating, randomly increasing or decreasing) LTD to a normal (e.g. stable) or partially normal (e.g., less fluctuating) LTD compared to the LTD of a normal individual or to that predicted from an animal model for a normal individual. In some embodiments, normalization or partial normalization of aberrant LTD in an individual suffering from, suspected of having, or pre-disposed to autism is a change from a non-stabilizing LTD to a normal (e.g., stable) or partially normal (e.g., partially stable) LTD compared to the LTD of a normal individual or to that predicted from an animal model for a normal individual.

As used herein, “normalization of aberrant sensorimotor gating” refers to a change in aberrant sensorimotor gating in an individual suffering from, suspected of having, or pre-disposed to autism to a level of sensorimotor gating that is substantially the same as the sensorimotor gating of a normal individual or to that predicted from an animal model for a normal individual. As used herein, substantially the same means, for example, about 90% to about 110% of the sensorimotor gating in a normal individual or to that predicted from an animal model for a normal individual. In other embodiments, substantially the same means, for example, about 80% to about 120% of the sensorimotor gating in a normal individual or to that predicted from an animal model for a normal individual. In yet other embodiments, substantially the same means, for example, about 70% to about 130% of the sensorimotor gating in a normal individual or to that predicted from an animal model for a normal individual. As used herein, “partial normalization of aberrant sensorimotor gating” refers to any change in aberrant sensorimotor gating in an individual suffering from, suspected of having, or pre-disposed to autism that trends towards sensorimotor gating of a normal individual or to that predicted from an animal model for a normal individual. As used herein “partially normalized sensorimotor gating” or “partially normal sensorimotor gating” is, for example, ±about 25%, ±about 35%, ±about 45%, ±about 55%, ±about 65%, or ±about 75% of the measured sensorimotor gating of a normal individual or to that predicted from an animal model for a normal individual. In some embodiments, normalization or partial normalization of aberrant sensorimotor gating in an individual suffering from, suspected of having, or pre-disposed to autism is lowering of aberrant sensorimotor gating where the aberrant sensorimotor gating is higher than the sensorimotor gating of a normal individual or to that predicted from an animal model for a normal individual. In some embodiments, normalization or partial normalization of aberrant sensorimotor gating in an individual suffering from, suspected of having, or pre-disposed to autism is an increase in aberrant sensorimotor gating where the aberrant sensorimotor gating is lower than the sensorimotor gating of a normal individual or to that predicted from an animal model for a normal individual. In some embodiments, normalization or partial normalization of sensorimotor gating in an individual suffering from, suspected of having, or pre-disposed to autism is a change from an erratic (e.g., fluctuating, randomly increasing or decreasing) sensorimotor gating to a normal (e.g. stable) or partially normal (e.g., less fluctuating) sensorimotor gating compared to the sensorimotor gating of a normal individual or to that predicted from an animal model for a normal individual. In some embodiments, normalization or partial normalization of aberrant sensorimotor gating in an individual suffering from, suspected of having, or pre-disposed to autism is a change from a non-stabilizing sensorimotor gating to a normal (e.g., stable) or partially normal (e.g., partially stable) sensorimotor gating compared to the sensorimotor gating of a normal individual or to that predicted from an animal model for a normal individual.

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; (4) post-translational modification of a polypeptide or protein.

As used herein the term “PAK polypeptide” or “PAK protein” or “PAK” refers to a protein that belongs in the family of p21-activated serine/threonine protein kinases. These include mammalian isoforms of PAK, e.g., the Group I PAK proteins (sometimes referred to as Group A PAK proteins), including PAK1, PAK2, PAK3, as well as the Group II PAK proteins (sometimes referred to as Group B PAK proteins), including PAK-4, PAK5, and/or PAK6 Also included as PAK polypeptides or PAK proteins are lower eukaryotic isoforms, such as the yeast Step 20 (Leberter et al., 1992, EMBO J., 11:4805; incorporated herein by reference) and/or the Dictyostelium single-headed myosin I heavy chain kinases (Wu et al., 1996, J. Biol. Chem., 271:31787; incorporated herein by reference). Representative examples of PAK amino acid sequences include, but are not limited to, human PAK 1 (GenBank Accession Number AAA65441), human PAK2 (GenBank Accession Number AAA65442), human PAK3 (GenBank Accession Number AAC36097), human PAK 4 (GenBank Accession Numbers NP_{—}005875 and CAA09820), human PAK5 (GenBank Accession Numbers CAC18720 and BAA94194), human PAK6 (GenBank Accession Numbers NP_{—}064553 and AAF82800), human PAK7 (GenBank Accession Number Q9P286), C. elegans PAK (GenBank Accession Number BAA 11844), D. melanogaster PAK (GenBank Accession Number AAC47094), and rat PAK1 (GenBank Accession Number AAB95646). In some embodiments, a PAK polypeptide comprises an amino acid sequence that is at least about 70% to about 100% identical, e.g., at least about 75%, about 80%, about 85%, about 86%, about 87%, about 88%, about 90%, about 91%, about 92%, about 94%, about 95%, about 96%, about 97%, about 98%, or any other percent from about 70% to about 100% identical to sequences of GenBank Accession Numbers AAA65441, AAA65442, AAC36097, NP_{—}005875, CAA09820, CAC18720, BAA94194, NP_{—}064553, AAF82800, Q9P286, BAA11844, AAC47094, and/or AAB95646. In some embodiments, a Group I PAK polypeptide comprises an amino acid sequence that is at least about 70% to about 100% identical, e.g., at least about 75%, about 80%, about 85%, about 86%, about 87%, about 88%, about 90%, about 91%, about 92%, about 94%, about 95%, about 96%, about 97%, about 98%, or any other percent from about 70% to about 100% identical to sequences of GenBank Accession Numbers AAA65441, AAA65442, and/or AAC36097.

Representative examples of PAK genes encoding PAK proteins include, but are not limited to, human PAK1 (GenBank Accession Number U24152), human PAK2 (GenBank Accession Number U24153), human PAK3 (GenBank Accession Number AF068864), human PAK-4 (GenBank Accession Number AJ011855), human PAK5 (GenBank Accession Number AB040812), and human PAK6 (GenBank Accession Number AF276893). In some embodiments, a PAK gene comprises a nucleotide sequence that is at least 70% to 100% identical, e.g., at least about 75%, about 80%, about 85%, about 86%, about 87%, about 88%, about 90%, about 91%, about 92%, about 94%, about 95%, about 96%, about 97%, about 98%, or any other percent from about 70% to about 100% identical to sequences of GenBank Accession Numbers U24152, U24153, AF068864, AJ011855, AB040812, and/or AF276893. In some embodiments, a Group I PAK gene comprises a nucleotide sequence that is at least 70% to 100% identical, e.g., at least about 75%, about 80%, about 85%, about 86%, about 87%, about 88%, about 90%, about 91%, about 92%, about 94%, about 95%, about 96%, about 97%, about 98%, or any other percent from about 70% to about 100% identical to sequences of GenBank Accession Numbers U24152, U24153, and/or AF068864.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent homology between the two sequences is a function of the number of identical positions shared by the sequences (i.e., % identity=# of identical positions/total # of positions (e.g., overlapping positions)×100). In one embodiment the two sequences are the same length.

To determine percent homology between two sequences, the algorithm of Karlin and Altschul (1990) Proc. Natl. Acad. Sci. USA 87:2264-2268, modified as in Karlin and Altschul (1993) Proc. Natl. Acad. Sci. USA 90:5873-5877 is used. Such an algorithm is incorporated into the NBLAST and XBLAST programs of Altschul, et al. (1990) J. Mol. Biol. 215:403-410. BLAST nucleotide searches are performed with the NBLAST program, score=100, wordlength=12 to obtain nucleotide sequences homologous to a nucleic acid molecules described or disclose herein. BLAST protein searches are performed with the XBLAST program, score=50, wordlength=3. To obtain gapped alignments for comparison purposes, Gapped BLAST is utilized as described in Altschul et al. (1997) Nucleic Acids Res. 25:3389-3402. When utilizing BLAST and Gapped BLAST programs, the default parameters of the respective programs (e.g., XBLAST and NBLAST) are used. See the website of the National Center for Biotechnology Information for further details (on the World Wide Web at ncbi.nlm.nih.gov). Proteins suitable for use in the methods described herein also includes proteins having between 1 to 15 amino acid changes, e.g., 1, 2, 3, 4, 5, 6, 7, 8, 9, 10, 11, 12, 13, 14, or 15 amino acid substitutions, deletions, or additions, compared to the amino acid sequence of any protein PAK inhibitor described herein. In other embodiments, the altered amino acid sequence is at least about 75% identical, e.g., about 77%, about 80%, about 82%, about 85%, about 88%, about 90%, about 92%, about 95%, about 97%, about 98%, about 99%, or about 100% identical to the amino acid sequence of any protein PAK inhibitor described herein. Such sequence-variant proteins are suitable for the methods described herein as long as the altered amino acid sequence retains sufficient biological activity to be functional in the compositions and methods described herein. Where amino acid substitutions are made, the substitutions should be conservative amino acid substitutions. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff et al (1992), Proc. Natl. Acad. Sci. USA, 89:10915-10919). Accordingly, the BLOSUM62 substitution frequencies are used to define conservative amino acid substitutions that may be introduced into the amino acid sequences described or described herein. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

As used herein, the term “PAK activity,” unless otherwise specified, includes, but is not limited to, at least one of PAK protein-protein interactions, PAK phosphotransferase activity (intermolecular or intermolecular), translocation, etc. of one or more PAK isoforms.

As used herein, a “PAK inhibitor” refers to any molecule, compound, or composition that directly or indirectly decreases the PAK activity. In some embodiments, PAK inhibitors inhibit, decrease, and/or abolish the level of a PAK mRNA and/or protein or the half-life of PAK mRNA and/or protein, such inhibitors are referred to as “clearance agents”. In some embodiments, a PAK inhibitor is a PAK antagonist that inhibits, decreases, and/or abolishes an activity of PAK. In some embodiments, a PAK inhibitor also disrupts, inhibits, or abolishes the interaction between PAK and its natural binding partners (e.g., a substrate for a PAK kinase, a Rac protein, a cdc42 protein, LIM kinase) or a protein that is a binding partner of PAK in a pathological condition, as measured using standard methods. In some embodiments, the PAK inhibitor is a Group I PAK inhibitor that inhibits, for example, one or more Group I PAK polypeptides, for example, PAK1, PAK2, and/or PAK3. In some embodiments, the PAK inhibitor is a PAK1 inhibitor. In some embodiments, the PAK inhibitor is a PAK2 inhibitor. In some embodiments, the PAK inhibitor is a PAK3 inhibitor. In some embodiments, the PAK inhibitor is a mixed PAK1/PAK3 inhibitor. In some embodiments, the PAK inhibitor inhibits all three Group I PAK isoforms (PAK1, PAK2 and PAK3) with equal or similar potency. In some embodiments, the PAK inhibitor is a Group II PAK inhibitor that inhibits one or more Group II PAK polypeptides, for example PAK-4, PAK5, and/or PAK6. In some embodiments, the PAK inhibitor is a PAK-4 inhibitor. In some embodiments, the PAK inhibitor is a PAK5 inhibitor. In some embodiments, the PAK inhibitor is a PAK6 inhibitor. In some embodiments, the PAK inhibitor is a PAK7 inhibitor. As used herein, a PAK5 polypeptide is substantially homologous to a PAK7 polypeptide.

In some embodiments, PAK inhibitors reduce, abolish, and/or remove the binding between PAK and at least one of its natural binding partners (e.g., Cdc42 or Rac). In some instances, binding between PAK and at least one of its natural binding partners is stronger in the absence of a PAK inhibitor (by e.g., about 90%, about 80%, about 70%, about 60%, about 50%, about 40%, about 30% or about 20%) than in the presence of a PAK inhibitor. In some embodiments, PAK inhibitors prevent, reduce, or abolish binding between PAK and a protein that abnormally accumulates or aggregates in cells or tissue in a disease state. In some instances, binding between PAK and at least one of the proteins that aggregates or accumulates in a cell or tissue is stronger in the absence of a PAK inhibitor (by e.g., about 90%, about 80%, about 70%, about 60%, about 50%, about 40%, about 30% or about 20%) than in the presence of an inhibitor.

A “individual” or an “individual,” as used herein, is a mammal. In some embodiments, an individual is an animal, for example, a rat, a mouse, a dog or a monkey. In some embodiments, an individual is a human patient. In some embodiments a “individual” or an “individual” is a human. In some embodiments, an individual suffers from autism or is suspected to be suffering from autism or is pre-disposed to autism.

As used herein, the terms “autism,” and “Autistic Spectrum Disorders” are used interchangeably to refer to a category of neurological disorders characterized by severe and pervasive impairment in several areas of development, including social interaction and communications skills. The neurological disorders include: (i) Autistic Disorder (classic autism), (ii) Asperger's Disorder, (iii) Childhood Disintegrative Disorder (CDD), (iv) Rett's Disorder (Rett Syndrome), and (v) PDD—Not Otherwise Specified (PDD-NOS). Specific diagnostic criteria for each of these disorders can be found in the Diagnostic & Statistical Manual of Mental Disorders (DSM-IV-TR) as distributed by the American Psychiatric Association (APA). Additional diagnostic criteria are known in the art and include, but are not limited to, the measurement of symptoms indicative of autism including irritability, aggression, agitation, and stereotypy as measured by the Aberrant Behavior Checklist (ABC), the Ritvo-Freeman Real Life Rating Scale, and the compulsions scale from the Children's Yale-Brown Obsessive Compulsive Scale (CY-BOCS).

The term “Autistic Disorder” refers to a neurological and developmental disorder that usually appears during the first three years of life. Typically, a child with autism appears to live in his/her own world, showing little interest in others, and a lack of social awareness. Often, the focus of an autistic child is a consistent routine and includes an interest in repeating odd and peculiar behaviors. Autistic children generally present with problems in communication, avoid eye contact, and show limited attachment to others.

The term “Asperger's Disorder” is an autistic disorder which typically displays a substantial discrepancy between the intellectual and social abilities of those who have it. It is a pervasive developmental disorder that is typically characterized by an inability to understand how to interact socially while at the same time having normal intelligence. Typical features of the syndrome may also include clumsy and uncoordinated motor movements, social impairment with extreme egocentricity, limited interests and unusual preoccupations, repetitive routines or rituals, speech and language peculiarities, and non-verbal communication problems

The term “Rett Disorder”, as used herein, refers to neurodevelopmental disorder that is classified as an autism spectrum disorder by the DSM-IV. It most often affects girls and clinical features include a deceleration of the rate of head growth (including microcephaly in some) and small hands and feet. Behavioral symptoms include stereotypic, repetitive hand movements such as mouthing or wringing are also noted. For children with Rett Disorder, development is typically normal until 6-18 months, when language and motor milestones regress, purposeful hand use is lost and acquired deceleration in the rate of head growth (resulting in microcephaly in some) is seen. Additional behavioral symptoms can include breathing irregularities such as hyperventilation, breath holding, or sighing.

The term “compulsive behavior”, as used herein, refers to intentional behaviors which appear to follow rules, such as arranging objects in stacks or lines.

The term “ritualistic behavior”, as used herein, refers to behaviors exhibiting the need of a person to maintain an unvarying pattern of daily activities, such as a dressing ritual.

The term “restricted behavior”, as used herein, refers to behaviors exhibiting a limitation in focus, interest or activity, such as preoccupation with a single toy or game.

The term “stereotypy”, as used herein, refers to repetitive movements, such as hand flapping, making sounds, head rolling, or body rocking.

The term “sameness”, as used herein, refers to behaviors exhibiting a strong resistance to change in order.

The term “self-injury”, as used herein, refers to movements that can injure the person, such as eye poking, skin picking, hand biting, and/or head banging.

In some embodiments, a pharmacological composition comprising a PAK inhibitor is “administered peripherally” or “peripherally administered.” As used herein, these terms refer to any form of administration of an agent, e.g., a therapeutic agent, to an individual that is not direct administration to the CNS, i.e., that brings the agent in contact with the non-brain side of the blood-brain barrier. “Peripheral administration,” as used herein, includes intravenous, intra-arterial, subcutaneous, intramuscular, intraperitoneal, transdermal, by inhalation, transbuccal, intranasal, rectal, oral, parenteral, sublingual, or trans-nasal. In some embodiments, a PAK inhibitor is administered by an intracerebral route.

The terms “polypeptide,” and “protein” are used interchangeably herein to refer to a polymer of amino acid residues. That is, a description directed to a polypeptide applies equally to a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “isolated” and “purified” refer to a material that is substantially or essentially removed from or concentrated in its natural environment. For example, an isolated nucleic acid is one that is separated from the nucleic acids that normally flank it or other nucleic acids or components (proteins, lipids, etc.) in a sample. In another example, a polypeptide is purified if it is substantially removed from or concentrated in its natural environment. Methods for purification and isolation of nucleic acids and proteins are documented methodologies.

The term “antibody” describes an immunoglobulin whether natural or partly or wholly synthetically produced. The term also covers any polypeptide or protein having a binding domain which is, or is homologous to, an antigen-binding domain. CDR grafted antibodies are also contemplated by this term.

The term antibody as used herein will also be understood to mean one or more fragments of an antibody that retain the ability to specifically bind to an antigen, (see generally, Holliger et al., Nature Biotech. 23 (9) 1126-1129 (2005)). Non-limiting examples of such antibodies include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544 546), which consists of a VH domain; and (vi) an isolated complementarity determining region (CDR). Furthermore, although the two domains of the Fv fragment, VL and VH, are coded for by separate genes, they are optionally joined, using recombinant methods, by a synthetic linker that enables them to be made as a single protein chain in which the VL and VH regions pair to form monovalent molecules (known as single chain Fv (scFv); see e.g., Bird et al. (1988) Science 242:423 426; and Huston et al. (1988) Proc. Natl. Acad. Sci. USA 85:5879 5883; and Osbourn et al. (1998) Nat. Biotechnol. 16:778). Such single chain antibodies are also intended to be encompassed within the term antibody. Any VI-1 and VL sequences of specific scFv is optionally linked to human immunoglobulin constant region cDNA or genomic sequences, in order to generate expression vectors encoding complete IgG molecules or other isotypes. VH and VL are also optionally used in the generation of Fab, Fv or other fragments of immunoglobulins using either protein chemistry or recombinant DNA technology. Other forms of single chain antibodies, such as diabodies are also encompassed.

“F(ab′)₂” and “Fab′” moieties are optionally produced by treating immunoglobulin (monoclonal antibody) with a protease such as pepsin and papain, and includes an antibody fragment generated by digesting immunoglobulin near the disulfide bonds existing between the hinge regions in each of the two H chains. For example, papain cleaves IgG upstream of the disulfide bonds existing between the hinge regions in each of the two H chains to generate two homologous antibody fragments in which an L chain composed of VL (L chain variable region) and CL (L chain constant region), and an H chain fragment composed of VH (H chain variable region) and CHγ1 (γ1 region in the constant region of H chain) are connected at their C terminal regions through a disulfide bond. Each of these two homologous antibody fragments is called Fab′. Pepsin also cleaves IgG downstream of the disulfide bonds existing between the hinge regions in each of the two H chains to generate an antibody fragment slightly larger than the fragment in which the two above-mentioned Fab′ are connected at the hinge region. This antibody fragment is called F(ab′)2.

The Fab fragment also contains the constant domain of the light chain and the first constant domain (CHI) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are documented.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “sFv” antibody fragments comprise a VH, a VL, or both a VH and VL domain of an antibody, wherein both domains are present in a single polypeptide chain. In some embodiments, the Fv polypeptide further comprises a polypeptide linker between the VH and VL domains which enables the sFv to form the desired structure for antigen binding. For a review of sFv see, e.g., Pluckthun in The Pharmacology of Monoclonal Antibodies, Vol. 113, Rosenburg and Moore eds. Springer-Verlag, New York, pp. 269 315 (1994).

A “chimeric” antibody includes an antibody derived from a combination of different mammals. The mammal is, for example, a rabbit, a mouse, a rat, a goat, or a human. The combination of different mammals includes combinations of fragments from human and mouse sources.

In some embodiments, an antibody described or described herein is a monoclonal antibody (MAb), typically a chimeric human-mouse antibody derived by humanization of a mouse monoclonal antibody. Such antibodies are obtained from, e.g., transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. In some embodiments, the transgenic mice synthesize human antibodies specific for human antigens, and the mice are used to produce human antibody-secreting hybridomas.

The term “optionally substituted” or “substituted” means that the referenced group substituted with one or more additional group(s). In certain embodiments, the one or more additional group(s) are individually and independently selected from amide, ester, alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, amido.

An “alkyl” group refers to an aliphatic hydrocarbon group. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “lower alkyl” is a C₁-C₆ alkyl. A “heteroalkyl” group substitutes any one of the carbons of the alkyl group with a heteroatom having the appropriate number of hydrogen atoms attached (e.g., a CH₂ group to an NH group or an 0 group).

An “alkoxy” group refers to a (alkyl)O— group, where alkyl is as defined herein.

The term “alkylamine” refers to the —N(alkyl)ₓH_{y} group, wherein alkyl is as defined herein and x and y are selected from the group x=1, y=1 and x=2, y=0. When x=2, the alkyl groups, taken together with the nitrogen to which they are attached, optionally form a cyclic ring system.

An “amide” is a chemical moiety with formula C(O)NHR or NHC(O)R, where R is selected from alkyl, cycloalkyl, aryl, heteroaryl (bonded through a ring carbon) and heteroalicyclic (bonded through a ring carbon).

The term “ester” refers to a chemical moiety with formula —C(═O)OR, where R is selected from the group consisting of alkyl, cycloalkyl, aryl, heteroaryl and heteroalicyclic.

As used herein, the term “aryl” refers to an aromatic ring wherein each of the atoms forming the ring is a carbon atom. Aryl rings described herein include rings having five, six, seven, eight, nine, or more than nine carbon atoms. Aryl groups are optionally substituted. Examples of aryl groups include, but are not limited to phenyl, and naphthalenyl.

The term “cycloalkyl” refers to a monocyclic or polycyclic non-aromatic radical, wherein each of the atoms forming the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

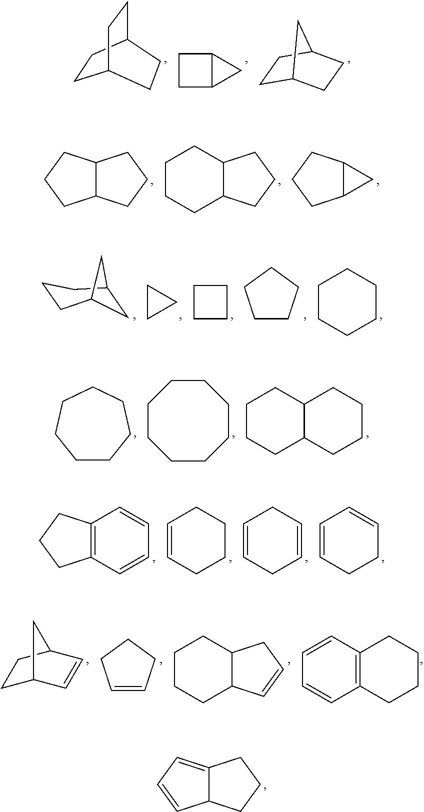

and the like. Monocyclic cycloalkyls include, but are not limited to, cyclopropyl, cyclobutyl, cyclopentyl, cyclohexyl, cycloheptyl, and cyclooctyl. Dicylclic cycloalkyls include, but are not limited to tetrahydronaphthyl, indanyl, tetrahydropentalene or the like. Polycyclic cycloalkyls include admantane, norbornane or the like. The term cycloalkyl includes “unsaturated nonaromatic carbocyclyl” or “nonaromatic unsaturated carbocyclyl” groups both of which refer to a nonaromatic carbocycle, as defined herein, that contains at least one carbon carbon double bond or one carbon carbon triple bond.

The term “heterocyclo” refers to heteroaromatic and heteroalicyclic groups containing one to four ring heteroatoms each selected from O, S and N. In certain instances, each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydropyranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are monocyclic or polycyclic. Examples of monocyclic heteroaryl groups include and are not limited to:

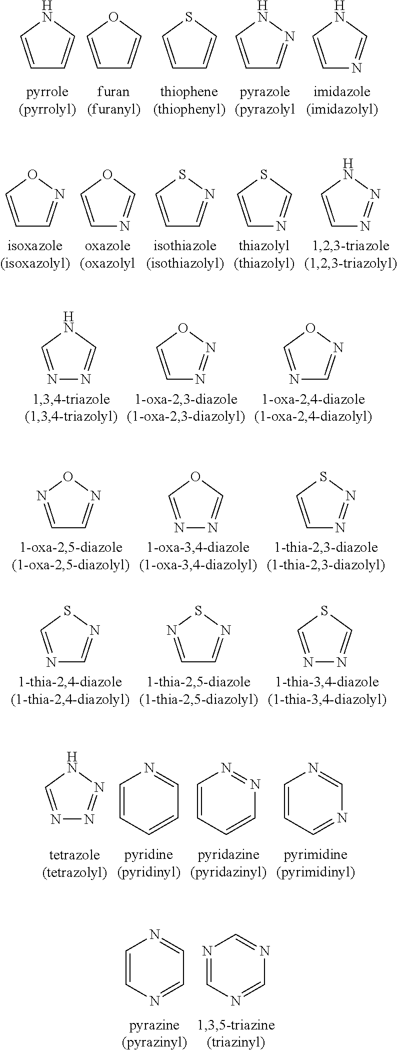

Examples of bicyclic heteroaryl groups include and are not limited to:

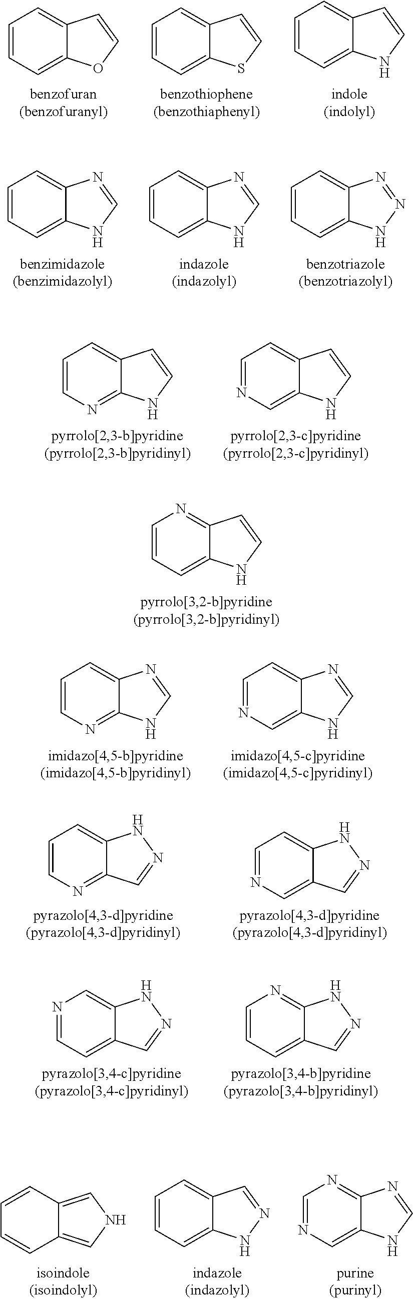

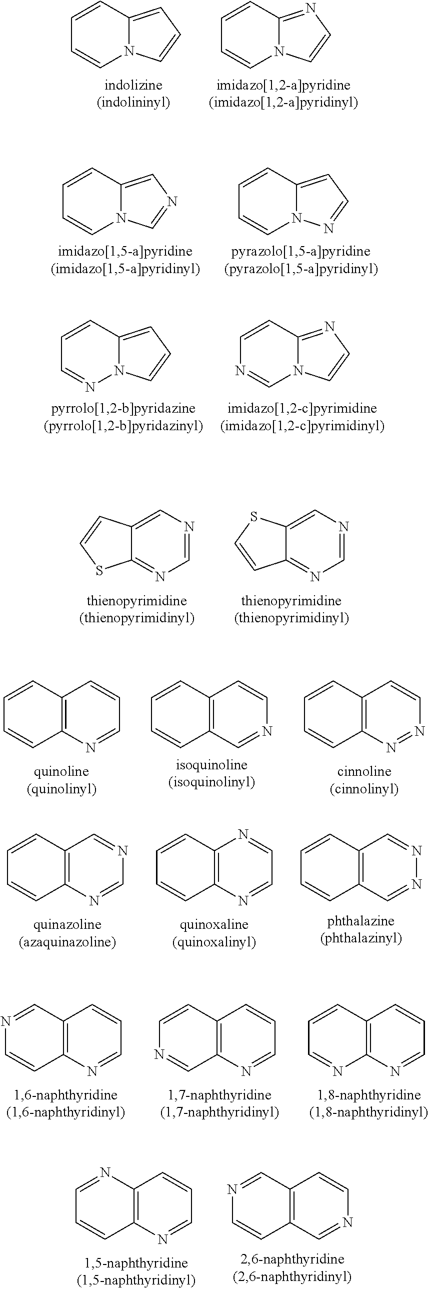

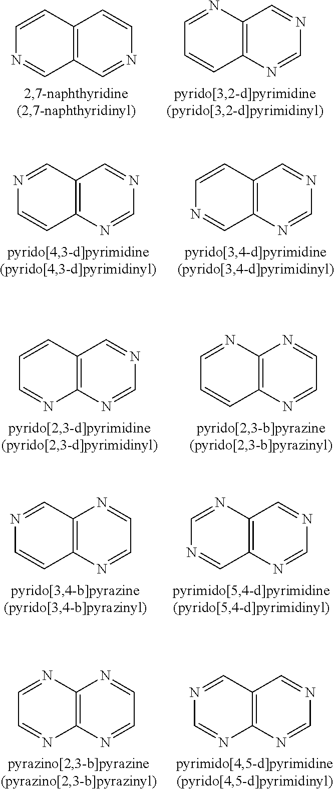

or the like.

A “heteroalicyclic” group or “heterocyclo” group or “heterocycloalkyl” group or “heterocyclyl” group refers to a cycloalkyl group, wherein at least one skeletal ring atom is a heteroatom selected from nitrogen, oxygen and sulfur. In some embodiments, the radicals are fused with an aryl or heteroaryl. Example of saturated heterocyloalkyl groups include

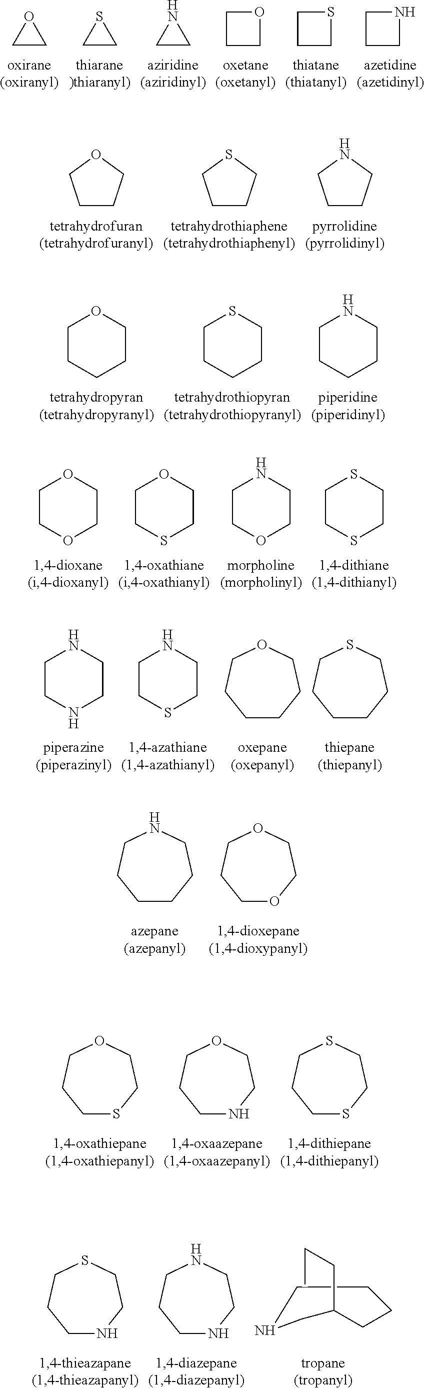

Examples of partially unsaturated heterocyclyl groups include

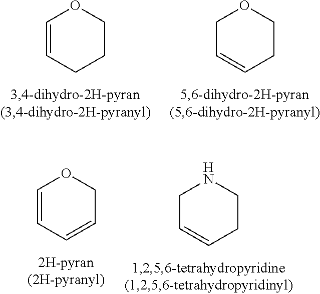 [SMILES: C1=COCCC1 ; C1=CCOCC1 ; C1=CCOC=C1 ; C1=CCNCC1]

Other illustrative examples of heterocyclo groups, also referred to as non-aromatic heterocycles, include:

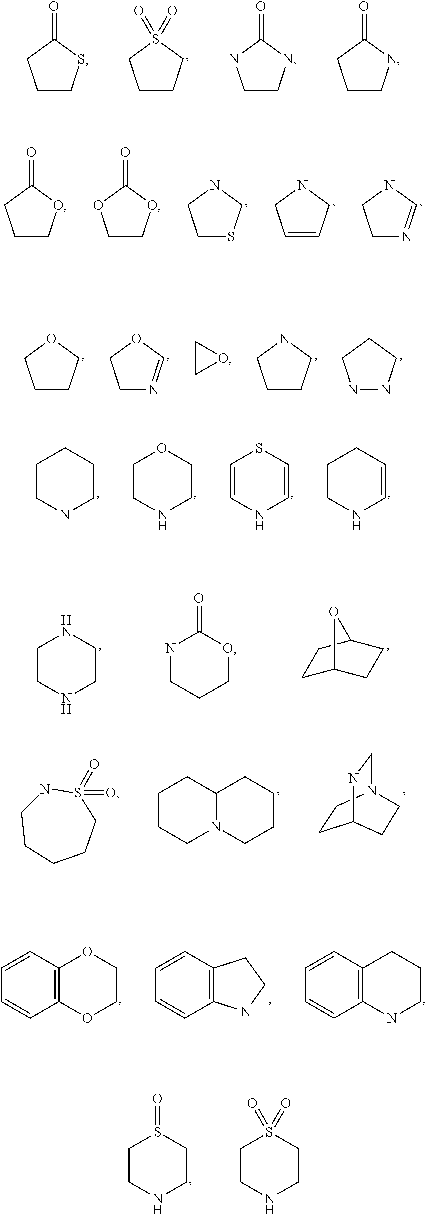

or the like.

The term heteroalicyclic also includes all ring forms of the carbohydrates, including but not limited to the monosaccharides, the disaccharides and the oligosaccharides.

The term “halo” or, alternatively, “halogen” means fluoro, chloro, bromo and iodo.

The terms “haloalkyl,” and “haloalkoxy” include alkyl and alkoxy structures that are substituted with one or more halogens. In embodiments, where more than one halogen is included in the group, the halogens are the same or they are different. The terms “fluoroalkyl” and “fluoroalkoxy” include haloalkyl and haloalkoxy groups, respectively, in which the halo is fluorine.

The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃.

A “cyano” group refers to a CN group.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

### Methods

Provided herein are methods of treating one or more symptoms of autism comprising administration of a therapeutically effective amount of a p21-activated kinase inhibitor (e.g., a compound of Formula I-XXIII) to an individual in need thereof. In some embodiments of the methods provided herein administration of a p21-activated kinase inhibitor stabilizes, alleviates, delays onset of, inhibits progression of, or reduces the severity of at least one symptom associated with autism. In some embodiments, the administration of a PAK inhibitors described herein alleviates, ameliorates, delays onset of, inhibits progression of, or reduces the severity of at least one behavorial symptom associated with autism.

In some embodiments, the PAK inhibitors described herein alleviate, ameliorate, delay onset of, inhibit progression of, or reduce the severity of, one or more of the following behavorial traits or symptoms: (i) insistence on sameness or resistance to change; (ii) difficulty in expressing needs (i.e. uses gestures or pointing instead of words); (iii) repeating words or phrases in place of normal, responsive language; (iv) laughing, crying, showing distress for reasons not apparent to others; (v) prefers to be alone or aloof manner; (vi) tantrums; (vii) difficulty in mixing with others; (viii) may not want to cuddle or be cuddled; (ix) little or no eye contact; (x) unresponsive to normal teaching methods; (xi) sustained odd play (e.g., spins objects and/or inappropriate attachments to objects); (xii) apparent over-sensitivity or under-sensitivity to pain; (xiii) little or no real fears of danger; (xiv) noticeable physical over-activity or extreme under-activity; (xv) uneven gross/fine motor skills; and/or (xvi) non-responsiveness to verbal cues (i.e., acts as if deaf although hearing tests in normal range).

In some embodiments, the administration of PAK inhibitors described herein alleviate, ameliorate, delay onset of, inhibit progression of, or reduce the severity of compulsive behavior associated with autism. In some embodiments, the administration of PAK inhibitors described herein alleviate, ameliorate, delay onset of, inhibit progression of, or reduce the severity of ritualistic behavior associated with autism. In some embodiments, the administration of PAK inhibitors described herein alleviate, ameliorate, delay onset of, inhibit progression of, or reduce the severity of restricted behavior associated with autism. In some embodiments, the administration of PAK inhibitors described herein alleviate, ameliorate, delay onset of, inhibit progression of, or reduce the severity of stereotypy associated with autism. In some embodiments, the administration of PAK inhibitors described herein alleviate, ameliorate, delay onset of, inhibit progression of, or reduce the severity of “sameness” associated with autism. In some embodiments, the administration of PAK inhibitors described herein alleviate, ameliorate, delay onset of, inhibit progression of, or reduce the severity of self-injury behavior associated with autism. In some embodiments, the administration of PAK inhibitors described herein alleviate, ameliorate, delay onset of, inhibit progression of, or reduce the severity of one or more behavioral symptoms associated with autism.

Also provided herein are methods for modulation of dendritic spine morphology and/or synaptic function comprising administering to an individual in need thereof (e.g., an individual suffering from autism) a therapeutically effective amount of a PAK inhibitor (e.g., a compound of Formula I-XXIII). In some embodiments, modulation of dendritic spine morphology and/or synaptic function stabilizes, alleviates or reverses behavioral symptoms associated with autism. In some embodiments, modulation of dendritic spine morphology and/or synaptic function halts or delays progression of behavioral symptoms associated with autism.

Provided herein are methods for modulation of synaptic function or synaptic plasticity comprising administering to an individual in need thereof (e.g., an individual suffering from autism) a therapeutically effective amount of a PAK inhibitor (e.g., a compound of Formula I-XXIII). Modulation of synaptic function or plasticity includes, for example, stabilization, alleviation or reversal of defects in LTP, LTD or the like.

Defects in LTP include, for example, an increase in LTP or a decrease in LTP in any region of the brain in an individual suffering from autism. Defects in LTD include for example a decrease in LTD or an increase in LTD in any region of the brain (e.g., the cerebellum, temporal lobe, parietal lobe, the frontal cortex, the cingulate gyrus, the prefrontal cortex, the cortex, or the hippocampus or any other region in the brain or a combination thereof) in an individual suffering from autism.

In some embodiments of the methods, administration of a PAK inhibitor (e.g., a compound of Formula I-XXIII) modulates synaptic function (e.g., synaptic transmission and/or plasticity) by increasing long term potentiation (LTP) in an individual suffering from autism. In some embodiments of the methods described herein, administration of a PAK inhibitor (e.g., a compound of Formula I-XXIII) to an individual in need thereof modulates synaptic function (e.g., synaptic transmission and/or plasticity) by increasing long term potentiation (LTP) in the prefrontal cortex, or the cortex, or the hippocampus or any other region in the brain or a combination thereof. In some embodiments of the methods described herein, administration of a PAK inhibitor modulates synaptic function (e.g., synaptic transmission and/or plasticity) by decreasing long term depression (LTD) in an individual suffering from autism. In some embodiments of the methods described herein, administration of a PAK inhibitor to an individual in need thereof modulates synaptic function (e.g., synaptic transmission and/or plasticity) by decreasing long term depression (LTD) in the cerebellum, temporal lobe, parietal lobe, the frontal cortex, the cingulate gyrus, the prefrontal cortex, the cortex, or the hippocampus or any other region in the brain or a combination thereof.

Provided herein are methods for stabilization and/or normalization and/or partial normalization of synaptic plasticity comprising the administration to an individual in need thereof (e.g., an individual suffering from autism) a therapeutically effective amount of a PAK inhibitor (e.g., a compound of Formula I-XXIII). In some embodiments of the methods described herein, administration of a PAK inhibitor (e.g., a compound of Formula I-XXIII) stabilizes LTP or LTD following induction (e.g., by theta-burst stimulation, high-frequency stimulation).

Provided herein are methods for stabilization and/or normalization and/or partial normalization of synaptic transmission comprising the administration to an individual in need thereof (e.g., in an individual suffering from autism) a therapeutically effective amount of a PAK inhibitor (e.g., a compound of Formula I-XXIII). In some embodiments of the methods described herein, administration of a PAK inhibitor (e.g., a compound of Formula I-XXIII) stabilizes LTP or LTD following induction (e.g., by theta-burst stimulation, high-frequency stimulation).

Provided herein are methods for stabilizing, reducing or reversing abnormalities in dendritic spine morphology or synaptic function that may be caused by mutations in high-risk genes that predispose an individual for developing autism, e.g., mutations at the 15q11-q13 locus (“chromosome 15 phenotype”), including the GABA_{A} receptor gene cluster; mutations at the q22-q33 region of chromosome 7, including the reelin gene, FOXP2, NPTX2, IMMP2L, RAY1/ST7, GRM8, CADPS2, and WNT2; NLGN3 and NLGN4; CDH9 and CDH10; CNTNAP2; the SHANK gene family of genes, PCDH10, Neurexin 1 (NRX1), NHE9/SLC9A9, DIA1, SCN7A, contactin 3, MeCP2, A2BP1C, UBE3A, SCN7A, or any other high-risk gene that is known to pre-dispose an individual to autism comprising administering to an individual in need thereof a therapeutically effective amount of a PAK inhibitor (e.g., a compound of Formula I-XXIII). In some embodiments of the methods described herein, prophylactic administration of a PAK inhibitor to an individual at a high risk for developing autism (e.g., an individual with a mutation in the 15q11-q13 locus or a high-risk allele that pre-disposes the individual to autism) reverses abnormalities in dendritic spine morphology and/or synaptic function and prevents development of autism. In some embodiments, methods are provided herein for halting or delaying the onset of autism comprising administering to an individual in need thereof (e.g., an individual with a mutation in the 15q11-q13 locus, or an individual with a high-risk mutation) a therapeutically effective amount of a PAK inhibitor (e.g., a compound of Formula I-XXIII). Provided herein are methods for delaying the loss of dendritic spine density comprising administering to an individual in need thereof (e.g., an individual with a mutation in the 15q11-q13 locus, or an individual with a high-risk mutation) a therapeutically effective amount of a PAK inhibitor (e.g., a compound of Formula I-XXIII).

Provided herein are methods for stabilizing, reducing or reversing abnormalities in dendritic spine morphology or synaptic function that caused by increased activation of PAK at the synapse, comprising administering of a therapeutically effective amount of a PAK inhibitor (e.g., a compound of Formula I-XXIII) to an individual in need thereof (e.g., an individual suffering from or suspected of having autism).

Provided herein are methods for stabilizing, reducing or reversing neuronal withering and/or atrophy or nervous tissue and/or degeneration of nervous tissue that is associated with autism. In some embodiments of the methods described herein, administration of a PAK inhibitor (e.g., a compound of Formula I-XXIII) to an individual suffering from autism stabilizes, alleviates or reverses neuronal withering and/or atrophy and/or degeneration in the cerebellum, temporal lobe, parietal lobe, the frontal cortex, the cingulate gyrus or the like.

Provided herein are methods for modulation of spine density, shape, spine length, spine head diameter, spine head volume, or spine neck diameter or the like comprising administering to an individual in need thereof (e.g., an individual suffering from autism) a therapeutically effective amount of a PAK inhibitor (e.g., a compound of Formula I-XXIII). Provided herein are methods of modulating the ratio of mature dendritic spines to immature dendritic spines comprising administering to an individual in need thereof (e.g., an individual suffering from autism) a therapeutically effective amount of a PAK inhibitor (e.g., a compound of Formula I-XXIII). Provided herein are methods of modulating the ratio of dendritic spines head volume to dendritic spines length comprising administering to an individual in need thereof (e.g., an individual suffering from autism) a therapeutically effective amount of a PAK inhibitor (e.g., a compound of Formula I-XXIII).

In some embodiments of the methods described herein, administration of a PAK inhibitor (e.g., a maintenance dose of a PAK inhibitor) halts or delays the progression of autism symptoms or pathologies in an individual. In some embodiments of the methods described herein, administration of a PAK inhibitor causes substantially complete inhibition of PAK and restores dendritic spine morphology and/or synaptic function to normal or partially normal levels. In some embodiments of the methods described herein, administration of a PAK inhibitor causes partial inhibition of PAK and restores dendritic spine morphology and/or synaptic function to normal or partially normal levels.

In some instances, autism is associated with a decrease in dendritic spine density. In some embodiments of the methods described herein, administration of a PAK inhibitor increases dendritic spine density. In some instances, autism is associated with an increase in dendritic spine length. In some embodiments of the methods described herein, administration of a PAK inhibitor decreases dendritic spine length. In some instances, autism is associated with a decrease in dendritic spine head diameter. In some embodiments of the methods described herein, administration of a PAK inhibitor increases dendritic spine head diameter. In some instances, autism is associated with a decrease in dendritic spine neck diameter. In some embodiments of the methods described herein, administration of a PAK inhibitor increases dendritic spine neck diameter. In some instances, autism is associated with a decrease in dendritic spine head volume and/or dendritic spine head surface area. In some embodiments of the methods described herein, administration of a PAK inhibitor increases dendritic spine head volume and/or dendritic spine head surface area.

In some instances, autism is associated with an increase in immature spines and/or a decrease in mature spines. In some embodiments of the methods described herein, administration of a PAK inhibitor modulates the ratio of immature spines to mature spines. In some instances, autism is associated with an increase in stubby spines and a decrease in mushroom-shaped spines. In some embodiments of the methods described herein, administration of a PAK inhibitor modulates the ratio of stubby spines to mushroom-shaped spines.

In some embodiments of the methods described herein, administration of a PAK inhibitor modulates a spine:head ratio, e.g., ratio of the volume of the spine to the volume of the head, ratio of the length of a spine to the length of a head of the spine, ratio of the surface area of a spine to the surface area of the head of a spine, or the like, compared to a spine:head ratio in the absence of a PAK inhibitor. In certain embodiments, a PAK inhibitor suitable for the methods described herein modulates the volume of the spine head, the width of the spine head, the surface area of the spine head, the length of the spine shaft, the diameter of the spine shaft, or a combination thereof. In some embodiments, provided herein is a method of modulating the volume of a spine head, the width of a spine head, the surface area of a spine head, the length of a spine shaft, the diameter of a spine shaft, or a combination thereof, by contacting a neuron comprising the dendritic spine with an effective amount of a PAK inhibitor described herein. In specific embodiments, the neuron is contacted with the PAK inhibitor in vivo.

In certain embodiments, a compound or a composition comprising a compound described herein is administered for prophylactic and/or therapeutic treatments. In therapeutic applications, the compositions are administered to an individual already suffering from a disease or condition, in an amount sufficient to cure or at least partially arrest the symptoms of the disease or condition. In various instances, amounts effective for this use depend on the severity and course of the disease or condition, previous therapy, an individual's health status, weight, and response to the drugs, and the judgment of the treating physician.

In some embodiments, a composition containing a therapeutically effective amount of a PAK inhibitor is administered prophylactically to an individual that, while not overtly manifesting symptoms of autism, has been identified as having a high risk of developing autism, e.g., an individual is identified as being a carrier of a mutation or polymorphism associated with a higher risk to develop autism, or an individual that is from a family that has a high incidence of autism. In some instances, the typical age of onset for autism is prior to 3 years of age. Accordingly, in some embodiments, a PAK inhibitor is administered prophylactically to an individual at risk between about 1 to about 3 years, e.g., 1, 2, or 3 years prior to the established age range of onset for autism.

In prophylactic applications, compounds or compositions containing compounds described herein are administered to an individual susceptible to or otherwise at risk of a particular disease, disorder or condition. In certain embodiments of this use, the precise amounts of compound administered depend on an individual's state of health, weight, and the like. Furthermore, in some instances, when a compound or composition described herein is administered to an individual, effective amounts for this use depend on the severity and course of the disease, disorder or condition, previous therapy, an individual's health status and response to the drugs, and the judgment of the treating physician.

In certain instances, wherein following administration of a selected dose of a compound or composition described herein, an individual's condition does not improve, upon the doctor's discretion the administration of a compound or composition described herein is optionally administered chronically, that is, for an extended period of time, including throughout the duration of an individual's life in order to ameliorate or otherwise control or limit the symptoms of an individual's disorder, disease or condition.

In certain embodiments, an effective amount of a given agent varies depending upon one or more of a number of factors such as the particular compound, disease or condition and its severity, the identity (e.g., weight) of an individual or host in need of treatment, and is determined according to the particular circumstances surrounding the case, including, e.g., the specific agent being administered, the route of administration, the condition being treated, and an individual or host being treated. In some embodiments, doses administered include those up to the maximum tolerable dose. In certain embodiments, about 0.02-5000 mg per day, from about 1-1500 mg per day, about 1 to about 100 mg/day, about 1 to about 50 mg/day, or about 1 to about 30 mg/day, or about 5 to about 25 mg/day of a compound described herein is administered. In various embodiments, the desired dose is conveniently be presented in a single dose or in divided doses administered simultaneously (or over a short period of time) or at appropriate intervals, for example as two, three, four or more sub-doses per day.

In certain instances, there are a large number of variables in regard to an individual treatment regime, and considerable excursions from these recommended values are considered within the scope described herein. Dosages described herein are optionally altered depending on a number of variables such as, by way of non-limiting example, the activity of the compound used, the disease or condition to be treated, the mode of administration, the requirements of an individual, the severity of the disease or condition being treated, and the judgment of the practitioner.

Toxicity and therapeutic efficacy of such therapeutic regimens are optionally determined by pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. In certain embodiments, data obtained from cell culture assays and animal studies are used in formulating a range of dosage for use in human. In specific embodiments, the dosage of compounds described herein lies within a range of circulating concentrations that include the ED₅₀ with minimal toxicity. The dosage optionally varies within this range depending upon the dosage form employed and the route of administration utilized.

### Combination Therapy

In some embodiments, one or more PAK inhibitors are used in combination with one or more other therapeutic agents to treat an individual suffering from autism. The combination of PAK inhibitors with a second therapeutic agent (e.g., an anticholinergic agent) allows a reduced dose of both agents to be used thereby reducing the likelihood of side effects associated with higher dose monotherapies. In one embodiment, the dose of a second active agent (e.g., an anticholinergic) is reduced in the combination therapy by at least 50% relative to the corresponding monotherapy dose, whereas the PAK inhibitor dose is not reduced relative to the monotherapy dose; in further embodiments, the reduction in dose of a second active agent is at least 75%; in yet a further embodiment, the reduction in dose of a second active agent is at least 90%. In some embodiments, the second therapeutic agent is administered at the same dose as a monotherapy dose, and the addition of a PAK inhibitor to the treatment regimen alleviates symptoms of autism that are not treated by monotherapy with the second therapeutic agent.

In some embodiments, the combination of a PAK inhibitor and a second therapeutic agent is synergistic (e.g., the effect of the combination is better than the effect of each agent alone). In some embodiments, the combination of a PAK inhibitor and a second therapeutic agent is additive (e.g., the effect of the combination of active agents is about the same as the effect of each agent alone). In some embodiments, an additive effect is due to the PAK inhibitor and the second therapeutic agent modulating the same regulatory pathway. In some embodiments, an additive effect is due to the PAK inhibitor and the second therapeutic agent modulating different regulatory pathways. In some embodiments, an additive effect is due to the PAK inhibitor and the second therapeutic agent treating different symptom groups of autism (e.g., a PAK inhibitor treats cognitive symptoms and the second therapeutic agent treats loss of acetylcholine due to death of cholinergic neurons). In some embodiments, administration of a second therapeutic agent treats the remainder of the same or different symptoms or groups of symptoms that are not treated by administration of a PAK inhibitor alone.

In some embodiments, administration of a combination of a PAK inhibitor and a second therapeutic agent alleviates side effects that are caused by the second therapeutic agent (e.g., side effects caused by an anti-psychotic agent). In some embodiments, administration of the second therapeutic agent inhibits metabolism of an administered PAK inhibitor (e.g., the second therapeutic agent blocks a liver enzyme that degrades the PAK inhibitor) thereby increasing efficacy of a PAK inhibitor. In some embodiments, administration of a combination of a PAK inhibitor and a second therapeutic agent (e.g. a second agent that modulates dendritic spine morphology (e.g., minocyline)) improves the therapeutic index of a PAK inhibitor.

### Anti-Psychotic Agents

Where a subject is suffering from or at risk of suffering from autism, a PAK inhibitor composition described herein is optionally used together with one or more agents or methods for treating autism in any combination. In some embodiments, a PAK inhibitor composition described herein is administered to a patient in combination with an antipsychotic agent. Examples of antipsychotic agents include, for example, Droperidol Chlorpromazine (Largactil, Thorazine), Fluphenazine (Prolixin), Haloperidol (Haldol, Serenace), Molindone, Thiothixene (Navane), Thioridazine (Mellaril), Trifluoperazine (Stelazine), Loxapine, Perphenazine, Prochlorperazine (Compazine, Buccastem, Stemetil), Pimozide (Orap), Zuclopenthixol; LY2140023, Clozapine, Risperidone, Olanzapine, Quetiapine, Ziprasidone, Aripiprazole, Paliperidone, Asenapine, Iloperidone, Sertindole, Zotepine, Amisulpride, Bifeprunox, Melperone or the like.

### Serotonin Re-Uptake Inhibitors

Where a subject is suffering from or at risk of suffering from autism, a PAK inhibitor composition described herein is optionally used together with one or more agents or methods for treating autism in any combination. In some embodiments, a PAK inhibitor composition described herein is administered to a patient in combination with a serotonin re-uptake inhibitor. Examples of serotonin re-uptake inhibitors include, for example, clomipramine (Anafranil), citalopram (Celexa), escitalopram (Lexapro), fluoxetine (Prozac), fluvoxamine (Luvox), paroxetine (Paxil), sertraline (Zoloft), zimelidine (Zelmid) or the like.

### Stimulants

Where a subject is suffering from or at risk of suffering from autism, a PAK inhibitor composition described herein is optionally used together with one or more agents or methods for treating autism in any combination. In some embodiments, a PAK inhibitor composition described herein is administered to a patient in combination with a stimulant. Examples of stimulants include, for example, methylphenidate (Ritalin), dexmethylphenidate HCl (Focalin), dextroamphetamine sulfate (Dexedine), mixed salts amphetamine (Adderall) or the like.

### NMDA Receptor Antagonists

Where a subject is suffering from or at risk of suffering from autism, a PAK inhibitor composition described herein is optionally used together with one or more agents or methods for treating autism in any combination. In some embodiments, a PAK inhibitor composition described herein is administered to a patient who has been prescribed an NMDA receptor antagonist. Examples of NMDA receptor antagonists useful in the methods and compositions described herein include and are not limited to amantadine, memantine, tramadol (Ultracet) or the like.

### Dopamine Receptor Agonists

Where a subject is suffering from or at risk of suffering from autism, a PAK inhibitor composition described herein is optionally used together with one or more agents or methods for treating autism in any combination. In some embodiments, a PAK inhibitor composition described herein is administered to a patient in combination with a dopamine receptor agonist bromocriptine (Parlodel), cabergoline (Dostinex), piribedil (Trivastal), pramipexole (Mirapex), ropinirole (Requip), apomorphine (Apokyn), rotigotine (Neupro) or the like.

### Antioxidants

Where a subject is suffering from or at risk of suffering from autism, a PAK inhibitor composition described herein is optionally used together with one or more agents or methods for treating autism in any combination. In some embodiments, a PAK inhibitor composition described herein is administered to a patient who is taking or has been prescribed an antioxidant. Examples of antioxidants useful in the methods and compositions described herein include and are not limited to ubiquinone, aged garlic extract, curcumin, lipoic acid, beta-carotene, melatonin, resveratrol, Ginkgo biloba extract, vitamin C, vitamin E or the like.

### Neuroprotectants

In some embodiments, a PAK inhibitor or a composition thereof described herein is administered in combination with a neuroprotectant such as, for example, minocycline, resveratrol or the like.

### Trophic Factors

In some embodiments, a PAK inhibitor or a composition thereof described herein is administered in combination with a trophic agent including, by way of example, glial derived nerve factor (GDNF), brain derived nerve factor (BDNF) or the like.

### Metal Protein Attenuating Compounds

In some embodiments, a PAK inhibitor composition described herein is optionally used together with one or more agents or methods for treating autism in any combination. In some embodiments, a PAK inhibitor composition described herein is administered to a patient who has been prescribed a Metal Protein Attenuating agent. Examples of Metal Protein Attenuating agents useful in the methods and compositions described herein include and are not limited to 8-Hydroxyquinoline, iodochlorhydroxyquin or the like and derivatives thereof.

### Beta-Secretase Inhibitors

In some embodiments, a PAK inhibitor composition described herein is optionally used together with one or more agents or methods for treating autism in any combination. In some embodiments, a PAK inhibitor composition described herein is administered to a patient who has been prescribed a beta secretase inhibitor. Examples of beta secretase inhibitors useful in the methods and compositions described herein include and are not limited to LY450139, 2-Aminoquinazolines compounds described in J. Med. Chem. 50 (18): 4261-4264, beta secretase inhibitors described therein are incorporated herein by reference, or the like.

### Gamma Secretase Inhibitors

In some embodiments, a PAK inhibitor composition described herein is optionally used together with one or more agents or methods for treating autism in any combination. In some embodiments, a PAK inhibitor composition described herein is administered to a patient who has been prescribed a beta secretase inhibitor. Examples of beta secretase inhibitors useful in the methods and compositions described herein include and are not limited to LY-411575, (2S)-2-hydroxy-3-methyl-N-((15)-1-methyl-2-([(15)-3-methyl-2-oxo-2,3,4,5-tetrahydro-1H-3-benzazepin-1-yl]amino-2-oxoethyl)butanamide (semagacestat), (R)-2-(3-Fluoro-4-phenylphenyl)propanoic acid (Tarenflurbil), or the like.

### Antibodies

In some embodiments, a PAK inhibitor composition described herein is optionally used together with one or more agents or methods for treating autism in any combination. In some embodiments, a PAK inhibitor composition described herein is administered to a patient who has been prescribed an Abeta antibody. Examples of antibodies useful in the methods and compositions described herein include and are not limited to PAK antibodies (e.g., AB1N237914) or the like.

### Other Agents

In some embodiments, one or more PAK inhibitors are used in combination with one or more agents that treat behavioral symptoms of autism. Examples of agents that modulate behavioral symptoms are Elavil, Wellbutrin, Valium and other benzidiazapine-based agents modulating GABA receptors, Ativan and Xanax. In some embodiments, one or more PAK inhibitors are used in combination with one or more agents that modulate dendritic spine morphology or synaptic function. Examples of agents that modulate dendritic spine morphology include minocycline, trophic factors (e.g., brain derived neutrophic factor, glial cell-derived neurtrophic factor), or anesthetics that modulate spine motility, or the like. In some embodiments, one or more PAK inhibitors are used in combination with one or more agents that modulate cognition. In some embodiments, a second therapeutic agent is a nootropic agent that enhances cognition. Examples of nootropic agents include and are not limited to piracetam, pramiracetam, oxiracetam, and aniracetam.

### Blood Brain Barrier Facilitators

In some instances, a PAK inhibitor is optionally administered in combination with a blood brain barrier facilitator. In certain embodiments, an agent that facilitates the transport of a PAK inhibitor is covalently attached to the PAK inhibitor. In some instances, PAK inhibitors described herein are modified by covalent attachment to a lipophilic carrier or co-formulation with a lipophilic carrier. In some embodiments, a PAK inhibitor is covalently attached to a lipophilic carrier, such as e.g., DHA, or a fatty acid. In some embodiments, a PAK inhibitor is covalently attached to artificial low density lipoprotein particles. In some instances, carrier systems facilitate the passage of PAK inhibitors described herein across the blood-brain barrier and include but are not limited to, the use of a dihydropyridine pyridinium salt carrier redox system for delivery of drug species across the blood brain barrier. In some instances a PAK inhibitor described herein is coupled to a lipophilic phosphonate derivative. In certain instances, PAK inhibitors described herein are conjugated to PEG-oligomers/polymers or aprotinin derivatives and analogs. In some instances, an increase in influx of a PAK inhibitor described herein across the blood brain barrier is achieved by modifying A PAK inhibitor described herein (e.g., by reducing or increasing the number of charged groups on the compound) and enhancing affinity for a blood brain barrier transporter. In certain instances, a PAK inhibitor is co-administered with an an agent that reduces or inhibits efflux across the blood brain barrier, e.g. an inhibitor of P-glycoprotein pump (PGP) mediated efflux (e.g., cyclosporin, SCH66336 (lonafarnib, Schering)).

In some instances, a PAK inhibitor polypeptide is delivered to one or more brain regions of an individual by administration of a viral expression vector, e.g., an AAV vector, a lentiviral vector, an adenoviral vector, or a HSV vector. A number of viral vectors for delivery of therapeutic proteins are described in, e.g., U.S. Pat. Nos. 7,244,423, 6,780,409, 5,661,033. In some embodiments, the PAK inhibitor polypeptide to be expressed is under the control of an inducible promoter (e.g., a promoter containing a tet-operator). Inducible viral expression vectors include, for example, those described in U.S. Pat. No. 6,953,575. Inducible expression of a PAK inhibitor polypeptide allows for tightly controlled and reversible increases of PAK inhibitor polypeptide expression by varying the dose of an inducing agent (e.g., tetracycline) administered to an individual.

Any combination of one or more PAK inhibitors and a second therapeutic agent is compatible with any method described herein. The PAK inhibitor compositions described herein are also optionally used in combination with other therapeutic reagents that are selected for their therapeutic value for the condition to be treated. In general, the compositions described herein and, in embodiments where combinational therapy is employed, other agents do not have to be administered in the same pharmaceutical composition, and, because of different physical and chemical characteristics, are optionally administered by different routes. The initial administration is generally made according to established protocols, and then, based upon the observed effects, the dosage, modes of administration and times of administration subsequently modified.

In certain instances, it is appropriate to administer at least one PAK inhibitor composition described herein in combination with another therapeutic agent. By way of example only, if one of the side effects experienced by a patient upon receiving one of the PAK inhibitor compositions described herein is nausea, then it is appropriate to administer an anti-nausea agent in combination with the initial therapeutic agent. Or, by way of example only, the therapeutic effectiveness of a PAK inhibitor is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by a patient is increased by administering a PAK inhibitor with another therapeutic agent (which also includes a therapeutic regimen) that also has therapeutic benefit. In any case, regardless of the disease, disorder or condition being treated, the overall benefit experienced by the patient is either simply additive of the two therapeutic agents or the patient experiences a synergistic benefit.

Therapeutically-effective dosages vary when the drugs are used in treatment combinations. Suitable methods for experimentally determining therapeutically-effective dosages of drugs and other agents include, e.g., the use of metronomic dosing, i.e., providing more frequent, lower doses in order to minimize toxic side effects. Combination treatment further includes periodic treatments that start and stop at various times to assist with the clinical management of the patient.

In any case, the multiple therapeutic agents (one of which is a PAK inhibitor described herein) are administered in any order, or even simultaneously. If simultaneously, the multiple therapeutic agents are optionally provided in a single, unified form, or in multiple forms (by way of example only, either as a single pill or as two separate pills). In some embodiments, one of the therapeutic agents is given in multiple doses, or both are given as multiple doses. If not simultaneous, the timing between the multiple doses optionally varies from more than zero weeks to less than four weeks. In addition, the combination methods, compositions and formulations are not to be limited to the use of only two agents; the use of multiple therapeutic combinations is also envisioned.

The pharmaceutical agents which make up the combination therapy disclosed herein are optionally a combined dosage form or in separate dosage forms intended for substantially simultaneous administration. The pharmaceutical agents that make up the combination therapy are optionally also be administered sequentially, with either therapeutic compound being administered by a regimen calling for two-step administration. The two-step administration regimen optionally calls for sequential administration of the active agents or spaced-apart administration of the separate active agents. The time period between the multiple administration steps ranges from, a few minutes to several hours, depending upon the properties of each pharmaceutical agent, such as potency, solubility, bioavailability, plasma half-life and kinetic profile of the pharmaceutical agent. Circadian variation of the target molecule concentration can optionally be used to determine the optimal dose interval.

In addition, a PAK inhibitor is optionally used in combination with procedures that provide additional or synergistic benefit to the patient. By way of example only, patients are expected to find therapeutic and/or prophylactic benefit in the methods described herein, wherein pharmaceutical composition of a PAK inhibitor and/or combinations with other therapeutics are combined with genetic testing to determine whether that individual is a carrier of a mutant gene that is correlated with autism.

A PAK inhibitor and the additional therapy(ies) are optionally administered before, during or after the occurrence of a disease or condition, and the timing of administering the composition containing a PAK inhibitor varies in some embodiments. Thus, for example, the PAK inhibitor is used as a prophylactic and administered continuously to individuals with a propensity to develop conditions or diseases in order to prevent the occurrence of the disease or condition. The PAK inhibitors and compositions are optionally administered to an individual during or as soon as possible after the onset of the symptoms. The administration of the compounds are optionally initiated within the first 48 hours of the onset of the symptoms, preferably within the first 48 hours of the onset of the symptoms, more preferably within the first 6 hours of the onset of the symptoms, and most preferably within 3 hours of the onset of the symptoms. The initial administration is optionally via any route practical, such as, for example, an intravenous injection, a bolus injection, infusion over 5 minutes to about 5 hours, a pill, a capsule, transdermal patch, buccal delivery, and the like, or combination thereof. A PAK inhibitor is optionally administered as soon as is practicable after the onset of a disease or condition is detected or suspected, and for a length of time necessary for the treatment of the disease, such as, for example, from about 1 month to about 3 months. The length of treatment optionally varies for each individual, and the length is then determined using the known criteria. For example, the PAK inhibitor or a formulation containing the PAK inhibitor is administered for at least 2 weeks, preferably about 1 month to about 5 years, and more preferably from about 1 month to about 3 years.

In some embodiments, the particular choice of compounds depends upon the diagnosis of the attending physicians and their judgment of the condition of an individual and the appropriate treatment protocol. The compounds are optionally administered concurrently (e.g., simultaneously, essentially simultaneously or within the same treatment protocol) or sequentially, depending upon the nature of the disease, disorder, or condition, the condition of an individual, and the actual choice of compounds used. In certain instances, the determination of the order of administration, and the number of repetitions of administration of each therapeutic agent during a treatment protocol, is based on an evaluation of the disease being treated and the condition of an individual.

In some embodiments, therapeutically-effective dosages vary when the drugs are used in treatment combinations. Methods for experimentally determining therapeutically-effective dosages of drugs and other agents for use in combination treatment regimens are described in the literature.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a therapeutic compound described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing, e.g., from more than zero weeks to less than four weeks. In some embodiments, the additional therapeutic agent is utilized to achieve reversal or amelioration of autism, whereupon the therapeutic agent described herein (e.g., a compound of any one of Formulas I-XXIII) is subsequently administered. In addition, the combination methods, compositions and formulations are not to be limited to the use of only two agents; the use of multiple therapeutic combinations are also envisioned (including two or more compounds described herein).

In certain embodiments, a dosage regimen to treat, prevent, or ameliorate the condition(s) for which relief is sought, is modified in accordance with a variety of factors. These factors include the disorder from which an individual suffers, as well as the age, weight, sex, diet, and medical condition of an individual. Thus, in various embodiments, the dosage regimen actually employed varies and deviates from the dosage regimens set forth herein.

### Examples of Pharmaceutical Compositions and Methods of Administration

Provided herein, in certain embodiments, are compositions comprising a therapeutically effective amount of any compound described herein (e.g., a compound of Formula I-XXIII).

Pharmaceutical compositions are formulated using one or more physiologically acceptable carriers including excipients and auxiliaries which facilitate processing of the active compounds into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include one or more PAK inhibitors (e.g., a compound of Formula I-XXIII) and a pharmaceutically acceptable diluent(s), excipient(s), or carrier(s). In addition, the PAK inhibitor is optionally administered as pharmaceutical compositions in which it is mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of a PAK inhibitor with other chemical components, such as carriers, stabilizers, diluents, dispersing agents, suspending agents, thickening agents, and/or excipients. The pharmaceutical composition facilitates administration of the PAK inhibitor to an organism. In practicing the methods of treatment or use provided herein, therapeutically effective amounts of a PAK inhibitor are administered in a pharmaceutical composition to a mammal having a condition, disease, or disorder to be treated. Preferably, the mammal is a human. A therapeutically effective amount varies depending on the severity and stage of the condition, the age and relative health of an individual, the potency of the PAK inhibitor used and other factors. The PAK inhibitor is optionally used singly or in combination with one or more therapeutic agents as components of mixtures.

The pharmaceutical formulations described herein are optionally administered to a individual by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions will include at least one PAK inhibitor, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these PAK inhibitors having the same type of activity. In some situations, PAK inhibitors exist as tautomers. All tautomers are included within the scope of the compounds presented herein. Additionally, the PAK inhibitor exists in unsolvated as well as solvated forms with pharmaceutically acceptable solvents such as water, ethanol, and the like. The solvated forms of the PAK inhibitors presented herein are also considered to be disclosed herein.

“Carrier materials” include any commonly used excipients in pharmaceutics and should be selected on the basis of compatibility with compounds disclosed herein, such as, a PAK inhibitor, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

Moreover, the pharmaceutical compositions described herein, which include a PAK inhibitor, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, a formulation comprising a PAK inhibitor is a solid drug dispersion. A solid dispersion is a dispersion of one or more active ingredients in an inert carrier or matrix at solid state prepared by the melting (or fusion), solvent, or melting-solvent methods. (Chiou and Riegelman, Journal of Pharmaceutical Sciences, 60, 1281 (1971)). The dispersion of one or more active agents in a solid diluent is achieved without mechanical mixing. Solid dispersions are also called solid-state dispersions. In some embodiments, any compound described herein (e.g., a compound of Formula I-XXIII) is formulated as a spray dried dispersion (SDD). An SDD is a single phase amorphous molecular dispersion of a drug in a polymer matrix. It is a solid solution prepared by dissolving the drug and a polymer in a solvent (e.g., acetone, methanol or the like) and spray drying the solution. The solvent rapidly evaporates from droplets which rapidly solidifies the polymer and drug mixture trapping the drug in amorphous form as an amorphous molecular dispersion. In some embodiments, such amorphous dispersions are filled in capsules and/or constituted into oral powders for reconstitution. Solubility of an SDD comprising a drug is higher than the solubility of a crystalline form of a drug or a non-SDD amorphous form of a drug. In some embodiments of the methods described herein, PAK inhibitors are administered as SDDs constituted into appropriate dosage forms described herein.

Pharmaceutical preparations for oral use are optionally obtained by mixing one or more solid excipient with a PAK inhibitor, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate.

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active compound doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of a PAK inhibitor are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a PAK inhibitor, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

The pharmaceutical solid oral dosage forms including formulations described herein, which include a PAK inhibitor, are optionally further formulated to provide a controlled release of the PAK inhibitor. Controlled release refers to the release of the PAK inhibitor from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a individual over an extended period of time according to a predetermined profile. Such release rates provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In other embodiments, the formulations described herein, which include a PAK inhibitor, are delivered using a pulsatile dosage form. A pulsatile dosage form is capable of providing one or more immediate release pulses at predetermined time points after a controlled lag time or at specific sites. Pulsatile dosage forms including the formulations described herein, which include a PAK inhibitor, are optionally administered using a variety of pulsatile formulations that include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the PAK inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the PAK inhibitor is optionally in a form such as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of the PAK inhibitor and a suitable powder base such as lactose or starch.

Buccal formulations that include a PAK inhibitor include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period, wherein the delivery of the PAK inhibitor, is provided essentially throughout. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of a PAK inhibitor are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) a formulation of a PAK inhibitor (e.g., a compound of Formula I-XXIII); (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration of a PAK inhibitor employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery of the PAK inhibitor is optionally accomplished by means of iontophoretic patches and the like. Additionally, transdermal patches provide controlled delivery of the PAK inhibitor. The rate of absorption is optionally slowed by using rate-controlling membranes or by trapping the PAK inhibitor within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing the PAK inhibitor optionally with carriers, optionally a rate controlling barrier to deliver the PAK inhibitor to the skin of the host at a controlled and predetermined rate over a prolonged period of time, and means to secure the device to the skin.

Formulations that include a PAK inhibitor suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, a PAK inhibitor is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of the PAK inhibitor in water soluble form. Additionally, suspensions of the PAK inhibitor are optionally prepared as appropriate oily injection suspensions.

In some embodiments, the PAK inhibitor is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

The PAK inhibitor is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Methods of Dosing and Treatment Regimens

The PAK inhibitor is optionally used in the preparation of medicaments for the prophylactic and/or therapeutic treatment of autism that would benefit, at least in part, from amelioration of symptoms. In addition, a method for treating any of the diseases or conditions described herein in a individual in need of such treatment, involves administration of pharmaceutical compositions containing at least one PAK inhibitor described herein (e.g., a compound of Formula I-XXIII), or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

In the case wherein the patient's condition does not improve, upon the doctor's discretion the administration of the PAK inhibitor is optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the PAK inhibitor is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In some embodiments, the pharmaceutical compositions described herein are in unit dosage forms suitable for single administration of precise dosages. In unit dosage form, the formulation is divided into unit doses containing appropriate quantities of one or more PAK inhibitor. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

The daily dosages appropriate for the PAK inhibitor are from about 0.01 to about 2.5 mg/kg per body weight. An indicated daily dosage in the larger mammal, including, but not limited to, humans, is in the range from about 0.5 mg to about 1000 mg, conveniently administered in divided doses, including, but not limited to, up to four times a day or in extended release form. Suitable unit dosage forms for oral administration include from about 1 to about 500 mg active ingredient, from about 1 to about 250 mg of active ingredient, or from about 1 to about 100 mg active ingredient. The foregoing ranges are merely suggestive, as the number of variables in regard to an individual treatment regime is large, and considerable excursions from these recommended values are not uncommon. Such dosages are optionally altered depending on a number of variables, not limited to the activity of the PAK inhibitor used, the disease or condition to be treated, the mode of administration, the requirements of an individual, the severity of the disease or condition being treated, and the judgment of the practitioner.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. PAK inhibitors exhibiting high therapeutic indices are preferred. The data obtained from cell culture assays and animal studies is optionally used in formulating a range of dosage for use in human. The dosage of such PAK inhibitors lies preferably within a range of circulating concentrations that include the ED50 with minimal toxicity. The dosage optionally varies within this range depending upon the dosage form employed and the route of administration utilized.

### Assays for Identification and Characterization of PAK Inhibitors

Small molecule PAK inhibitors are optionally identified in high-throughput in vitro or cellular assays as described in, e.g., Yu et al (2001), J Biochem (Tokyo); 129(2):243-251; Rininsland et al (2005), BMC Biotechnol, 5:16; and Allen et al (2006), ACS Chem Biol; 1(6):371-376. PAK inhibitors suitable for the methods described herein are available from a variety of sources including both natural (e.g., plant extracts) and synthetic. For example, candidate PAK inhibitors are isolated from a combinatorial library, i.e., a collection of diverse chemical compounds generated by either chemical synthesis or biological synthesis by combining a number of chemical “building blocks.” For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks, as desired. Theoretically, the systematic, combinatorial mixing of 100 interchangeable chemical building blocks results in the synthesis of 100 million tetrameric compounds or 10 billion pentameric compounds. See Gallop et al. (1994), J. Med. Chem. 37(9), 1233. Each member of a library may be singular and/or may be part of a mixture (e.g. a “compressed library”). The library may comprise purified compounds and/or may be “dirty” (i.e., containing a quantity of impurities). Preparation and screening of combinatorial chemical libraries are documented methodologies. See Cabilly, ed., Methods in Molecular Biology, Humana Press, Totowa, N.J., (1998). Combinatorial chemical libraries include, but are not limited to: diversomers such as hydantoins, benzodiazepines, and dipeptides, as described in, e.g., Hobbs et al. (1993), Proc. Natl. Acad. Sci. U.S.A. 90, 6909; analogous organic syntheses of small compound libraries, as described in Chen et al. (1994), J. Amer. Chem. Soc., 116: 2661; Oligocarbamates, as described in Cho, et al. (1993), Science 261, 1303; peptidyl phosphonates, as described in Campbell et al. (1994), J. Org. Chem., 59: 658; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514). In addition, numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.).

Devices for the preparation of combinatorial libraries are commercially available (see, e.g., 357 MPS, 390 MPS from Advanced Chem Tech, Louisville, Ky.; Symphony from Rainin, Woburn, Mass.; 433A from Applied Biosystems, Foster City, Calif.; and 9050 Plus from Millipore, Bedford, Mass.). A number of robotic systems have also been developed for solution phase chemistries. These systems include automated workstations like the automated synthesis apparatus developed by Takeda Chemical Industries, LTD (Osaka, Japan), and many robotic systems utilizing robotic arms (Zymate II). Any of the above devices are optionally used to generate combinatorial libraries for identification and characterization of PAK inhibitors which mimic the manual synthetic operations performed by small molecule PAK inhibitors suitable for the methods described herein. Any of the above devices are optionally used to identify and characterize small molecule PAK inhibitors suitable for the methods disclosed herein. In many of the embodiments disclosed herein, PAK inhibitors, PAK binding molecules, and PAK clearance agents are disclosed as polypeptides or proteins (where polypeptides comprise two or more amino acids). In these embodiments, the inventors also contemplate that PAK inhibitors, binding molecules, and clearance agents also include peptide mimetics based on the polypeptides, in which the peptide mimetics interact with PAK or its upstream or downstream regulators by replicating the binding or substrate interaction properties of PAK or its regulators. Nucleic acid aptamers are also contemplated as PAK inhibitors, binding molecules, and clearance agents, as are small molecules other than peptides or nucleic acids. For example, in some embodiments small molecule PAK binding partners, inhibitors, or clearance agents, or small molecule agonists or antagonists of PAK modulators or targets, are designed or selected based on analysis of the structure of PAK or its modulators or targets and binding interactions with interacting molecules, using “rational drug design” (see, for example Jacobsen et al. (2004) Molecular Interventions 4:337-347; Shi et al. (2007) Bioorg. Med. Chem. Lett. 17:6744-6749).

The identification of potential PAK inhibitors is determined by, for example, assaying the in vitro kinase activity of PAK in the presence of candidate inhibitors. In such assays, PAK and/or a characteristic PAK fragment produced by recombinant means is contacted with a substrate in the presence of a phosphate donor (e.g., ATP) containing radiolabeled phosphate, and PAK-dependent incorporation is measured. “Substrate” includes any substance containing a suitable hydroxyl moiety that can accept the γ-phosphate group from a donor molecule such as ATP in a reaction catalyzed by PAK. The substrate may be an endogenous substrate of PAK, i.e. a naturally occurring substance that is phosphorylated in unmodified cells by naturally-occurring PAK or any other substance that is not normally phosphorylated by PAK in physiological conditions, but may be phosphorylated in the employed conditions. The substrate may be a protein or a peptide, and the phosphrylation reaction may occur on a serine and/or threonine residue of the substrate. For example, specific substrates, which are commonly employed in such assays include, but are not limited to, histone proteins and myelin basic protein. In some embodiments, PAK inhibitors are identified using IMAP® technology.

Detection of PAK dependent phosphorylation of a substrate can be quantified by a number of means other than measurement of radiolabeled phosphate incorporation. For example, incorporation of phosphate groups may affect physiochemical properties of the substrate such as electrophoretic mobility, chromatographic properties, light absorbance, fluorescence, and phosphorescence. Alternatively, monoclonal or polyclonal antibodies can be generated which selectively recognize phosphorylated forms of the substrate from non-phosphorylated forms whereby allowing antibodies to function as an indicator of PAK kinase activity.

High-throughput PAK kinase assays can be performed in, for example, microtiter plates with each well containing PAK kinase or an active fragment thereof, substrate covalently linked to each well, P³² radiolabled ATP and a potential PAK inhibitor candidate. Microtiter plates can contain 96 wells or 1536 wells for large scale screening of combinatorial library compounds. After the phosphorylation reaction has completed, the plates are washed leaving the bound substrate. The plates are then detected for phosphate group incorporation via autoradiography or antibody detection. Candidate PAK inhibitors are identified by their ability to decease the amount of PAK phosphotransferase ability upon a substrate in comparison with PAK phosphotransferase ability alone.

In some embodiments, the identification of potential PAK inhibitors may also be determined, for example, via in vitro competitive binding assays on the catalytic sites of PAK such as the ATP binding site and/or the substrate binding site. For binding assays on the ATP binding site, a known protein kinase inhibitor with high affinity to the ATP binding site is used such as staurosporine. Staurosporine is immobilized and may be fluorescently labeled, radiolabeled or in any manner that allows detection. The labeled staurosporine is introduced to recombinantly expressed PAK protein or a fragment thereof along with potential PAK inhibitor candidates. The candidate is tested for its ability to compete, in a concentration-dependant manner, with the immobolized staurosporine for binding to the PAK protein. The amount of staurosporine bound PAK is inversely proportional to the affinity of the candidate inhibitor for PAK. Potential inhibitors would decrease the quantifiable binding of staurosporine to PAK. See e.g., Fabian et al (2005) Nat. Biotech., 23:329. Candidates identified from this competitive binding assay for the ATP binding site for PAK would then be further screened for selectivity against other kinases for PAK specificity.

In some embodiments, the identification of potential PAK inhibitors may also be determined, for example, by in cyto assays of PAK activity in the presence of the inhibitor candidate. Various cell lines and tissues may be used, including cells specifically engineered for this purpose. In cyto screening of inhibitor candidates may assay PAK activity by monitoring the downstream effects of PAK activity. Such effects include, but are not limited to, the formation of peripheral actin microspikes and or associated loss of stress fibers as well as other cellular responses such as growth, growth arrest, differentiation, or apoptosis. See e.g., Zhao et al., (1998) Mol. Cell. Biol. 18:2153. For example in a PAK yeast assay, yeast cells grow normally in glucose medium. Upon exposure to galactose however, intracellular PAK expression is induced, and in turn, the yeast cells die. Candidate compounds that inhibit PAK activity are identified by their ability to prevent the yeast cells from dying from PAK activation.

Alternatively, PAK-mediated phosphorylation of a downstream target of PAK can be observed in cell based assays by first treating various cell lines or tissues with PAK inhibitor candidates followed by lysis of the cells and detection of PAK mediated events. Cell lines used in this experiment may include cells specifically engineered for this purpose. PAK mediated events include, but are not limited to, PAK mediated phosphorylation of downstream PAK mediators. For example, phosphorylation of downstream PAK mediators can be detected using antibodies that specifically recognize the phosphorylated PAK mediator but not the unphosphorylated form. These antibodies have been described in the literature and have been extensively used in kinase screening campaigns. In some instances a phospho LIMK antibody is used after treatment of HeLa cells stimulated with EGF or sphingosine to detect downstream PAK signaling events.

The identification of potential PAK inhibitors may also be determined, for example, by in vivo assays involving the use of animal models, including transgenic animals that have been engineered to have specific defects or carry markers that can be used to measure the ability of a candidate substance to reach and/or affect different cells within the organism.

For example, suitable animal models for Alzheimer's disease are knock-ins or transgenes of the human mutated genes including transgenes of the “swedish” mutation of APP (APPswe), and transgenes expressing the mutant form of presenilin 1 and presenilin 2 found in familial/early onset AD. Thus, identification of PAK inhibitors can comprise 15, administering a candidate to a knock-in animal and observing for reversals in synaptic plasticity and behavior defects as a readout for PAK inhibition. Administration of the candidate to the animal is via any clinical or non-clinical route, including but not limited to oral, nasal, buccal and/or topical administrations. Additionally or alternatively, administration may be intratracheal instillation, bronchial instillation, intradermal, subcutaneous, intramuscular, intraperitoneal, inhalation, and/or intravenous injection.

Changes in spine morphology are detected using any suitable method, e.g., by use of 3D and/or 4D real time interactive imaging and visualization. In some instances, the Imaris suite of products (available from Bitplane Scientific Solutions) provides functionality for visualization, segmentation and interpretation of 3D and 4D microscopy datasets obtained from confocal and wide field microscopy data.

### EXAMPLES

The following specific examples are to be construed as merely illustrative, and not limitative of the remainder of the disclosure in any way whatsoever.

All synthetic chemistry was performed in standard laboratory glassware unless indicated otherwise in the examples. Commercial reagents were used as received. Analytical LC/MS was performed on an Agilent 1200 system with a variable wavelength detector and Agilent 6140 Single quadrupole mass spectrometer, alternating positive and negative ion scans. Retention times were determined from the extracted 220 nm chromatogram. ¹H NMR was performed on a Bruker DRX-400 at 400 MHz. Microwave reactions were performed in a Biotage Initiator using the instrument software to control heating time and pressure.

Hydrogenation reactions were performed on a H-Cube using the commercially available catalyst cartridges. Silica gel chromatography was performed manually.

Preparative HPLC was performed on a Waters 1525/2487 with UV detection at 220 nm and manual collection.

### Analytical LC/MS Method:

HPLC column: Zorbax SB-C18, 3.5 μm, 2.1 mm×30 mm, maintained at 40° C.

HPLC Gradient: 0.4 mL/min, 95:5:0.1 water:acetonitrile:formic acid for 0.1 min then to 5:95:0.1 water:acetonitrile:formic acid in 3.9 min, maintaining for 0.5 min.

Preparative HPLC Method:

HPLC column: Zorbax SB-C18 21.2×100 mm.

HPLC Gradient: 20 mL/min, 95:5:0.1 water:methanol:formic acid to 5:95:0.1 water:methanol:formic acid; the gradient shape was optimized for individual separations.

### Example 1

### Synthesis of 8-(7-methoxy-2,3-dihydro-1H-inden-1-yl)-2-(4-(4-methylpiperazin-1-yl)phenylamino)pyrido[2,3-d]pyrimidin-7(8H)-one

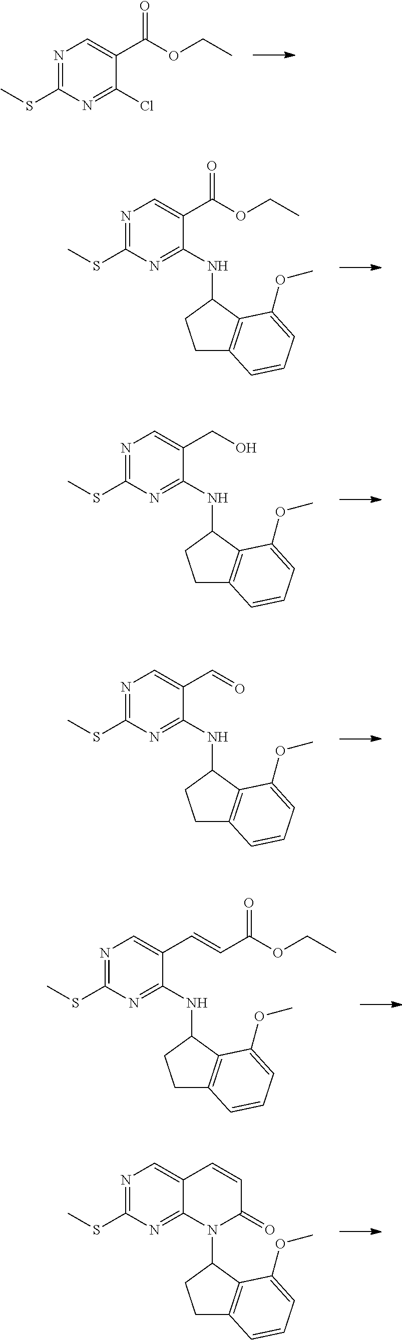

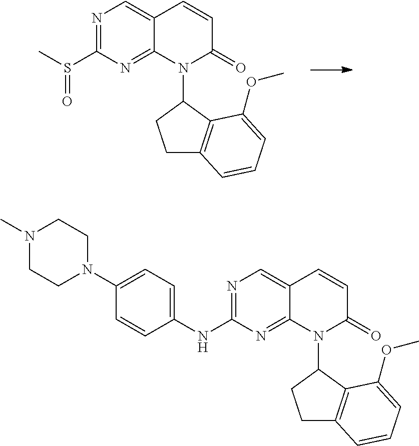

### Intermediate 1: Synthesis of 7-methoxy-1-aminoindane hydrochloride

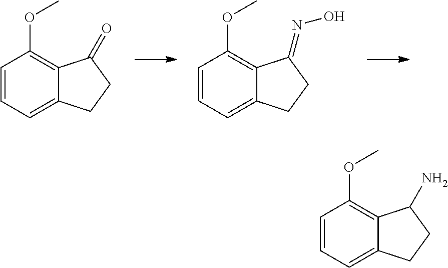

### Step 1: Synthesis of 7-methoxyindan-1-one oxime

To a suspension of 7-methoxyindanone (5.0 g, 31 mmol) and hydroxylamine hydrochloride (12.9 g, 185 mmol) in 100 mL ethanol was added the solution of sodium acetate (11.4 g, 139 mmol) in 35 mL water at room temperature. The reaction mixture was heated at reflux for 4 h, then stirred at room temperature for 18 h. The suspension was filtered, the white solid was washed with water, ethanol and diethyl ether to give the title compound (5.4 g, 31 mmol, 98%). ESMS m/z 178 (M+H)⁺.

### Step 2: Synthesis of 7-methoxy-1-aminoindane hydrochloride

7-methoxyindan-1-one oxime (2.92 g, 16 mmol) was dissolved in acetic acid (150 mL) and hydrogenated on the H-Cube: 1 mL/min flow rate, 80° C., 100 bar with 10% Pd/C. The reaction mixture was evaporated, the residue was dissolved in methanol and 1 equivalent of hydrochloric acid in methanol was added. The solvent was evaporated and the residue was triturated with diethyl ether to give 7-methoxy-1-aminoindane hydrochloride (2.38 g, 12 mmol, 75%). ESMS m/z 147 (M+H)⁺.

### Step 3: Synthesis of ethyl 4-(7-methoxy-2,3-dihydro-1H-inden-1-ylamino)-2-(methylthio)pyrimidine-5-carboxylate

To a stirred solution of ethyl 4-chloro-2-methylthiopyrimidine-5-carboxylate (2.24 g, 9.62 mmol) in 35 mL of anhydrous tetrahydrofuran was added triethylamine (4.00 mL, 2.90 g, 28.72 mmol). The solution was cooled to 0-5° C. and 7-methoxy-1-aminoindane hydrochloride (2.00 g, 10.01 mmol) was added. The reaction mixture was allowed to warm to room temperature and stirred 48 h. The precipitate was filtered off, washed with ethyl acetate (1×25 mL), and the combined filtrates were evaporated to dryness. The residue was dissolved in dichloromethane (35 mL) washed with saturated sodium bicarbonate solution (1×17 mL), dried over magnesium sulfate, filtered and concentrated to give 4-(7-methoxy-2,3-dihydro-1H-inden-1-ylamino)-2-(methylthio)pyrimidine-5-carboxylate as an oil (3.31 g, 9.21 mmol, 95%). ESMS m/z 360 (M+H)⁺; ¹H NMR (400 MHz, CDCl₃) δ ppm 8.63 (s, 1H), 8.43 (d, J=6.5 Hz, 1H), 7.21-7.26 (m, 1H), 6.88 (d, J=7.5 Hz, 1H), 6.72 (d, J=8.3 Hz, 1H), 5.69-5.78 (m, 1H), 4.26 (q, J=7.2 Hz, 2H), 3.78 (s, 3H), 3.01-3.13 (m, 1H), 2.82-2.94 (m, 1H), 2.59-2.67 (m, 1H), 2.56 (s, 3H), 2.04-2.14 (m, 1H), 1.33 (t, J=7.2 Hz, 3H).

### Step 4: Synthesis of (4-(7-methoxy-2,3-dihydro-1H-inden-1-ylamino)-2-(methylthio)pyrimidin-5-yl)methanol

A solution of 4-(7-methoxy-2,3-dihydro-1H-inden-1-ylamino)-2-(Methylthio)pyrimidine-5-carboxylate (3.25 g, 9.04 mmol) in anhydrous tetrahydrofuran (30 mL) was added dropwise to a suspension of lithium aluminum hydride (0.54 g, 14.25 mmol) in anhydrous tetrahydrofuran (8 mL) at 0-5° C. The reaction mixture was allowed to slowly warm to room temperature and stirred for 18 h, then the mixture was cooled to 0-5° C. and quenched with water:tetrahydrofuran (15 mL:5 mL), followed by a 10% sodium hydroxide solution (11 mL). After stirring for 1 h, the precipitate was filtered off and washed with ethyl acetate (5×25 mL). The combined filtrates were diluted with saturated brine solution (20 mL) and water (15 mL), the two phases were separated, and the organic layer was washed with water (1×25 mL), dried over magnesium sulfate, filtered and evaporated to a light brown solid (2.43 g, 7.65 mmol, 84%). ESMS m/z 318 (M+H)⁺.

### Step 5: Synthesis of 4-(7-methoxy-2,3-dihydro-1H-inden-1-ylamino)-2-(methylthio)pyrimidine-5-carbaldehyde

To a solution of (4-(7-methoxy-2,3-dihydro-1H-inden-1-ylamino)-2-(methylthio)pyrimidin-5-yl)methanol (2.36 g, 7.43 mmol) in dichloromethane (80 mL) was added manganese dioxide (90%, 3.87 g, 40 mmol) in small portions. The resulting suspension was stirred for 18 h. Additional manganese dioxide (90%, 3.87 g, 40 mmol) was added and the mixture was stirred for an additional 18 h. The mixture was filtered through Celite and washed with dichloromethane (5×10 mL). The combined filtrates were evaporated in vacuo to give 4-(7-methoxy-2,3-dihydro-1H-inden-1-ylamino)-2-(methylthio)pyrimidine-5-carbaldehyde as a light brown solid (1.89 g, 5.99 mmol, 80%). ESMS m/z 316 (M+H)⁺.

### Step 6: Synthesis of (E)-ethyl 3-(4-(7-methoxy-2,3-dihydro-1H-inden-1-ylamino)-2-(methylthio)pyrimidin-5-yl)acrylate

To a suspension of sodium hydride (60% dispersion, 0.21 g, 5.25 mmol) in anhydrous tetrahydrofuran (21 mL) was added dropwise a solution of triethyl phosphonoacetate (1.03 mL, 1.16 g, 5.19 mmol) in anhydrous tetrahydrofuran (5 mL) at 0-5° C. and the reaction mixture was stirred for 30 min at this temperature. To this suspension was added carefully 4-(7-methoxy-2,3-dihydro-1H-inden-1-ylamino)-2-(methylthio)pyrimidine-5-carbaldehyde (1.48 g, 4.69 mmol) in anhydrous tetrahydrofuran (25 mL) below 5° C. The reaction mixture was allowed to warm to room temperature and stirred for 18 h. The mixture was cooled to below 5° C. and water (22 mL) was added dropwise. It was diluted further with ethyl acetate (25 mL) and saturated brine solution (15 mL), the two phases were separated, and the organic layer was washed with saturated sodium carbonate solution (1×30 mL), water (1×30 mL), dried over sodium sulfate, filtered and evaporated to give (E)-ethyl 3-(4-(7-methoxy-2,3-dihydro-1H-inden-1-ylamino)-2-(methylthio)pyrimidin-5-yl)acrylate as a light brown solid (2.26 g, 5.86 mmol, quant.). ESMS m/z 386 (M+H)⁺; ¹H NMR (400 MHz, CDCl₃), E/Z isomers in a ratio of 90:10, δ ppm 8.15 (s, 0.9H, E), 8.13 (s, 0.1H, Z), 7.46 (d, J=16. e.g., 1 Hz, 0.9H, E), 7.27-7.31 (m, 1H, E+Z), 6.92 (d, J=7.5 Hz, 1H, E+Z), 6.76 (d, J=8.3 Hz, 0.9H, E), 6.73 (d, J=8.3 Hz, 0.1H, Z), 6.57 (d, J=11.8 Hz, 0.1H, Z) 6.27 (d, J=16. e.g., 1 Hz, 0.9H, E), 5.98 (d, J=11.8 Hz, 0.1H, Z) 5.77 (d, J=4.5 Hz, 0.9H, E), 5.58-5.65 (m, 1H, E+Z), 5.17 (d, J=4.5 Hz, 0.1H, Z) 4.23 (q, J=7.2 Hz, 2H, E+Z), 3.83 (s, 2.7H, E), 3.78 (s, 0.3H, Z), 2.99-3.11 (m, 1H, E+Z), 2.85-2.95 (m, 1H, E+Z), 2.71-2.82 (m, 1H, E+Z), 2.57 (s, 2.9H, E), 2.56 (s, 0.3H, Z) 1.99-2.11 (m, 1H, E+Z), 1.30 (t, J=7.2 Hz, 3H, E+Z).

### Step 7: Synthesis of 8-(7-methoxy-2, H-inden-1-yl)-2-(methylthio)pyrido[2,3-d]pyrimidin-7(8H)-one

To a solution of (E)-ethyl 3-(4-(7-methoxy-2,3-dihydro-1H-inden-1-ylamino)-2-(methylthio)pyrimidin-5-yl)acrylate (0.30 g, 0.78 mmol) in N-methylpyrrolidinone (1.8 mL) was added 1,8-diazabicyclo[5.4.0]undec-7-ene (0.35 mL, 0.35 g, 2.29 mmol) and the reaction was stirred for 4 h at 120° C. The reaction mixture was poured onto ice water and diluted with ethyl acetate (8 mL) and saturated brine solution (2.5 mL). The two phases were separated, and the organic layer was washed with 1 M hydrochloric acid (1×7 mL), water (1×7 mL), dried over sodium sulfate, filtered and evaporated to 8-(7-methoxy-2,3-dihydro-1H-inden-1-yl)-2-(methylthio)pyrido[2,3-d]pyrimidin-7(8H)-one as a brown oil (0.40 g, 1.18 mmol, quant.). ESMS m/z 340 (M+H)⁺.

### Step 8: Synthesis of 8-(7-methoxy-2,3-dihydro-1H-inden-1-yl)-2-(methylsulfinyl)pyrido[2,3-d]pyrimidin-7(8H)-one

To a solution of 8-(7-methoxy-2,3-dihydro-1H-inden-1-yl)-2-(methylthio)pyrido[2,3-d]pyrimidin-7(8H)-one (0.32 g, 0.94 mmol) in dichloromethane (3 mL) was added 3-chloroperbenzoic acid (70%, 0.18 g, 0.73 mmol) and the mixture was stirred at room temperature for 5 h. The reaction mixture was extracted with saturated sodium bicarbonate solution (2×1.5 mL), the organic layer was dried over sodium sulfate, then filtered and evaporated to give 8-(7-methoxy-2,3-dihydro-1H-inden-1-yl)-2-(methylsulfinyl)pyrido[2,3-d]pyrimidin-7(8H)-one as a light brown oil (0.29 g, 0.82 mmol, 87%). ESMS m/z 356 (M+H)⁺.

### Step 9: Synthesis of 8-(7-methoxy-2,3-dihydro-1H-inden-1-yl)-2-(4-(4-methylpiperazin-1-yl)phenylamino)pyrido[2,3-d]pyrimidin-7(8H)-one

8-(7-methoxy-2,3-dihydro-1H-inden-1-yl)-2-(methylsulfinyl)pyrido[2,3-d]pyrimidin-7(8H)-one (0.29 g, 0.81 mmol) and 4-(4-methylpiperazino)aniline (0.15 g, 0.81 mmol) were stirred at 140° C. for 4 h. The reaction mixture was dissolved in dichloromethane (35 mL) and washed with 10% sodium hydroxide solution (1×15 mL) then with water (1×15 mL). The organic layer was dried over sodium sulfate, filtered and evaporated. The residue was purified by silica gel column chromatography using dichloromethane:methanol (9:1) to give 8-(7-methoxy-2,3-dihydro-1H-inden-1-yl)-2-(4-(4-methylpiperazin-1-yl)phenylamino)pyrido[2,3-d]pyrimidin-7(8H)-one (32 mg, 0.07 mmol, 8.6%) as the major product. ESMS m/z 483 (M+H)⁺; ¹H NMR (400 MHz, CDCl₃) δ ppm 9.81 (br. s., 1H), 8.73 (s, 1H), 7.77 (d, J=9.3 Hz, 1H), 7.61 (d, J=9.0 Hz, 2H), 7.14 (t, J=7.5 Hz, 1H), 6.89 (d, J=9.0 Hz, 2H), 6.87 (br. s., 1H), 6.84 (d, J=7.3 Hz; 1H), 6.65 (d, J=8.3 Hz, 1H), 6.13 (d, J=9.3 Hz, 1H), 3.43 (s, 3H), 3.10-3.00 (m, 4H), 3.00-2.85 (m, 2H), 2.45-2.38 (m, 4H), 2.35-2.25 (m, 2H), 2.20 (s, 3H).

### Examples 2-27

### Compounds 1-25

The following compounds were made by the method of Example 1 using the appropriate amine at Step 1 and aniline at Step 9. If necessary, the amine was synthesized by the method used for Intermediate 1. Compounds containing secondary amines on the aniline were synthesized using the appropriate Boc protected aminoaniline and in the final step were treated with a solution of hydrogen chloride in an organic solvent to produce the compound, optionally isolated as the hydrochloride salt.

| No. | Structure | MW | LCMS Ion | Rt |
|---|---|---|---|---|
| 1 | 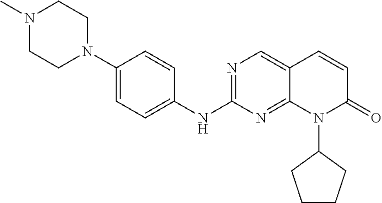 [SMILES: CN1CCN(c2ccc(Nc3ncc4ccc(=O)n(C5CCCC5)c4n3)cc2)CC1] | 404.5 | 405 | 1.87 |
| 2 | 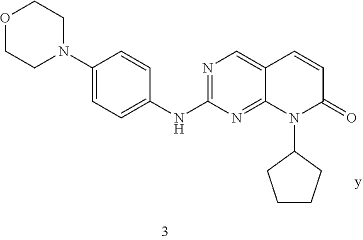 [SMILES: O=c1ccc2cnc(Nc3ccc(N4CCOCC4)cc3)nc2n1C1CCCC1] | 391.5 | 392 | 2.64 |
| 3 | 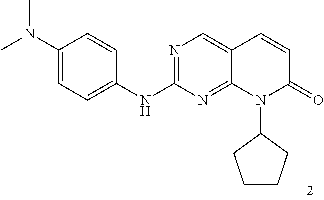 [SMILES: CN(C)c1ccc(Nc2ncc3ccc(=O)n(C4CCCC4)c3n2)cc1] | 349.4 | 350 | 2.68 |
| 4 | 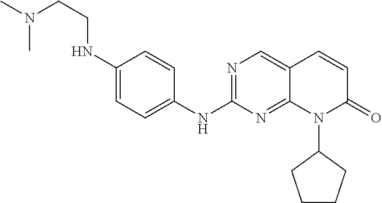 [SMILES: CN(C)CCNc1ccc(Nc2ncc3ccc(=O)n(C4CCCC4)c3n2)cc1] | 392.5 | 393 | 2.48 |
| 5 | 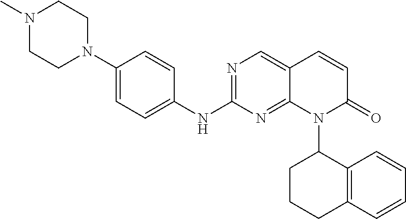 [SMILES: CN1CCN(c2ccc(Nc3ncc4ccc(=O)n(C5CCCc6ccccc65)c4n3)cc2)CC1] | 466.6 | 467 | 2.88 |
| 6 | 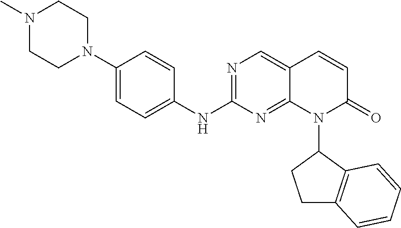 [SMILES: CN1CCN(c2ccc(Nc3ncc4ccc(=O)n(C5CCc6ccccc65)c4n3)cc2)CC1] | 452.6 | 453 | 2.82 |
| 7 | 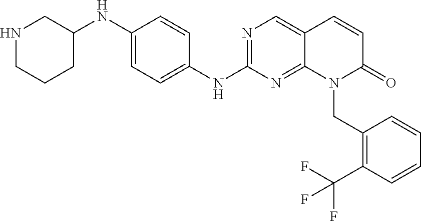 [SMILES: O=c1ccc2cnc(Nc3ccc(NC4CCCNC4)cc3)nc2n1Cc1ccccc1C(F)(F)F] | 494.5 | 495 | 2.89 |
| 8 | 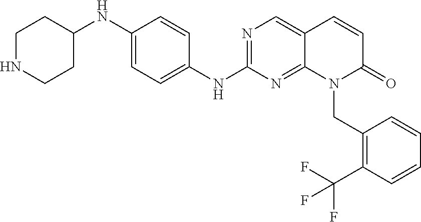 [SMILES: O=c1ccc2cnc(Nc3ccc(NC4CCNCC4)cc3)nc2n1Cc1ccccc1C(F)(F)F] | 494.5 | 495 | 2.75 |
| 9 | 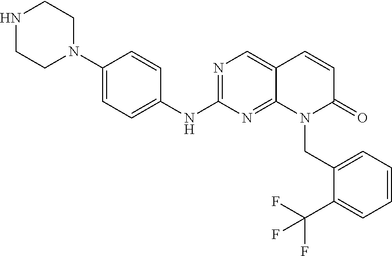 [SMILES: O=c1ccc2cnc(Nc3ccc(N4CCNCC4)cc3)nc2n1Cc1ccccc1C(F)(F)F] | 480.5 | 481 | 2.84 |
| 10 | 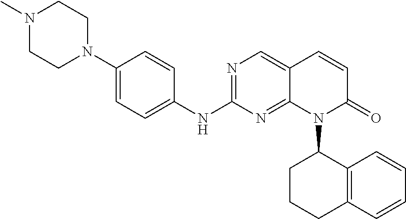 [SMILES: CN1CCN(c2ccc(Nc3ncc4ccc(=O)n([C@@H]5CCCc6ccccc65)c4n3)cc2)CC1] | 466.6 | 467 | 2.90 |
| 11 | 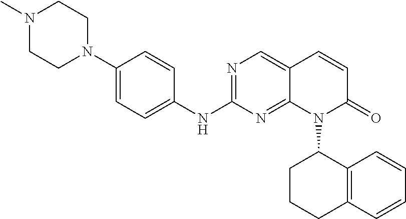 [SMILES: CN1CCN(c2ccc(Nc3ncc4ccc(=O)n([C@H]5CCCc6ccccc65)c4n3)cc2)CC1] | 466.6 | 467 | 2.91 |
| 12 | 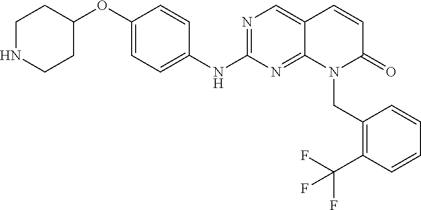 [SMILES: O=c1ccc2cnc(Nc3ccc(OC4CCNCC4)cc3)nc2n1Cc1ccccc1C(F)(F)F] | 495.5 | 496 | 3.66 |
| 13 | 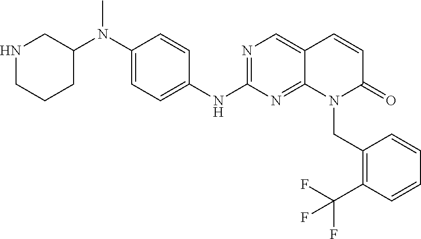 [SMILES: CN(c1ccc(Nc2ncc3ccc(=O)n(Cc4ccccc4C(F)(F)F)c3n2)cc1)C1CCCNC1] | 508.5 | 509 | 2.92 |
| 14 | 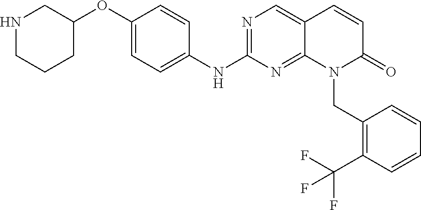 [SMILES: O=c1ccc2cnc(Nc3ccc(OC4CCCNC4)cc3)nc2n1Cc1ccccc1C(F)(F)F] | 495.5 | 496 | 2.95 |
| 15 | 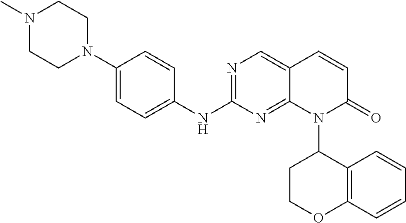 [SMILES: CN1CCN(c2ccc(Nc3ncc4ccc(=O)n(C5CCOc6ccccc65)c4n3)cc2)CC1] | 468.6 | 469 | 2.74 |
| 16 | 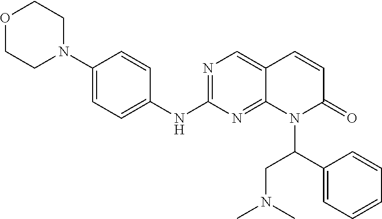 [SMILES: CN(C)CC(c1ccccc1)n1c(=O)ccc2cnc(Nc3ccc(N4CCOCC4)cc3)nc21] | 470.6 | 471 | 2.76 |
| 17 | 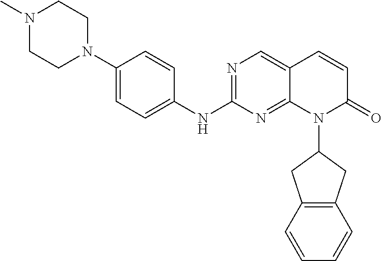 [SMILES: CN1CCN(c2ccc(Nc3ncc4ccc(=O)n(C5Cc6ccccc6C5)c4n3)cc2)CC1] | 452.6 | 453 | 2.92 |
| 18 | 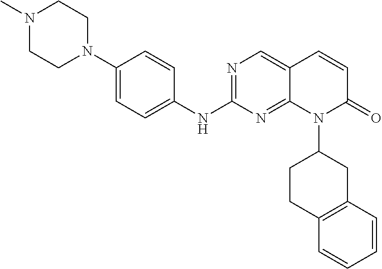 [SMILES: CN1CCN(c2ccc(Nc3ncc4ccc(=O)n(C5CCc6ccccc6C5)c4n3)cc2)CC1] | 466.6 | 465 | 2.97 |
| 19 | 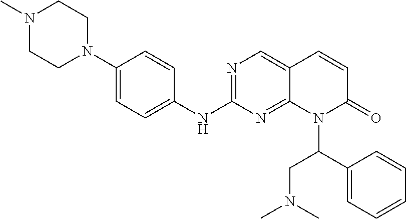 [SMILES: CN(C)CC(c1ccccc1)n1c(=O)ccc2cnc(Nc3ccc(N4CCN(C)CC4)cc3)nc21] | 483.6 | 484 | 2.34 |
| 20 | 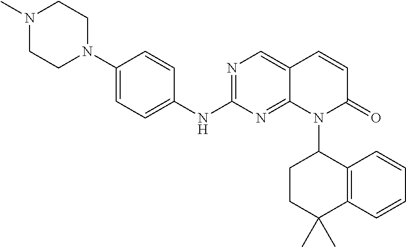 [SMILES: CN1CCN(c2ccc(Nc3ncc4ccc(=O)n(C5CCC(C)(C)c6ccccc65)c4n3)cc2)CC1] | 494.6 | 495 | 3.31 |
| 21 | 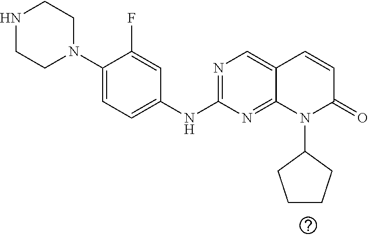 [SMILES: O=c1ccc2cnc(Nc3ccc(N4CCNCC4)c(F)c3)nc2n1C1CCCC1] | 456.5 | 457 | 3.11 |
| 22 | 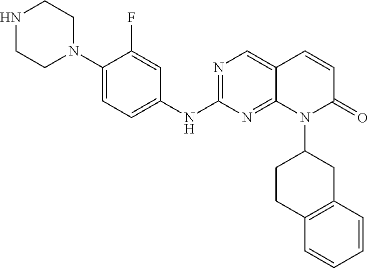 [SMILES: O=c1ccc2cnc(Nc3ccc(N4CCNCC4)c(F)c3)nc2n1C1CCc2ccccc2C1] | 470.6 | 471 | 3.15 |
| 23 | 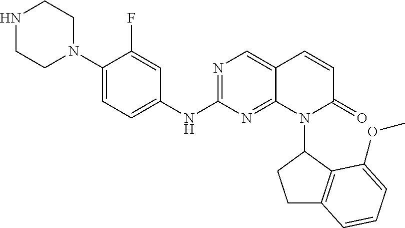 [SMILES: COc1cccc2c1C(n1c(=O)ccc3cnc(Nc4ccc(N5CCNCC5)c(F)c4)nc31)CC2] | 486.6 | 487 | 3.19 |
| 24 | 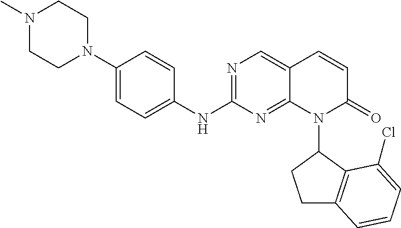 [SMILES: CN1CCN(c2ccc(Nc3ncc4ccc(=O)n(C5CCc6cccc(Cl)c65)c4n3)cc2)CC1] | 487.0 | 487 | 3.20 |
| 25 | 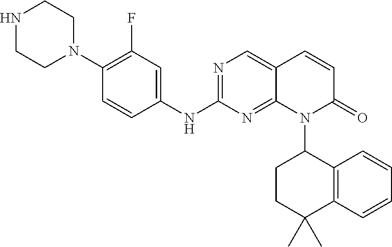 [SMILES: CC1(C)CCC(n2c(=O)ccc3cnc(Nc4ccc(N5CCNCC5)c(F)c4)nc32)c2ccccc21] | 498.6 | 499 | 3.36 |

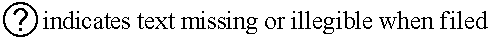

### Example 28

### Synthesis of 8-(2-bromobenzyl)-2-(4-(4-methylpiperazin-1-yl)phenylamino)pyrido[2,3-d]pyrimidin-7(8H)-one

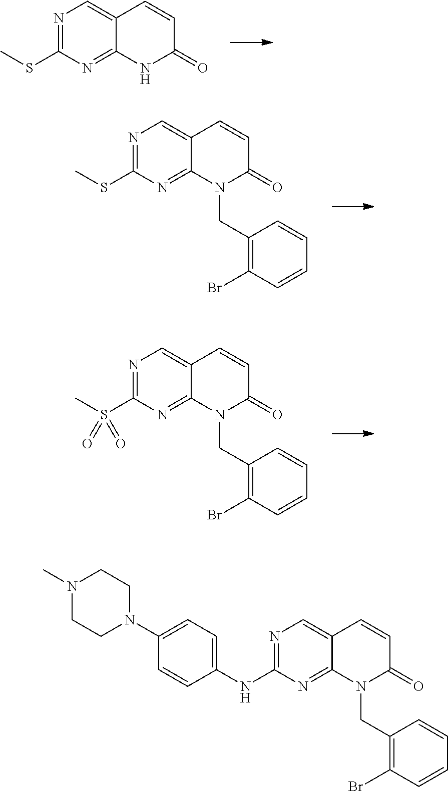 [SMILES: CSc1ncc2ccc(=O)[nH]c2n1 ; CSc1ncc2ccc(=O)n(Cc3ccccc3Br)c2n1 ; CS(=O)(=O)c1ncc2ccc(=O)n(Cc3ccccc3Br)c2n1 ; CN1CCN(c2ccc(Nc3ncc4ccc(=O)n(Cc5ccccc5Br)c4n3)cc2)CC1]

### Step 1: Synthesis of 8-(2-bromobenzyl)-2-(methylthio)pyrido[2,3-d]pyrimidin-7(8H)-one

To a suspension of NaH (60%, 47 mg, 1.19 mmol) in anhydrous dimethylformamide (2 mL) was added 2-(methylthio)pyrido[2,3-d]pyrimidin-7(8H)-one (150 mg, 0.78 mmol, prepared by the method in example 1, Steps 1-5, using ammonia in the first step) at room temperature and stirred at 60° C. for 0.5 h. The reaction mixture was cooled down to room temperature and 2-bromobenzyl bromide was added and stirred for 48 h. The mixture was diluted with ethyl acetate (20 mL) and 10% brine solution (10 mL), the two phases were separated, the aqueous layer was washed with ethyl acetate (1×20 mL), the combined organic layer was dried over sodium sulfate, filtered and evaporated to give 8-(2-bromobenzyl)-2-(methylthio)pyrido[2,3-d]pyrimidin-7(8H)-one, an orange oil (0.24 g, 0.66 mmol, 84%) ESMS m/z 362 (M+H)⁺; ¹H NMR (400 MHz, CDCl₃) δ ppm 8.63 (s, 1H), 7.69 (d, J=9.5 Hz, 1H), 7.59 (dd, J=7.4, 1.4 Hz, 1H), 7.05-7.15 (m, 2H), 6.74 (d, J=9.5 Hz, 1H), 6.65 (d, J=7.0 Hz, 1H), 5.69 (s, 2H), 2.38 (s, 3H).

### Step 2: Synthesis of 8-(2-bromobenzyl)-2-(methylsulfonyl)pyrido[2,3-d]pyrimidin-7(8H)-one

To a solution of 8-(2-bromobenzyl)-2-(methylthio)pyrido[2,3-d]pyrimidin-7(8H)-one (0.24 g, 0.66 mmol) in methanol (20 mL) was added the solution of Oxone (720 mg, 1.17 mmol) in water (10 mL). The mixture was stirred for 18 h, then evaporated to dryness. The residue was dissolved in the mixture of dichloromethane (20 mL) and water (20 mL), separated, and the aqueous layer was extracted with dichloromethane (1×20 mL), and the combined organic layers were dried over sodium sulfate, filtered and concentrated in vacuo to give 8-(2-bromobenzyl)-2-(methylsulfonyl)pyrido[2,3-d]pyrimidin-7(8H)-one as a beige solid (0.17 g, 0.43 mmol, 65%). ESMS m/z 394 (M+H)⁺.

### Step 3: Synthesis of 8-(2-bromobenzyl)-2-(4-(4-methylpiperazin-1-yl)phenylamino)pyrido[2,3-d]pyrimidin-7C8H)-one

8-(2-bromobenzyl)-2-(methylsulfonyl)pyrido[2,3-d]pyrimidin-7(8H)-one (0.17 g, 0.43 mmol) and 4-(4-methylpiperazino)aniline (0.08 g, 0.43 mmol) were stirred at 140° C. for 4 h. The reaction mixture was dissolved in dichloromethane (20 mL) and washed with 10% sodium hydroxide solution (1×10 mL) then with water (1×10 mL). The organic layer was dried over sodium sulfate, filtered and evaporated. The residue was purified by silica gel column chromatography using dichloromethane:methanol (95:5) and the product was recrystallized from isopropanol to give the title compound (19 mg, 0.04 mmol, 9.3%). ESMS m/z 505 (M+H)⁺; ¹H NMR (400 MHz, CDCl₃) δ ppm 8.53 (s, 1H), 7.64 (dd, J=7.7, 1.4 Hz, 1H), 7.61 (d, J=9.3 Hz, 1H), 7.06-7.25 (m, 5H), 6.82 (d, J=8.8 Hz, 2H), 6.66 (br. s., 1H), 6.54 (d, J=9.3 Hz, 1H), 5.59 (s, 2H), 3.14-3.21 (m, 4H), 2.55-2.63 (m, 4H), 2.36 (s, 3H).

### Examples 29-72

### Compounds 26-71

The following compounds were made by the method of Example 28 using the appropriate benzyl bromide, benzyl chloride or phenethyl bromide at Step 1 and aniline at Step 3. If necessary, the benzyl chloride was made by reduction of the appropriate acid or aldehyde to the alcohol followed by conversion to the benzyl chloride with thionyl chloride. Compounds containing secondary amines on the aniline were synthesized using the appropriate Boc protected aminoaniline and in the final step were treated with a solution of hydrogen chloride in an organic solvent to produce the compound, optionally isolated as the hydrochloride salt.

| No. | Structure | MW | LCMS Ion | Rt |
|---|---|---|---|---|
| 26 | 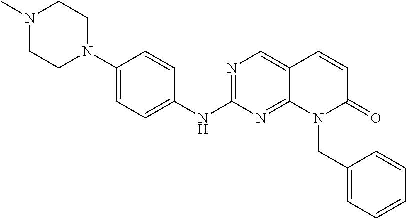 [SMILES: CN1CCN(c2ccc(Nc3ncc4ccc(=O)n(Cc5ccccc5)c4n3)cc2)CC1] | 426.5 | 427 | 2.42 |
| 27 | 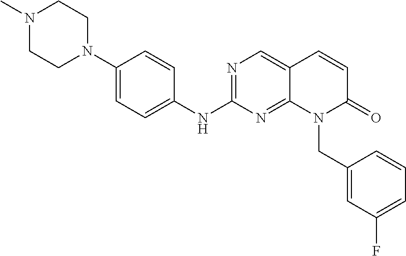 [SMILES: CN1CCN(c2ccc(Nc3ncc4ccc(=O)n(Cc5cccc(F)c5)c4n3)cc2)CC1] | 444.5 | 445 | 2.77 |
| 28 | 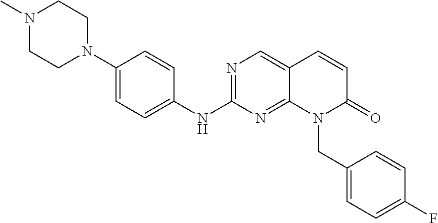 [SMILES: CN1CCN(c2ccc(Nc3ncc4ccc(=O)n(Cc5ccc(F)cc5)c4n3)cc2)CC1] | 444.5 | 445 | 2.79 |
| 29 | 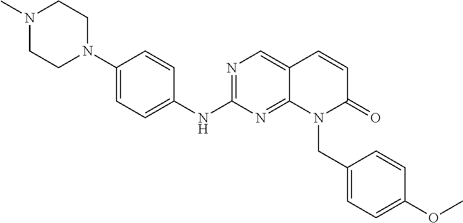 | 456.5 | 457 | 2.78 |
| 30 | 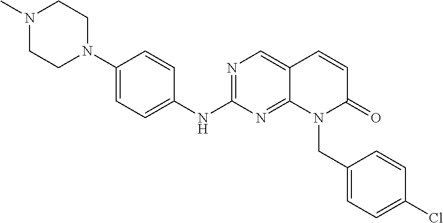 [SMILES: CN1CCN(c2ccc(Nc3ncc4ccc(=O)n(Cc5ccc(Cl)cc5)c4n3)cc2)CC1] | 461.0 | 461 | 2.91 |
| 31 | 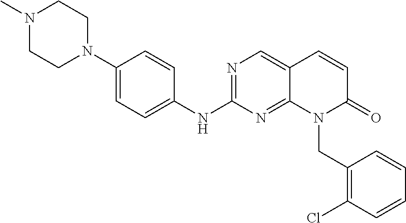 [SMILES: CN1CCN(c2ccc(Nc3ncc4ccc(=O)n(Cc5ccccc5Cl)c4n3)cc2)CC1] | 461.0 | 461 | 2.84 |
| 32 | 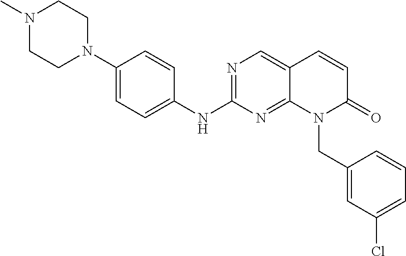 [SMILES: CN1CCN(c2ccc(Nc3ncc4ccc(=O)n(Cc5cccc(Cl)c5)c4n3)cc2)CC1] | 461.0 | 461 | 2.90 |
| 33 | 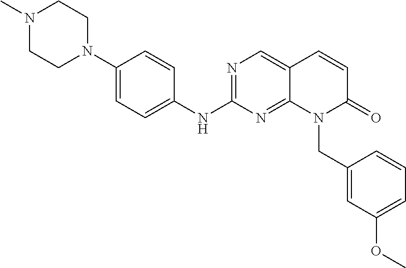 | 456.5 | 457 | 2.78 |
| 34 | 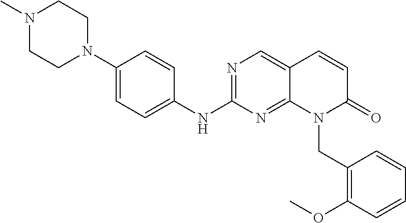 [SMILES: COc1ccccc1Cn1c(=O)ccc2cnc(Nc3ccc(N4CCN(C)CC4)cc3)nc21] | 456.5 | 457 | 2.78 |
| 35 | 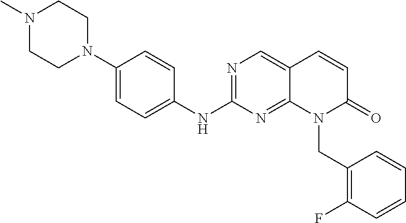 [SMILES: CN1CCN(c2ccc(Nc3ncc4ccc(=O)n(Cc5ccccc5F)c4n3)cc2)CC1] | 444.5 | 445 | 2.75 |
| 36 | 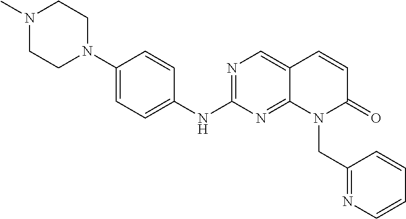 [SMILES: CN1CCN(c2ccc(Nc3ncc4ccc(=O)n(Cc5ccccn5)c4n3)cc2)CC1] | 427.5 | 428 | 2.32 |
| 37 | 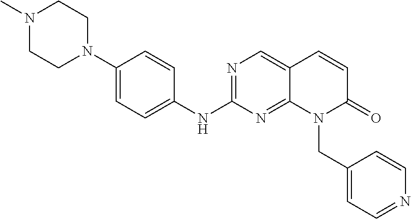 [SMILES: CN1CCN(c2ccc(Nc3ncc4ccc(=O)n(Cc5ccncc5)c4n3)cc2)CC1] | 427.5 | 428 | 1.88 |
| 38 | 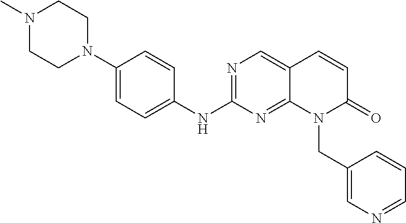 [SMILES: CN1CCN(c2ccc(Nc3ncc4ccc(=O)n(Cc5cccnc5)c4n3)cc2)CC1] | 427.5 | 428 | 2.08 |
| 39 | 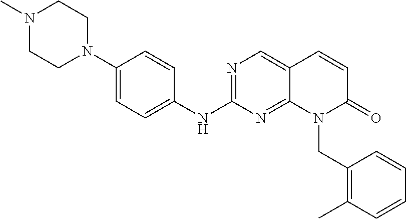 [SMILES: Cc1ccccc1Cn1c(=O)ccc2cnc(Nc3ccc(N4CCN(C)CC4)cc3)nc21] | 440.6 | 441 | 2.74 |
| 40 | 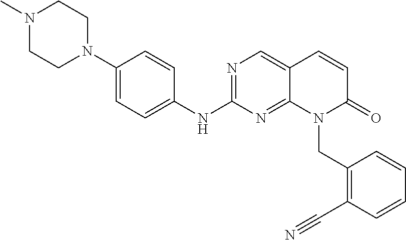 [SMILES: CN1CCN(c2ccc(Nc3ncc4ccc(=O)n(Cc5ccccc5C#N)c4n3)cc2)CC1] | 451.5 | 452 | 2.63 |
| 41 | 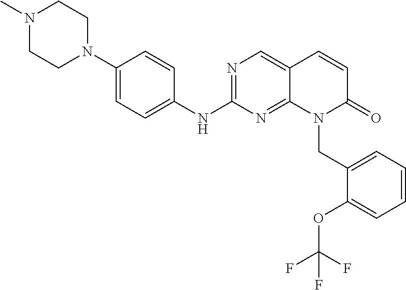 [SMILES: CN1CCN(c2ccc(Nc3ncc4ccc(=O)n(Cc5ccccc5OC(F)(F)F)c4n3)cc2)CC1] | 510.5 | 511 | 2.91 |
| 42 | 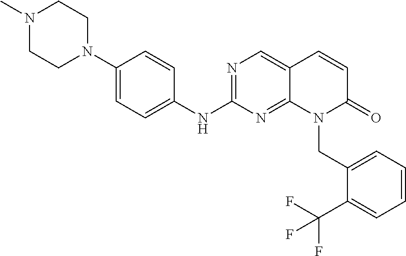 [SMILES: CN1CCN(c2ccc(Nc3ncc4ccc(=O)n(Cc5ccccc5C(F)(F)F)c4n3)cc2)CC1] | 494.5 | 495 | 2.87 |
| 43 | 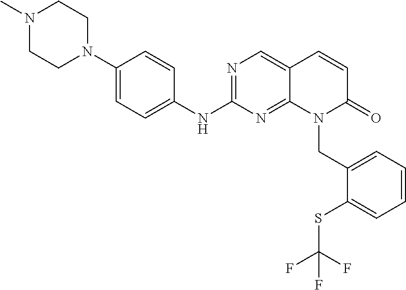 [SMILES: CN1CCN(c2ccc(Nc3ncc4ccc(=O)n(Cc5ccccc5SC(F)(F)F)c4n3)cc2)CC1] | 526.6 | 527 | 3.01 |
| 44 | 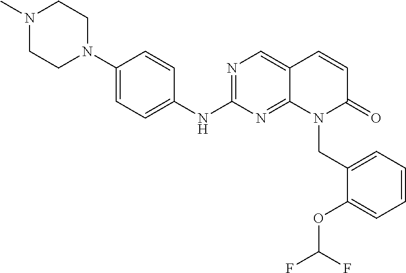 [SMILES: CN1CCN(c2ccc(Nc3ncc4ccc(=O)n(Cc5ccccc5OC(F)F)c4n3)cc2)CC1] | 492.5 | 493 | 2.79 |
| 45 | 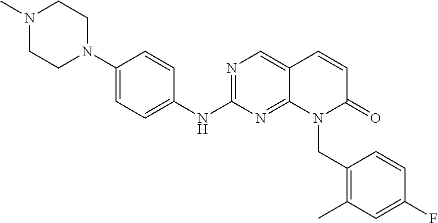 [SMILES: Cc1cc(F)ccc1Cn1c(=O)ccc2cnc(Nc3ccc(N4CCN(C)CC4)cc3)nc21] | 458.5 | 459 | 2.78 |
| 46 | 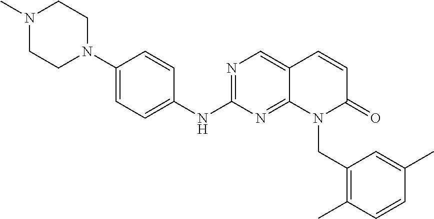 | 454.6 | 455 | 2.81 |
| 47 | 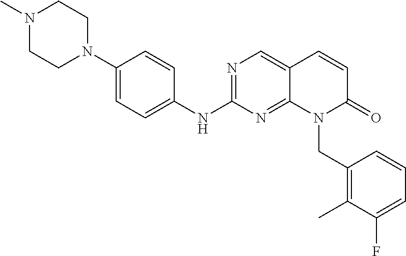 [SMILES: Cc1c(F)cccc1Cn1c(=O)ccc2cnc(Nc3ccc(N4CCN(C)CC4)cc3)nc21] | 458.5 | 459 | 2.79 |
| 48 | 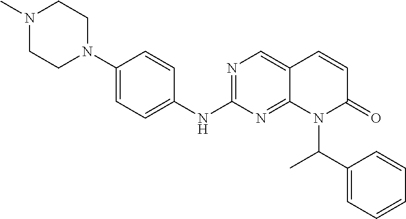 [SMILES: CC(c1ccccc1)n1c(=O)ccc2cnc(Nc3ccc(N4CCN(C)CC4)cc3)nc21] | 440.6 | 441 | 2.72 |
| 49 | 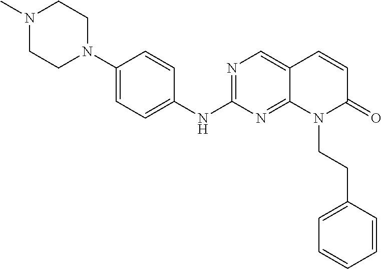 [SMILES: CN1CCN(c2ccc(Nc3ncc4ccc(=O)n(CCc5ccccc5)c4n3)cc2)CC1] | 440.6 | 441 | 2.79 |
| 50 | 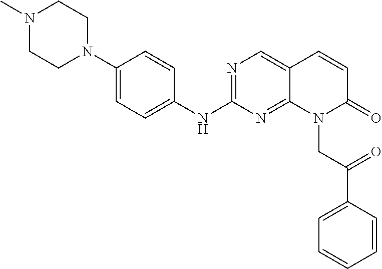 [SMILES: CN1CCN(c2ccc(Nc3ncc4ccc(=O)n(CC(=O)c5ccccc5)c4n3)cc2)CC1] | 454.5 | 455 | 2.55 |
| 51 | 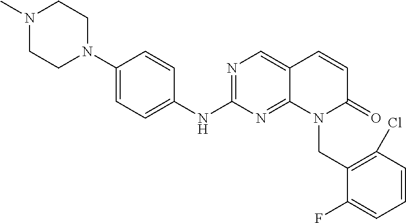 [SMILES: CN1CCN(c2ccc(Nc3ncc4ccc(=O)n(Cc5c(F)cccc5Cl)c4n3)cc2)CC1] | 479.0 | 479 | 2.77 |
| 52 | 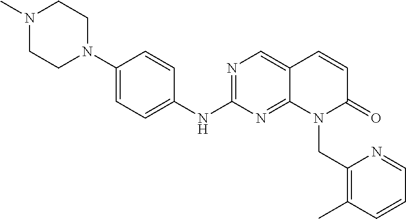 [SMILES: Cc1cccnc1Cn1c(=O)ccc2cnc(Nc3ccc(N4CCN(C)CC4)cc3)nc21] | 441.5 | 442 | 2.23 |
| 53 | 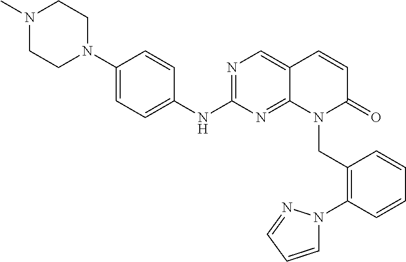 [SMILES: CN1CCN(c2ccc(Nc3ncc4ccc(=O)n(Cc5ccccc5-n5cccn5)c4n3)cc2)CC1] | 492.6 | 493 | 2.65 |
| 54 | 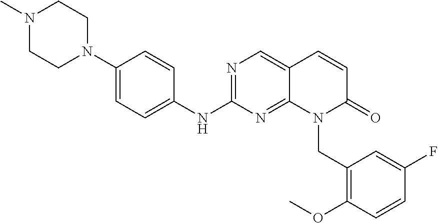 [SMILES: COc1ccc(F)cc1Cn1c(=O)ccc2cnc(Nc3ccc(N4CCN(C)CC4)cc3)nc21] | 474.5 | 475 | 2.75 |
| 55 | 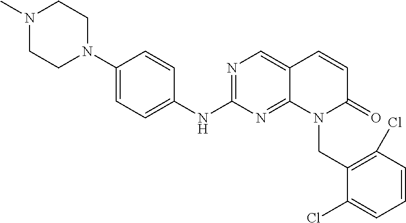 [SMILES: CN1CCN(c2ccc(Nc3ncc4ccc(=O)n(Cc5c(Cl)cccc5Cl)c4n3)cc2)CC1] | 495.4 | 495 | 2.84 |
| 56 | 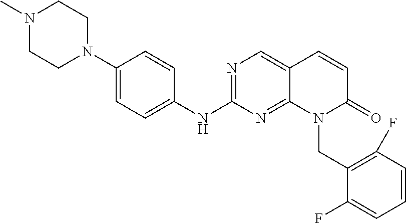 [SMILES: CN1CCN(c2ccc(Nc3ncc4ccc(=O)n(Cc5c(F)cccc5F)c4n3)cc2)CC1] | 462.5 | 463 | 2.72 |
| 57 | 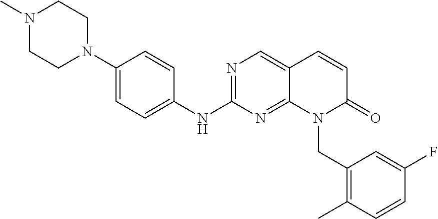 [SMILES: Cc1ccc(F)cc1Cn1c(=O)ccc2cnc(Nc3ccc(N4CCN(C)CC4)cc3)nc21] | 458.5 | 459 | 2.78 |
| 58 | 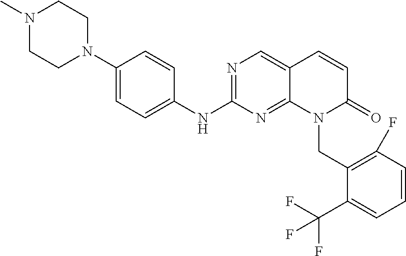 [SMILES: CN1CCN(c2ccc(Nc3ncc4ccc(=O)n(Cc5c(F)cccc5C(F)(F)F)c4n3)cc2)CC1] | 512.5 | 513 | 2.91 |
| 59 | 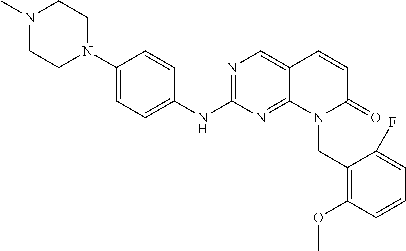 [SMILES: COc1cccc(F)c1Cn1c(=O)ccc2cnc(Nc3ccc(N4CCN(C)CC4)cc3)nc21] | 474.5 | 475 | 2.74 |
| 60 | 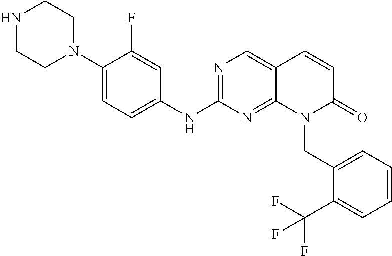 [SMILES: O=c1ccc2cnc(Nc3ccc(N4CCNCC4)c(F)c3)nc2n1Cc1ccccc1C(F)(F)F] | 498.5 | 499 | 2.85 |
| 61 | 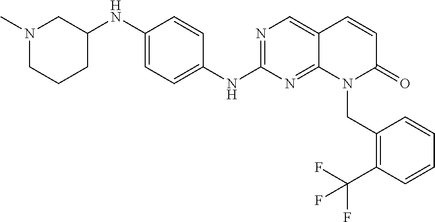 [SMILES: CN1CCCC(Nc2ccc(Nc3ncc4ccc(=O)n(Cc5ccccc5C(F)(F)F)c4n3)cc2)C1] | 508.5 | 509 | 2.90 |
| 62 | 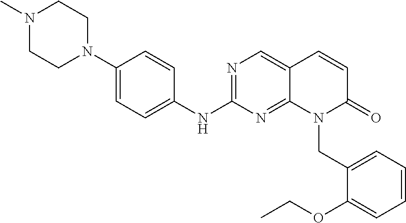 [SMILES: CCOc1ccccc1Cn1c(=O)ccc2cnc(Nc3ccc(N4CCN(C)CC4)cc3)nc21] | 470.6 | 471 | 2.83 |
| 63 | 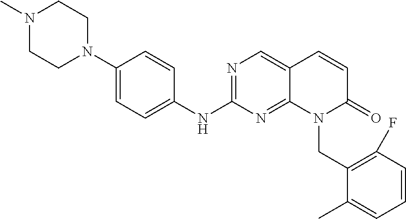 [SMILES: Cc1cccc(F)c1Cn1c(=O)ccc2cnc(Nc3ccc(N4CCN(C)CC4)cc3)nc21] | 458.5 | 459 | 2.75 |
| 64 | 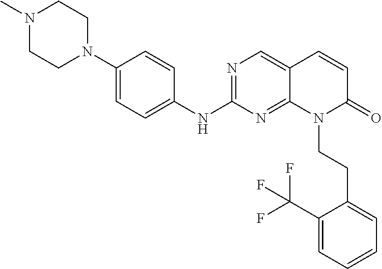 [SMILES: CN1CCN(c2ccc(Nc3ncc4ccc(=O)n(CCc5ccccc5C(F)(F)F)c4n3)cc2)CC1] | 508.5 | 509 | 2.98 |
| 65 | 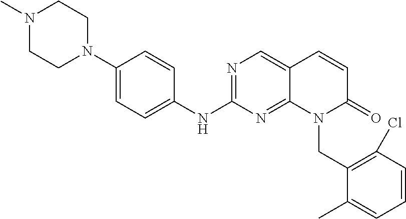 [SMILES: Cc1cccc(Cl)c1Cn1c(=O)ccc2cnc(Nc3ccc(N4CCN(C)CC4)cc3)nc21] | 475.0 | 475 | 2.88 |
| 66 | 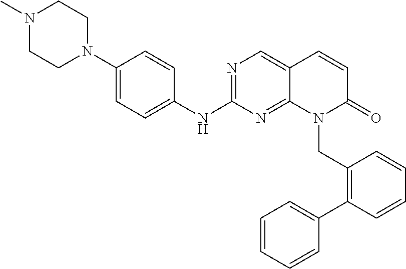 [SMILES: CN1CCN(c2ccc(Nc3ncc4ccc(=O)n(Cc5ccccc5-c5ccccc5)c4n3)cc2)CC1] | 502.6 | 503 | 2.99 |
| 67 | 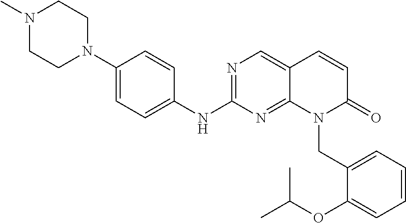 [SMILES: CC(C)Oc1ccccc1Cn1c(=O)ccc2cnc(Nc3ccc(N4CCN(C)CC4)cc3)nc21] | 484.6 | 485 | 2.92 |
| 68 | 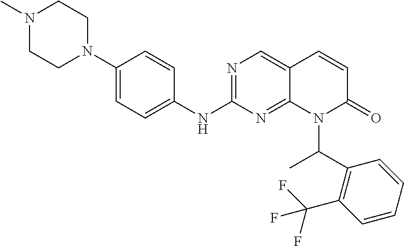 [SMILES: CC(c1ccccc1C(F)(F)F)n1c(=O)ccc2cnc(Nc3ccc(N4CCN(C)CC4)cc3)nc21] | 508.5 | 509 | 2.95 |
| 69 | 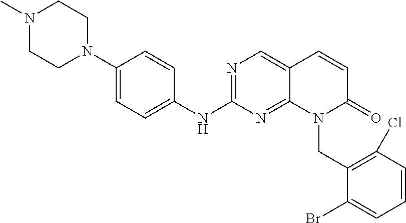 [SMILES: CN1CCN(c2ccc(Nc3ncc4ccc(=O)n(Cc5c(Cl)cccc5Br)c4n3)cc2)CC1] | 539.9 | 539 | 2.97 |
| 70 | 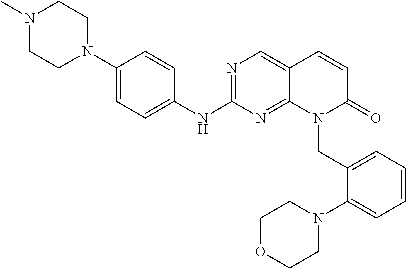 [SMILES: CN1CCN(c2ccc(Nc3ncc4ccc(=O)n(Cc5ccccc5N5CCOCC5)c4n3)cc2)CC1] | 511.6 | 512 | 2.80 |
| 71 | 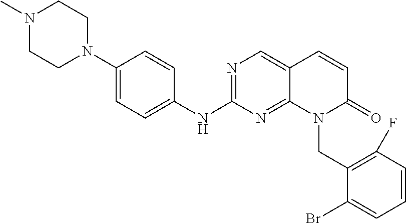 [SMILES: CN1CCN(c2ccc(Nc3ncc4ccc(=O)n(Cc5c(F)cccc5Br)c4n3)cc2)CC1] | 523.4 | 523 | 2.96 |

### Example 73

### Synthesis of N-(5-{2-[4-(4-Methyl-piperazin-1-yl)-phenylamino]-pyrimidin-4-yl}-pyridin-2-yl)-ethane-1,2-diamine hydrochloride

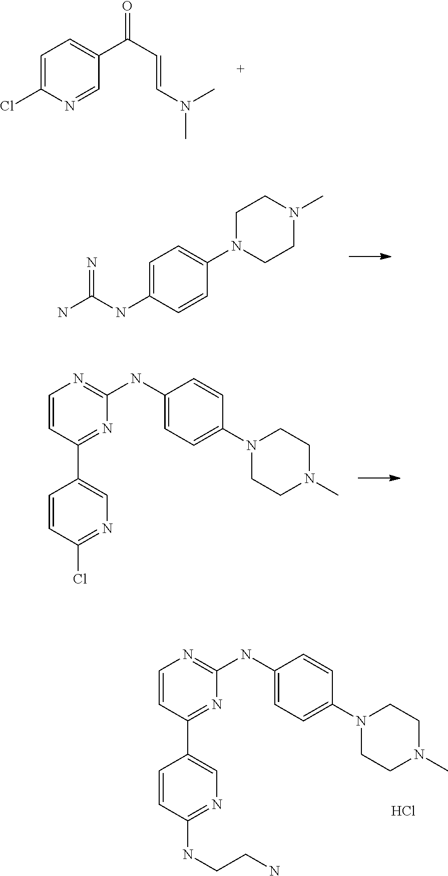 [SMILES: CN(C)/C=C/C(=O)c1ccc(Cl)nc1 ; CN1CCN(c2ccc(NC(=N)N)cc2)CC1 ; CN1CCN(c2ccc(Nc3nccc(-c4ccc(Cl)nc4)n3)cc2)CC1 ; CN1CCN(c2ccc(Nc3nccc(-c4ccc(NCCN)nc4)n3)cc2)CC1 ; Cl]

### Step 1: Synthesis of 1-(6-Chloro-pyridin-3-yl)-3-dimethylamino-propenone

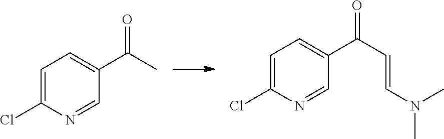

5.00 g (32.2 mmol) 1-(6-Chloro-pyridin-3-yl)-ethanone was dissolved in 40 mL dimethylformamide dimethylacetal, and stirred at 105° C. for 2 h. The solution was cooled to room temperature, and the yellow precipitate was filtered to give 1-(6-Chloro-pyridin-3-yl)-3-dimethylamino-propenone (4.565 g, Y=67%) that was used without further purification.

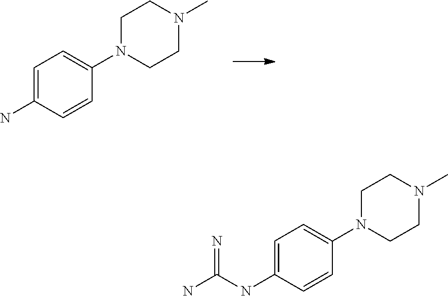 [SMILES: CN1CCN(c2ccc(N)cc2)CC1 ; CN1CCN(c2ccc(NC(=N)N)cc2)CC1]

### Step 2: Synthesis of N-[4-(4-Methyl-piperazin-1-yl)-phenyl]-guanidine hydrochloride

10.00 g 4-(4-Methyl-piperazin-1-yl)-aniline (52 mmol) was dissolved in 30 mL ethanol, 4.37 g cyanamide (104 mmol) and 7.3 mL of 65% nitric acid (114 mmol) were added. The reaction was stirred at 85° C. for 18 h under a nitrogen atmosphere. It was concentrated in vacuo, and the black residue was washed with isopropanol at reflux (3×25 mL). The solid was cooled to room temperature and ground under isopropanol in a ceramic mortar to give N-[4-(4-Methyl-piperazin-1-yl)-phenyl]-guanidine hydrochloride (12.0 g, Y=77%) as a hygroscopic black powder.

### Step 3: Synthesis of [4-(6-Chloro-pyridin-3-yl)-pyrimidin-2-yl]-[4-(4-methyl-piperazin-1-yl)-phenyl]-amine

4.22 g 1-(6-Chloro-pyridin-3-yl)-3-dimethylamino-propenone (20 mmol) was dissolved in 100 mL isopropanol, 5.92 g N-[4-(4-Methyl-piperazin-1-yl)-phenyl]-guanidine hydrochloride (20 mmol) and 0.96 g sodium hydroxide (24 mmol) were added and heated at reflux for 18 h. The mixture was allowed to cool to room temperature and stirred at room temperature for three days. The yellow-green precipitate was filtered to give [4-(6-Chloro-pyridin-3-yl)-pyrimidin-2-yl]-[4-(4-methyl-piperazin-1-yl)-phenyl]amine (2.50 g, Y=33%). Purity: 94% (LCMS); ¹H NMR (400 MHz, DMSO-d₆) δ ppm 9.49 (s, 1H), 9.14 (d, J=2.5 Hz, 1H), 8.54 (d, J=5.3 Hz, 1H), 8.52 (dd, J=8.5, 2.5 Hz, 1H), 7.70 (d, J=8.5 Hz, 1H), 7.60 (d, J=9.0 Hz, 2H), 7.41 (d, J=5.3 Hz, 1H), 6.91 (d, J=9.0 Hz, 2H), 3.03-3.10 (m, 4H), 2.42-2.47 (m, 4H), 2.22 (s, 3H).

### Step 4: Synthesis of N-(5-{2-[4-(4-Methyl-piperazin-1-yl)-phenylamino]-pyrimidin-4-yl}-pyridin-2-yl)-ethane-1,2-diamine

0.15 g (0.39 mmol) of [4-(6-Chloro-pyridin-3-yl)-pyrimidin-2-yl]-[4-(4-methyl-piperazin-1-yl)-phenyl]amine was dissolved in 2.5 mL ethylenediamine and heated in a sealed tube at 120° C. for 18 h. The reaction was cooled and evaporated to dryness and the crude product was purified by silica gel column chromatography using dichloromethane:methanol:triethylamine (9:1:0.05 to 1:1:0.05) to give the title compound as a pale yellow solid (58 mg, 0.14 mmol, 36%). The product was dissolved in dichloromethane (2 mL) then 0.51 M hydrochloric acid:diethyl ether (0.275 mL, 0.14 mmol) was added, it was stirred for 0.5 h. The mixture was evaporated and the residue was suspended in methanol and filtered to give N-(5-{2-[4-(4-Methyl-piperazin-1-yl)-phenylamino]-pyrimidin-4-yl}-pyridin-2-yl)-ethane-1,2-diamine hydrochloride (35.5 mg, 0.08 mmol, 21%). ESMS m/z 405 (M+H)⁺; ¹H NMR (400 MHz, DMSO-d₆) δ ppm 9.23 (s, 1H), 8.84 (d, J=2.0 Hz, 1H), 8.36 (d, J=5.3 Hz, 1H), 8.15 (dd, J=8.8, 2.3 Hz, 1H), 7.87 (br. S., 3H), 7.62 (d, J=9.3 Hz, 2H), 7.33 (t, J=6.3 Hz, 1H), 7.19 (d, J=5.3 Hz, 1H), 6.91 (d, J=9.3 Hz, 2H), 6.65 (d, J=8.5 Hz, 1H), 3.57 (q, J=6.3 Hz, 2H), 3.05-3.12 (m, 4H), 3.01 (t, J=6.3 Hz, 2H), 2.46-2.49 (m, 4H), 2.25 (s, 3H).

### Examples 74-94

### Compounds 72-92

The following compounds were made by the method of Example 73 using the appropriate guanidine at Step 1, and the appropriate amine at Step 4. Example 87 was synthesized using (2-methylaminoethyl)-carbamic acid tert-butyl ester followed by deprotection with hydrochloric acid in diethyl ether.

| No. | Structure | MW | LCMS Ion | Rt |
|---|---|---|---|---|
| 72 | 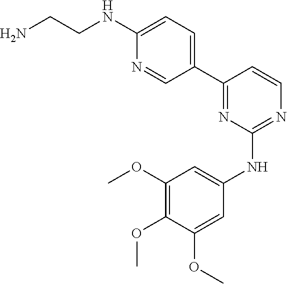 [SMILES: COc1cc(Nc2nccc(-c3ccc(NCCN)nc3)n2)cc(OC)c1OC] | 396.5 | 397 | 2.12 |
| 73 | 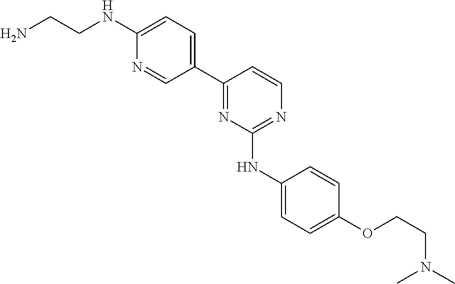 [SMILES: CN(C)CCOc1ccc(Nc2nccc(-c3ccc(NCCN)nc3)n2)cc1] | 393.5 | 394 | 0.93 |
| 74 | 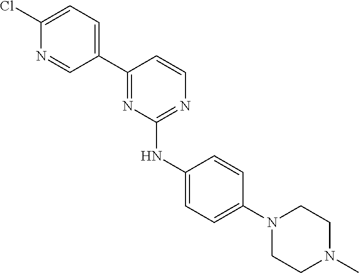 [SMILES: CN1CCN(c2ccc(Nc3nccc(-c4ccc(Cl)nc4)n3)cc2)CC1] | 380.9 | 381 | 2.78 |
| 75 | 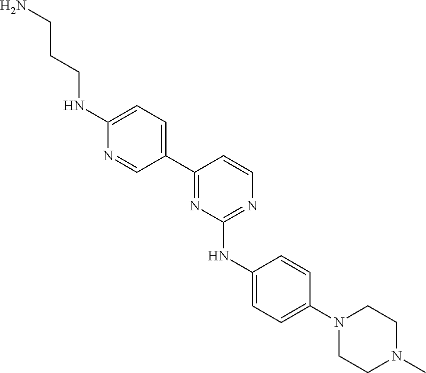 [SMILES: CN1CCN(c2ccc(Nc3nccc(-c4ccc(NCCCN)nc4)n3)cc2)CC1] | 418.5 | 419 | 1.10 |
| 76 | 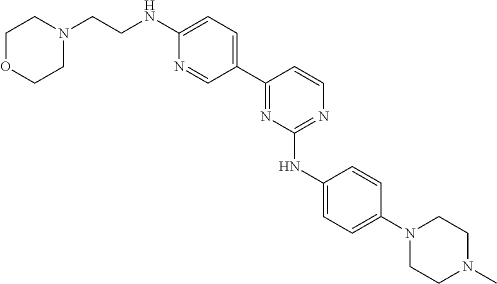 [SMILES: CN1CCN(c2ccc(Nc3nccc(-c4ccc(NCCN5CCOCC5)nc4)n3)cc2)CC1] | 474.6 | 475 | 2.10 |
| 77 | 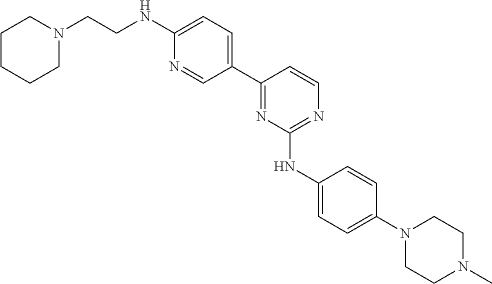 [SMILES: CN1CCN(c2ccc(Nc3nccc(-c4ccc(NCCN5CCCCC5)nc4)n3)cc2)CC1] | 472.6 | 473 | 2.19 |
| 78 | 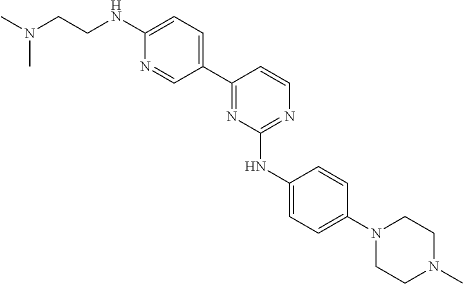 | 432.6 | 433 | 1.29 |
| 79 | 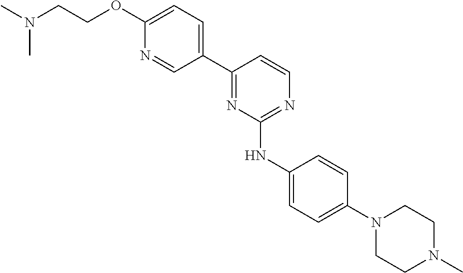 | 433.6 | 434 | 2.17 |
| 80 | 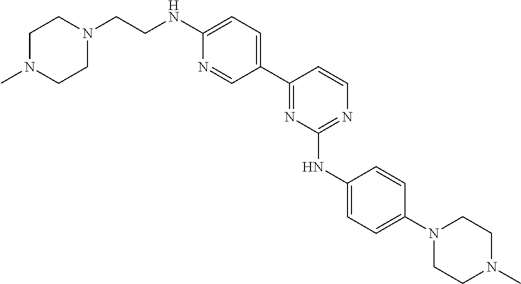 | 487.7 | 488 | 1.14 |
| 81 | 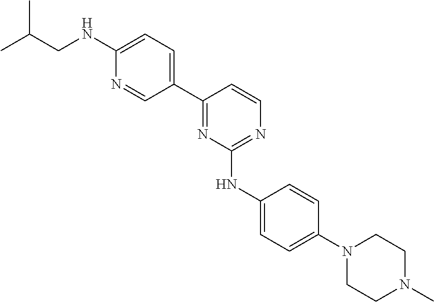 [SMILES: CC(C)CNc1ccc(-c2ccnc(Nc3ccc(N4CCN(C)CC4)cc3)n2)cn1] | 417.6 | 418 | 2.45 |
| 82 | 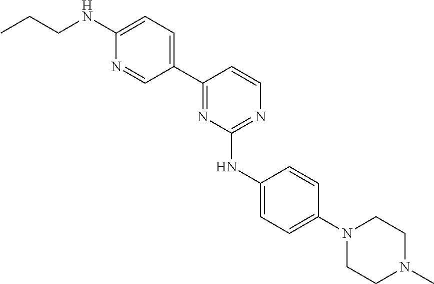 [SMILES: CCCNc1ccc(-c2ccnc(Nc3ccc(N4CCN(C)CC4)cc3)n2)cn1] | 403.5 | 404 | 2.24 |
| 83 | 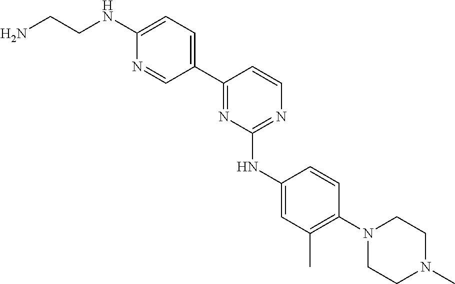 [SMILES: Cc1cc(Nc2nccc(-c3ccc(NCCN)nc3)n2)ccc1N1CCN(C)CC1] | 418.5 | 419 | 2.16 |
| 84 | 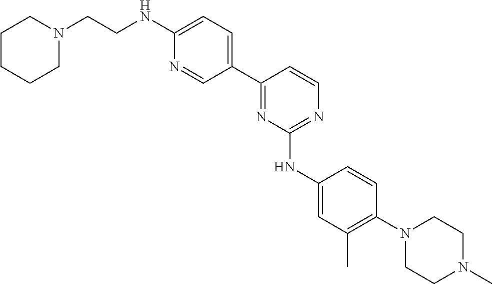 [SMILES: Cc1cc(Nc2nccc(-c3ccc(NCCN4CCCCC4)nc3)n2)ccc1N1CCN(C)CC1] | 486.7 | 487 | 2.32 |
| 85 | 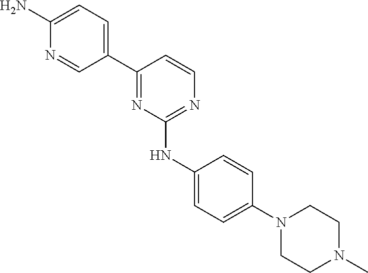 [SMILES: CN1CCN(c2ccc(Nc3nccc(-c4ccc(N)nc4)n3)cc2)CC1] | 361.5 | 362 | 2.31 |
| 86 | 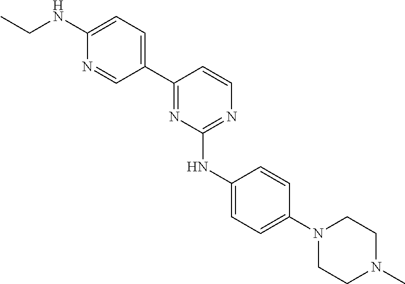 [SMILES: CCNc1ccc(-c2ccnc(Nc3ccc(N4CCN(C)CC4)cc3)n2)cn1] | 389.5 | 390 | 2.17 |
| 87 | 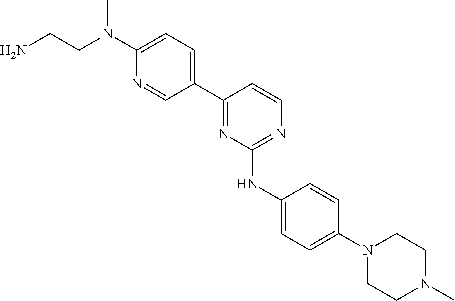 [SMILES: CN1CCN(c2ccc(Nc3nccc(-c4ccc(N(C)CCN)nc4)n3)cc2)CC1] | 418.5 | 419 | 2.11 |
| 88 | 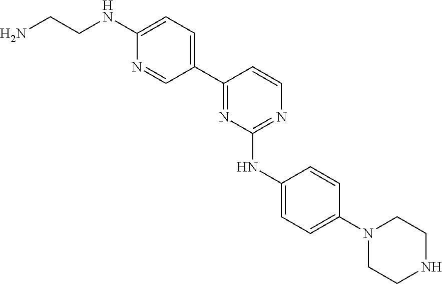 [SMILES: NCCNc1ccc(-c2ccnc(Nc3ccc(N4CCNCC4)cc3)n2)cn1] | 390.5 | 391 | 0.83 |
| 89 | 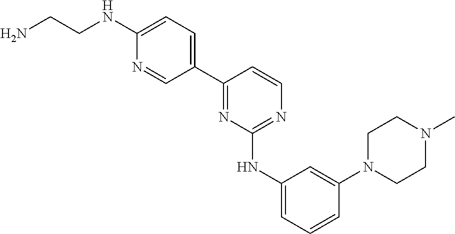 [SMILES: CN1CCN(c2cccc(Nc3nccc(-c4ccc(NCCN)nc4)n3)c2)CC1] | 404.5 | 405 | 3.44 |
| 90 | 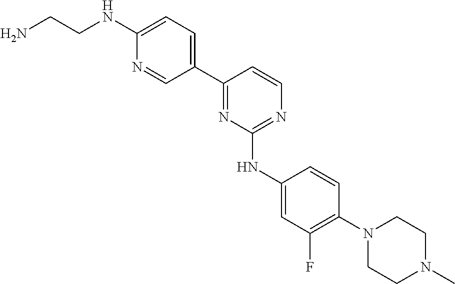 [SMILES: CN1CCN(c2ccc(Nc3nccc(-c4ccc(NCCN)nc4)n3)cc2F)CC1] | 422.5 | 423 | 2.02 |
| 91 | 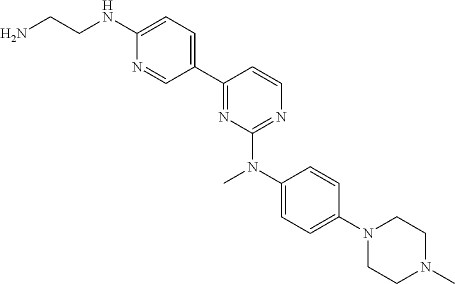 [SMILES: CN1CCN(c2ccc(N(C)c3nccc(-c4ccc(NCCN)nc4)n3)cc2)CC1] | 418.5 | 419 | 1.35 |
| 92 | 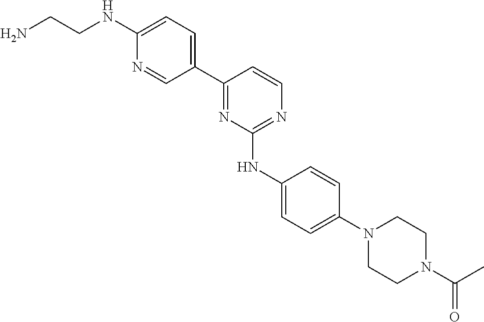 [SMILES: CC(=O)N1CCN(c2ccc(Nc3nccc(-c4ccc(NCCN)nc4)n3)cc2)CC1] | 432.5 | 433 | 2.23 |

### Example 95

### Synthesis of 8-ethyl-2-(3-fluoro-4-(piperazin-1-yl)phenylamino)-6-phenylpyrido[2,3-d]pyrimidin-7(8H)-one hydrochloride

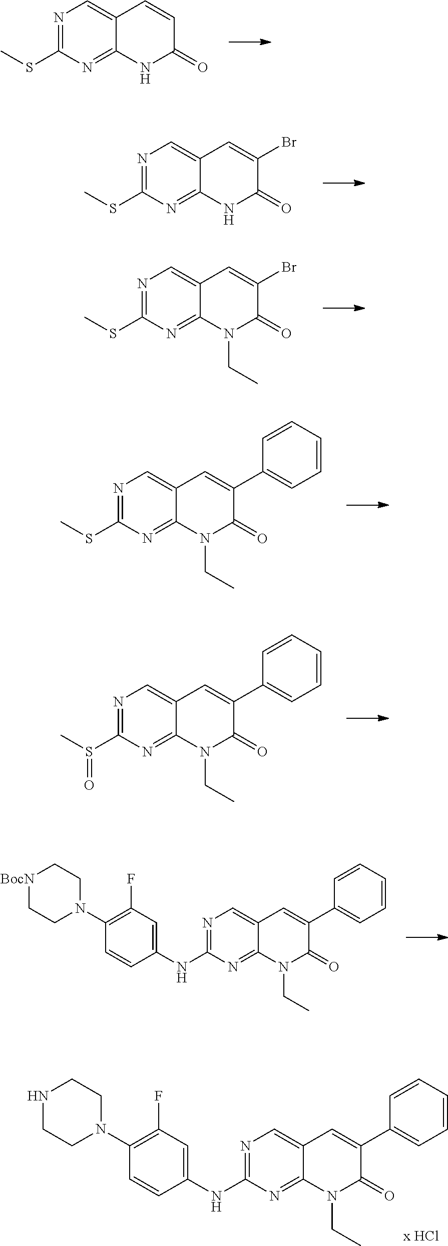

### Step 1: Synthesis of 6-bromo-2-(methylthio)pyrido[2,3-d]pyrimidin-7(8H)-one

To a solution of 2-(methylthio)pyrido[2,3-d]pyrimidin-7(8H)-one (1.00 g, 5.18 mmol) in anhydrous dimethylformamide (25 mL) was added N-bromosuccinimide (0.99 g, 5.59 mmol) portionwise at room temperature, and the reaction mixture was stirred for 18 h. The mixture was concentrated, and the solid was triturated with hot water (1×20 mL), filtered, and washed with isopropanol to give title compound as a pale yellow solid (0.68 g, 2.50 mmol, 48%). ESMS m/z 272 (M+H)⁺; ¹H NMR (400 MHz, DMSO-d₆) δ ppm 12.88 (br. S., 1H), 8.84 (s, 1H), 8.47 (s, 1H), 2.57 (s, 3H).

### Step 2: Synthesis of 6-bromo-8-ethyl-2-(methylthio)pyrido[2,3-d]pyrimidin-7(8H)-one

To a suspension of NaH (60%, 0.15 g, 3.75 mmol) in anhydrous dimethylformamide (10 mL) was added 6-bromo-2-(methylthio)pyrido[2,3-d]pyrimidin-7(8H)-one (0.68 g, 2.50 mmol) at room temperature and the reaction was stirred at 50° C. for 0.5 h. The reaction mixture was cooled down to room temperature and ethyl bromide (0.22 mL, 0.32 g, 2.93 mmol) was added and stirred at 50° C. for 1.5 h. After completion, the mixture was poured onto ice water (10 g), and the white precipitate was filtered off to give 6-bromo-8-ethyl-2-(methylthio)pyrido[2,3-d]pyrimidin-7(8H)-one (0.57 g, 1.90 mmol, 76%). ESMS m/z 300 (M+H)⁺.

### Step 3: Synthesis of 8-ethyl-2-(methylthio)-6-phenylpyrido[2,3-d]pyrimidin-7(8H)-one

6-bromo-8-ethyl-2-(methylthio)pyrido[2,3-d]pyrimidin-7(8H)-one (150 mg, 0.50 mmol), phenylboronic acid (183 mg, 1.50 mmol), K₃PO₄ (318 mg, 1.50 mmol) and Pd(PPh₃)₄ (29 mg, 0.02 mmol) were mixed as solids and placed under argon. Argon was bubbled through the mixture of dimethoxyethane:ethanol:water (1:1:1, 2.0 mL) for 20 min. The solvent was added to the solid and the suspension was heated under microwave irradiation at 120° C. for 1 h. After completion, the reaction mixture evaporated to dryness, the crude product was purified by silica gel column chromatography using dichloromethane:ethyl acetate (100:0.5) to yield 8-ethyl-2-(methylthio)-6-phenylpyrido[2,3-d]pyrimidin-7(8H)-one as an off-white solid (121 mg, 0.41 mmol, 81%). ESMS m/z 298 (M+H)⁺; ¹H NMR (400 MHz, CDCl₃) δ ppm 8.59 (s, 1H), 8.03 (s, 1H), 4.55 (q, J=7.2 Hz, 2H), 2.63 (s, 3H), 1.35 (t, J=7.2 Hz, 3H).

### Step 4: Synthesis of 8-ethyl-2-(methylsulfinyl)-6-phenylpyrido[2,3-d]pyrimidin-7(8H)-one

To a solution of 8-ethyl-2-(methylthio)-6-phenylpyrido[2,3-d]pyrimidin-7(8H)-one (127 mg, 0.43 mmol) in dichloromethane (2 mL) was added 3-chloroperbenzoic acid (70%, 95 mg, 0.38 mmol) at 0-5° C. and the mixture was stirred at room temperature for 18 h. The reaction was diluted with dichloromethane (5 mL) and washed with saturated sodium bicarbonate solution (1×3 mL) then with water (1×3 mL). The organic layer was dried over sodium sulfate, filtered and evaporated to get 8-ethyl-2-(methylsulfinyl)-6-phenylpyrido[2,3-d]pyrimidin-7(8H)-one as a pale yellow solid (120 mg, 0.38 mmol, 88%). ESMS m/z 314 (M+H)⁺.

### Step 5: Synthesis of tert-butyl 4-(4-(8-ethyl-7-oxo-6-phenyl-7,8-dihydropyrido[2,3-d]pyrimidin-2-ylamino)-2-fluorophenyl)piperazine-1-carboxylate

8-ethyl-2-(methylsulfinyl)-6-phenylpyrido[2,3-d]pyrimidin-7(8H)-one (120 mg, 0.38 mmol) and 4-(4-amino-2-fluorophenyl)piperazine-1-carboxylic acid tert-butyl ester (113 mg, 0.38 mmol) were stirred at 120° C. for 3 h. The reaction mixture was purified by silica gel column chromatography using hexane:ethyl acetate (3:2). The isolated product was recrystallized from isopropanol to give the title compound (45 mg, 0.08 mmol, 21%) as a pale yellow solid. ESMS m/z 545 (M+H)⁺.

### Step 6: Synthesis of 8-ethyl-2-(3-fluoro-4-(piperazin-1-yl)phenylamino)-6-phenylpyrido[2,3-d]pyrimidin-7(8H)-one hydrochloride

To a stirred solution of tert-butyl 4-(4-(8-ethyl-7-oxo-6-phenyl-7,8-dihydropyrido[2,3-d]pyrimidin-2-ylamino)-2-fluorophenyl)piperazine-1-carboxylate (45 mg, 0.08 mmol) in ethyl acetate (5 mL) was added a 4M solution of hydrochloric acid in diethyl ether (5 mL) and the reaction was stirred for 18 h. The precipitate was filtered off to give 8-ethyl-2-(3-fluoro-4-(piperazin-1-yl)phenylamino)-6-phenylpyrido[2,3-d]pyrimidin-7(8H)-one hydrochloride as an off-white solid (36 mg, 0.07 mmol, 87%). ESMS m/z 445 (M+H)⁺; ¹H NMR (400 MHz, DMSO-d₆) δ ppm 10.21 (br. S., 1H), 9.22 (br. S., 2H), 8.85 (s, 1H), 8.02 (s, 1H), 7.84 (d, J=15. e.g., 1 Hz, 1H), 7.68 (d, J=7.5 Hz, 2H), 7.52 (d, J=8.5 Hz, 1H), 7.43 (t, 177.3 Hz, 2H), 7.37 (t, J=7.3 Hz, 1H), 7.11 (t, J=8.5 Hz, 1H), 4.41 (q, J=6.7 Hz, 2H), 3.22 (br. S., 8H), 1.31 (t, J=6.7 Hz, 3H).

### Examples 96-99

### Compounds 93-95

The following compounds were made by the described herein.

| No. | Structure | MW | LCMS Ion | Rt |
|---|---|---|---|---|
| 93 | 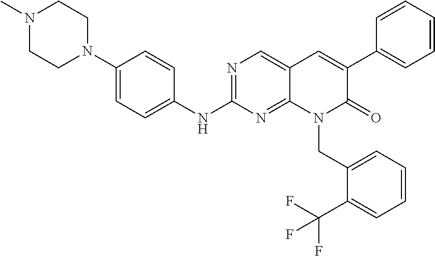 [SMILES: CN1CCN(c2ccc(Nc3ncc4cc(-c5ccccc5)c(=O)n(Cc5ccccc5C(F)(F)F)c4n3)cc2)CC1] | 570.6 | 571 | 3.46 |
| 94 | 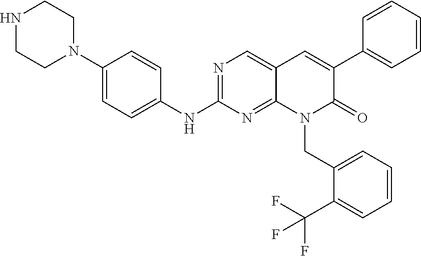 [SMILES: O=c1c(-c2ccccc2)cc2cnc(Nc3ccc(N4CCNCC4)cc3)nc2n1Cc1ccccc1C(F)(F)F] | 556.6 | 557 | 3.37 |
| 95 | 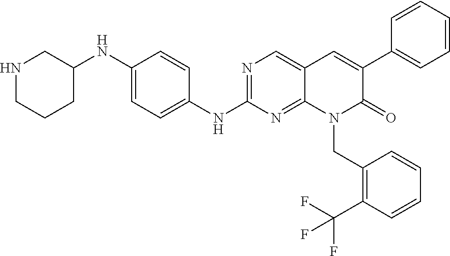 [SMILES: O=c1c(-c2ccccc2)cc2cnc(Nc3ccc(NC4CCCNC4)cc3)nc2n1Cc1ccccc1C(F)(F)F] | 570.6 | 571 | 3.49 |

### Example 100

### Identification of Compounds Having High Affinity for PAK Active Sites

A fluorescence-based assay format is used to determine IC₅₀ values of test compounds in vitro. Purified PAK kinase is incubated with ATP, and a test compound at various concentrations and a substrate peptide containing two fluorophores. In a second step, the reaction mix is incubated with a site-specific protease that cleaves non-phosphorylated but not phosphorylated substrate peptide, disrupting the FRET signal generated by the two fluorophores in the cleaved peptide (Z'Lyte™ Kinase assay platform; Life Technologies).

Reagents: 50 mM HEPES, pH 7.5; 0.01% BRIJ-35; 10 mM MgCl₂; 1 mM EGTA, 2 uM substrate peptide Ser/Thr20 (proprietary Life Technologies Sequence), PAK enzyme [2.42-30.8 ng for PAK1, 0.29-6 ng for PAK2, 1.5-20 ng for PAK3 and 0.1-0.86 ng for PAK-4; actual enzyme amounts depend on lot activity of the enzyme preparation]

Test compounds are dissolved in DMSO at various concentrations; the final DMSO concentration in the assay reaction is 1%.

ATP concentration at Km apparent is used in the assay [50 μM ATP for PAK1 assay, 75 μM ATP for PAK2 assay, 100 μM ATP for PAK3 assay, 5 μM ATP for PAK-4 assay] in a total assay volume of 10 μl. Assay reactions are incubated at room temperature for 1 hr. Following the kinase reaction, 5 μM of 1:256 dilution of development solution A (Life Technologies) is added and the reaction mix is incubated for an additional 1 hr at room temperature.

Plates are analyzed in a standard fluorescence plate reader (Tecan or equivalent) using an excitation wavelength of 400 nm and emission wavelengths of 445 nm and 520 nm. Inhibition of kinase reaction is determined by emission ratio=emission@ 445 nm/emission@ 520 nm

Based on these data, specific compounds have been identified that have relatively high affinity for the catalytic domain of at least one PAK isoform, and are therefore useful inhibitors, as described herein.

TABLE 1

| | | PAK1 | PAK2 | PAK3 | PAK4 |
|---|---|---|---|---|---|
| Compd. | Structure | IC₅₀ μM | IC₅₀ μM | IC₅₀ μM | IC₅₀ μM |
| 1 | 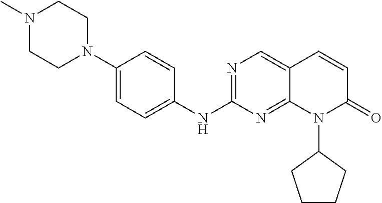 [SMILES: CN1CCN(c2ccc(Nc3ncc4ccc(=O)n(C5CCCC5)c4n3)cc2)CC1] | A | B | B | B |
| 2 | 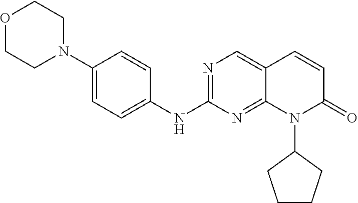 [SMILES: O=c1ccc2cnc(Nc3ccc(N4CCOCC4)cc3)nc2n1C1CCCC1] | C | C | | B |
| 3 | 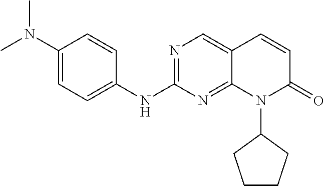 [SMILES: CN(C)c1ccc(Nc2ncc3ccc(=O)n(C4CCCC4)c3n2)cc1] | C | C | | B |
| 4 | 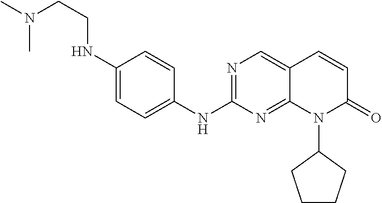 [SMILES: CN(C)CCNc1ccc(Nc2ncc3ccc(=O)n(C4CCCC4)c3n2)cc1] | B | B | | B |
| 5 | 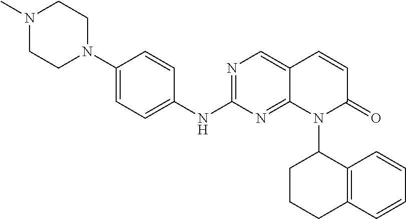 [SMILES: CN1CCN(c2ccc(Nc3ncc4ccc(=O)n(C5CCCc6ccccc65)c4n3)cc2)CC1] | B | B | B | B |
| 6 | 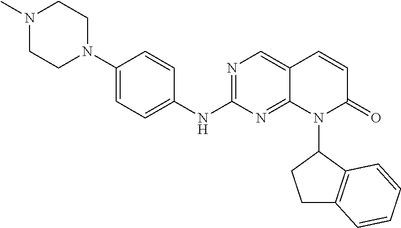 [SMILES: CN1CCN(c2ccc(Nc3ncc4ccc(=O)n(C5CCc6ccccc65)c4n3)cc2)CC1] | A | B | B | B |
| 7 | 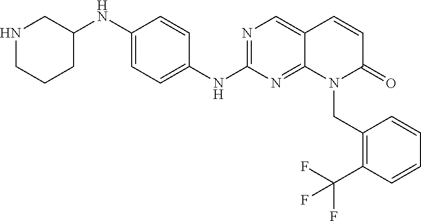 [SMILES: O=c1ccc2cnc(Nc3ccc(NC4CCCNC4)cc3)nc2n1Cc1ccccc1C(F)(F)F] | A | A | A | A |
| 8 | 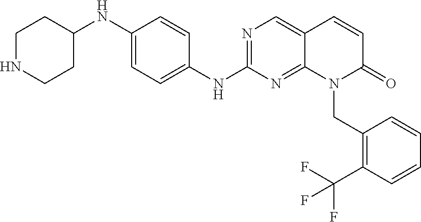 [SMILES: O=c1ccc2cnc(Nc3ccc(NC4CCNCC4)cc3)nc2n1Cc1ccccc1C(F)(F)F] | A | A | A | A |
| 9 | 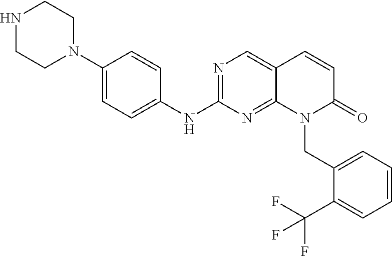 [SMILES: O=c1ccc2cnc(Nc3ccc(N4CCNCC4)cc3)nc2n1Cc1ccccc1C(F)(F)F] | A | A | A | A |
| 10 | 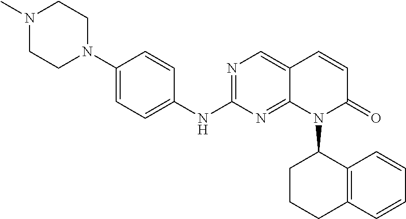 [SMILES: CN1CCN(c2ccc(Nc3ncc4ccc(=O)n([C@@H]5CCCc6ccccc65)c4n3)cc2)CC1] | B | B | B | B |
| 11 | 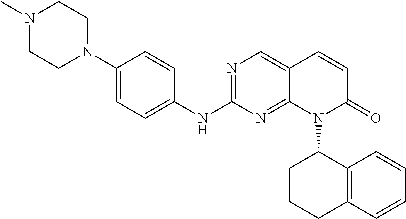 [SMILES: CN1CCN(c2ccc(Nc3ncc4ccc(=O)n([C@H]5CCCc6ccccc65)c4n3)cc2)CC1] | A | B | B | B |
| 12 | 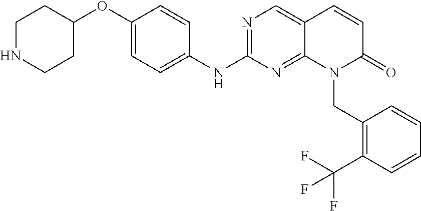 [SMILES: O=c1ccc2cnc(Nc3ccc(OC4CCNCC4)cc3)nc2n1Cc1ccccc1C(F)(F)F] | A | A | A | A |
| 13 | 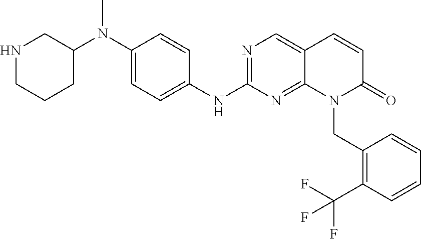 [SMILES: CN(c1ccc(Nc2ncc3ccc(=O)n(Cc4ccccc4C(F)(F)F)c3n2)cc1)C1CCCNC1] | A | A | A | A |
| 14 | 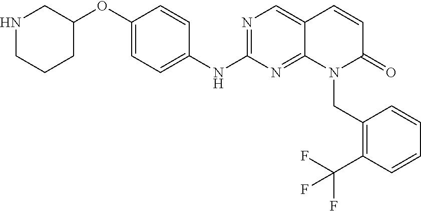 [SMILES: O=c1ccc2cnc(Nc3ccc(OC4CCCNC4)cc3)nc2n1Cc1ccccc1C(F)(F)F] | A | A | A | A |
| 15 | 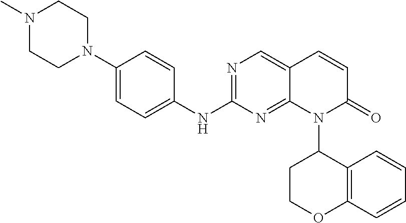 [SMILES: CN1CCN(c2ccc(Nc3ncc4ccc(=O)n(C5CCOc6ccccc65)c4n3)cc2)CC1] | B | B | B | B |
| 16 | 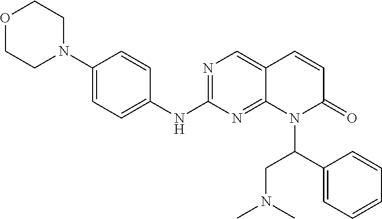 [SMILES: CN(C)CC(c1ccccc1)n1c(=O)ccc2cnc(Nc3ccc(N4CCOCC4)cc3)nc21] | C | C | C | C |
| 17 | 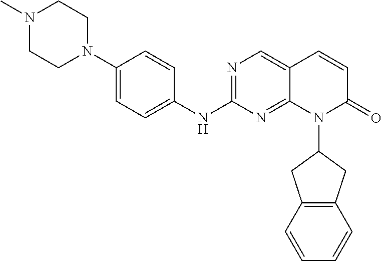 [SMILES: CN1CCN(c2ccc(Nc3ncc4ccc(=O)n(C5Cc6ccccc6C5)c4n3)cc2)CC1] | A | B | B | A |
| 18 | 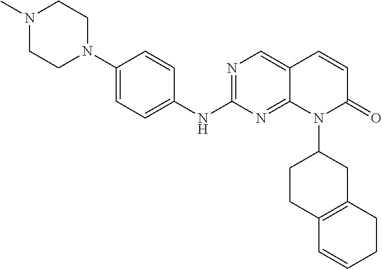 [SMILES: CN1CCN(c2ccc(Nc3ncc4ccc(=O)n(C5CCC6=C(CCC=C6)C5)c4n3)cc2)CC1] | A | A | A | A |
| 19 | 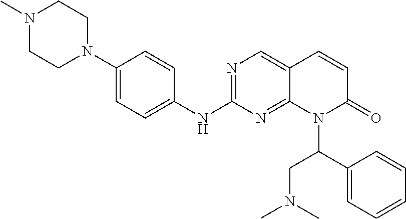 [SMILES: CN(C)CC(c1ccccc1)n1c(=O)ccc2cnc(Nc3ccc(N4CCN(C)CC4)cc3)nc21] | C | C | C | C |
| 20 | 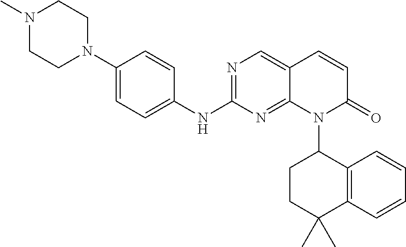 [SMILES: CN1CCN(c2ccc(Nc3ncc4ccc(=O)n(C5CCC(C)(C)c6ccccc65)c4n3)cc2)CC1] | A | A | B | B |
| 21 | 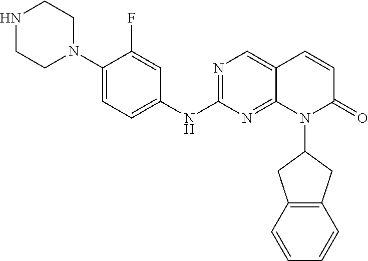 [SMILES: O=c1ccc2cnc(Nc3ccc(N4CCNCC4)c(F)c3)nc2n1C1Cc2ccccc2C1] | A | A | B | A |
| 22 | 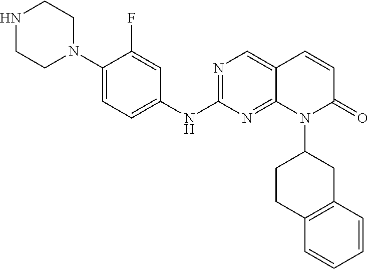 [SMILES: O=c1ccc2cnc(Nc3ccc(N4CCNCC4)c(F)c3)nc2n1C1CCc2ccccc2C1] | A | A | A | A |
| 26 | 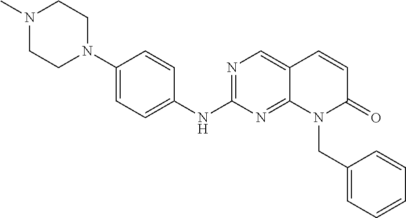 [SMILES: CN1CCN(c2ccc(Nc3ncc4ccc(=O)n(Cc5ccccc5)c4n3)cc2)CC1] | B | B | | C |
| 27 | 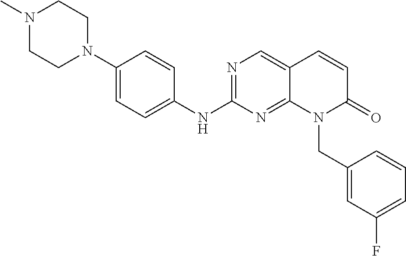 [SMILES: CN1CCN(c2ccc(Nc3ncc4ccc(=O)n(Cc5cccc(F)c5)c4n3)cc2)CC1] | B | B | | B |
| 28 | 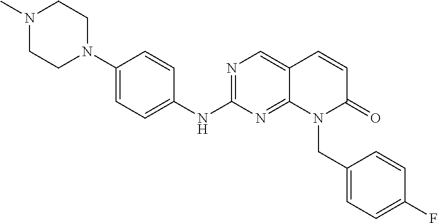 [SMILES: CN1CCN(c2ccc(Nc3ncc4ccc(=O)n(Cc5ccc(F)cc5)c4n3)cc2)CC1] | B | C | | C |
| 29 | 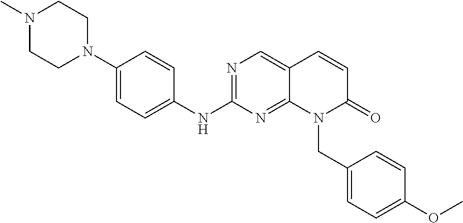 | C | C | | C |
| 30 | 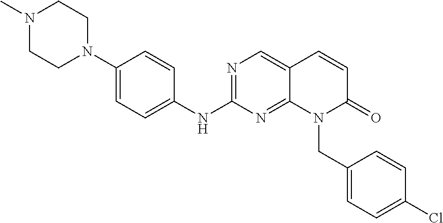 [SMILES: CN1CCN(c2ccc(Nc3ncc4ccc(=O)n(Cc5ccc(Cl)cc5)c4n3)cc2)CC1] | B | C | | C |
| 31 | 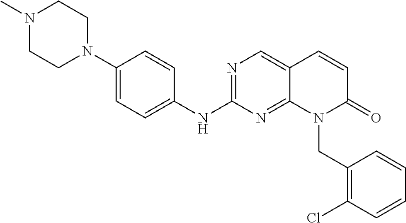 [SMILES: CN1CCN(c2ccc(Nc3ncc4ccc(=O)n(Cc5ccccc5Cl)c4n3)cc2)CC1] | A | A | | B |
| 32 | 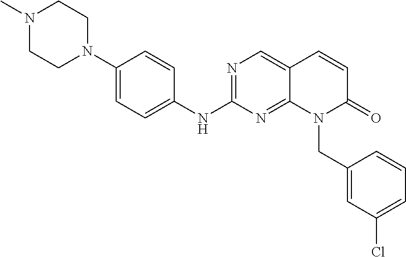 [SMILES: CN1CCN(c2ccc(Nc3ncc4ccc(=O)n(Cc5cccc(Cl)c5)c4n3)cc2)CC1] | B | C | | C |
| 33 | 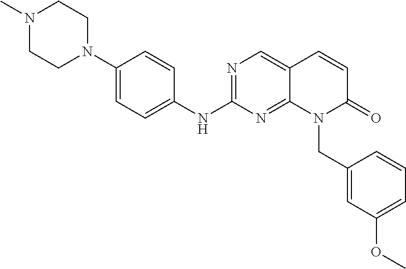 | B | C | | C |
| 34 | 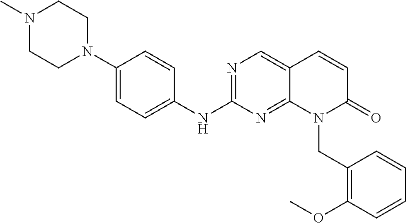 [SMILES: COc1ccccc1Cn1c(=O)ccc2cnc(Nc3ccc(N4CCN(C)CC4)cc3)nc21] | A | B | | B |
| 35 | 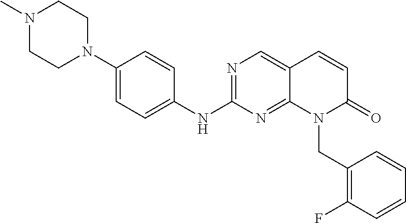 [SMILES: CN1CCN(c2ccc(Nc3ncc4ccc(=O)n(Cc5ccccc5F)c4n3)cc2)CC1] | B | B | | B |
| 36 | 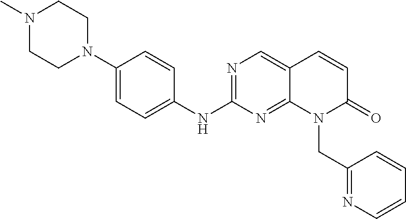 [SMILES: CN1CCN(c2ccc(Nc3ncc4ccc(=O)n(Cc5ccccn5)c4n3)cc2)CC1] | B | C | | B |
| 37 | 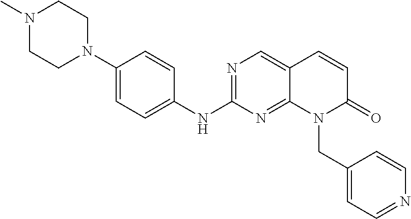 [SMILES: CN1CCN(c2ccc(Nc3ncc4ccc(=O)n(Cc5ccncc5)c4n3)cc2)CC1] | B | C | | C |
| 38 | 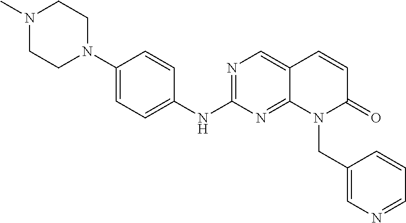 [SMILES: CN1CCN(c2ccc(Nc3ncc4ccc(=O)n(Cc5cccnc5)c4n3)cc2)CC1] | B | C | | C |
| 39 | 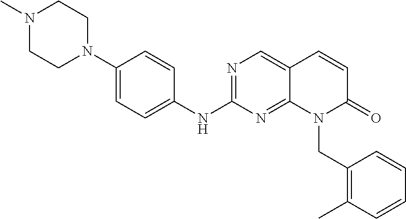 [SMILES: Cc1ccccc1Cn1c(=O)ccc2cnc(Nc3ccc(N4CCN(C)CC4)cc3)nc21] | A | A | | B |
| 40 | 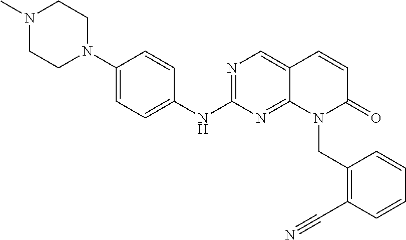 [SMILES: CN1CCN(c2ccc(Nc3ncc4ccc(=O)n(Cc5ccccc5C#N)c4n3)cc2)CC1] | A | A | | B |
| 41 | 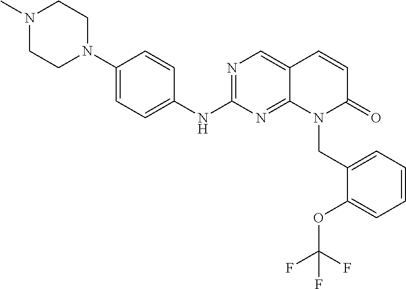 [SMILES: CN1CCN(c2ccc(Nc3ncc4ccc(=O)n(Cc5ccccc5OC(F)(F)F)c4n3)cc2)CC1] | A | A | | B |
| 42 | 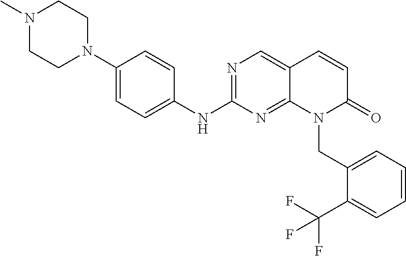 [SMILES: CN1CCN(c2ccc(Nc3ncc4ccc(=O)n(Cc5ccccc5C(F)(F)F)c4n3)cc2)CC1] | A | A | A | A |
| 43 | 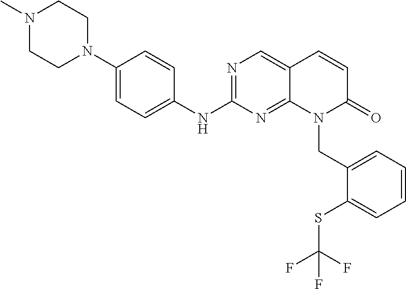 [SMILES: CN1CCN(c2ccc(Nc3ncc4ccc(=O)n(Cc5ccccc5SC(F)(F)F)c4n3)cc2)CC1] | A | A | A | A |
| 44 | 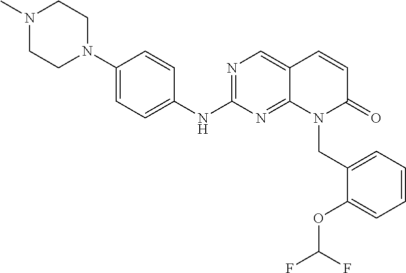 [SMILES: CN1CCN(c2ccc(Nc3ncc4ccc(=O)n(Cc5ccccc5OC(F)F)c4n3)cc2)CC1] | A | A | A | A |
| 45 | 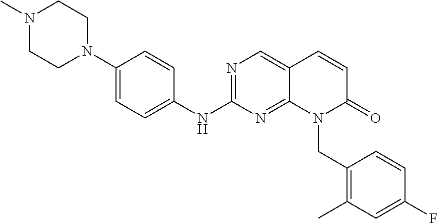 [SMILES: Cc1cc(F)ccc1Cn1c(=O)ccc2cnc(Nc3ccc(N4CCN(C)CC4)cc3)nc21] | B | B | B | B |
| 46 | 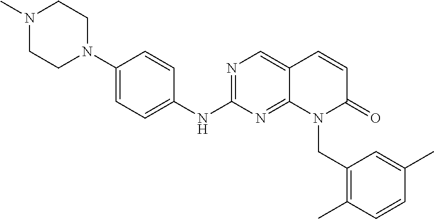 | B | B | B | B |
| 47 | 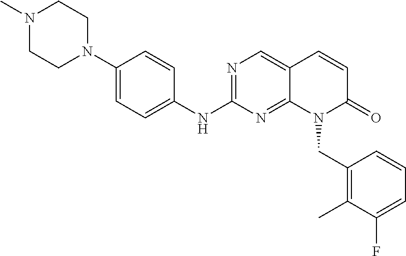 | A | B | C | B |
| 48 | 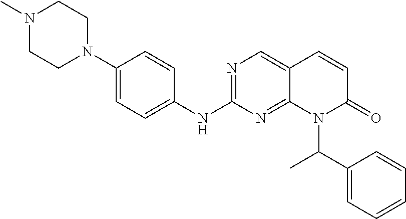 [SMILES: CC(c1ccccc1)n1c(=O)ccc2cnc(Nc3ccc(N4CCN(C)CC4)cc3)nc21] | B | B | C | B |
| 49 | 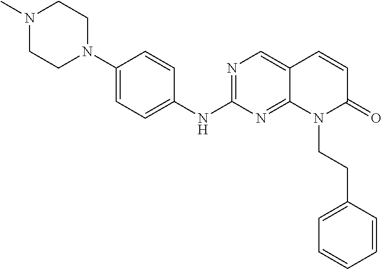 [SMILES: CN1CCN(c2ccc(Nc3ncc4ccc(=O)n(CCc5ccccc5)c4n3)cc2)CC1] | A | A | B | A |
| 50 | 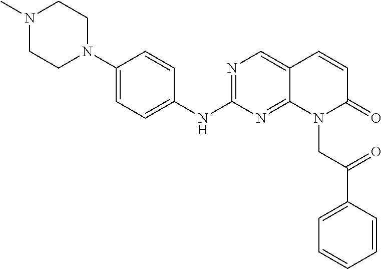 [SMILES: CN1CCN(c2ccc(Nc3ncc4ccc(=O)n(CC(=O)c5ccccc5)c4n3)cc2)CC1] | B | B | B | B |
| 51 | 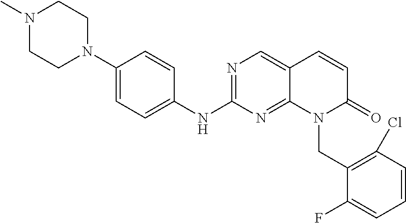 [SMILES: CN1CCN(c2ccc(Nc3ncc4ccc(=O)n(Cc5c(F)cccc5Cl)c4n3)cc2)CC1] | A | A | A | A |
| 52 | 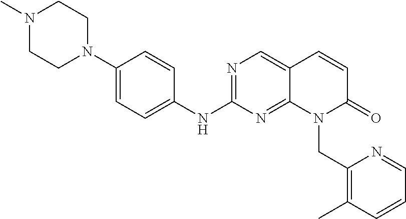 [SMILES: Cc1cccnc1Cn1c(=O)ccc2cnc(Nc3ccc(N4CCN(C)CC4)cc3)nc21] | B | B | C | B |
| 53 | 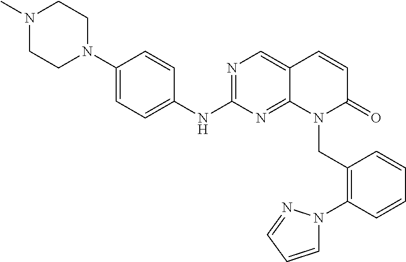 [SMILES: CN1CCN(c2ccc(Nc3ncc4ccc(=O)n(Cc5ccccc5-n5cccn5)c4n3)cc2)CC1] | A | B | B | B |
| 54 | 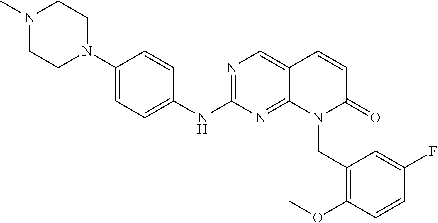 [SMILES: COc1ccc(F)cc1Cn1c(=O)ccc2cnc(Nc3ccc(N4CCN(C)CC4)cc3)nc21] | A | B | B | B |
| 55 | 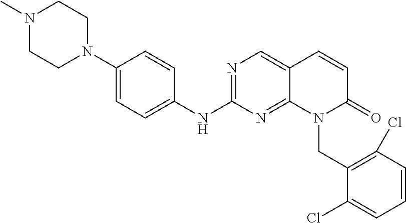 [SMILES: CN1CCN(c2ccc(Nc3ncc4ccc(=O)n(Cc5c(Cl)cccc5Cl)c4n3)cc2)CC1] | A | A | B | A |
| 56 | 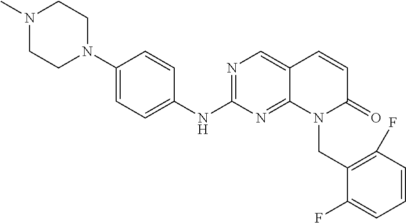 [SMILES: CN1CCN(c2ccc(Nc3ncc4ccc(=O)n(Cc5c(F)cccc5F)c4n3)cc2)CC1] | A | B | B | B |
| 57 | 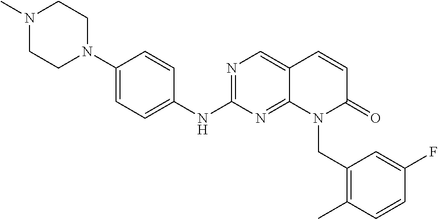 [SMILES: Cc1ccc(F)cc1Cn1c(=O)ccc2cnc(Nc3ccc(N4CCN(C)CC4)cc3)nc21] | A | B | B | A |
| 58 | 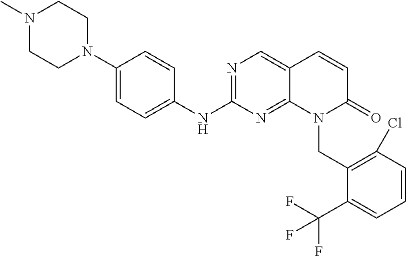 [SMILES: CN1CCN(c2ccc(Nc3ncc4ccc(=O)n(Cc5c(Cl)cccc5C(F)(F)F)c4n3)cc2)CC1] | A | A | A | A |
| 59 | 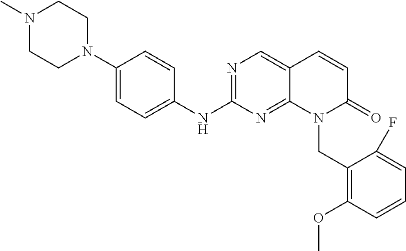 [SMILES: COc1cccc(F)c1Cn1c(=O)ccc2cnc(Nc3ccc(N4CCN(C)CC4)cc3)nc21] | A | B | B | A |
| 60 | 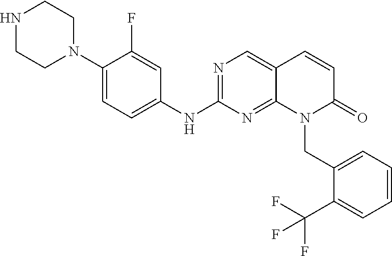 [SMILES: O=c1ccc2cnc(Nc3ccc(N4CCNCC4)c(F)c3)nc2n1Cc1ccccc1C(F)(F)F] | A | A | A | A |
| 61 | 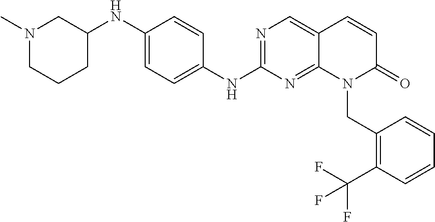 [SMILES: CN1CCCC(Nc2ccc(Nc3ncc4ccc(=O)n(Cc5ccccc5C(F)(F)F)c4n3)cc2)C1] | A | A | A | A |
| 62 | 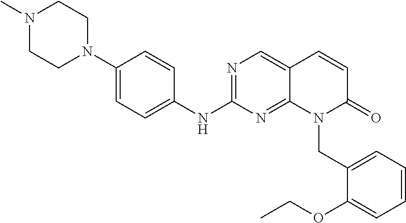 [SMILES: CCOc1ccccc1Cn1c(=O)ccc2cnc(Nc3ccc(N4CCN(C)CC4)cc3)nc21] | A | A | A | B |
| 63 | 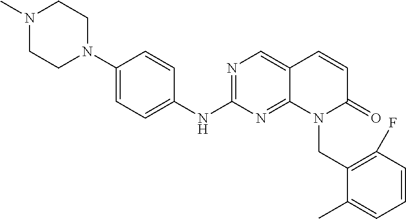 [SMILES: Cc1cccc(F)c1Cn1c(=O)ccc2cnc(Nc3ccc(N4CCN(C)CC4)cc3)nc21] | A | A | A | A |
| 64 | 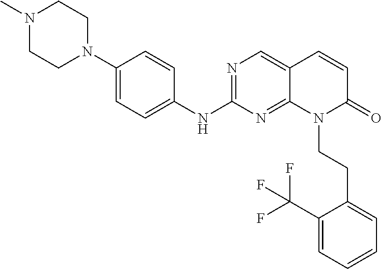 [SMILES: CN1CCN(c2ccc(Nc3ncc4ccc(=O)n(CCc5ccccc5C(F)(F)F)c4n3)cc2)CC1] | A | A | B | A |
| 65 | 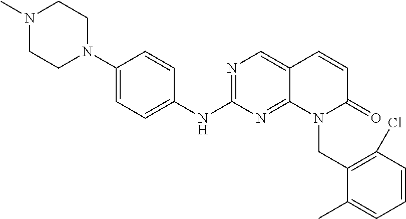 [SMILES: Cc1cccc(Cl)c1Cn1c(=O)ccc2cnc(Nc3ccc(N4CCN(C)CC4)cc3)nc21] | A | B | B | A |
| 66 | 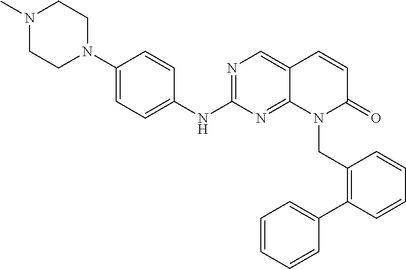 [SMILES: CN1CCN(c2ccc(Nc3ncc4ccc(=O)n(Cc5ccccc5-c5ccccc5)c4n3)cc2)CC1] | A | A | A | B |
| 67 | 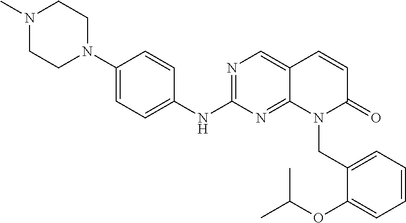 [SMILES: CC(C)Oc1ccccc1Cn1c(=O)ccc2cnc(Nc3ccc(N4CCN(C)CC4)cc3)nc21] | A | A | A | A |
| 68 | 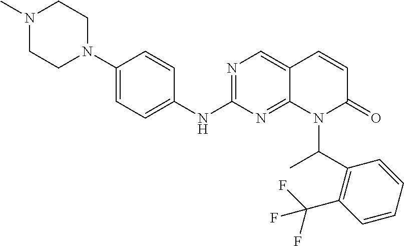 [SMILES: CC(c1ccccc1C(F)(F)F)n1c(=O)ccc2cnc(Nc3ccc(N4CCN(C)CC4)cc3)nc21] | B | C | C | B |
| 69 | 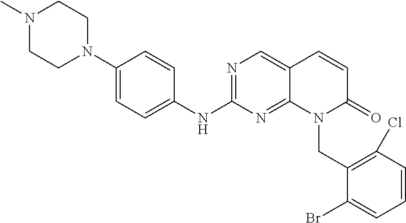 [SMILES: CN1CCN(c2ccc(Nc3ncc4ccc(=O)n(Cc5c(Cl)cccc5Br)c4n3)cc2)CC1] | A | A | A | A |
| 70 | 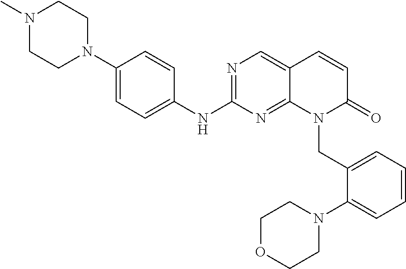 [SMILES: CN1CCN(c2ccc(Nc3ncc4ccc(=O)n(Cc5ccccc5N5CCOCC5)c4n3)cc2)CC1] | A | A | A | A |
| 72 | 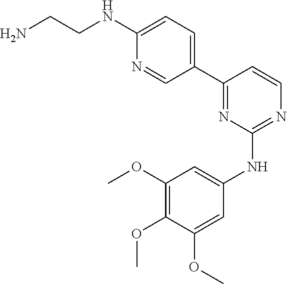 [SMILES: COc1cc(Nc2nccc(-c3ccc(NCCN)nc3)n2)cc(OC)c1OC] | A | B | B | A |
| 73 | 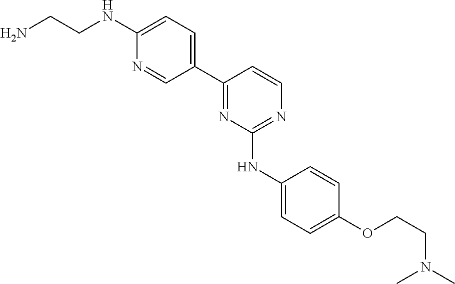 [SMILES: CN(C)CCOc1ccc(Nc2nccc(-c3ccc(NCCN)nc3)n2)cc1] | B | B | | B |
| 74 | 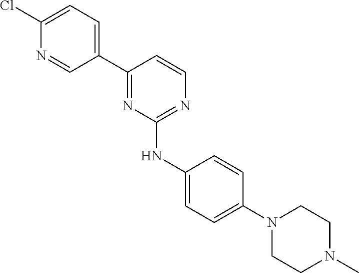 [SMILES: CN1CCN(c2ccc(Nc3nccc(-c4ccc(Cl)nc4)n3)cc2)CC1] | B | C | | C |
| 75 | 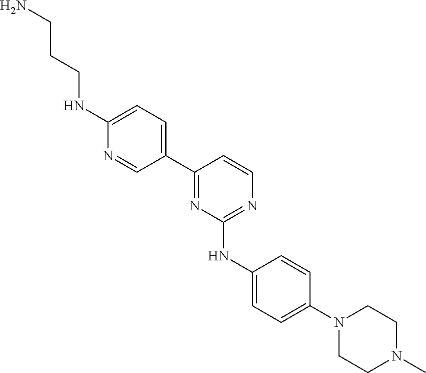 [SMILES: CN1CCN(c2ccc(Nc3nccc(-c4ccc(NCCCN)nc4)n3)cc2)CC1] | A | B | | B |
| 76 | 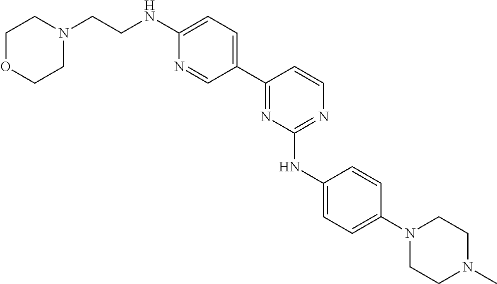 [SMILES: CN1CCN(c2ccc(Nc3nccc(-c4ccc(NCCN5CCOCC5)nc4)n3)cc2)CC1] | B | C | | B |
| 77 | 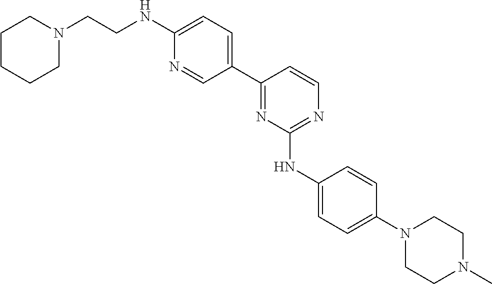 [SMILES: CN1CCN(c2ccc(Nc3nccc(-c4ccc(NCCN5CCCCC5)nc4)n3)cc2)CC1] | B | B | C | A |
| 78 | 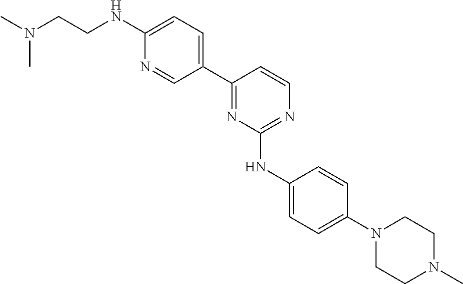 | B | B | | B |
| 79 | 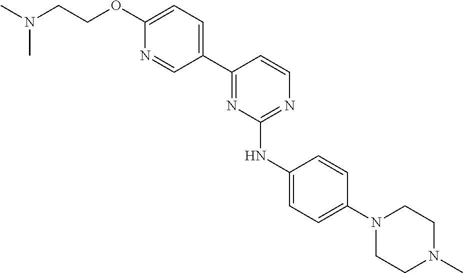 | B | C | | C |
| 80 | 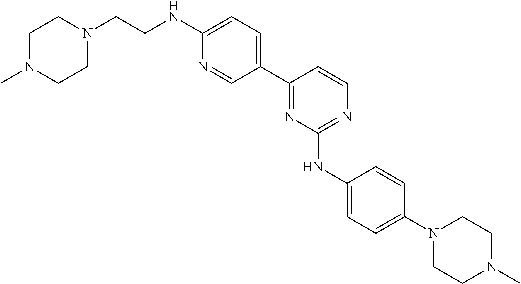 | B | C | | C |
| 81 | 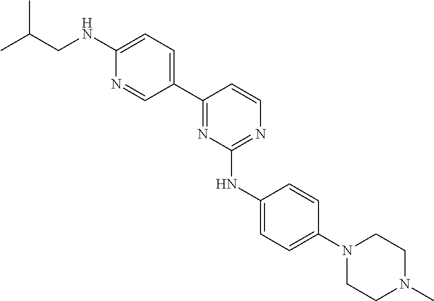 [SMILES: CC(C)CNc1ccc(-c2ccnc(Nc3ccc(N4CCN(C)CC4)cc3)n2)cn1] | B | B | C | A |
| 82 | 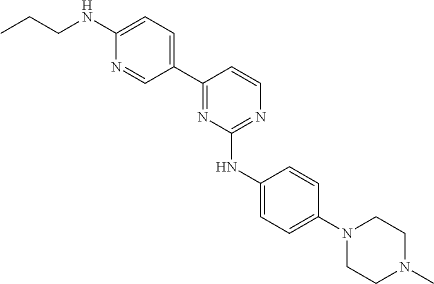 [SMILES: CCCNc1ccc(-c2ccnc(Nc3ccc(N4CCN(C)CC4)cc3)n2)cn1] | B | B | | B |
| 83 | 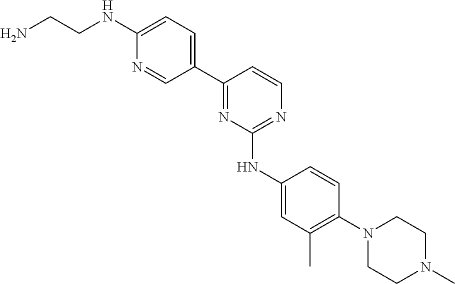 [SMILES: Cc1cc(Nc2nccc(-c3ccc(NCCN)nc3)n2)ccc1N1CCN(C)CC1] | A | B | B | A |
| 84 | 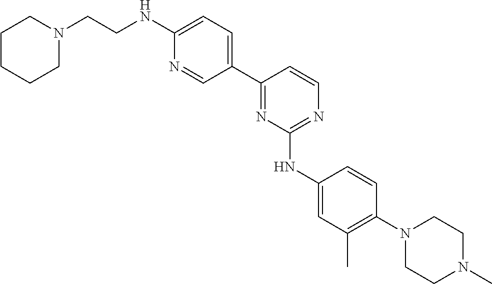 [SMILES: Cc1cc(Nc2nccc(-c3ccc(NCCN4CCCCC4)nc3)n2)ccc1N1CCN(C)CC1] | B | B | C | A |
| 85 | 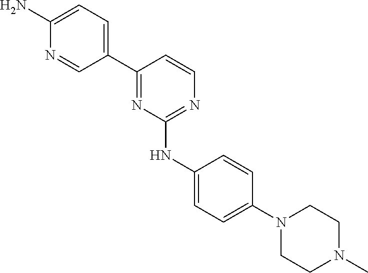 [SMILES: CN1CCN(c2ccc(Nc3nccc(-c4ccc(N)nc4)n3)cc2)CC1] | B | B | C | B |
| 86 | 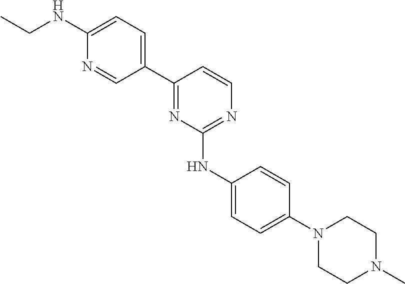 [SMILES: CCNc1ccc(-c2ccnc(Nc3ccc(N4CCN(C)CC4)cc3)n2)cn1] | B | C | C | A |
| 87 | 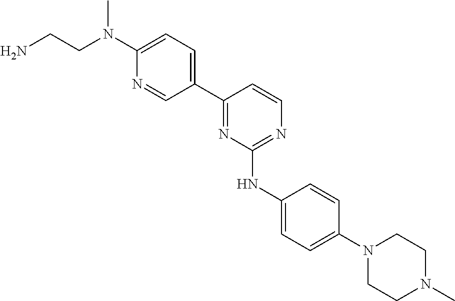 [SMILES: CN1CCN(c2ccc(Nc3nccc(-c4ccc(N(C)CCN)nc4)n3)cc2)CC1] | B | B | C | B |
| 88 | 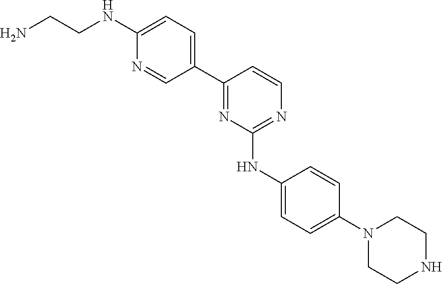 [SMILES: NCCNc1ccc(-c2ccnc(Nc3ccc(N4CCNCC4)cc3)n2)cn1] | A | B | B | A |
| 89 | 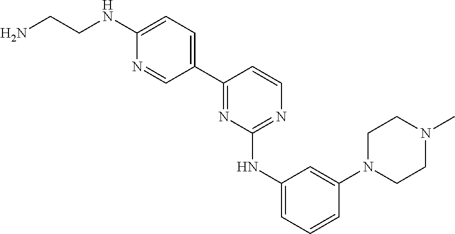 [SMILES: CN1CCN(c2cccc(Nc3nccc(-c4ccc(NCCN)nc4)n3)c2)CC1] | B | B | C | A |
| 90 | 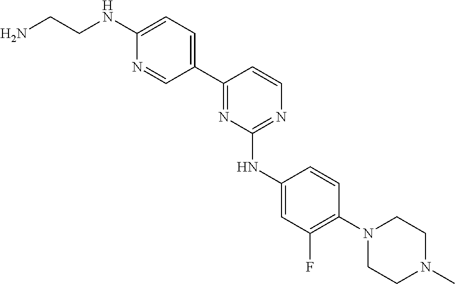 [SMILES: CN1CCN(c2ccc(Nc3nccc(-c4ccc(NCCN)nc4)n3)cc2F)CC1] | A | B | B | A |
| 91 | 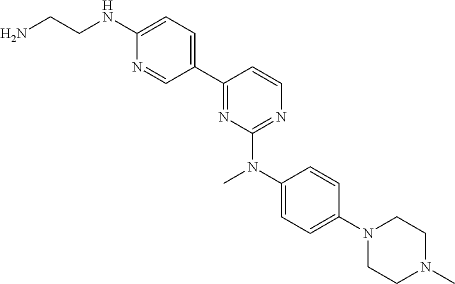 [SMILES: CN1CCN(c2ccc(N(C)c3nccc(-c4ccc(NCCN)nc4)n3)cc2)CC1] | C | C | | C |
| 92 | 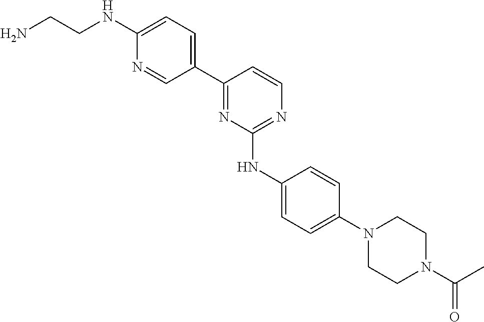 [SMILES: CC(=O)N1CCN(c2ccc(Nc3nccc(-c4ccc(NCCN)nc4)n3)cc2)CC1] | C | C | C | A |
| 93 | 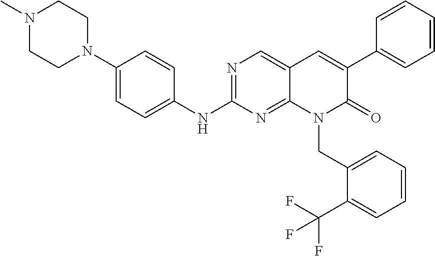 [SMILES: CN1CCN(c2ccc(Nc3ncc4cc(-c5ccccc5)c(=O)n(Cc5ccccc5C(F)(F)F)c4n3)cc2)CC1] | A | A | A | C |
| 94 | 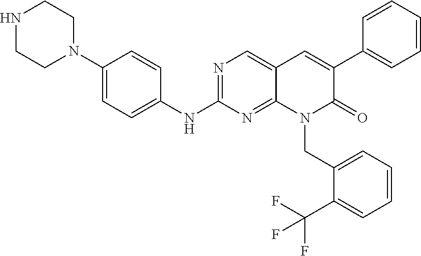 [SMILES: O=c1c(-c2ccccc2)cc2cnc(Nc3ccc(N4CCNCC4)cc3)nc2n1Cc1ccccc1C(F)(F)F] | A | A | A | B |
| 95 | 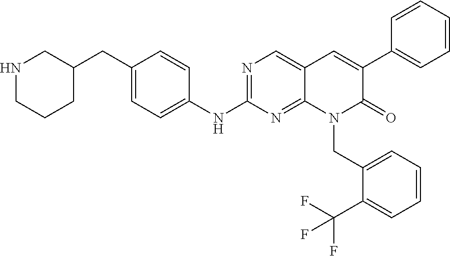 [SMILES: O=c1c(-c2ccccc2)cc2cnc(Nc3ccc(CC4CCCNC4)cc3)nc2n1Cc1ccccc1C(F)(F)F] | A | A | A | B |
| 96 | 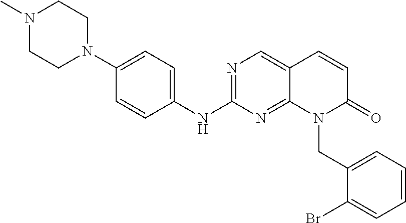 [SMILES: CN1CCN(c2ccc(Nc3ncc4ccc(=O)n(Cc5ccccc5Br)c4n3)cc2)CC1] | A | A | A | A |
| 97 | 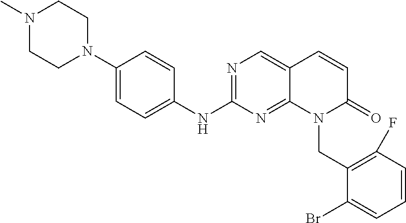 [SMILES: CN1CCN(c2ccc(Nc3ncc4ccc(=O)n(Cc5c(F)cccc5Br)c4n3)cc2)CC1] | A | A | A | A |
| 98 | 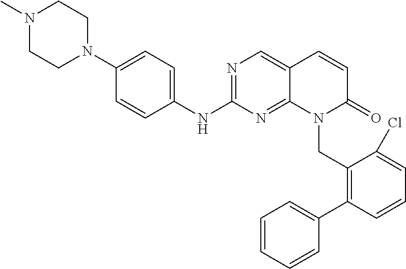 [SMILES: CN1CCN(c2ccc(Nc3ncc4ccc(=O)n(Cc5c(Cl)cccc5-c5ccccc5)c4n3)cc2)CC1] | B | C | C | B |
| 99 | 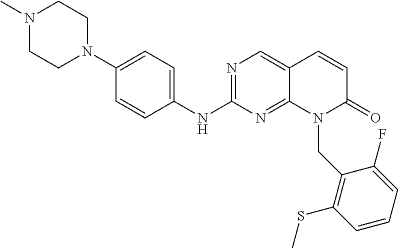 [SMILES: CSc1cccc(F)c1Cn1c(=O)ccc2cnc(Nc3ccc(N4CCN(C)CC4)cc3)nc21] | A | A | A | A |
| 100 | 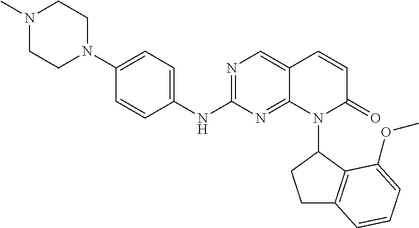 | B | C | C | B |

A: IC₅₀ < 1 μM;
B: IC₅₀ > 1 μM and <10 μM
C: IC₅₀ > 10 μM

### Example 101

### Identification of Compounds Having High Affinity for PAK Active Sites

A fluorescence-based assay format is used to determine IC₅₀ values of test compounds in vitro. Purified PAK kinase is incubated with ATP, and a test compound at various concentrations and a substrate peptide containing two fluorophores. In a second, step, the reaction mix is incubated with a site-specific protease that cleaves non-phosphorylated but not phosphorylated substrate peptide, disrupting the FRET signal generated by the two fluorophores in the cleaved peptide (Z'Lyte™ Kinase assay platform; Life Technologies).

Reagents: 50 mM HEPES, pH 7.5; 0.01% BRIJ-35; 10 mM MgCl₂; 1 mM EGTA, 2 uM substrate peptide Ser/Thr20 (proprietary Life Technologies Sequence), PAK enzyme [2.42-30.8 ng for PAK1, 0.29-6 ng for PAK2, 1.5-20 ng for PAK3 and 0.1-0.86 ng for PAK-4; actual enzyme amounts depend on lot activity of the enzyme preparation]

Test compounds are dissolved in DMSO at various concentrations; the final DMSO concentration in the assay reaction is 1%.

ATP concentration at Km apparent is used in the assay [50 μM ATP for PAK1 assay, 75 μM ATP for PAK2 assay, 100 μM ATP for PAK3 assay, 5 μM ATP for PAK-4 assay] in a total assay volume of 10 μl. Assay reactions are incubated at room temperature for 1 hr. Following the kinase reaction, 5 μM of 1:256 dilution of development solution A (Life Technologies) is added and the reaction mix is incubated for an additional 1 hr at room temperature.

Plates are analyzed in a standard fluorescence plate reader (Tecan or equivalent) using an excitation wavelength of 400 nm and emission wavelengths of 445 nm and 520 nm. Inhibition of kinase reaction is determined by

emission ratio=emission@445 nm/emission@520 nm

Based on these data, specific compounds have been identified that have relatively high affinity for the catalytic domain of at least one PAK isoform, and are therefore useful inhibitors, as described herein.

TABLE 1

| | | | | PA K3 | PAK4 |
|---|---|---|---|---|---|
| | | PAK1 | PAK2 | IC₅₀ | IC₅₀ |
| Compd. | Structure | IC₅₀ μM | IC₅₀ μM | μM | μM |
| 56 | 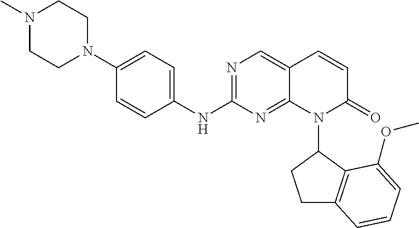 | B | C | C | B |
| 57 | 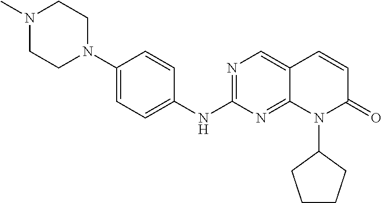 [SMILES: CN1CCN(c2ccc(Nc3ncc4ccc(=O)n(C5CCCC5)c4n3)cc2)CC1] | A | B | B | B |
| 58 | 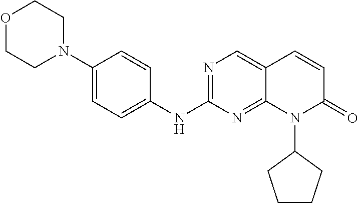 [SMILES: O=c1ccc2cnc(Nc3ccc(N4CCOCC4)cc3)nc2n1C1CCCC1] | C | C | | B |
| 59 | 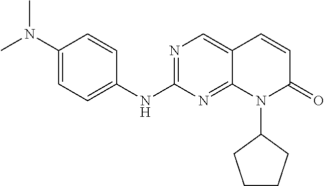 [SMILES: CN(C)c1ccc(Nc2ncc3ccc(=O)n(C4CCCC4)c3n2)cc1] | C | C | | B |
| 60 | 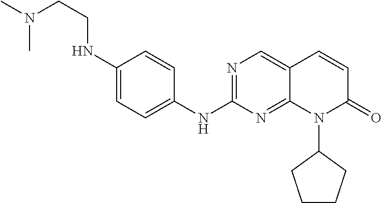 [SMILES: CN(C)CCNc1ccc(Nc2ncc3ccc(=O)n(C4CCCC4)c3n2)cc1] | B | B | | B |
| 61 | 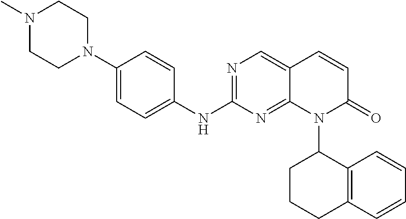 [SMILES: CN1CCN(c2ccc(Nc3ncc4ccc(=O)n(C5CCCc6ccccc65)c4n3)cc2)CC1] | B | B | B | B |
| 62 | 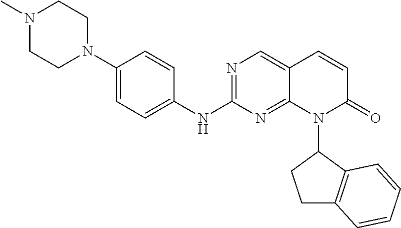 [SMILES: CN1CCN(c2ccc(Nc3ncc4ccc(=O)n(C5CCc6ccccc65)c4n3)cc2)CC1] | A | B | B | B |
| 63 | 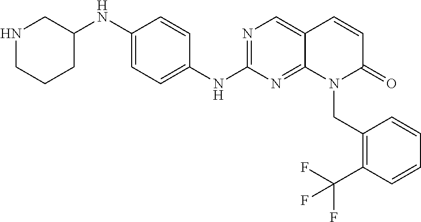 [SMILES: O=c1ccc2cnc(Nc3ccc(NC4CCCNC4)cc3)nc2n1Cc1ccccc1C(F)(F)F] | A | A | A | A |
| 64 | 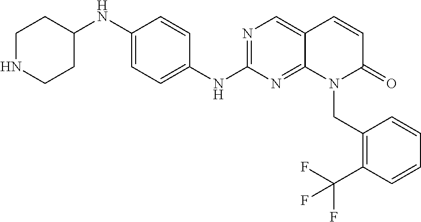 [SMILES: O=c1ccc2cnc(Nc3ccc(NC4CCNCC4)cc3)nc2n1Cc1ccccc1C(F)(F)F] | A | A | A | A |
| 65 | 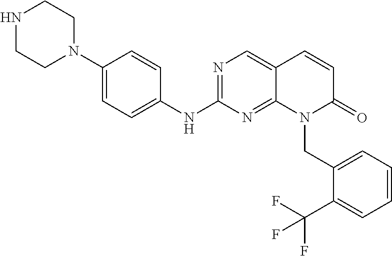 [SMILES: O=c1ccc2cnc(Nc3ccc(N4CCNCC4)cc3)nc2n1Cc1ccccc1C(F)(F)F] | A | A | A | A |
| 66 | 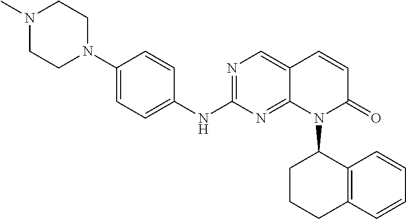 [SMILES: CN1CCN(c2ccc(Nc3ncc4ccc(=O)n([C@@H]5CCCc6ccccc65)c4n3)cc2)CC1] | B | B | B | B |
| 67 | 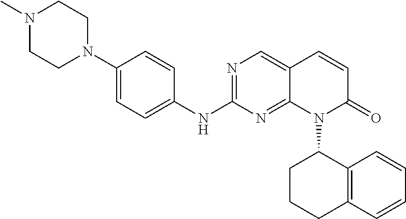 [SMILES: CN1CCN(c2ccc(Nc3ncc4ccc(=O)n([C@H]5CCCc6ccccc65)c4n3)cc2)CC1] | A | B | B | B |
| 68 | 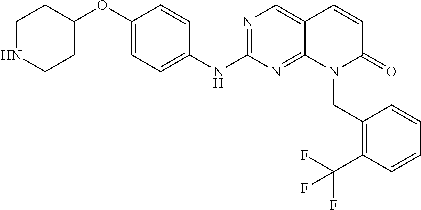 [SMILES: O=c1ccc2cnc(Nc3ccc(OC4CCNCC4)cc3)nc2n1Cc1ccccc1C(F)(F)F] | A | A | A | A |
| 69 | 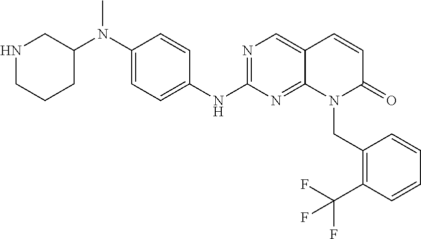 [SMILES: CN(c1ccc(Nc2ncc3ccc(=O)n(Cc4ccccc4C(F)(F)F)c3n2)cc1)C1CCCNC1] | A | A | A | A |
| 70 | 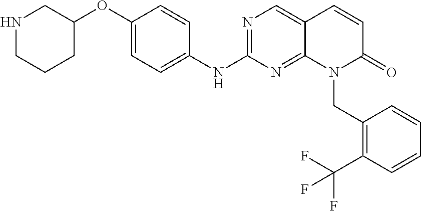 [SMILES: O=c1ccc2cnc(Nc3ccc(OC4CCCNC4)cc3)nc2n1Cc1ccccc1C(F)(F)F] | A | A | A | A |
| 71 | 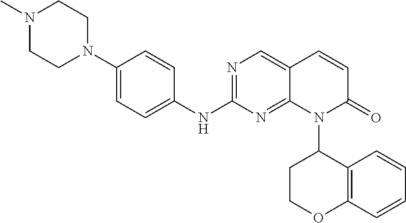 [SMILES: CN1CCN(c2ccc(Nc3ncc4ccc(=O)n(C5CCOc6ccccc65)c4n3)cc2)CC1] | B | B | B | B |
| 72 | 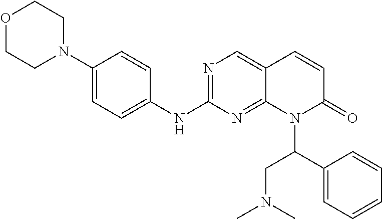 [SMILES: CN(C)CC(c1ccccc1)n1c(=O)ccc2cnc(Nc3ccc(N4CCOCC4)cc3)nc21] | C | C | C | C |
| 73 | 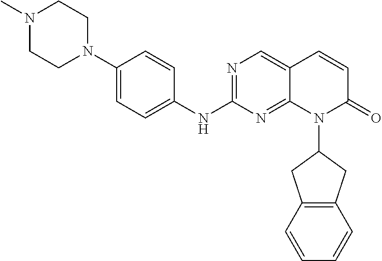 [SMILES: CN1CCN(c2ccc(Nc3ncc4ccc(=O)n(C5Cc6ccccc6C5)c4n3)cc2)CC1] | A | B | B | A |
| 74 | 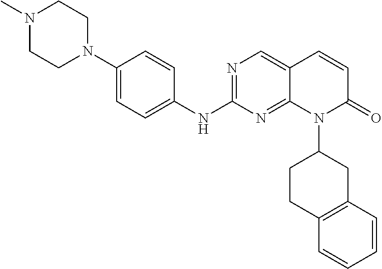 [SMILES: CN1CCN(c2ccc(Nc3ncc4ccc(=O)n(C5CCc6ccccc6C5)c4n3)cc2)CC1] | A | A | A | A |
| 75 | 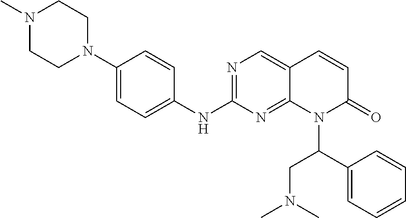 [SMILES: CN(C)CC(c1ccccc1)n1c(=O)ccc2cnc(Nc3ccc(N4CCN(C)CC4)cc3)nc21] | C | C | C | C |
| 76 | 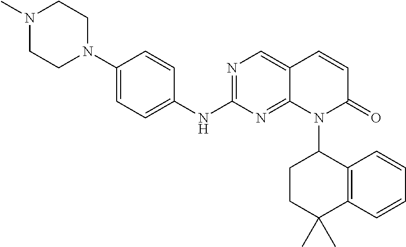 [SMILES: CN1CCN(c2ccc(Nc3ncc4ccc(=O)n(C5CCC(C)(C)c6ccccc65)c4n3)cc2)CC1] | A | A | B | B |
| 77 | 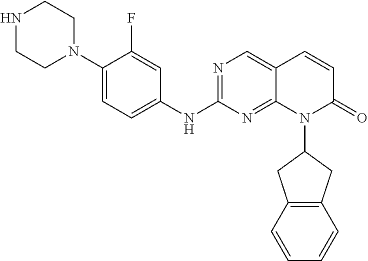 [SMILES: O=c1ccc2cnc(Nc3ccc(N4CCNCC4)c(F)c3)nc2n1C1Cc2ccccc2C1] | A | A | B | A |
| 78 | 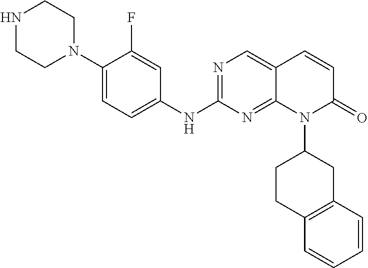 [SMILES: O=c1ccc2cnc(Nc3ccc(N4CCNCC4)c(F)c3)nc2n1C1CCc2ccccc2C1] | A | A | A | A |
| 79 | 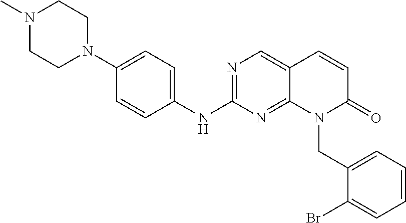 [SMILES: CN1CCN(c2ccc(Nc3ncc4ccc(=O)n(Cc5ccccc5Br)c4n3)cc2)CC1] | A | A | A | A |
| 80 | 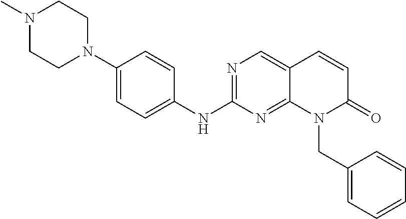 [SMILES: CN1CCN(c2ccc(Nc3ncc4ccc(=O)n(Cc5ccccc5)c4n3)cc2)CC1] | B | B | | C |
| 81 | 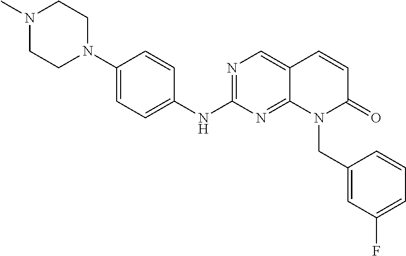 [SMILES: CN1CCN(c2ccc(Nc3ncc4ccc(=O)n(Cc5cccc(F)c5)c4n3)cc2)CC1] | B | B | | B |
| 82 | 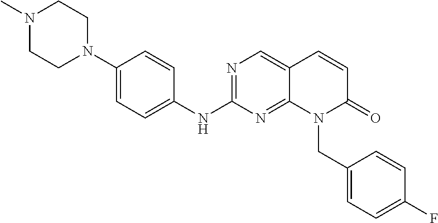 [SMILES: CN1CCN(c2ccc(Nc3ncc4ccc(=O)n(Cc5ccc(F)cc5)c4n3)cc2)CC1] | B | C | | C |
| 83 | 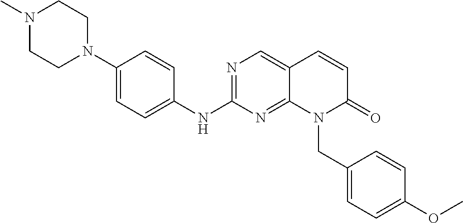 | C | C | | C |
| 84 | 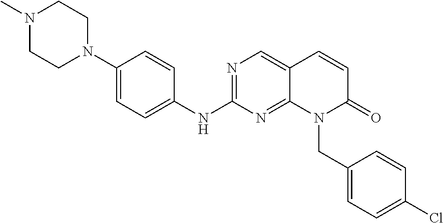 [SMILES: CN1CCN(c2ccc(Nc3ncc4ccc(=O)n(Cc5ccc(Cl)cc5)c4n3)cc2)CC1] | B | C | | C |
| 85 | 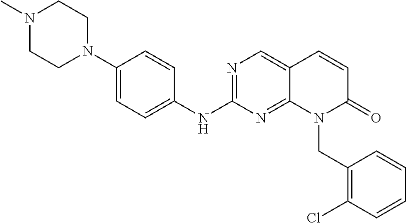 [SMILES: CN1CCN(c2ccc(Nc3ncc4ccc(=O)n(Cc5ccccc5Cl)c4n3)cc2)CC1] | A | A | | B |
| 86 | 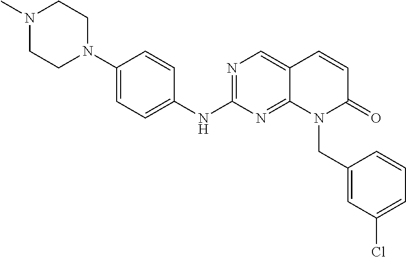 [SMILES: CN1CCN(c2ccc(Nc3ncc4ccc(=O)n(Cc5cccc(Cl)c5)c4n3)cc2)CC1] | B | C | | C |
| 87 | 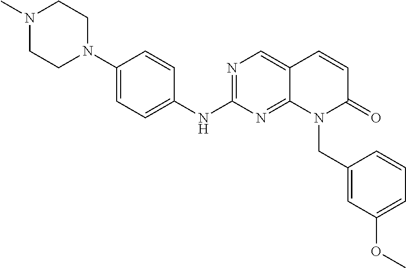 | B | C | | C |
| 88 | 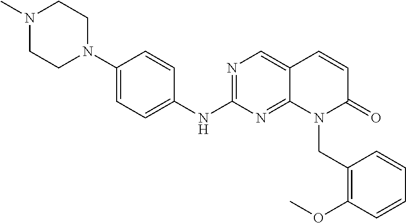 [SMILES: COc1ccccc1Cn1c(=O)ccc2cnc(Nc3ccc(N4CCN(C)CC4)cc3)nc21] | A | B | | B |
| 89 | 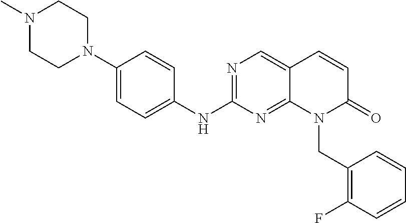 [SMILES: CN1CCN(c2ccc(Nc3ncc4ccc(=O)n(Cc5ccccc5F)c4n3)cc2)CC1] | B | B | | B |
| 90 | 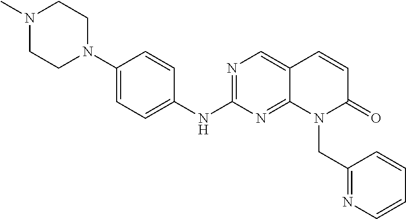 [SMILES: CN1CCN(c2ccc(Nc3ncc4ccc(=O)n(Cc5ccccn5)c4n3)cc2)CC1] | B | C | | B |
| 91 | 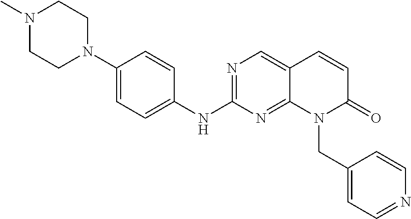 [SMILES: CN1CCN(c2ccc(Nc3ncc4ccc(=O)n(Cc5ccncc5)c4n3)cc2)CC1] | B | C | | C |
| 92 | 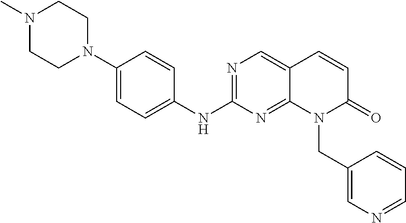 [SMILES: CN1CCN(c2ccc(Nc3ncc4ccc(=O)n(Cc5cccnc5)c4n3)cc2)CC1] | B | C | | C |
| 93 | 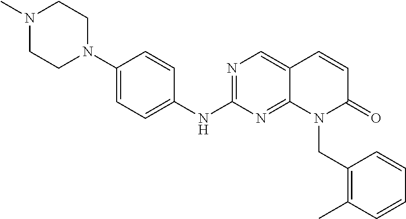 [SMILES: Cc1ccccc1Cn1c(=O)ccc2cnc(Nc3ccc(N4CCN(C)CC4)cc3)nc21] | A | A | | B |
| 94 | 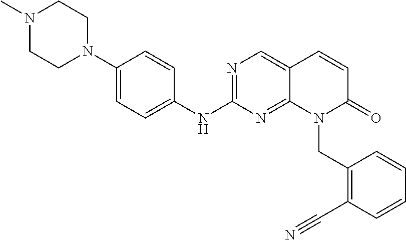 [SMILES: CN1CCN(c2ccc(Nc3ncc4ccc(=O)n(Cc5ccccc5C#N)c4n3)cc2)CC1] | A | A | | B |
| 95 | 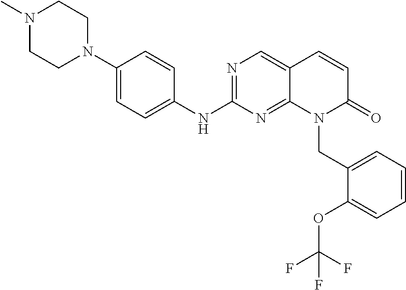 [SMILES: CN1CCN(c2ccc(Nc3ncc4ccc(=O)n(Cc5ccccc5OC(F)(F)F)c4n3)cc2)CC1] | A | A | | B |
| 96 | 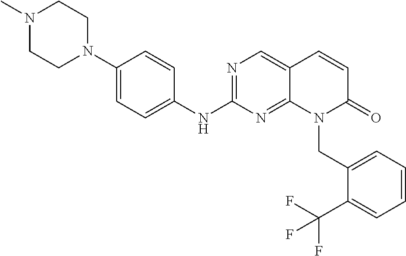 [SMILES: CN1CCN(c2ccc(Nc3ncc4ccc(=O)n(Cc5ccccc5C(F)(F)F)c4n3)cc2)CC1] | A | A | A | A |
| 97 | 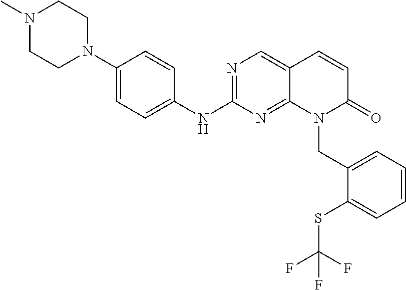 [SMILES: CN1CCN(c2ccc(Nc3ncc4ccc(=O)n(Cc5ccccc5SC(F)(F)F)c4n3)cc2)CC1] | A | A | A | A |
| 98 | 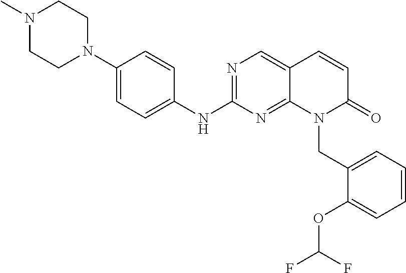 [SMILES: CN1CCN(c2ccc(Nc3ncc4ccc(=O)n(Cc5ccccc5OC(F)F)c4n3)cc2)CC1] | A | A | A | A |
| 99 | 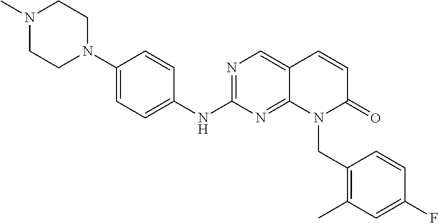 [SMILES: Cc1cc(F)ccc1Cn1c(=O)ccc2cnc(Nc3ccc(N4CCN(C)CC4)cc3)nc21] | B | B | B | B |
| 100 | 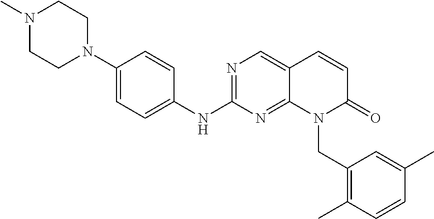 | B | B | B | B |
| 101 | 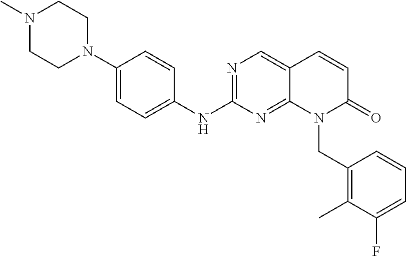 [SMILES: Cc1c(F)cccc1Cn1c(=O)ccc2cnc(Nc3ccc(N4CCN(C)CC4)cc3)nc21] | A | B | C | B |
| 102 | 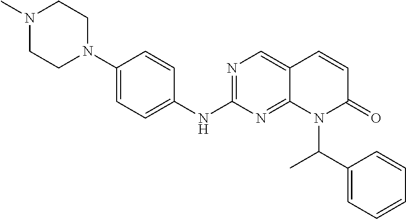 [SMILES: CC(c1ccccc1)n1c(=O)ccc2cnc(Nc3ccc(N4CCN(C)CC4)cc3)nc21] | B | B | C | B |
| 103 | 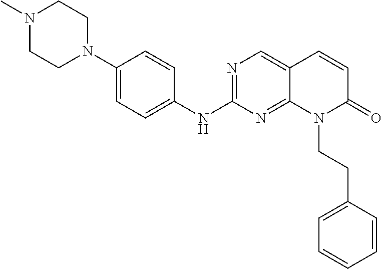 [SMILES: CN1CCN(c2ccc(Nc3ncc4ccc(=O)n(CCc5ccccc5)c4n3)cc2)CC1] | A | A | B | A |
| 104 | 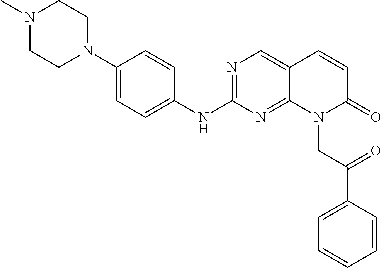 [SMILES: CN1CCN(c2ccc(Nc3ncc4ccc(=O)n(CC(=O)c5ccccc5)c4n3)cc2)CC1] | B | B | B | B |
| 105 | 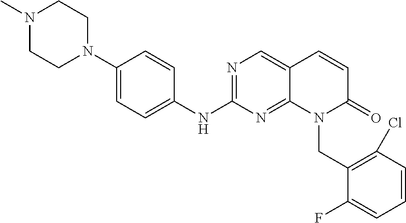 [SMILES: CN1CCN(c2ccc(Nc3ncc4ccc(=O)n(Cc5c(F)cccc5Cl)c4n3)cc2)CC1] | A | A | A | A |
| 106 | 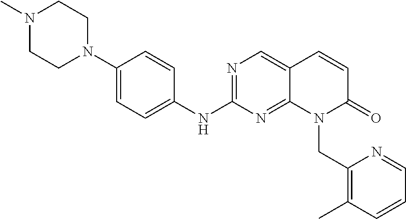 [SMILES: Cc1cccnc1Cn1c(=O)ccc2cnc(Nc3ccc(N4CCN(C)CC4)cc3)nc21] | B | B | C | B |
| 107 | 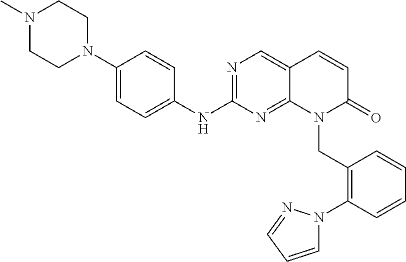 [SMILES: CN1CCN(c2ccc(Nc3ncc4ccc(=O)n(Cc5ccccc5-n5cccn5)c4n3)cc2)CC1] | A | B | B | B |
| 108 | 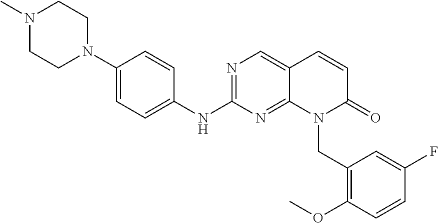 [SMILES: COc1ccc(F)cc1Cn1c(=O)ccc2cnc(Nc3ccc(N4CCN(C)CC4)cc3)nc21] | A | B | B | B |
| 109 | 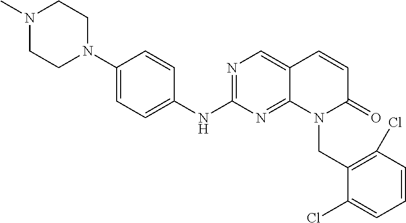 [SMILES: CN1CCN(c2ccc(Nc3ncc4ccc(=O)n(Cc5c(Cl)cccc5Cl)c4n3)cc2)CC1] | A | A | B | A |
| 110 | 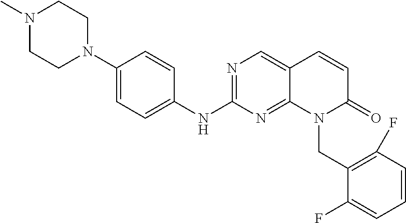 [SMILES: CN1CCN(c2ccc(Nc3ncc4ccc(=O)n(Cc5c(F)cccc5F)c4n3)cc2)CC1] | A | B | B | B |
| 111 | 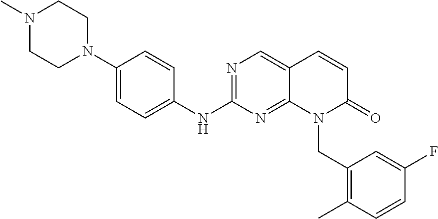 [SMILES: Cc1ccc(F)cc1Cn1c(=O)ccc2cnc(Nc3ccc(N4CCN(C)CC4)cc3)nc21] | A | B | B | A |
| 112 | 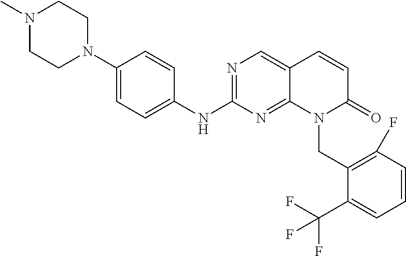 [SMILES: CN1CCN(c2ccc(Nc3ncc4ccc(=O)n(Cc5c(F)cccc5C(F)(F)F)c4n3)cc2)CC1] | A | A | A | A |
| 113 | 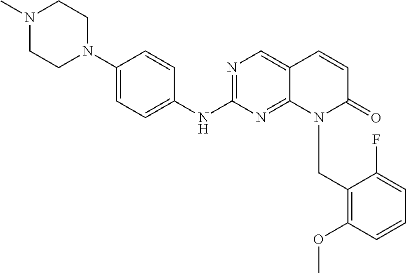 [SMILES: COc1cccc(F)c1Cn1c(=O)ccc2cnc(Nc3ccc(N4CCN(C)CC4)cc3)nc21] | A | B | B | A |
| 114 | 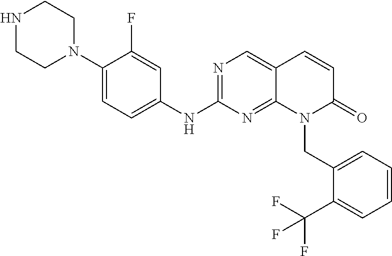 [SMILES: O=c1ccc2cnc(Nc3ccc(N4CCNCC4)c(F)c3)nc2n1Cc1ccccc1C(F)(F)F] | A | A | A | A |
| 115 | 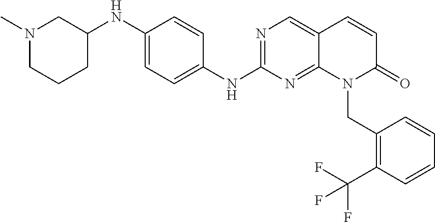 [SMILES: CN1CCCC(Nc2ccc(Nc3ncc4ccc(=O)n(Cc5ccccc5C(F)(F)F)c4n3)cc2)C1] | A | A | A | A |
| 116 | 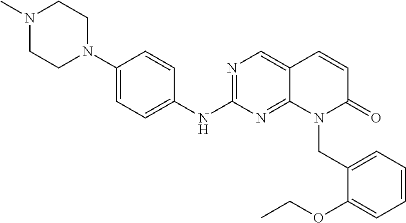 [SMILES: CCOc1ccccc1Cn1c(=O)ccc2cnc(Nc3ccc(N4CCN(C)CC4)cc3)nc21] | A | A | A | B |
| 117 | 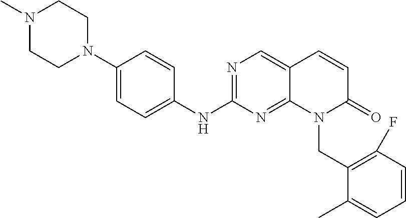 [SMILES: Cc1cccc(F)c1Cn1c(=O)ccc2cnc(Nc3ccc(N4CCN(C)CC4)cc3)nc21] | A | A | A | A |
| 118 | 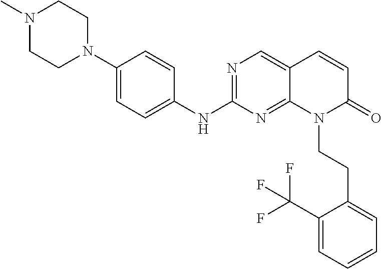 [SMILES: CN1CCN(c2ccc(Nc3ncc4ccc(=O)n(CCc5ccccc5C(F)(F)F)c4n3)cc2)CC1] | A | A | B | A |
| 119 | 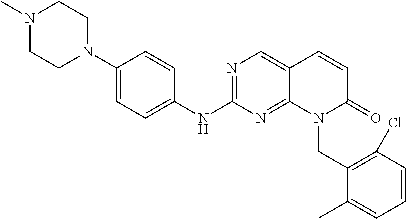 [SMILES: Cc1cccc(Cl)c1Cn1c(=O)ccc2cnc(Nc3ccc(N4CCN(C)CC4)cc3)nc21] | A | B | B | A |
| 120 | 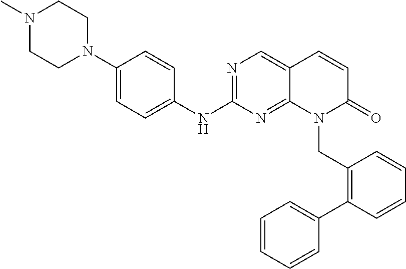 [SMILES: CN1CCN(c2ccc(Nc3ncc4ccc(=O)n(Cc5ccccc5-c5ccccc5)c4n3)cc2)CC1] | A | A | A | B |
| 121 | 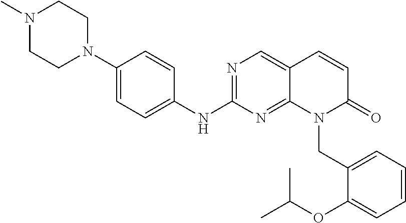 [SMILES: CC(C)Oc1ccccc1Cn1c(=O)ccc2cnc(Nc3ccc(N4CCN(C)CC4)cc3)nc21] | A | A | A | A |
| 122 | 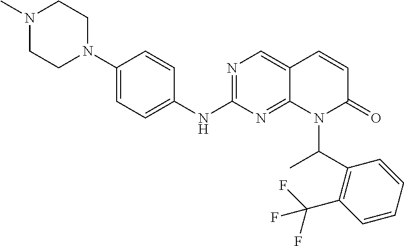 [SMILES: CC(c1ccccc1C(F)(F)F)n1c(=O)ccc2cnc(Nc3ccc(N4CCN(C)CC4)cc3)nc21] | B | C | C | B |
| 123 | 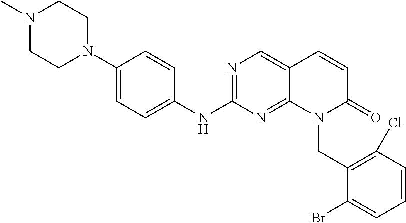 [SMILES: CN1CCN(c2ccc(Nc3ncc4ccc(=O)n(Cc5c(Cl)cccc5Br)c4n3)cc2)CC1] | A | A | A | A |
| 124 | 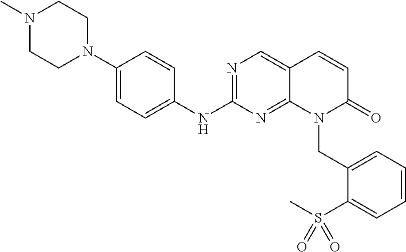 [SMILES: CN1CCN(c2ccc(Nc3ncc4ccc(=O)n(Cc5ccccc5S(C)(=O)=O)c4n3)cc2)CC1] | A | A | A | A |
| 125 | 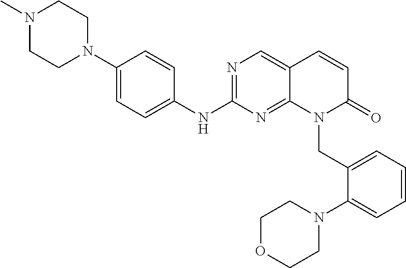 [SMILES: CN1CCN(c2ccc(Nc3ncc4ccc(=O)n(Cc5ccccc5N5CCOCC5)c4n3)cc2)CC1] | A | A | A | A |
| 126 | 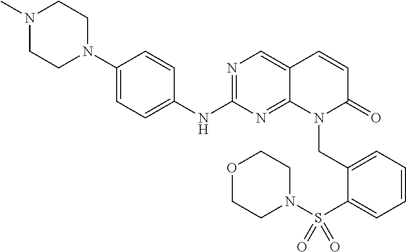 [SMILES: CN1CCN(c2ccc(Nc3ncc4ccc(=O)n(Cc5ccccc5S(=O)(=O)N5CCOCC5)c4n3)cc2)CC1] | A | A | A | A |
| 127 | 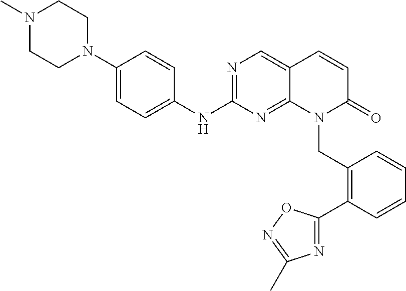 [SMILES: Cc1noc(-c2ccccc2Cn2c(=O)ccc3cnc(Nc4ccc(N5CCN(C)CC5)cc4)nc32)n1] | A | B | B | B |
| 128 | 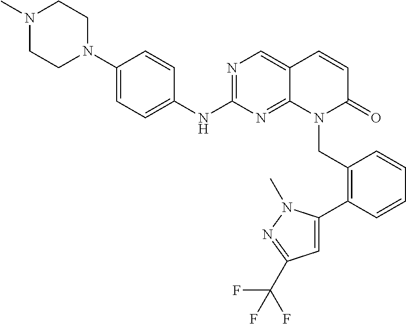 [SMILES: CN1CCN(c2ccc(Nc3ncc4ccc(=O)n(Cc5ccccc5-c5cc(C(F)(F)F)nn5C)c4n3)cc2)CC1] | B | B | B | C |
| 129 | 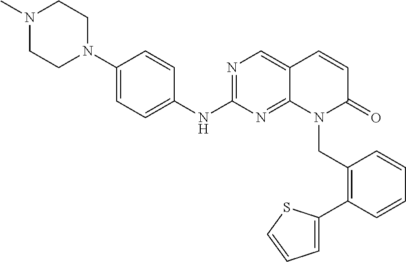 [SMILES: CN1CCN(c2ccc(Nc3ncc4ccc(=O)n(Cc5ccccc5-c5cccs5)c4n3)cc2)CC1] | A | A | A | B |
| 130 | 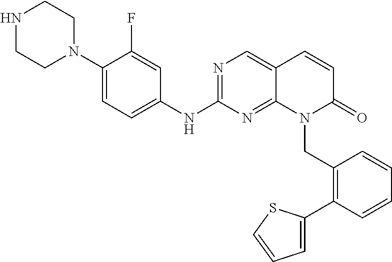 [SMILES: O=c1ccc2cnc(Nc3ccc(N4CCNCC4)c(F)c3)nc2n1Cc1ccccc1-c1cccs1] | A | A | A | A |
| 131 | 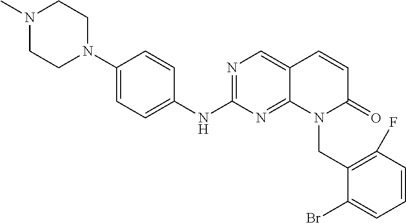 [SMILES: CN1CCN(c2ccc(Nc3ncc4ccc(=O)n(Cc5c(F)cccc5Br)c4n3)cc2)CC1] | A | A | A | A |
| 132 | 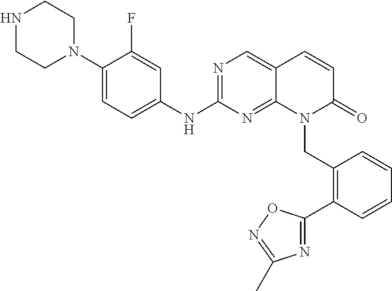 [SMILES: Cc1noc(-c2ccccc2Cn2c(=O)ccc3cnc(Nc4ccc(N5CCNCC5)c(F)c4)nc32)n1] | A | B | B | B |
| 133 | 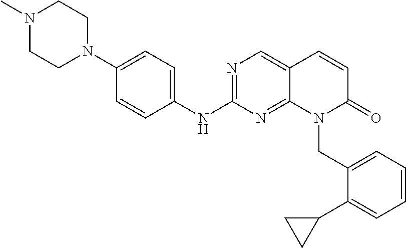 [SMILES: CN1CCN(c2ccc(Nc3ncc4ccc(=O)n(Cc5ccccc5C5CC5)c4n3)cc2)CC1] | A | A | A | A |
| 134 | 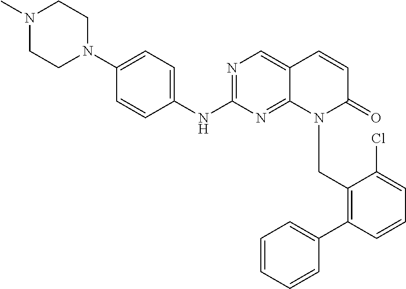 [SMILES: CN1CCN(c2ccc(Nc3ncc4ccc(=O)n(Cc5c(Cl)cccc5-c5ccccc5)c4n3)cc2)CC1] | B | C | C | B |
| 135 | 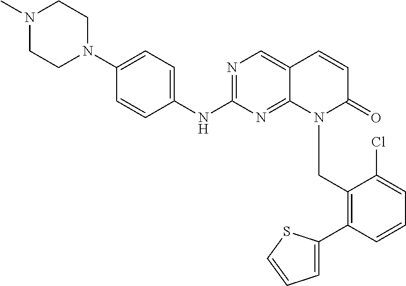 [SMILES: CN1CCN(c2ccc(Nc3ncc4ccc(=O)n(Cc5c(Cl)cccc5-c5cccs5)c4n3)cc2)CC1] | B | C | C | C |
| 136 | 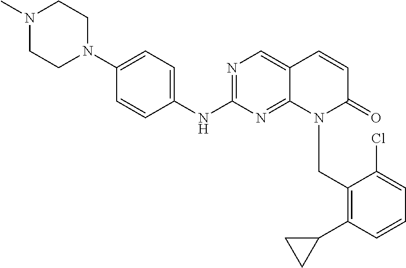 [SMILES: CN1CCN(c2ccc(Nc3ncc4ccc(=O)n(Cc5c(Cl)cccc5C5CC5)c4n3)cc2)CC1] | A | A | A | B |
| 137 | 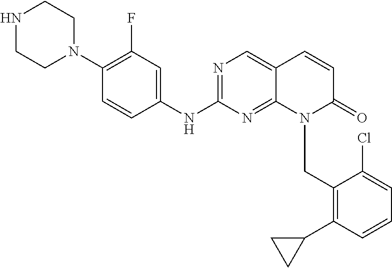 [SMILES: O=c1ccc2cnc(Nc3ccc(N4CCNCC4)c(F)c3)nc2n1Cc1c(Cl)cccc1C1CC1] | A | A | A | B |
| 138 | 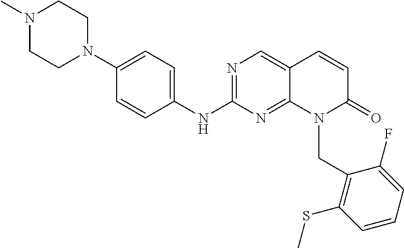 [SMILES: CSc1cccc(F)c1Cn1c(=O)ccc2cnc(Nc3ccc(N4CCN(C)CC4)cc3)nc21] | A | A | A | A |
| 139 | 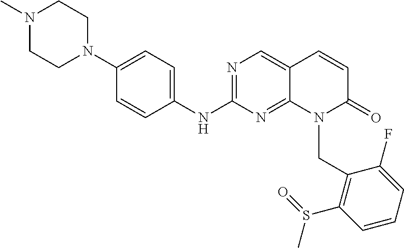 [SMILES: CN1CCN(c2ccc(Nc3ncc4ccc(=O)n(Cc5c(F)cccc5S(C)=O)c4n3)cc2)CC1] | A | B | A | B |
| 140 | 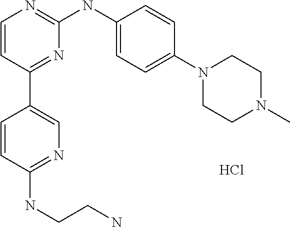 [SMILES: CN1CCN(c2ccc(Nc3nccc(-c4ccc(NCCN)nc4)n3)cc2)CC1 ; Cl] | A | B | B | A |
| 141 | 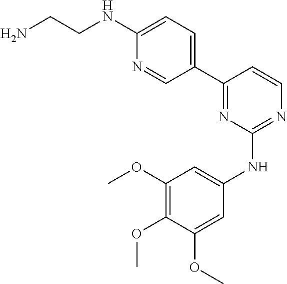 [SMILES: COc1cc(Nc2nccc(-c3ccc(NCCN)nc3)n2)cc(OC)c1OC] | A | B | B | A |
| 142 | 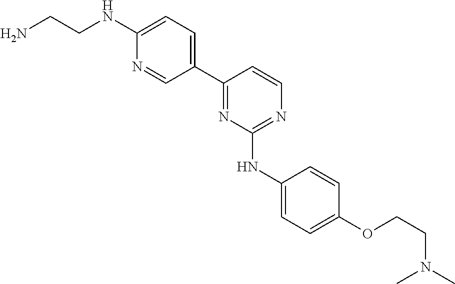 [SMILES: CN(C)CCOc1ccc(Nc2nccc(-c3ccc(NCCN)nc3)n2)cc1] | B | B | | B |
| 143 | 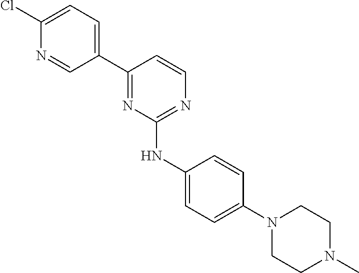 [SMILES: CN1CCN(c2ccc(Nc3nccc(-c4ccc(Cl)nc4)n3)cc2)CC1] | B | C | | C |
| 144 | 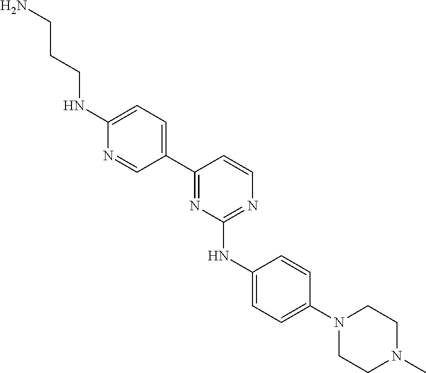 [SMILES: CN1CCN(c2ccc(Nc3nccc(-c4ccc(NCCCN)nc4)n3)cc2)CC1] | A | B | | B |
| 145 | 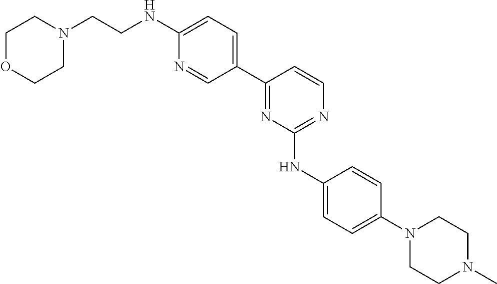 [SMILES: CN1CCN(c2ccc(Nc3nccc(-c4ccc(NCCN5CCOCC5)nc4)n3)cc2)CC1] | B | C | | B |
| 146 | 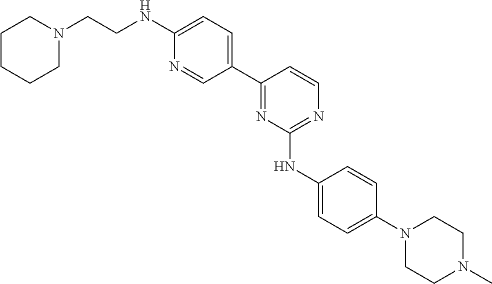 [SMILES: CN1CCN(c2ccc(Nc3nccc(-c4ccc(NCCN5CCCCC5)nc4)n3)cc2)CC1] | B | B | C | A |
| 147 | 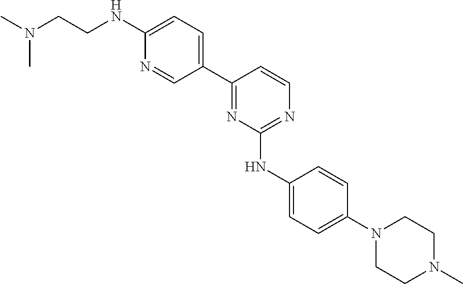 | B | B | | B |
| 148 | 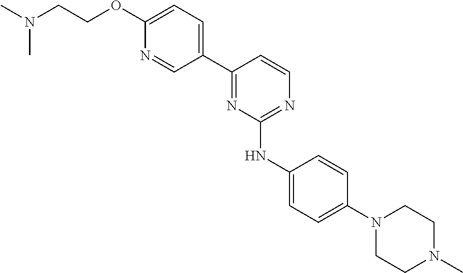 | B | C | | C |
| 149 | 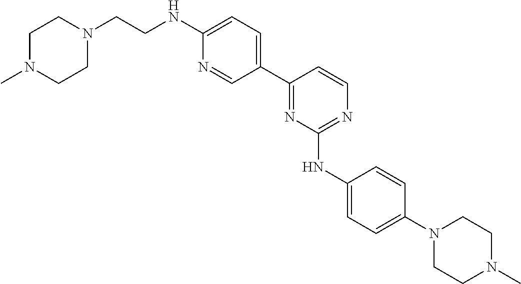 | B | C | | C |
| 150 | 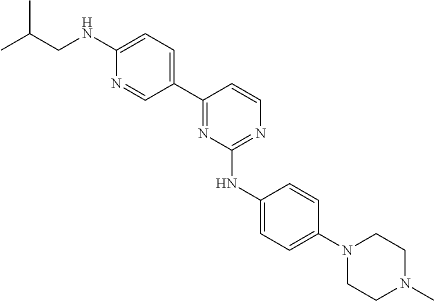 [SMILES: CC(C)CNc1ccc(-c2ccnc(Nc3ccc(N4CCN(C)CC4)cc3)n2)cn1] | B | B | C | A |
| 151 | 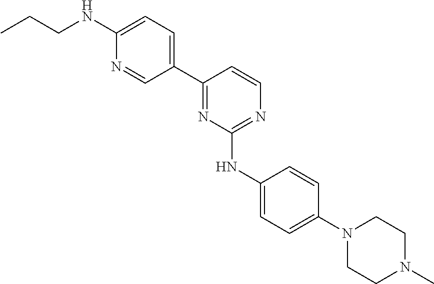 [SMILES: CCCNc1ccc(-c2ccnc(Nc3ccc(N4CCN(C)CC4)cc3)n2)cn1] | B | B | | B |
| 152 | 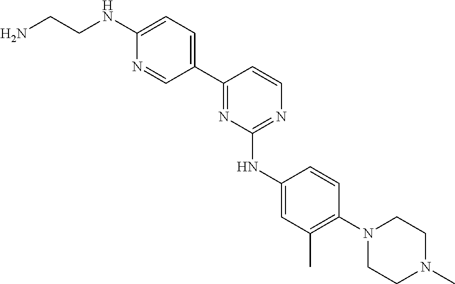 [SMILES: Cc1cc(Nc2nccc(-c3ccc(NCCN)nc3)n2)ccc1N1CCN(C)CC1] | A | B | B | A |
| 153 | 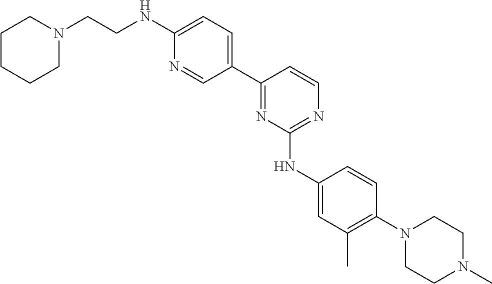 [SMILES: Cc1cc(Nc2nccc(-c3ccc(NCCN4CCCCC4)nc3)n2)ccc1N1CCN(C)CC1] | B | B | C | A |
| 154 | 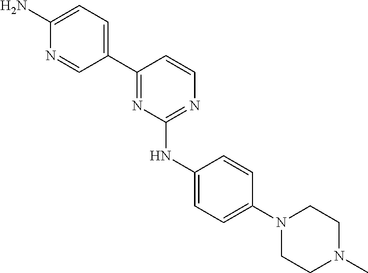 [SMILES: CN1CCN(c2ccc(Nc3nccc(-c4ccc(N)nc4)n3)cc2)CC1] | B | B | C | B |
| 155 | 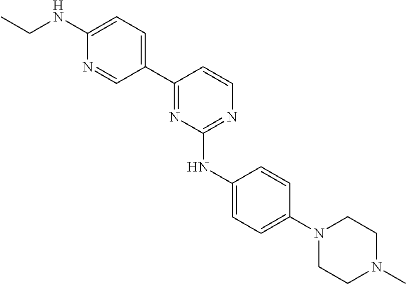 [SMILES: CCNc1ccc(-c2ccnc(Nc3ccc(N4CCN(C)CC4)cc3)n2)cn1] | B | C | C | A |
| 156 | 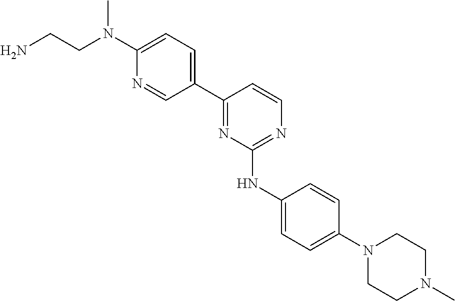 [SMILES: CN1CCN(c2ccc(Nc3nccc(-c4ccc(N(C)CCN)nc4)n3)cc2)CC1] | B | B | C | B |
| 157 | 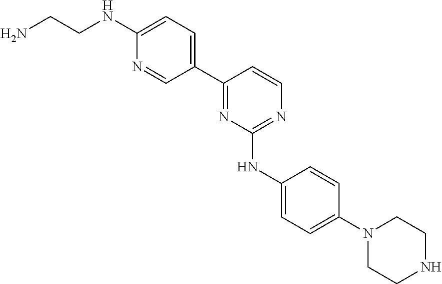 [SMILES: NCCNc1ccc(-c2ccnc(Nc3ccc(N4CCNCC4)cc3)n2)cn1] | A | B | B | A |
| 158 | 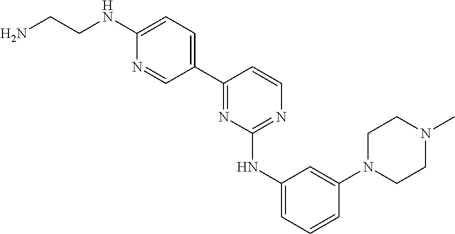 [SMILES: CN1CCN(c2cccc(Nc3nccc(-c4ccc(NCCN)nc4)n3)c2)CC1] | B | B | C | A |
| 159 | 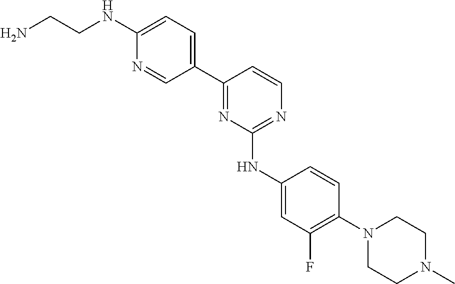 [SMILES: CN1CCN(c2ccc(Nc3nccc(-c4ccc(NCCN)nc4)n3)cc2F)CC1] | A | B | B | A |
| 160 | 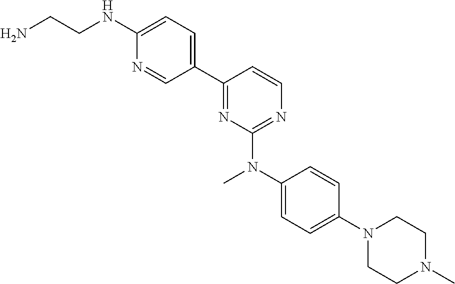 [SMILES: CN1CCN(c2ccc(N(C)c3nccc(-c4ccc(NCCN)nc4)n3)cc2)CC1] | C | C | | C |
| 161 | 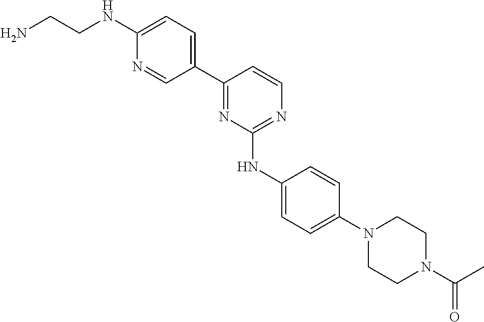 [SMILES: CC(=O)N1CCN(c2ccc(Nc3nccc(-c4ccc(NCCN)nc4)n3)cc2)CC1] | C | C | C | A |
| 162 | 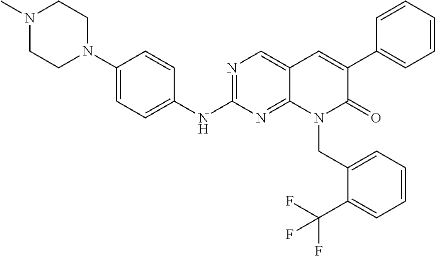 [SMILES: CN1CCN(c2ccc(Nc3ncc4cc(-c5ccccc5)c(=O)n(Cc5ccccc5C(F)(F)F)c4n3)cc2)CC1] | A | A | A | C |
| 163 | 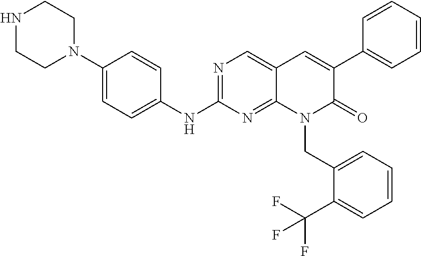 [SMILES: O=c1c(-c2ccccc2)cc2cnc(Nc3ccc(N4CCNCC4)cc3)nc2n1Cc1ccccc1C(F)(F)F] | A | A | A | B |
| 167 | 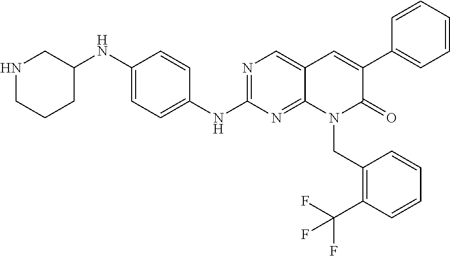 [SMILES: O=c1c(-c2ccccc2)cc2cnc(Nc3ccc(NC4CCCNC4)cc3)nc2n1Cc1ccccc1C(F)(F)F] | A | A | A | B |

A: IC₅₀ <1 μM;
B: IC₅₀ >1 μM and <10 μM;
C: IC₅₀ >10 μM

### Example 102

### Slice electrophysiology assay for determination of PAK inhibitory activity

Materials: coronal cortical slices (400 μm) containing temporal cortex from 2- to 3-month-old C57-Black-6 mice male littermates (from Elevage Janvier, FRANCE) are prepared and allowed to recover in oxygenated (95% O₂ and 5% CO₂) warm (30° C.) artificial cerebrospinal fluid (ACSF) containing 124 mM NaCl, 5 mM KCl, 1.25 mM, NaH₂PO₄, 1 mM MgCl₂, 2 mM CaCl₂, 26 mM NaHCO₃, and 10 mM dextrose.

Compound dilution: a 10 mM DMSO stock solution is prepared for each test compound and 100 μL aliquots are stored at −20° C. On the day of experiment an aliquot is thawed and vortexed for fresh solutions preparation. The final concentration of DMSO is adjusted to 0.1% in all solutions, including control ACSF solution.

Perfusion: Artificial Cerebro-Spinal Fluid (ACSF) is perfused at 3 mL/min. The recording chamber has a volume of 1 mL. Then the chamber medium is renewed every 20 s. The perfusion liquid is maintained at 30±0.1° C.

Data acquisition: evoked-responses are sampled at 5 kHz before recording on the harddisk of the computer

Recording in cortical layer II/III: The recording is carried out on a Multi Electrode Array. Responses (field portentials) in layer II/III are evoked by layer IV stimulation between one MEA electrode and the GND electrode. I/O curve is first performed to define evoked responses for stimulation intensities between 100 and 800 μA, by 100 μA steps. The stimulus consists in a monopolar biphasic current pulse (negative for 60 μs and then positive for 60 μs) which is applied every 30 s to evoke “responses” (field Excitatory Post Synaptic Potentials; (fEPSP) in cortical layer II/III.

Basal synaptic transmission: a monopolar stimulation (a bi-phasic stimulus:±300 mA for 120 ms between one MEA electrode and the GND) is applied every 30 s on the MPP fibres to evoke “responses” (field potentials: fEPSP) in the DG region. The basal stimulation intensity will be set to evoke 40% of maximal amplitude response. The same stimulation intensity will be used in the 100 Hz stimulation protocol.

LTP: a stimulus is applied every 30 s with an intensity settled at 40% of the maximal amplitude responses. LTP is then induced by TBS, which consists of eight brief bursts (each with four pulses at 100 Hz) of stimuli delivered every 200 ms. Potentiation of synaptic transmission is then monitored for an additional 40 minutes period. Since fEPSP result from glutamatergic synaptic transmission consecutive to afferent pathway stimulation, 10 μM NBQX are perfused on the slice, at the end of each experiment, to validate the glutamatergic nature of synaptic transmission as well as to subtract background noise at individual electrode level.

Compound evaluation: following a 10 minutes control recording period (to verify baseline stability), the compound is perfused for 20 minutes. Then, LTP is triggered and the fEPSP amplitude will be recorded for an additional 40 minutes period in the presence of compound.

Data analysis: fEPSP amplitudes are measured as the difference between the baseline (before stimulation) and the maximal peak amplitude. The fEPSP are normalized as a percent of the meanaveraged amplitude recorded over a 10 min control period, before compound application. Normalized fEPSP values are then averaged for each experiment carried out in control conditions and with the test compound. The fEPSP mean values (+/−SEM) are expressed as a function of time before and after LTP induction.

An increased LTP, indicates an increase in synaptic plasticity mediated by inhibition of PAK. Compounds are tested using the procedure described above to determine the effect of PAK inhibitors on synaptic plasticity.

### Example 103

### Treatment of Autism by Administration of a PAK Inhibitor in an Animal Model

The ability of a compound of Formula I-XXIII described herein (a PAK inhibitor) to alleviate, reduce the severity of, or inhibit the progression of symptoms of autism (i.e., their mouse analogs) is tested in a FMRI KO mouse model.

Twenty-four FMRI KO male mice (age 2 months) are divided into Group 1 (n=6) and Group 2 (n=6) treatment groups (1 mg/kg oral gavage of a compound of Formula I-XXIII described herein), a placebo Group (Group 3) (n=6) (0.1% DMSO in physiological saline solution) and wild-type (Group 4) (n=6) and are analyzed for behavioral differences using the Open Field Test.

Open Field Test. The mice in Groups 1-4 are subjected to the open field test according to standard procedures. Each of the mice ran for 60 minutes in a VersaMax activity monitor chamber (Accuscan Instruments). Open field activity is detected by photobeam breaks and is analyzed by the VersaMax software. Stereotypy is recorded when the mouse breaks the same beam (or set of beams) repeatedly. Stereotypy count is the number of beam breaks that occur during this period of stereotypic activity.

FMR1 KO mice are known to exhibit three abnormal behaviors compared to wild-type mice (Peier et., 2000, Hum. Mol. Genet., 9:1145): (i) hyperactivity—they travel a longer distance and move for a longer period of time than wild-type; (ii) stereotypy—they exhibit a higer number of repetitive behaviors than wild-type; and (iii) hypo-anxiety—they stay in the center field for a longer period of time and in the corners of the field for shorter periods of time than wild-type.

It is expected that the FMR1 mice in treatment Group 1 and treatment Group 2 will perform comparable to the wild-type controls (Group 4) for: (i) hyperactivity; (ii) stereotypy; and (iii) hypo-anxiety as measured in the Open Field Test, whereas the FMR1 mice in Group 3 will exhibit abnormal behavior. This indicates that treatment of FMR1 KO mice with PAK inhibitors of a compound of Formula I-XXIII described herein restores activity, repetitive behavior, and anxiety to wild-type levels.

Statistical Analysis. Statistical analysis is performed by ANOVA or repeated ANOVA. Differences between groups are considered significant at p<0.05.

### Example 104

### Treatment of Autism by Administration of a PAK Inhibitor in an Animal Model

The ability of a compound of Formula I-XXIII described herein (a PAK inhibitor) to delay or halt the progression of behavorial symptoms symptoms of autism (i.e., their mouse analogs) is tested in a BTBR T1tfJ mouse model of autism syndrome (McFarlane et al., Genes, brain, and behavior (2007)).

BTBR T1tfJ is an inbred mouse strain that shows robust behavioral phenotypes with analogies to all three of the diagnostic symptoms of autism, including well-replicated deficits in reciprocal social interactions and social approach, unusual patterns of ultrasonic vocalization, and high levels of repetitive self-grooming.

Twenty BTBR T1tfJ male mice (age 2 months) are divided into Group 1 (n=5) and Group 2 (n=5) treatment groups (1 mg/kg oral gavage of a compound of Formula I-XXIII described herein), a placebo Group (Group 3) (n=5) (0.1% DMSO in physiological saline solution) and wild-type (Group 4) (n=5) and are analyzed for behavioral differences using the sociability test and self grooming test described below.

Sociability Test. Social approach behaviors are tested in an automated 3-chambered apparatus using methods similar to those previously described (Moy et al., 2004; Nadler et al., 2004; Crawley et al., 2007; McFarlane et al., 2007; Moy et al., 2007). Briefly, the apparatus is a rectangular, three-chambered box made from clear polycarbonate. Retractable doorways built in the two dividing walls allow access to the side chambers. Quantification of entries and duration in the chambers is automatically measured by photocells embedded in the doorways. The apparatus is cleaned with 70% ethanol and water between subjects.

Animals to be used as “strangers” are male 129Sv/ImJ and AJ mice, aged 8-14 weeks old (The Jackson Laboratory (Bar Harbor, Me.)). Strangers are habituated to the apparatus and to the wire cup enclosure before the start of experiments, for 10 min per day for three consecutive days. The subject mouse is allowed to acclimate to the apparatus for 20 min before the sociability test, 10 min in the central chamber with the doors closed and another 10 min in the entire empty arena with the doors open. The subject is then briefly confined to the center chamber while a novel object (inverted wire cup, Galaxy Cup) is introduced into one of the side chambers. A stranger mouse enclosed in an identical wire cup is placed in the other side chamber. An upright plastic drinking cup, held in place by a lead weight in the cup, is placed on the top of each inverted wire cup to prevent the subject from climbing onto the top of the wire cup. The location for the novel object and the stranger mouse alternates between the left and right chambers across subjects. After both stimuli are positioned, the doors are simultaneously re-opened and the subject is allowed access to all three chambers for 10 min. Measures to be taken include time spent in each chamber, time spent sniffing each cup, and number of entries. An observer uninformed of the genotypes scores time spent sniffing with a stopwatch.

Self-Grooming. The test is performed as previously described (McFarlane et al., 2007). Each subject is placed individually in a clean standard mouse cage and allowed to acclimate for 10 min. Following this habituation period, subjects are observed for another 10 min, during which time cumulative time spent in self-grooming is scored by an experimenter sitting approximately 2 meters from the test cage. A silenced stopwatch is used for scoring cumulative time spent grooming during the 10 min test session.

It is expected that the BTBR T1tfJ mice in treatment Group 1 and treatment Group 2 will perform comparable to the wild-type controls (Group 4) for: (i) sociability and (ii) self-grooming, whereas the BTBR T1tfJ mice in Group 3 will exhibit abnormal behavior. This indicates that treatment of BTBR T1tfJ mice with PAK inhibitors of a compound of Formula I-XXIII described herein restores low sociability and repetitive self-grooming behavior to wild-type levels.

Statistical Analysis. Statistical analysis is performed by ANOVA or repeated ANOVA. Differences between groups are considered significant at p<0.05.

### Example 105

### In Vivo Monitoring of Dendritic Spine Plasticity in Double Transgenic GFP-M/DN-DISC1 Mice Treated with a PAK Inhibitor

In the following experiment, dendritic spine plasticity is directly monitored in vivo by two photon laser scanning microscopy (TPLSM) in double transgenic GFP-M/DN-DISC1 mice treated with a PAK inhibitor (Compound 2) or a placebo. Mice (C57BL/6) expressing GFP in a subset of cortical layer 5 neurons (transgenic line GFP-M described in Feng et al, 2000, Neuron 28:41-51) are crossed with DN-DISC1 C57BL/6 DN-DISC1 mice (Hikida et al (2007), Proc Natl Acad Sci USA, 104(36):14501-14506) to obtain heterozygous transgenic mice, which are then crossed to obtain homozygous double transgenic GFPM/DN-DISC1 mice used in this study.

GFP-M/DN-DISC1 animals aged 28-61 d are anesthetized using avertin (16 μl/g body weight; Sigma, St. Louis, Mo.). The skull is exposed, scrubbed, and cleaned with ethanol. Primary visual, somatosensory, auditory, and motor cortices are identified based on stereotaxic coordinates, and their location is confirmed with tracer injections (see below).

Long-term imaging experiments are started at P40. The skull is thinned over the imaging area as described in Grutzendler et al, (2002), Nature, 420:812-816. A small metal bar is affixed to the skull. The metal bar is then screwed into a plate that connected directly to the microscope stage for stability during imaging. The metal bar also allows for maintaining head angle and position during different imaging sessions. At the end of the imaging session, animals are sutured and returned to their cage. Thirty animals previously imaged at P40 are then divided into a control group receiving a 1% sugar solution (oral gavage once per day) and a treatment group administered Compound 2, a PAK inhibitor, in 0.1% DMSO (oral gavage. 1 mg/kg, once per day). During the subsequent imaging sessions (at P45, P50, P55, or P70), animals are reanesthetized and the skull is rethinned. The same imaging area is identified based on the blood vessel pattern and gross dendritic pattern, which generally remains stable over this time period.

At the end of the last imaging session, injections of cholera toxin subunit B coupled to Alexa Fluor 594 are made adjacent to imaged areas to facilitate identification of imaged cells and cortical areas after fixation. Mice are transcardially perfused and fixed with paraformaldehyde, and coronal sections are cut to verify the location of imaged cells. Sections are then mounted in buffer, coverslipped, and sealed. Images are collected using a Fluoview confocal microscope (Olympus Optical, Melville, N.Y.).

For in vivo two photon imaging, a two-photon laser scanning microscope is used as described in Majewska et al., (2000), Pflügers Arch, 441:398-408. The microscope consists of a modified Fluoview confocal scan head (Olympus Optical) and a titanium/sulphur laser providing 100 fs pulses at 80 MHz at a wavelength of 920 nm (Tsunami; Spectra-Physics, Menlo Park, Calif.) pumped by a 10 W solid-state source (Millenia; Spectra-Physics). Fluorescence is detected using photomultiplier tubes (HC125-02; Hamamatsu, Shizouka, Japan) in whole-field detection mode. The craniotomy over the visual cortex is initially identified under whole-field fluorescence illumination, and areas with superficial dendrites are identified using a 20×, 0.95 numerical aperture lens (IR2; Olympus Optical). Spiny dendrites are further identified under digital zoom (7-10×) using two-photon imaging, and spines 50-200 μm below the pial surface are studied. Image acquisition is accomplished using Fluoview software. For motility measurements, Z stacks taken 0.5-1 μM apart are acquired every 5 min for 2 h. For synapse turnover experiments, Z stacks of dendrites and axons are acquired at P40 and then again at P50 or P70. Dendrites and axons located in layers 1-3 are studied. Although both layer 5 and layer 6 neurons are labeled in the mice used in this study, only layer 5 neurons send a clear apical dendrite close to the pial surface thus, the data will come from spines on the apical tuft of layer 5 neurons and axons in superficial cortical layers.

Images are exported to Matlab (Math Works, Natick, Mass.) in which they are processed using custom-written algorithms for image enhancement and alignment of the time series. For motility measurements (see Majewska et al, (2003), Proc Nail Acad Sci USA, 100:16024-16029) spines are analyzed on two-dimensional projections containing between 5 and 30 individual images; therefore, movements in the z dimension are not analyzed. Spine motility is defined as the average change in length per unit time (micrometers per minute). Lengths are measured from the base of the protrusion to its tip. The position of spines are compared on different imaging days. Spines that are farther than 0.5 μm laterally from their previous location are considered to be different spines. Values for stable spines are defined as the percentage of the original spine population present on the second day of imaging. Only areas that show high signal-to-noise ratio in all imaging sessions will be considered for analysis. Analysis is performed blind with respect to animal age and sensory cortical area. Spine motility (e.g., spine turnover), morphology, and density are then compared between control and treatment groups. It is expected that treatment with the PAK inhibitor SU14813 will rescue defective spine morphology relative to that observed in untreated control animals.

### Example 106

### Clinical Trial: Treatment of Autism with a PAK Inhibitor

The following human clinical trial is performed to determine the safety and efficacy of a PAK inhibitor compound of Formula I-XXIII described herein for the treatment of autistic spectrum disorders. The study aims to provide preliminary estimates of effect of administration of a PAK inhibitor (of Formula I-XXIII described herein) in alleviating, inhibiting the progression of, or reducing the severity of at least one behavioral symptom associated autistic spectrum disorders over a three month study period. Clinical observations of global function in language and/or behavior pattern are assessed.

Twenty-four patients, including 20 males and 4 females with an average age of 9 years and meeting DSM-IV criteria for ASD, are treated with a compound of Formula I-XXIII described herein for up to three months. Patients assigned to the Experimental group will receive 1.5 mg twice a day for the first 2 weeks, 3 mg twice a day over the next 2 weeks, 4.5 mg twice a day dose for the next 2 weeks and then 6 mg twice a day for the remaining period so at the time of the 12 weeks behavioral assessments, all patients are on the maximum dose.

The patients are evaluated using a global clinical improvement scale rating for improvement in language and behaviors based on parental observation and clinical appearance. Improvements are rated as follows: moderate to significant, mild to moderate, or no improvement.

After the twenty-four patients are treated for more up to three months with a compound of Formula I-XXIII described herein, parents report improvements in 20 of the 24 patients in one or more categories: attention, motor planning, language function (both receptively and expressively), and self-stimulatory behaviors.

No side effects were reported.

### Example 107

### Clinical Trial: Treatment of Amnestic Autism with a PAK1/PAK3 Inhibitor

This study is designed to determine the effectiveness of a PAK1/PAK3 inhibitor compound of Formula I-XXIII described herein for the treatment of behavioral symptoms of Autistic Disorder in children and adolescents between the ages of 5 and 17. Approximately 100 patients will be participating in this research study. The primary aim of the treatment is to reduce impairing behavioral symptoms such as aggression, explosive outbursts, or self-injurious behavior, without significant side effects. A secondary aim is to evaluate possible improvement in the level of social relatedness, attention, motor coordination, and short-term memory.

Study Type: Interventional

Study Design: Treatment, Randomized, Double-Blind, Placebo Control, Safety/Efficacy Study

### Primary Outcome Measures:

To provide preliminary estimates of dose of a PAK1/PAK3 inhibitor on behavioral symptoms treated with the PAK1/PAK3 inhibitor, and autism patients treated with placebo. Significantly decreased likelihood to experience exacerbation of symptoms of irritability, aggression, agitation, and stereotypy than those randomized to placebo, as measured by the Aberrant Behavior Checklist (ABC), the Ritvo-Freeman Real Life Rating Scale, and the compulsions scale from the Children's Yale-Brown Obsessive Compulsive Scale (CY-BOCS).

### Inclusion Criteria:

Subjects between ages 5 and 17, both males and females. Weight of 15 kg or greater. DSM-IV diagnosis of Autistic Disorder. Medication free for at least 2 weeks for all psychotropic medications (4 weeks for fluoxetine or depot neuroleptics). Anticonvulsants used for treatment of seizure disorder permitted if the dosage has been stable for 4 weeks and patient seizure free for at least 6 months. Clinical Global Impression Severity score of at least 4 and a) 18 or greater on the Irritability Scale of the Aberrant Behavior Checklist or b) 0.5 total score on the Ritvo-Freeman scale. Mental age of at least 18 months. Negative pregnancy test

### Exclusion Criteria:

IQ below 18 months. Females with a positive pregnancy test. Past history of neuroleptic malignant syndrome. DSM-1V diagnosis of Pervasive Developmental Disorder other than Autistic Disorder. Significant medical condition such as heart disease, hypertension, liver or renal failure, pulmonary disease, or unstable seizure disorder. Weight less than 15 kg.

### Experimental Design

Patients are asked to participate for 6 to 8 months. For the first 8 weeks, patients will receive either a compound of Formula I-XXIII described herein or placebo, randomly chosen.

At the end of the 8 weeks, those patients who have improved and were on a compound of Formula I-XXIII described herein will be asked to continue on this medication for another 4 months. The last two months of the study are again double-blind (neither patients nor investigators know treatment). Patients will either continue a compound of Formula I-XXIII described herein treatment or be gradually tapered from said treatment regimen (placebo-substitution). This blinded discontinuation phase will last 2 months during which patients will be closely monitored for recurrence or worsening of symptoms. Patients who have been treated with placebo in the first 8 weeks of the study and have not improved will be treated with a compound of Formula I-XXIII described herein. Weekly visits are required for the first 8 weeks of the study, monthly visits for the following 4 months, and weekly visits during the last 2 months of the study.

The clinical evaluation will show that a compound of Formula I-XXIII described herein will be more effective than placebo in reducing impulsive aggression, agitation, self-injurious behavior, and troublesome repetitive behavior associated with autism.

### Example 108

### Pharmaceutical Compositions

### Example 108a

### Parenteral Composition

To prepare a parenteral pharmaceutical composition suitable for administration by injection, 100 mg of a water-soluble salt of a compound of Formula I-XXIII is dissolved in DMSO and then mixed with 10 mL of 0.9% sterile saline. The mixture is incorporated into a dosage unit form suitable for administration by injection.

### Example 108b

### Oral Composition

To prepare a pharmaceutical composition for oral delivery, 100 mg of a compound of Formula I-XXIII is mixed with 750 mg of starch. The mixture is incorporated into an oral dosage unit for, such as a hard gelatin capsule, which is suitable for oral administration.

### Example 108c

### Sublingual (Hard Lozenge) Composition

To prepare a pharmaceutical composition for buccal delivery, such as a hard lozenge, mix 100 mg of a compound of Formula I-XXIII with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 108d

### Fast-Disintegrating Sublingual Tablet

A fast-disintegrating sublingual tablet is prepared by mixing 48.5% by weigh of a compound of Formula I-XXIII, 44.5% by weight of microcrystalline cellulose (KG-802), 5% by weight of low-substituted hydroxypropyl cellulose (50 μm), and 2% by weight of magnesium stearate. Tablets are prepared by direct compression (AAPS PharmSciTech. 2006; 7(2):E41). The total weight of the compressed tablets is maintained at 150 mg. The formulation is prepared by mixing the amount of compound of Formula I-XXIII with the total quantity of microcrystalline cellulose (MCC) and two-thirds of the quantity of low-substituted hydroxypropyl cellulose (L-HPC) by using a three dimensional manual mixer (Inversina®, Bioengineering AG, Switzerland) for 4.5 minutes. All of the magnesium stearate (MS) and the remaining one-third of the quantity of L-1-IPC are added 30 seconds before the end of mixing.

### Example 108e

### Inhalation Composition

To prepare a pharmaceutical composition for inhalation delivery, 20 mg of a compound of Formula I-XXIII is mixed with 50 mg of anhydrous citric acid and 100 mL of 0.9% sodium chloride solution. The mixture is incorporated into an inhalation delivery unit, such as a nebulizer, which is suitable for inhalation administration.

### Example 108f

### Rectal Gel Composition

To prepare a pharmaceutical composition for rectal delivery, 100 mg of a compound of Formula is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 108 g

### Topical Gel Composition

To prepare a pharmaceutical topical gel composition, 100 mg of a compound of Formula I-XXIII is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topical administration.

### Example 108 h

### Ophthalmic Solution Composition

To prepare a pharmaceutical opthalmic solution composition, 100 mg of a compound of Formula I-XXIII is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Example 108i

### Nasal Spray Solution

To prepare a pharmaceutical nasal spray solution, 10 g of a compound of Formula I-XXIII is mixed with 30 mL of a 0.05M phosphate buffer solution (pH 4.4). The solution is placed in a nasal administrator designed to deliver 100 μl of spray for each application.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating autism comprising administering to an individual in need thereof a therapeutically effective amount of a p21-activated kinase (PAK) inhibitor.

2. The method of claim 1, wherein the PAK inhibitor modulates dendritic spine morphology or synaptic function.

3. The method of claim 2, wherein the PAK inhibitor modulates dendritic spine density.

4. The method of claim 2, wherein the PAK inhibitor modulates dendritic spine length.

5. The method of claim 2, wherein the PAK inhibitor modulates dendritic spine neck diameter.

6. The method of claim 2, wherein the PAK inhibitor modulates dendritic spine head volume.

7. The method of claim 2, wherein the PAK inhibitor modulates dendritic spine head diameter.

8. The method of claim 1, wherein the PAK inhibitor modulates the ratio of the number of mature dendritic spines to the number of immature dendritic spines.

9. The method of claim 1, wherein the PAK inhibitor modulates the ratio of the dendritic spine head diameter to dendritic spine length.

10. The method of claim 1, wherein the PAK inhibitor modulates synaptic function.

11. The method of claim 1, wherein the PAK inhibitor normalizes or partially normalizes aberrant baseline synaptic transmission associated with autism.

12. The method of claim 1, wherein the PAK inhibitor normalizes or partially normalizes aberrant synaptic plasticity associated with autism.

13. The method of claim 1 wherein the PAK inhibitor causes partial inhibition of one or more PAK kinases.

14. The method of claim 1 wherein the PAK kinase inhibitor is a PAK1 inhibitor.

15. The method of claim 1 wherein the PAK kinase inhibitor is a PAK2 inhibitor.

16. The method of claim 1 wherein the PAK kinase inhibitor is a PAK3 inhibitor.

17. The method of claim 1, wherein the treatment comprises administering the PAK inhibitor to an individual with two or more or the following symptoms: (i) insistence on sameness or resistance to change; (ii) difficulty in expressing needs; (iii) repeating words or phrases in place of normal, responsive language; (iv) laughing, crying, showing distress for reasons not apparent to others; (v) prefers to be alone or aloof manner; (vi) tantrums; (vii) difficulty in mixing with others; (viii) may not want to cuddle or be cuddled; (ix) little or no eye contact; (x) unresponsive to normal teaching methods; (xi) sustained odd play; (xii) apparent over-sensitivity or under-sensitivity to pain; (xiii) little or no real fears of danger; (xiv) noticeable physical over-activity or extreme under-activity; (xv) uneven gross/fine motor skills; and/or (xvi) non-responsiveness to verbal cues.

18. The method of claim 1, wherein the treatment comprises administering the PAK inhibitor to an individual with three or more or the following symptoms: (i) insistence on sameness or resistance to change; (ii) difficulty in expressing needs; (iii) repeating words or phrases in place of normal, responsive language; (iv) laughing, crying, showing distress for reasons not apparent to others; (v) prefers to be alone or aloof manner; (vi) tantrums; (vii) difficulty in mixing with others; (viii) may not want to cuddle or be cuddled; (ix) little or no eye contact; (x) unresponsive to normal teaching methods; (xi) sustained odd play; (xii) apparent over-sensitivity or under-sensitivity to pain; (xiii) little or no real fears of danger; (xiv) noticeable physical over-activity or extreme under-activity; (xv) uneven gross/fine motor skills; and/or (xvi) non-responsiveness to verbal cues.

19. The method of claim 1, wherein the treatment alleviates, delays the onset of, inhibits the progression of, or reduces the severity of one or more symptoms associated with Autism Disorder.

20. The method of claim 1, wherein the treatment alleviates, delays the onset of, inhibits the progression of, or reduces the severity of one or more symptoms associated with Asperger's Disorder.

21.-33. (canceled)
